(19) United States
(12) Patent Application Publication
(10) Pub. No.: US 2025/0019380 A1
(43) Pub. Date: Jan. 16, 2025

(54) PI3K-ALPHA INHIBITORS AND METHODS OF USE THEREOF

(71) Applicant: RELAY THERAPEUTICS, INC., Cambridge, MA (US)

(72) Inventors: Alessandro BOEZIO, Cambridge, MA (US); Alexander M. TAYLOR, Cambridge, MA (US); Cary Griffin FRIDRICH, Cambridge, MA (US); Hakan GUNAYDIN, Cambridge, MA (US); Lucian V. DIPIETRO, Cambridge, MA (US); Levi Charles Thomas PIERCE, Cambridge, MA (US); Mary M. MADER, Cambridge, MA (US); Ravi KURUKULASURIYA, Cambridge, MA (US); Thomas H. MCLEAN, Cambridge, MA (US); Yue PAN, Cambridge, MA (US); Michael Paul DENINNO, Gales Ferry, CT (US); Alexandre LARIVEE, Montreal (CA); Andrew j. BURNIE, Montreal (CA); Caleb MEDENA, Montreal (CA); Gaetan MAERTENS, Montreal (CA); Kashif TANVEER, Montreal (CA); Mohan PAL, Monteal (CA); Tarek MOHAMED, Montreal (CA); Thomas LEPITRE, Montreal (CA); Bren-Jordan ATIENZA, Montreal (CA); Naresh VEMULA, Montreal (CA); Shorena GELOZIA, Montreal (CA)

(21) Appl. No.: 18/578,082

(22) PCT Filed: Jul. 13, 2022
(86) PCT No.: PCT/US2022/073672

§ 371 Date: Jan. 10, 2024

Related U.S. Application Data
(60) Provisional application No. 63/203,220, filed on Jul. 13, 2021.

(51) Int. Cl. C07D 487/04; A61K 31/4162; A61K 31/4188; A61K 31/437; A61K 31/4985; A61K 31/499; A61K 31/551; A61K 31/675; C07D 471/04; C07D 487/16; C07D 487/20; C07D 519/00; C07F 9/6561

(52) U.S. Cl. CPC C07D 487/04; A61K 31/4162; A61K 31/4188; A61K 31/437; A61K 31/4985; A61K 31/499; A61K 31/551; A61K 31/675; C07D 471/04; C07D 487/16; C07D 487/20; C07D 519/00; C07F 9/6561

(57) ABSTRACT

The present disclosure relates to novel compounds and pharmaceutical compositions thereof, and methods for inhibiting the activity of PI3Kα enzymes with the compounds and compositions of the disclosure. The present disclosure further relates to, but is not limited to, methods for treating disorders associated with PI3Kα signaling with the compounds and compositions of the disclosure.

### CROSS-REFERENCE TO RELATED APPLICATIONS

This application claims the benefit of U.S. Provisional Application No. 63/203,220 filed on Jul. 13, 2021, the entirety of which is hereby incorporated by reference.

### BACKGROUND

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃), which, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997); Katso et al., Annu. Rev. Cell Dev. Biol. 17:615-675 (2001)).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP₂ and PIP₃ recruit Aid, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)).

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Kα would be of particular value in the treatment of proliferative disease and other disorders. While multiple inhibitors of PI3Ks have been developed (for example, taselisib, alpelisib, buparlisib and others), these molecules inhibit multiple Class 1A PI3K isoforms. Inhibitors that are active against multiple Class 1A PI3K isoforms are known as “pan-PI3K” inhibitors. A major hurdle for the clinical development of existing PI3K inhibitors has been the inability to achieve the required level of target inhibition in tumors while avoiding toxicity in cancer patients. Pan-PI3K inhibitors share certain target-related toxicities including diarrhea, rash, fatigue, and hyperglycemia. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (Hanker et al., Cancer Discovery (2019) PMID: 30837161. Therefore, selective inhibitors of PI3Kα may increase the therapeutic window, enabling sufficient target inhibition in the tumor while avoiding dose-limiting toxicity in cancer patients.

### SUMMARY

In some embodiments, the present disclosure provides a compound of formula I:

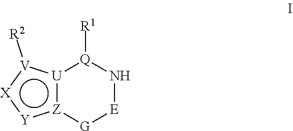

or a pharmaceutically acceptable salt thereof, wherein each of R¹, R², Q, E, G, U, V, X, Y, and Z is as defined in embodiments and classes and subclasses herein.

In some embodiments, the present disclosure provides a pharmaceutical composition comprising a compound of formula I, or a pharmaceutically acceptable salt thereof, and a pharmaceutically acceptable carrier, adjuvant, or diluent.

In some embodiments, the present disclosure provides a method of treating a PI3Kα-mediated disorder comprising administering to a patient in need thereof a compound of formula I, or composition comprising said compound.

In some embodiments, the present disclosure provides a process for providing a compound of formula I, or synthetic intermediates thereof.

In some embodiments, the present disclosure provides a process for providing pharmaceutical compositions comprising compounds of formula I.

### DETAILED DESCRIPTION

### 1. General Description of Certain Embodiments of the Disclosure

Compounds of the present disclosure, and pharmaceutical compositions thereof, are useful as inhibitors of PI3Kα. In some embodiments, the present disclosure provides a compound of formula I:

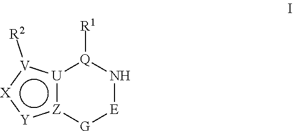

or a pharmaceutically acceptable salt thereof, wherein:
E is —C(O)—, —C(R^{E})₂—, —C(R^{E})₂C(R^{E})₂—, —C(S)—, —S(O)₂—, —OC(O)—, —N(R^{E})C(O)—, —C(O)N(R^{E})—, or —C(R^{E})₂C(O)—;
G is CH₂, CH(R^{G}), C(R^{G})₂, or a covalent bond;
Q is CH, C(R^{Q}), or N;
X is CH, C(R^{X}), or N;
Y is CH, C(R^{Y}), N, or N(R^{Y});
Z is C or N;
U is C or N;
V is C or N; provided that at least one of X, Y, Z, U, and V is N;
R¹ is -L¹-R^{1A};
R² is -L²-R^{2A};
each instance of R^{E} is independently H or -L^{E}-R^{EA};
each instance of R^{G} is independently -L^{G}-R^{GA};
R^{Q} is -L^{Q}-R^{QA};
R^{X} is -L^{X}-R^{XA};
R^{Y} is -L^{Y}-R^{YA}; or
   two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n instances of R^{EEC};
   R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p instances of R^{Q1C};
each of L¹, L², L^{E}, L^{G}, L^{Q}, LX, and L^{Y} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
each instance of R^{EA} is independently R^{A} or R^{B} substituted by r³ instances of R^{EC};
each instance of R^{GA} is independently R^{A} or R^{B} substituted by r⁴ instances of R^{GC}.

R^{QA} is R^{A} or R^{B} substituted by r⁵ instances of R^{QC};
R^{XA} is R^{A} or R^{B} substituted by r⁶ instances of R^{XC};
R^{YA} is R^{A} or R^{B} substituted by r⁷ instances of R^{YC};
R^{L} is R^{A} or R^{B} substituted by r⁸ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{EC}, R^{GC}, R^{QC}, R^{XC}, R^{YC}, R^{LC}, R^{EEC}, and R^{Q1C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, p, r¹, r², r³, r⁴, r⁵, r⁶, r⁷, and r⁸ is independently 0, 1, 2, 3, 4, or 5.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

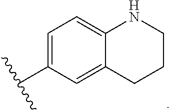 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

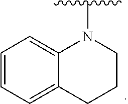 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

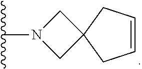 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

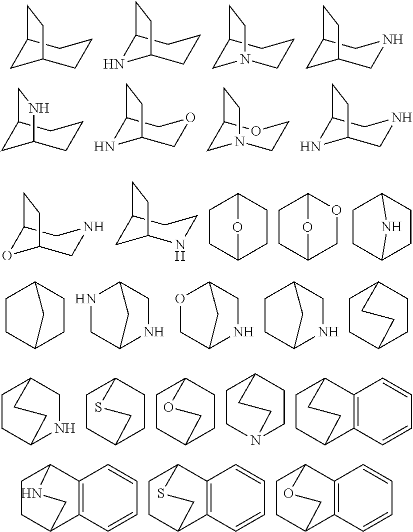

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR⁰²; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure. Such compounds are useful, for example, as analytical tools, as probes in biological assays, or as therapeutic agents in accordance with the present disclosure. In certain embodiments, for example, a warhead moiety, R^{W}, of a provided compound comprises one or more deuterium atoms.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to PI3Kα signaling, or one or more symptoms thereof, prevent the advancement of conditions or symptoms related to afflictions related to PI3Kα signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to PI3Kα signaling activity or that may otherwise be relieved by the compounds and/or compositions of the disclosure.

### 3. Description of Exemplary Embodiments

As described above, in some embodiments, the present disclosure provides a compound of formula I:

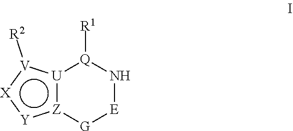

or a pharmaceutically acceptable salt thereof, wherein:
E is —C(O)—, —C(R^{E})₂—, —C(R^{E})₂C(R^{E})₂—, —C(S)—, —S(O)₂—, —OC(O)—, —N(R^{E})C(O)—, —C(O)N(R^{E})—, or —C(R^{E})₂C(O)—;
G is CH₂, CH(R^{G}), C(R^{G})₂, or a covalent bond;
Q is CH, C(R^{Q}), or N;
X is CH, C(R^{X}), or N;
Y is CH, C(R^{Y}), N, or N(R^{Y});
Z is C or N;
U is C or N;
V is C or N; provided that at least one of X, Y, Z, U, and V is N;
R¹ is -L¹-R^{1A};
R² is -L²-R^{2A};
each instance of R^{E} is independently H or -L^{E}-R^{EA};
each instance of R^{G} is independently -L^{G}-R^{GA};
R^{Q} is -L^{Q}-R^{QA};
R^{X} is -LX-R^{XA};
R^{Y} is -LY-R^{YA}; or
   two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n instances of R^{EE}C;
   R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p instances of R^{Q1C};
each of L¹, L², L^{E}, L^{G}, L^{Q}, LX, and L^{Y} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
each instance of R^{EA} is independently R^{A} or R^{B} substituted by r³ instances of R^{EC};
each instance of R^{GA} is independently R^{A} or R^{B} substituted by r⁴ instances of R^{GC}.
R^{QA} is R^{A} or R^{B} substituted by r⁵ instances of R^{QC};
R^{XA} is R^{A} or R^{B} substituted by r⁶ instances of R^{XC};
R^{YA} is R^{A} or R^{B} substituted by r⁷ instances of R^{YC};
R^{L} is R^{A} or R^{B} substituted by r⁸ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{EC}, R^{GC}, R^{QC}, R^{XC}, R^{YC}, R^{LC}, R^{EEC}, and R^{Q1C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, p, r¹, r², r³, r⁴, r⁵, r⁶, r⁷, and r⁸ is independently 0, 1, 2, 3, 4, or 5.

As defined generally above, E is —C(O)—, —C(R^{E})₂—, —C(R^{E})₂C(R^{E})₂—, C₃₋₆ cycloalkylene, C₃₋₆ heterocycloalkylene, —C(S)—, —S(O)₂—, —OC(O)—, —N(R^{E})C(O)—, —C(O)N(R^{E})—, or —C(R^{E})₂C(O)—. In some embodiments, E is —C(O)—. In some embodiments, E is —OC(O)— or —N(R^{E})C(O)—. In some embodiments, E is —C(R^{E})₂—, C₃₋₆ cycloalkylene, or C₃₋₆ heterocycloalkylene.

In some embodiments, E is —C(O)—, —OC(O)—, —N(R^{E})C(O)—, or —C(R^{E})₂C(O)—. In some embodiments, E is —OC(O)—, —N(R^{E})C(O)—, or —C(R^{E})₂C(O)—. In some embodiments, E is —C(O)— or —N(R^{E})C(O)—.

In some embodiments, E is —C(O)—, —C(R^{E})₂—, —C(S)—, or —S(O)₂—. In some embodiments, E is —C(O)—, —C(R^{E})₂—, or —C(S)—. In some embodiments, E is —C(O)— or —C(S)—.

In some embodiments, E is —C(R^{E})₂C(R^{E})₂—, C₃₋₆ cycloalkylene, C₃₋₆ heterocycloalkylene, —OC(O)—, —N(R^{E})C(O)—, —C(O)N(R^{E})—, or —C(R^{E})₂C(O)—. In some embodiments, E is C₃₋₆ cycloalkylene or C₃₋₆ heterocycloalkylene. In some embodiments, E is —C(R^{E})₂C(R^{E})₂—, —OC(O)—, —N(R^{E})C(O)—, —C(O)N(R^{E})—, or —C(R^{E})₂C(O)—. In some embodiments, E is —OC(O)—, —N(R^{E})C(O)—, —C(O)N(R^{E})—, or —C(R^{E})₂C(O)—. In some embodiments, E is —OC(O)—, —N(R^{E})C(O)—, or —C(O)N(R^{E})—. In some embodiments, E is —N(R^{E})C(O)— or —C(O)N(R^{E})—. In some embodiments, E is —N(H)C(O)— or —C(O)N(H)—. In some embodiments, E is —N(CH₃)C(O)— or —C(O)N(CH₃)—.

In some embodiments, E is —S(O)₂—, —OC(O)—, —N(R^{E})C(O)—, or —C(O)N(R^{E})—. In some embodiments, E is —C(O)—, —C(R^{E})₂—, —C(R^{E})₂C(R^{E})₂—, C₃₋₆ cycloalkylene, C₃₋₆ heterocycloalkylene, —C(S)—, or —C(R^{E})₂C(O)—. In some embodiments, E is —C(O)—, —C(R^{E})₂—, —C(R^{E})₂C(R^{E})₂—, —C(S)—, or —C(R^{E})₂C(O)—. In some embodiments, E is —C(O)—, —C(S)—, or —C(R^{E})₂C(O)—. In some embodiments, E is —C(R^{E})₂—, —C(R^{E})₂C(R^{E})₂—, or —C(R^{E})₂C(O)—. In some embodiments, E is —C(R^{E})₂— or —C(R^{E})₂C(R^{E})₂—.

In some embodiments, E is —C(R^{E})₂—. In some embodiments, E is —C(R^{E})₂C(R^{E})₂—. In some embodiments, E is C₃₋₆ cycloalkylene. In some embodiments, E is C₃₋₆ heterocycloalkylene. In some embodiments, E is —C(S)—. In some embodiments, E is —S(O)₂—. In some embodiments, E is —OC(O)—. In some embodiments, E is —N(R^{E})C(O)—. In some embodiments, E is —N(H)C(O)—. In some embodiments, E is —N(CH₃)C(O)—. In some embodiments, E is —C(O)N(R^{E})—. In some embodiments, E is —C(O)N(H)—. In some embodiments, E is —C(O)N(CH₃)—. In some embodiments, E is —C(R^{E})₂C(O)—.

In some embodiments, E is selected from the groups depicted in the compounds in Table 1.

As defined generally above, G is CH₂, CH(R^{G}), C(R^{G})₂, or a covalent bond. In some embodiments, G is CH₂, CH(R^{G}), or C(R^{G})₂. In some embodiments, G is CH₂ or CH(R^{G}). In some embodiments, G is CH(R^{G}) or C(R^{G})₂. In some embodiments, G is CH₂. In some embodiments, G is CH(R^{G}). In some embodiments, G is C(R^{G})₂. In some embodiments, G is a covalent bond. In some embodiments, G is selected from the groups depicted in the compounds in Table 1.

As defined generally above, Q is CH, C(R^{Q}), or N. In some embodiments, Q is CH. In some embodiments, Q is C(R^{Q}). In some embodiments, Q is N. In some embodiments, Q is CH or C(R^{Q}). In some embodiments, Q is CH or N. In some embodiments, Q is C(R^{Q}) or N. In some embodiments, Q is selected from the groups depicted in the compounds in Table 1.

As defined generally above, X is CH, C(R^{X}), or N; provided that at least one of X, Y, Z, U, and V is N. In some embodiments, X is CH. In some embodiments, X is C(R^{X}). In some embodiments, X is N. In some embodiments, X is CH or C(R^{X}). In some embodiments, X is CH or N. In some embodiments, X is C(R^{X}) or N. In some embodiments, X is selected from the groups depicted in the compounds in Table 1.

As defined generally above, Y is CH, C(R^{Y}), N, or N(R^{Y}); provided that at least one of X, Y, Z, U, and V is N. In some embodiments, Y is CH. In some embodiments, Y is C(R^{Y}). In some embodiments, Y is N. In some embodiments, Y is N(R^{Y}). In some embodiments, Y is CH or C(R^{Y}). In some embodiments, Y is CH or N. In some embodiments, Y is C(R^{Y}) or N. In some embodiments, Y is C(R^{Y}) or N(R^{Y}). In some embodiments, Y is N or N(R^{Y}). In some embodiments, Y is selected from the groups depicted in the compounds in Table 1.

As defined generally above, Z is C or N; provided that at least one of X, Y, Z, U, and V is N. In some embodiments, Z is C. In some embodiments, Z is N. In some embodiments, Z is selected from the groups depicted in the compounds in Table 1.

As defined generally above, U is C or N; provided that at least one of X, Y, Z, U, and V is N. In some embodiments, U is C. In some embodiments, U is N. In some embodiments, U is selected from the groups depicted in the compounds in Table 1.

As defined generally above, V is C or N; provided that at least one of X, Y, Z, U, and V is N. In some embodiments, V is C. In some embodiments, V is N. In some embodiments, V is selected from the groups depicted in the compounds in Table 1.

As defined generally above, R¹ is -L¹-R^{1A} or R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p instances of R^{Q1C}. In some embodiments, R¹ is -L¹-R^{1A}. In some embodiments, R¹ is —R^{1A}.

In some embodiments, R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p instances of R^{Q1C}. In some embodiments, R^{Q} and R¹ are taken together with their intervening atoms to form a 4-8 membered saturated or partially unsaturated monocyclic ring having 0-4 heteroatoms independently selected from nitrogen, oxygen, and sulfur; wherein said ring is substituted with p instances of R^{Q1C}.

In some embodiments, R^{Q} and R¹ are taken together with their intervening atoms to form an 8-12 membered saturated or partially unsaturated bicyclic ring having 0-4 heteroatoms independently selected from nitrogen, oxygen, and sulfur; wherein said ring is substituted with p instances of R^{Q1C}. In some embodiments, R^{Q} and R¹ are taken together with their intervening atoms to form a 9- or 10-membered partially unsaturated bicyclic ring having 0, 1, or 2 heteroatoms independently selected from nitrogen, oxygen, and sulfur; wherein said ring is substituted with p instances of R^{Q1C}. In some embodiments, R^{Q} and R¹ are taken together with their intervening atoms to form an indolin-2-one ring; wherein said ring is substituted with 0, 1, 2, or 3 instances of R^{Q1C}. In some embodiments, R^{Q} and R¹ are taken together with their intervening atoms to form an indolin-2-one ring; wherein the aromatic ring is substituted with 0, 1, 2, or 3 instances of R^{Q1C}.

In some embodiments, R¹ (i.e. -L¹-R^{1A} taken together) is

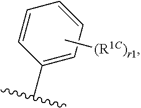

wherein R^{1C} and r¹ are as defined in the embodiments and classes and subclasses herein. In some embodiments, R¹ (i.e. -L¹-R^{1A} taken together) is

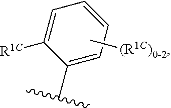

wherein R^{1C} is as defined in the embodiments and classes and subclasses herein. In some embodiments, R¹ (i.e. -L¹-R^{1A} taken together) is

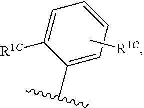

wherein R^{1C} is as defined in the embodiments and classes and subclasses herein. In some embodiments, R¹ (i.e. -L¹-R^{1A} taken together) is

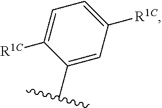

wherein R^{1C} is as defined in the embodiments and classes and subclasses herein. In some embodiments, R¹ (i.e. -L¹-R^{1A} taken together) is

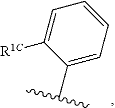

wherein R^{1C} is as defined in the embodiments and classes and subclasses herein.

In some embodiments, R¹ (i.e. -L¹-R^{1A} taken together) is

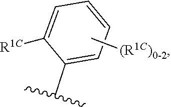

wherein each instance of R^{1C} is independently halogen, —CN, —O-(optionally substituted C₁₋₆ aliphatic), or an optionally substituted C₁₋₆ aliphatic. In some embodiments, R¹ (i.e. -L¹-R^{1A} taken together) is

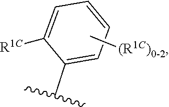

wherein each instance of R^{1C} is independently halogen or C₁₋₃ aliphatic optionally substituted with 1-3 halogen. In some embodiments, R¹ (i.e. -L¹-R^{1A} taken together) is

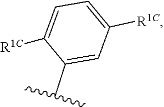

wherein each instance of R^{1C} is independently halogen or C₁₋₃ aliphatic optionally substituted with 1-3 halogen. In some embodiments, R¹ (i.e. -L¹-R^{1A} taken together) is

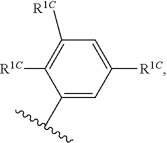

wherein each instance of R^{1C} is independently halogen or C₁₋₃ aliphatic optionally substituted with 1-3 halogen. In some embodiments, R¹ (i.e. -L¹-R^{1A} taken together) is

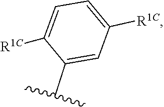

wherein each instance of R^{1C} is independently fluorine, chlorine, —CH₃, —CHF₂, or —CF₃. In some embodiments, R¹ (i.e. -L¹-R^{1A} taken together) is

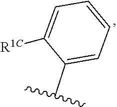

wherein R^{1C} is halogen or C₁₋₃ aliphatic optionally substituted with 1-3 halogen.

In some embodiments, R¹ (i.e. -L¹-R^{1A} taken together) is

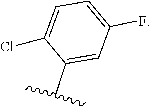 [SMILES: *c1cc(F)ccc1Cl]

In some embodiments, R¹ (i.e. -L¹-R^{1A} taken together) is

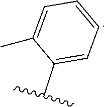 [SMILES: *c1ccccc1C]

In some embodiments, R¹ (i.e. -L¹-R^{1A} taken together) is

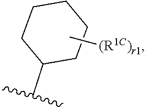

wherein R^{1C} and r¹ are as defined in the embodiments and classes and subclasses herein. In some embodiments, R¹ (i.e. -L¹-R^{1A} taken together) is

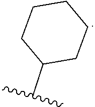 [SMILES: *C1CCCCC1]

In some embodiments, R¹ (i.e. -L¹-R^{1A} taken together) is

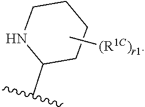

In some embodiments, R¹ (i.e. -L¹-R^{1A} taken together) is

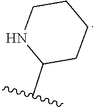 [SMILES: *C1CCCCN1]

In some embodiments, R¹ is selected from the groups depicted in the compounds in Table 1.

As defined generally above, R² is -L²-R^{2A}. In some embodiments, R² (i.e. -L²-R^{2A} taken together) is —N(R)C(O)—R^{2A} or —R^{2A}, wherein R and R^{2A} are as defined in the embodiments and classes and subclasses herein. In some embodiments, R² (i.e. -L²-R^{2A} taken together) is —N(R)C(O)—R^{2A}, wherein R and R^{2A} are as defined in the embodiments and classes and subclasses herein. In some embodiments, R² (i.e. -L²-R^{2A} taken together) is —N(H)C(O)—R^{2A}, wherein R^{2A} is as defined in the embodiments and classes and subclasses herein. In some embodiments, R² (i.e. -L²-R^{2A} taken together) is —N(H)C(O)—R^{2A}, wherein R^{2A} is R substituted by r² instances of R^{2C}. In some embodiments, R² is -R^{2A}.

In some embodiments, R² is —N(H)C(O)—R^{2A}, —N(H)C(O)N(H)—R^{2A}, —C(O)N(H)—R^{2A}, —N(H)—R^{2A}, —S(O)₂CH₂—R^{2A}, —CH₂S(O)₂—R^{2A}, or —C(H)(CH₃)OH. In some embodiments, R² is —N(H)C(O)—R^{2A}, —N(H)C(O)N(H)—R^{2A}, or —N(H)—R^{2A}. In some embodiments, R² is —C(O)N(H)—R^{2A}, —CH₂S(O)₂—R^{2A}, or —C(H)(CH₃)OH. In some embodiments, R² is —S(O)₂CH₂—R^{2A} or —CH₂S(O)₂—R^{2A}.

In some embodiments, R² is —N(H)C(O)N(H)—R^{2A}. In some embodiments, R² is —C(O)N(H)—R^{2A}. In some embodiments, R² is —N(H)—R^{2A}. In some embodiments, R² is —S(O)₂CH₂—R^{2A}. In some embodiments, R² is —CH₂S(O)₂—R^{2A}. In some embodiments, R² is —C(H)(CH₃)OH.

In some embodiments, R² (i.e. -L²-R^{2A} taken together) is

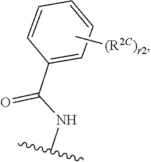

wherein R^{2C} and r² are as defined in the embodiments and classes and subclasses herein. In some embodiments, R² (i.e. -L²-R^{2A} taken together) is

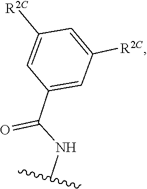

wherein R^{2C} is as defined in the embodiments and classes and subclasses herein.

In some embodiments, R² (i.e. -L²-R^{2A} taken together) is

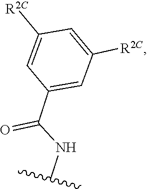

wherein each instance of R^{2C} is independently halogen, —CN, —O-(optionally substituted C₁₋₆ aliphatic), or an optionally substituted C₁₋₆ aliphatic. In some embodiments, R² (i.e. -L²-R^{2A} taken together) is

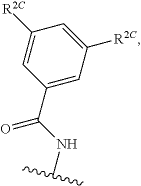

wherein each instance of R^{2C} is independently halogen or C₁₋₃ aliphatic optionally substituted with 1-3 halogen. In some embodiments, R² (i.e. -L²-R^{2A} taken together) is

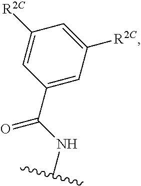

wherein each instance of R^{2C} is independently fluorine, chlorine, —CH₃, —CHF₂, or —CF₃. In some embodiments, R² (i.e. -L²-R^{2A} taken together) is

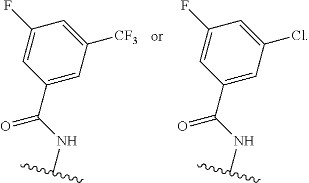 [SMILES: *NC(=O)c1cc(F)cc(C(F)(F)F)c1 ; *NC(=O)c1cc(F)cc(Cl)c1]

In some embodiments, R² (i.e. -L²-R^{2A} taken together) is

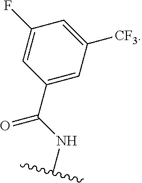 [SMILES: *NC(=O)c1cc(F)cc(C(F)(F)F)c1]

In some embodiments, R² (i.e. -L²-R^{2A} taken together) is

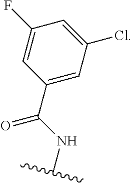 [SMILES: *NC(=O)c1cc(F)cc(Cl)c1]

In some embodiments, R² (i.e. -L²-R^{2A} taken together) is

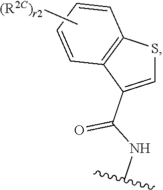

wherein R^{2C} and r² are as defined in the embodiments and classes and subclasses herein. In some embodiments, R² (i.e. -L²-R^{2A} taken together) is

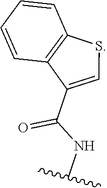 [SMILES: *NC(=O)c1csc2ccccc12]

In some embodiments, R² (i.e. -L²-R^{2A} taken together) is

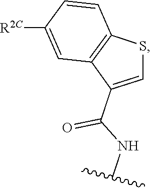

wherein R^{2C} is as defined in the embodiments and classes and subclasses herein.

In some embodiments, R² (i.e. -L²-R^{2A} taken together) is

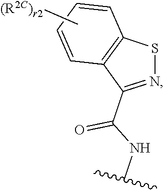

wherein R^{2C} and r² are as defined in the embodiments and classes and subclasses herein. In some embodiments, R² (i.e. -L²-R^{2A} taken together) is

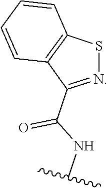 [SMILES: *NC(=O)c1nsc2ccccc12]

In some embodiments, R² (i.e. -L²-R^{2A} taken together) is

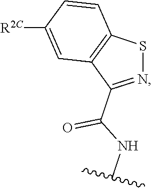

wherein R^{2C} is as defined in the embodiments and classes and subclasses herein.

In some embodiments, R² (i.e. -L²-R^{2A} taken together) is

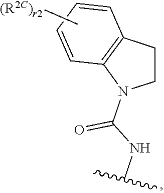

wherein R^{2C} and r² are as defined in the embodiments and classes and subclasses herein. In some embodiments, R² (i.e. -L²-R^{2A} taken together) is

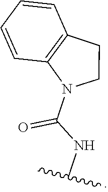 [SMILES: *NC(=O)N1CCc2ccccc21]

In some embodiments, R² (i.e. -L²-R^{2A} taken together) is

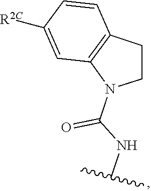

wherein R^{2C} is as defined in the embodiments and classes and subclasses herein.

In some embodiments, R² (i.e. -L²-R^{2A} taken together) is

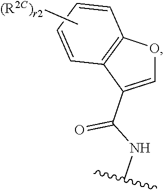

wherein R^{2C} and r² are as defined in the embodiments and classes and subclasses herein. In some embodiments, R² (i.e. -L²-R^{2A} taken together) is

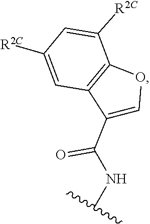

wherein R^{2C} is as defined in the embodiments and classes and subclasses herein.

In some embodiments, R² (i.e. -L²-R^{2A} taken together) is

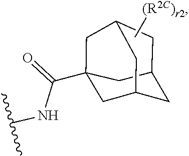

wherein R^{2C} and r² are as defined in the embodiments and classes and subclasses herein. In some embodiments, R² (i.e. -L²-R^{2A} taken together) is

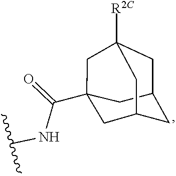

wherein R^{2C} is as defined in the embodiments and classes and subclasses herein.

In some embodiments, R² (i.e. -L²-R^{2A} taken together) is

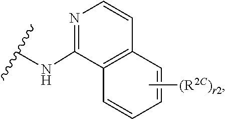

wherein R^{2C} and r² are as defined in the embodiments and classes and subclasses herein. In some embodiments, R² (i.e. -L²-R^{2A} taken together) is

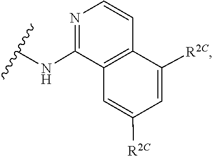

wherein R^{2C} is as defined in the embodiments and classes and subclasses herein.

In some embodiments, R² (i.e. -L²-R^{2A} taken together) is

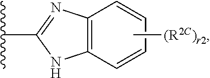

wherein R^{2C} and r² are as defined in the embodiments and classes and subclasses herein. In some embodiments, R² (i.e. -L²-R^{2A} taken together) is

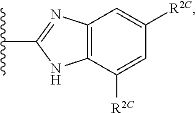

wherein R^{2C} is as defined in the embodiments and classes and subclasses herein.

In some embodiments, R² is

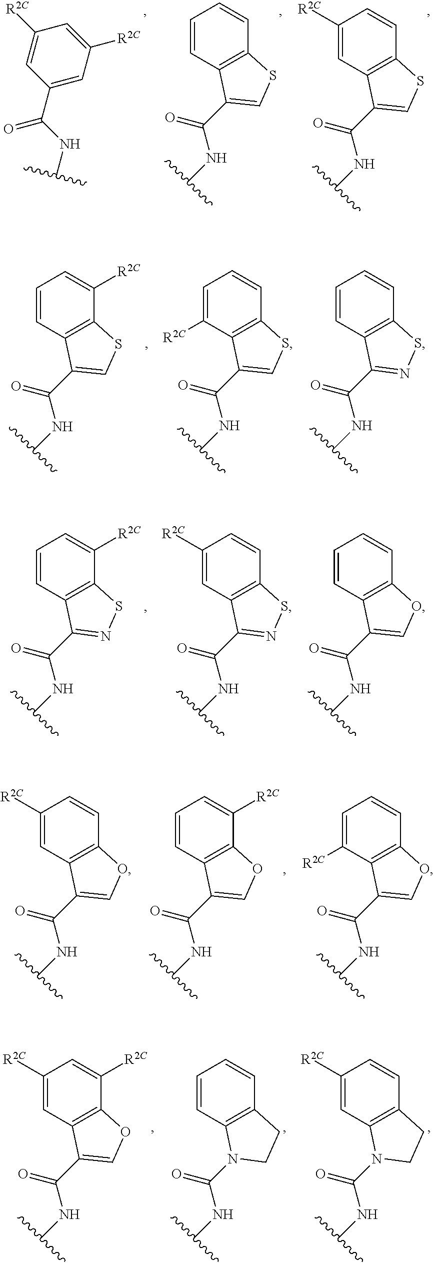

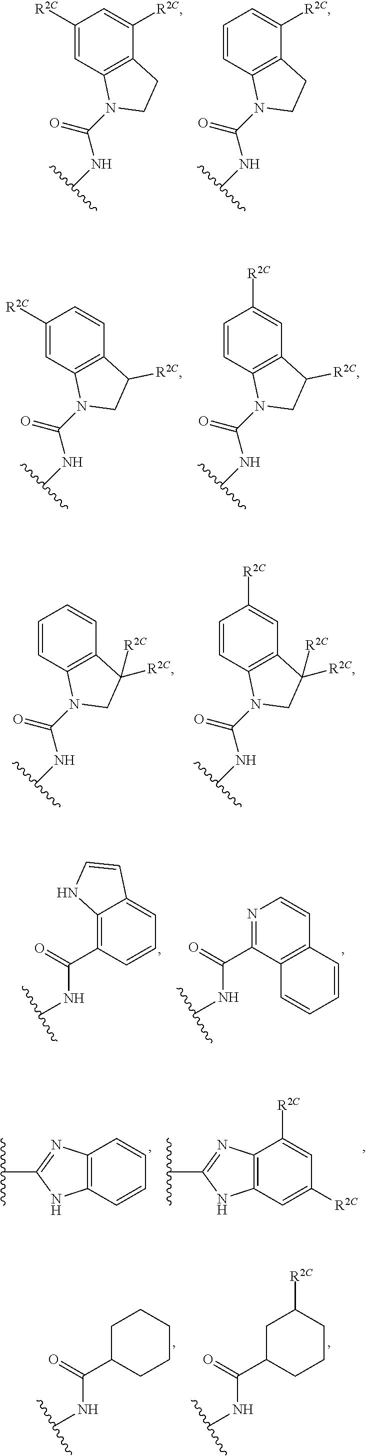

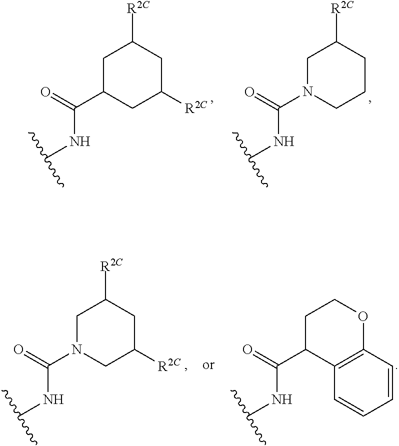

In some embodiments, R² is

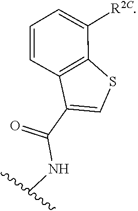

In some embodiments, R² is

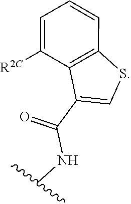

In some embodiments, R² is

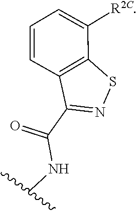

In some embodiments, R² is

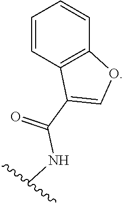 [SMILES: *NC(=O)c1coc2ccccc12]

In some embodiments, R² is

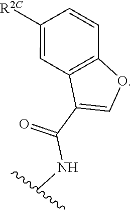

In some embodiments, R² is

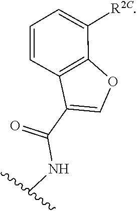

In some embodiments, R² is

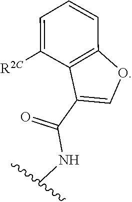

In some embodiments, R² is

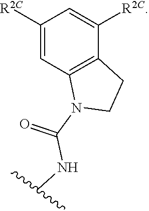

In some embodiments, R² is

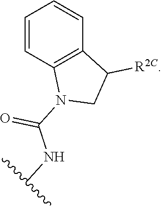

In some embodiments, R² is

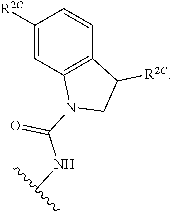

In some embodiments, R² is

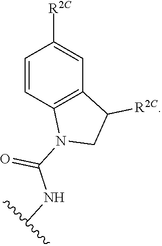

In some embodiments, R² is

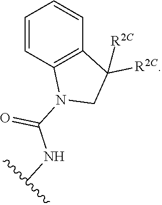

In some embodiments, R² is

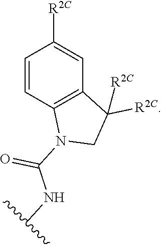

In some embodiments, R² is

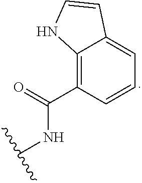 [SMILES: *NC(=O)c1cccc2cc[nH]c12]

In some embodiments, R² is

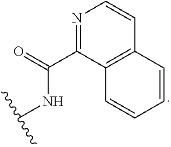 [SMILES: *NC(=O)c1nccc2ccccc12]

In some embodiments, R² is

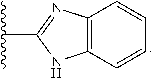 [SMILES: *c1nc2ccccc2[nH]1]

In some embodiments, R² is

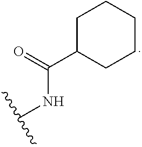 [SMILES: *NC(=O)C1CCCCC1]

In some embodiments, R² is

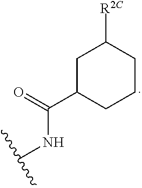

In some embodiments, R² is

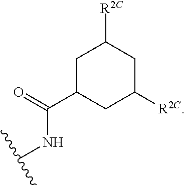

In some embodiments, R² is

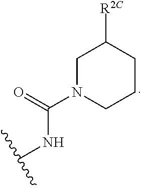

In some embodiments, R² is

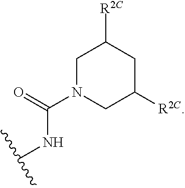

In some embodiments, R² is

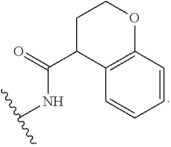 [SMILES: *NC(=O)C1CCOc2ccccc21]

In some embodiments, R² is selected from the groups depicted in the compounds in Table 1.

As defined generally above, each instance of R^{E} is independently H or -L^{E}-R^{EA}; or two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n instances of R^{EEC}.

In some embodiments, each instance of R^{E} is independently H or -L^{E}-R^{EA}. In some embodiments, R^{E} is H. In some embodiments, each instance of R^{E} is independently -L^{E}-R^{EA}In some embodiments, each instance of R^{E} is independently R^{EA}. In some embodiments, each instance of R^{E} is independently R^{A}. In some embodiments, each instance of R^{E} is independently R substituted by r³ instances of R^{EC}.

In some embodiments, each instance of R^{E} is independently H or C₁₋₆ aliphatic substituted by r³ instances of R^{EC}. In some embodiments, each instance of R^{E} is independently H or C₁₋₃ aliphatic substituted by r³ instances of R^{EC}. In some embodiments, each instance of R^{E} is independently H or C₁₋₃ aliphatic substituted by r³ instances of halogen. In some embodiments, each instance of R^{E} is independently H or C₁₋₃ aliphatic. In some embodiments, each instance of R^{E} is independently H, —CH₃, —CH₂F, —CHF₂—, or —CF₃. In some embodiments, each instance of R^{E} is independently H or —CH₃.

In some embodiments, each instance of R^{E} is independently C₁₋₆ aliphatic substituted by r³ instances of R^{EC}. In some embodiments, each instance of R^{E} is independently C₁₋₃ aliphatic substituted by r³ instances of R^{EC}. In some embodiments, each instance of R^{E} is independently C₁₋₃ aliphatic substituted by r³ instances of halogen. In some embodiments, each instance of R^{E} is independently C₁₋₃ aliphatic. In some embodiments, each instance of R^{E} is independently —CH₃, —CH₂F, —CHF₂—, or —CF₃. In some embodiments, R^{E} is —CH₃.

In some embodiments, two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n instances of R^{EEC}In some embodiments, two instances of R^{E} are taken together with their intervening atoms to form a 3-8 membered saturated or partially unsaturated monocyclic ring having 0-4 heteroatoms independently selected from nitrogen, oxygen, and sulfur; wherein said ring is substituted with n instances of R^{EEC}. In some embodiments, two instances of R^{E} are taken together with their intervening atoms to form an 8-12 membered saturated or partially unsaturated bicyclic ring having 0-4 heteroatoms independently selected from nitrogen, oxygen, and sulfur; wherein said ring is substituted with n instances of R^{EEC}.

In some embodiments, R^{E} is selected from the groups depicted in the compounds in Table 1.

As defined generally above, each instance of R^{G} is independently -L^{G}-R^{GA}. In some embodiments, each instance of R^{G} is independently —R^{GA}. In some embodiments, each instance of R^{G} is independently —CH₂—R^{GA}. In some embodiments, each instance of R^{G} is independently C₁₋₆ aliphatic. In some embodiments, each instance of R^{G} is independently C₁ 3 aliphatic. In some embodiments, R^{G} is —CH₃. In some embodiments, R^{G} is selected from the groups depicted in the compounds in Table 1.

As defined generally above, R^{Q} is -L^{Q}-R^{QA} or R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein said ring is substituted with p instances of R^{Q1C}. In some embodiments, R^{Q} is -L^{Q}-R^{QA}. In some embodiments, R^{Q} is —R^{QA}.

In some embodiments, R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein said ring is substituted with p instances of R^{Q1C}In some embodiments, R^{Q} and R¹ are taken together with their intervening atoms to form a 4-8 membered saturated or partially unsaturated monocyclic ring having 0-4 heteroatoms independently selected from nitrogen, oxygen, and sulfur; wherein said ring is substituted with p instances of R^{Q1C}.

In some embodiments, R^{Q} and R¹ are taken together with their intervening atoms to form an 8-12 membered saturated or partially unsaturated bicyclic ring having 0-4 heteroatoms independently selected from nitrogen, oxygen, and sulfur; wherein said ring is substituted with p instances of R^{Q1C}. In some embodiments, R^{Q} and R¹ are taken together with their intervening atoms to form a 9- or 10-membered partially unsaturated bicyclic ring having 0, 1, or 2 heteroatoms independently selected from nitrogen, oxygen, and sulfur; wherein said ring is substituted with p instances of R^{Q1C}. In some embodiments, R^{Q} and R¹ are taken together with their intervening atoms to form an indolin-2-one ring; wherein said ring is substituted with 0, 1, 2, or 3 instances of R^{Q1C}. In some embodiments, R^{Q} and R¹ are taken together with their intervening atoms to form an indolin-2-one ring; wherein the aromatic ring is substituted with 0, 1, 2, or 3 instances of R^{Q1C}.

In some embodiments, R^{Q} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{Q} is halogen, —CN, —OH, —O-(optionally substituted C₁₋₆ aliphatic), or an optionally substituted C₁₋₆ aliphatic. In some embodiments, R^{Q} is halogen, —OH, or C₁₋₃ aliphatic optionally substituted with 1-3 halogen. In some embodiments, R^{Q} is fluorine, chlorine, —OH, or —CH₃. In some embodiments, R^{Q} is deuterium. In some embodiments, R^{Q} is selected from the groups depicted in the compounds in Table 1.

As defined generally above, R^{X} is -LX-R^{XA}. In some embodiments, R^{X} is —R^{XA}. In some embodiments, R^{X} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{X} is halogen, —CN, —OH, —O-(optionally substituted C₁₋₆ aliphatic), or an optionally substituted C₁₋₆ aliphatic. In some embodiments, R^{X} is halogen, —OH, —O—(C₁₋₃ aliphatic), or C₁₋₃ aliphatic, wherein each C₁₋₃ aliphatic is optionally substituted with 1-3 halogen. In some embodiments, R^{X} is fluorine, chlorine, —OCH₃, or —CH₃. In some embodiments, R^{X} is selected from the groups depicted in the compounds in Table 1.

As defined generally above, R^{Y} is -L^{Y}-R^{YA}. In some embodiments, R^{Y} is —C(O)N(R)—R^{YA}, —C(O)N(R)CH₂—R^{YA}, or —R^{YA}. In some embodiments, R^{Y} is —C(O)N(H)—R^{YA}, —C(O)N(H)CH₂—R^{YA}, or —R^{YA}. In some embodiments, R^{Y} is —C(O)N(H)—R^{YA} or —C(O)N(H)CH₂—R^{YA}. In some embodiments, R^{Y} is —C(O)N(H)—R^{YA}. In some embodiments, R^{Y} is —C(O)N(H)CH₂—R^{YA}. In some embodiments, R^{Y} is —R^{YA}.

In some embodiments, R^{Y} is

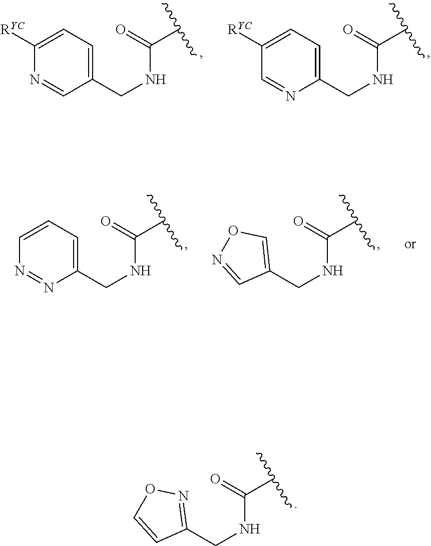

In some embodiments, R^{Y} is

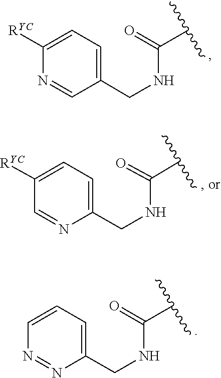

In some embodiments, R^{Y} is

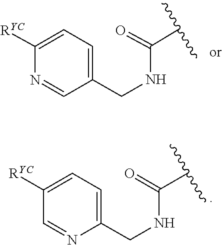

In some embodiments, R^{Y} is

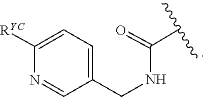

In some embodiments, R^{Y} is

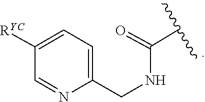

In some embodiments, R^{Y} is

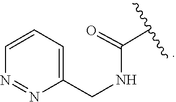 [SMILES: *C(=O)NCc1cccnn1]

In some embodiments, R^{Y} is

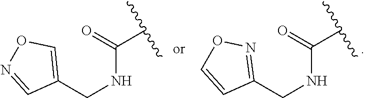 [SMILES: *C(=O)NCc1cnoc1 ; *C(=O)NCc1ccon1]

In some embodiments, R^{Y} is

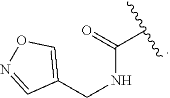 [SMILES: *C(=O)NCc1cnoc1]

In some embodiments, R^{Y} is

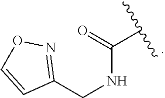 [SMILES: *C(=O)NCc1ccon1]

In some embodiments, R^{Y} is

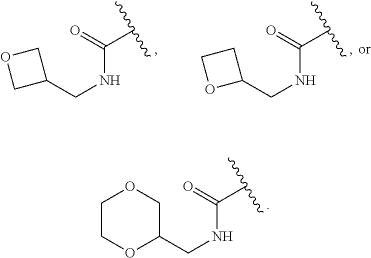 [SMILES: *C(=O)NCC1COC1 ; *C(=O)NCC1CCO1 ; *C(=O)NCC1COCCO1]

In some embodiments, R^{Y} is

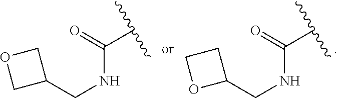 [SMILES: *C(=O)NCC1COC1 ; *C(=O)NCC1CCO1]

In some embodiments, R^{Y} is

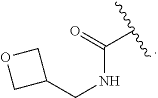 [SMILES: *C(=O)NCC1COC1]

In some embodiments, R^{Y} is

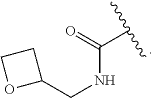 [SMILES: *C(=O)NCC1CCO1]

In some embodiments, R^{Y} is

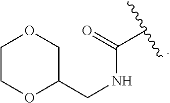 [SMILES: *C(=O)NCC1COCCO1]

In some embodiments, R^{Y} is —C(O)N(H)—(C₁₋₆ aliphatic), wherein said C₁₋₆ aliphatic is substituted by r⁷ instances of R^{YC}. In some embodiments, R^{Y} is —C(O)N(H)—(C₁₋₆ aliphatic chain), wherein said C₁₋₆ aliphatic chain is substituted by r⁷ instances of R^{YC}. In some embodiments, R is —C(O)N(H)—(C₁₋₆ aliphatic), wherein said C₁₋₆ aliphatic is optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, R^{Y} is —C(O)N(H)—(C₁₋₆ aliphatic), wherein said C₁₋₆ aliphatic is substituted with 1 or 2 groups independently selected from —O—(C₁₋₆ aliphatic), —OH, —N(C₁₋₆ aliphatic)₂, and —CN; and said C₁₋₆ aliphatic is optionally substituted with 1, 2, or 3 halogen atoms. In some embodiments, R^{Y} is —C(O)N(H)—(C₁₋₆ aliphatic), wherein said C₁₋₆ aliphatic is substituted with one —OH and 1, 2, or 3 halogen atoms. In some embodiments, R^{Y} is

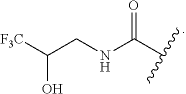 [SMILES: *C(=O)NCC(O)C(F)(F)F]

In some embodiments, R^{Y} is —C(O)N(H)—(C₁₋₆ aliphatic), wherein said C₁₋₆ aliphatic is substituted with one —CN. In some embodiments, R^{Y} is —C(O)N(H)—(CH₂)₂CN. In some embodiments, R^{Y} is —C(O)N(H)—(C₁₋₆ aliphatic), wherein said C₁₋₆ aliphatic is substituted with 1, 2, or 3 halogen atoms. In some embodiments, R^{Y} is —C(O)N(H)—CH₂CHF₂. In some embodiments, R^{Y} is —C(O)N(H)—(C₁₋₆ aliphatic), wherein said C₁₋₆ aliphatic is substituted with 1, 2, or 3 deuterium atoms. In some embodiments, R^{Y} is —C(O)N(H)—(C₁₋₆ aliphatic). In some embodiments, R^{Y} is —C(O)N(H)—(C₁₋₄ alkyl). In some embodiments, R is —C(O)N(H)CH₃. In some embodiments, R^{Y} is —C(O)N(H)CD₃.

In some embodiments, R^{Y} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, R is halogen, —CN, —S(O)₂R, —S(O)₂NR₂, —S(O)₂F, —S(O)R, —S(O)NR₂, —S(O)(NR)R, —C(O)R, —C(O)OR, —C(O)NR₂, or —C(O)N(R)OR. In some embodiments, R^{Y} is halogen or —CN. In some embodiments, R^{Y} is selected from the groups depicted in the compounds in Table 1.

As defined generally above, L¹ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a covalent bond. In some embodiments, L¹ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₄ bivalent saturated or unsaturated, straight or branched hydrocarbon chain.

In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L¹ is a C₁₋₂ bivalent saturated or unsaturated hydrocarbon chain. In some embodiments, L¹ is selected from the groups depicted in the compounds in Table 1.

As defined generally above, L² is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₄ bivalent saturated or unsaturated, straight or branched hydrocarbon chain.

In some embodiments, L² is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L² is a C₁₋₂ bivalent saturated or unsaturated hydrocarbon chain.

In some embodiments, L² is —N(R)C(O)— or —N(R)C(O)N(R)—. In some embodiments, L² is —N(H)C(O)— or —N(H)C(O)N(H)—. In some embodiments, L² is —N(R)C(O)—. In some embodiments, L² is —N(H)C(O)—. In some embodiments, L² is —N(R)C(O)N(R)—. In some embodiments, L² is —N(H)C(O)N(H)—. In some embodiments, L² is —N(R)—. In some embodiments, L² is —N(H)—. In some embodiments, L² is a covalent bond. In some embodiments, L² is selected from the groups depicted in the compounds in Table 1.

As defined generally above, L^{E} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E} is a covalent bond. In some embodiments, L^{E} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E} is a C₁₋₄ bivalent saturated or unsaturated, straight or branched hydrocarbon chain.

In some embodiments, L^{E} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{E} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{E} is a C₁₋₂ bivalent saturated or unsaturated hydrocarbon chain. In some embodiments, L^{E} IS Selected from the groups depicted in the compounds in Table 1.

As defined generally above, L^{G} is a covalent bond, or a C₁-4 bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G} is a covalent bond. In some embodiments, L^{G} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G} is a C₁-4 bivalent saturated or unsaturated, straight or branched hydrocarbon chain.

In some embodiments, L^{G} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{G} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{G} is a C₁₋₂ bivalent saturated or unsaturated hydrocarbon chain. In some embodiments, L^{G} is a —CH₂—. In some embodiments, L^{G} is selected from the groups depicted in the compounds in Table 1.

As defined generally above, L^{Q} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a covalent bond. In some embodiments, L^{Q} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₄ bivalent saturated or unsaturated, straight or branched hydrocarbon chain.

In some embodiments, L^{Q} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{Q} is a C₁₋₂ bivalent saturated or unsaturated hydrocarbon chain. In some embodiments, L^{Q} is selected from the groups depicted in the compounds in Table 1.

As defined generally above, L^{X} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a covalent bond. In some embodiments, L^{X} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₄ bivalent saturated or unsaturated, straight or branched hydrocarbon chain.

In some embodiments, L^{X} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{X} is a C₁₋₂ bivalent saturated or unsaturated hydrocarbon chain. In some embodiments, L^{X} is selected from the groups depicted in the compounds in Table 1.

As defined generally above, LY is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a covalent bond. In some embodiments, L^{Y} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a C₁₋₄ bivalent saturated or unsaturated, straight or branched hydrocarbon chain.

In some embodiments, L^{Y} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Y} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{Y} is a C₁₋₂ bivalent saturated or unsaturated hydrocarbon chain.

In some embodiments, L^{Y} is —C(O)N(R)—, —C(O)N(R)CH₂—, or a covalent bond. In some embodiments, L^{Y} is —C(O)N(H)—, —C(O)N(H)CH₂—, or a covalent bond. In some embodiments, L^{Y} is —C(O)N(H)— or —C(O)N(H)CH₂—. In some embodiments, L^{Y} is —C(O)N(H)—. In some embodiments, L^{Y} is —C(O)N(H)CH₂—. In some embodiments, L^{Y} is selected from the groups depicted in the compounds in Table 1.

As defined generally above, R^{1A} is R^{A} or R substituted by r¹ instances of R^{1C}. In some embodiments, R^{1A} is R^{A}. In some embodiments, R^{1A} is R substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{1A} is substituted by r¹ instances of R^{1C}.

In some embodiments, R^{1A} is phenyl substituted by r¹ instances of R^{1C}. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of R^{1C}. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{1A} is substituted by r¹ instances of R^{1C}.

In some embodiments, R^{1A} is phenyl substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{1A} is phenyl substituted by 1-3 instances of R^{1C}. In some embodiments, R^{1A} is phenyl substituted by 2 instances of R^{1C}. In some embodiments, R^{1A} is phenyl substituted by 1 instance of R^{1C}.

In some embodiments, R^{1A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{1A} is phenyl substituted by 1-3 instances of a group independently selected from halogen and C₁₋₃ aliphatic optionally substituted with 1-3 halogen. In some embodiments, R^{1A} is phenyl substituted by 1-3 instances of a group independently selected from fluorine, chlorine, —CH₃, —CHF₂, and —CF₃.

In some embodiments, R^{1A} is phenyl substituted by 2 instances of a group independently selected from halogen, —CN, —O-(optionally substituted C₁₋₆ aliphatic), and an optionally substituted C₁₋₆ aliphatic. In some embodiments, R^{1A} is phenyl substituted by 2 instances of a group independently selected from halogen and C₁₋₃ aliphatic optionally substituted with 1-3 halogen. In some embodiments, R^{1A} is phenyl substituted by 2 instances of a group independently selected from fluorine, chlorine, —CH₃, —CHF₂, and —CF₃.

In some embodiments, R^{1A} is phenyl substituted by one group selected from halogen, —CN, —O-(optionally substituted C₁₋₆ aliphatic), and an optionally substituted C₁₋₆ aliphatic. In some embodiments, R^{1A} is phenyl substituted by one halogen or C₁₋₃ aliphatic group optionally substituted with 1-3 halogen. In some embodiments, R^{1A} is phenyl substituted by one fluorine, chlorine, —CH₃, —CHF₂, or —CF₃.

In some embodiments, R^{1A} is

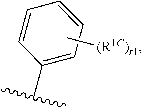

wherein R^{1C} and r¹ are as defined in the embodiments and classes and subclasses herein. In some embodiments, R^{1A} is

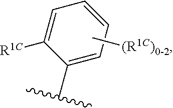

wherein R^{1C} is as defined in the embodiments and classes and subclasses herein. In some embodiments, R^{1A} is

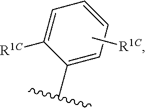

wherein R^{1C} is as defined in the embodiments and classes and subclasses herein. In some embodiments, R^{1A} is

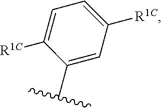

wherein R^{1C} is as defined in the embodiments and classes and subclasses herein. In some embodiments, R^{1A} is

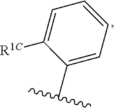

wherein R^{1C} is as defined in the embodiments and classes and subclasses herein.

In some embodiments, R^{1A} is

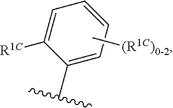

wherein each instance of R^{1C} is independently halogen, —CN, —O-(optionally substituted C₁₋₆ aliphatic), or an optionally substituted C₁₋₆ aliphatic. In some embodiments, R^{1A} is

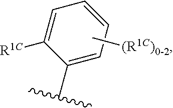

wherein each instance of R^{1C} is independently halogen or C₁₋₃ aliphatic optionally substituted with 1-3 halogen. In some embodiments, R^{1A} is

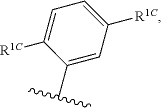

wherein each instance of R^{1C} is independently halogen or C₁₋₃ aliphatic optionally substituted with 1-3 halogen. In some embodiments, R^{1A} is

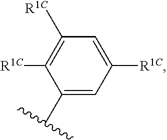

wherein each instance of R^{1C} is independently halogen or C₁₋₃ aliphatic optionally substituted with 1-3 halogen. In some embodiments, R^{1A} is

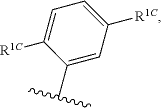

wherein each instance of R^{1C} is independently fluorine, chlorine, —CH₃, —CHF₂, or —CF₃. In some embodiments, R^{1A} is

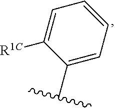

wherein R^{1C} is halogen or C₁₋₃ aliphatic optionally substituted with 1-3 halogen.

In some embodiments, R^{1A} is

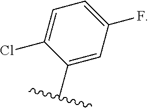 [SMILES: *c1cc(F)ccc1Cl]

In some embodiments, R is

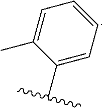 [SMILES: *c1ccccc1C]

In some embodiments, R^{1A} is

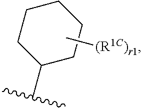

wherein R^{1C} and r¹ are as defined in the embodiments and classes and subclasses herein. In some embodiments, R^{1A} is

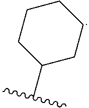 [SMILES: *C1CCCCC1]

### In

some embodiments, R^{1A} is

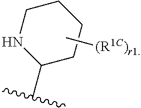

In some embodiments, R^{1A} is

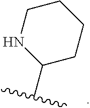 [SMILES: *C1CCCCN1]

In some embodiments, R^{1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{1A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is oxo. In some embodiments, R^{1A} is halogen. In some embodiments, R^{1A} is —CN. In some embodiments, R^{1A} is —NO₂. In some embodiments, R^{1A} is —OR. In some embodiments, R^{1A} is —SR. In some embodiments, R^{1A} is —NR₂. In some embodiments, R^{1A} is —S(O)₂R. In some embodiments, R^{1A} is —S(O)₂NR₂. In some embodiments, R^{1A} is —S(O)₂F. In some embodiments, R^{1A} is —S(O)R. In some embodiments, R^{1A} is —S(O)NR₂. In some embodiments, R^{1A} is —S(O)(NR)R. In some embodiments, R^{1A} is —C(O)R. In some embodiments, R^{1A} is —C(O)OR. In some embodiments, R^{1A} is —C(O)NR₂. In some embodiments, R^{1A} is —C(O)N(R)OR. In some embodiments, R^{1A} is —OC(O)R. In some embodiments, R^{1A} is —OC(O)NR₂. In some embodiments, R^{1A} is —N(R)C(O)OR. In some embodiments, R^{1A} is —N(R)C(O)R. In some embodiments, R^{1A} is —N(R)C(O)NR₂. In some embodiments, R^{1A} is —N(R)C(NR)NR₂. In some embodiments, R^{1A} is —N(R)S(O)₂NR₂. In some embodiments, R^{1A} is —N(R)S(O)₂R. In some embodiments, R^{1A} is —P(O)R₂. In some embodiments, R^{1A} is —P(O)(R)OR. In some embodiments, R^{1A} is —B(OR)₂. In some embodiments, R^{1A} is deuterium.

In some embodiments, R^{1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is halogen, —CN, or —NO₂. In some embodiments, R^{1A} is —OR, —SR, or —NR₂. In some embodiments, R^{1A} is —S(O)₂R, —S(O)₂NR₂, —S(O)₂F, —S(O)R, —S(O)NR₂, or —S(O)(NR)R. In some embodiments, R^{1A} is —C(O)R, —C(O)OR, —C(O)NR₂, or —C(O)N(R)OR. In some embodiments, R^{1A} is —OC(O)R or —OC(O)NR₂. In some embodiments, R^{1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{1A} is —P(O)R₂ or —P(O)(R)OR.

In some embodiments, R^{1A} is —OR, —OC(O)R, or —OC(O)NR₂. In some embodiments, R^{1A} is —SR, —S(O)₂R, —S(O)₂NR₂, —S(O)₂F, —S(O)R, —S(O)NR₂, or —S(O)(NR)R. In some embodiments, R^{1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{1A} is —S(O)₂R, —S(O)₂NR₂, or —S(O)₂F. In some embodiments, R^{1A} is —S(O)R, —S(O)NR₂, or —S(O)(NR)R. In some embodiments, R^{1A} is —SR, —S(O)₂R, or —S(O)R. In some embodiments, R^{1A} is —S(O)₂NR₂, —S(O)NR₂, or —S(O)(NR)R. In some embodiments, R^{1A} is —S(O)₂NR₂ or —S(O)NR₂. In some embodiments, R^{1A} is —SR, —S(O)₂R, —S(O)₂NR₂, or —S(O)R.

In some embodiments, R^{1A} is —N(R)C(O)OR, —N(R)C(O)R, or —N(R)C(O)NR₂. In some embodiments, R^{1A} is —N(R)S(O)₂NR₂ or —N(R)S(O)₂R. In some embodiments, R^{1A} is —N(R)C(O)OR or —N(R)C(O)R. In some embodiments, R^{1A} is —N(R)C(O)NR₂ or —N(R)S(O)₂NR₂. In some embodiments, R^{1A} is —N(R)C(O)OR, —N(R)C(O)R, or —N(R)S(O)₂R.

In some embodiments, R^{1A} is —NR₂, —N(R)C(O)OR, —N(R)C(O)R, or —N(R)C(O)NR₂. In some embodiments, R^{1A} is —NR₂, —N(R)C(O)OR, or —N(R)C(O)R. In some embodiments, R^{1A} is —NR₂, —N(R)C(O)OR, —N(R)C(O)R, or —N(R)S(O)₂R.

In some embodiments, R^{1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain substituted by r¹ instances of R^{1C}. In some embodiments, R^{1A} is phenyl substituted by r¹ instances of R^{1C}. In some embodiments, R^{1A} is naphthyl substituted by r¹ instances of R^{1C}. In some embodiments, R^{1A} is a 5-6 membered monocyclic heteroaryl ring having 1-4 heteroatoms independently selected from nitrogen, oxygen, and sulfur; wherein said ring is substituted by r¹ instances of R^{1C}. In some embodiments, R^{1A} is an 8-10 membered bicyclic heteroaryl ring having 1-4 heteroatoms independently selected from nitrogen, oxygen, and sulfur; wherein said ring is substituted by r¹ instances of R^{1C}. In some embodiments, R^{1A} is a 3-7 membered saturated or partially unsaturated monocyclic carbocyclic ring substituted by r¹ instances of R^{1C}. In some embodiments, R^{1A} is a 5-12 membered saturated or partially unsaturated bicyclic carbocyclic ring substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; wherein said ring is substituted by r¹ instances of R^{1C}. In some embodiments, R^{1A} is a 7-12 membered saturated or partially unsaturated bicyclic heterocyclic ring having 1-4 heteroatoms independently selected from nitrogen, oxygen, and sulfur; wherein said ring is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or naphthyl; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 5-12 membered saturated or partially unsaturated bicyclic carbocyclic ring; each of which is substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 5-6 membered monocyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 3-7 membered saturated or partially unsaturated monocyclic carbocyclic ring; each of which is substituted by r¹ instances of R^{1C}. In some embodiments, R^{1A} is naphthyl or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}. In some embodiments, R^{1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}.

In some embodiments, R^{1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r¹ instances of R^{1C}. In some embodiments, R^{1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is a C₁₋₆ aliphatic chain, phenyl, or a 3-7 membered saturated or partially unsaturated monocyclic carbocyclic ring; each of which is substituted by r¹ instances of R^{1C}.

In some embodiments, R^{1A} is selected from the groups depicted in the compounds in Table 1.

As defined generally above, R^{2A} is R^{A} or R substituted by r² instances of R^{2C}. In some embodiments, R^{2A} is R^{A}. In some embodiments, R^{2A} is R substituted by r² instances of R^{2C}.

In some embodiments, R^{2A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)O R, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by r² instances of R^{2C}. In some embodiments, R^{2A} is phenyl substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{2A} is phenyl substituted by 1-3 instances of a group independently selected from halogen and C₁₋₃ aliphatic optionally substituted with 1-3 halogen. In some embodiments, R^{2A} is phenyl substituted by 1-3 instances of a group independently selected from fluorine, chlorine, —CH₃, —CHF₂, and —CF₃.

In some embodiments, R^{2A} is phenyl substituted by 2 instances of a group independently selected from halogen, —CN, —O-(optionally substituted C₁₋₆ aliphatic), and an optionally substituted C₁₋₆ aliphatic. In some embodiments, R^{2A} is phenyl substituted by 2 instances of a group independently selected from halogen and C₁₋₃ aliphatic optionally substituted with 1-3 halogen. In some embodiments, R^{2A} is phenyl substituted by 2 instances of a group independently selected from fluorine, chlorine, —CH₃, —CHF₂, and —CF₃.

In some embodiments, R^{2A} is an 8-10 membered bicyclic heteroaryl ring having 1-4 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-2 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen, —CN, —O-(optionally substituted C₁₋₆ aliphatic), and an optionally substituted C₁₋₆ aliphatic. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen and C₁₋₃ aliphatic optionally substituted with 1-3 halogen. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from fluorine, chlorine, —CH₃, —CHF₂, and —CF₃.

In some embodiments, R^{2A} is:

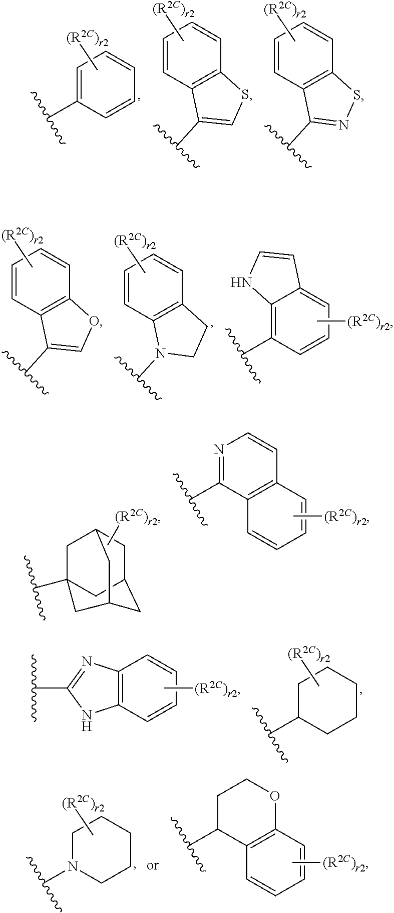

wherein R^{2C} and r² are as defined in the embodiments and classes and subclasses herein. In some embodiments, R^{2A} is

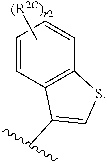

In some embodiments, R^{2A} is

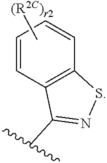

In some embodiments, R^{2A} is

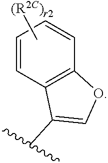

In some embodiments, R^{2A} is

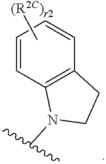

In some embodiments, R^{2A} is

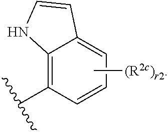

In some embodiments, R^{2A} is

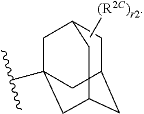

In some embodiments, R^{2A} is

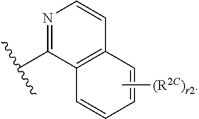

In some embodiments, R^{2A} is

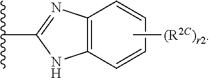

In some embodiments, R^{2A} is

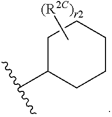

In some embodiments, R^{2A} is

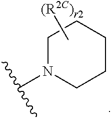

In some embodiments, R^{2A} is

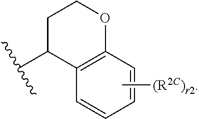

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is oxo. In some embodiments, R^{2A} is halogen. In some embodiments, R^{2A} is —CN. In some embodiments, R^{2A} is —NO₂. In some embodiments, R^{2A} is —OR. In some embodiments, R^{2A} is —SR. In some embodiments, R^{2A} is —NR₂. In some embodiments, R^{2A} is —S(O)₂R. In some embodiments, R^{2A} is —S(O)₂NR₂. In some embodiments, R^{2A} is —S(O)₂F. In some embodiments, R^{2A} is —S(O)R. In some embodiments, R^{2A} is —S(O)NR₂. In some embodiments, R^{2A} is —S(O)(NR)R. In some embodiments, R^{2A} is —C(O)R. In some embodiments, R^{2A} is —C(O)OR. In some embodiments, R^{2A} is —C(O)NR₂. In some embodiments, R^{2A} is —C(O)N(R)OR. In some embodiments, R^{2A} is —OC(O)R. In some embodiments, R^{2A} is —OC(O)NR₂. In some embodiments, R^{2A} is —N(R)C(O)OR. In some embodiments, R^{2A} is —N(R)C(O)R. In some embodiments, R^{2A} is —N(R)C(O)NR₂. In some embodiments, R^{2A} is —N(R)C(NR)NR₂. In some embodiments, R^{2A} is —N(R)S(O)₂NR₂. In some embodiments, R^{2A} is —N(R)S(O)₂R. In some embodiments, R^{2A} is —P(O)R₂. In some embodiments, R^{2A} is —P(O)(R)OR. In some embodiments, R^{2A} is —B(OR)₂. In some embodiments, R^{2A} is deuterium.

In some embodiments, R^{2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is halogen, —CN, or —NO₂. In some embodiments, R^{2A} is —OR, —SR, or —NR₂. In some embodiments, R^{2A} is —S(O)₂R, —S(O)₂NR₂, —S(O)₂F, —S(O)R, —S(O)NR₂, or —S(O)(NR)R. In some embodiments, R^{2A} is —C(O)R, —C(O)OR, —C(O)NR₂, or —C(O)N(R)OR. In some embodiments, R^{2A} is —OC(O)R or —OC(O)NR₂. In some embodiments, R^{2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{2A} is —P(O)R₂ or —P(O)(R)OR.

In some embodiments, R^{2A} is —OR, —OC(O)R, or —OC(O)NR₂. In some embodiments, R^{2A} is —SR, —S(O)₂R, —S(O)₂NR₂, —S(O)₂F, —S(O)R, —S(O)NR₂, or —S(O)(NR)R. In some embodiments, R^{2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{2A} is —S(O)₂R, —S(O)₂NR₂, or —S(O)₂F. In some embodiments, R^{2A} is —S(O)R, —S(O)NR₂, or —S(O)(NR)R. In some embodiments, R^{2A} is —SR, —S(O)₂R, or —S(O)R. In some embodiments, R^{2A} is —S(O)₂NR₂, —S(O)NR₂, or —S(O)(NR)R. In some embodiments, R^{2A} is —S(O)₂NR₂ or —S(O)NR₂. In some embodiments, R^{2A} is —SR, —S(O)₂R, —S(O)₂NR₂, or —S(O)R.

In some embodiments, R^{2A} is —N(R)C(O)OR, —N(R)C(O)R, or —N(R)C(O)NR₂. In some embodiments, R^{2A} is —N(R)S(O)₂NR₂ or —N(R)S(O)₂R. In some embodiments, R^{2A} is —N(R)C(O)OR or —N(R)C(O)R. In some embodiments, R^{2A} is —N(R)C(O)NR₂ or —N(R)S(O)₂NR₂. In some embodiments, R^{2A} is —N(R)C(O)OR, —N(R)C(O)R, or —N(R)S(O)₂R.

In some embodiments, R^{2A} is —NR₂, —N(R)C(O)OR, —N(R)C(O)R, or —N(R)C(O)NR₂. In some embodiments, R^{2A} is —NR₂, —N(R)C(O)OR, or —N(R)C(O)R. In some embodiments, R^{2A} is —NR₂, —N(R)C(O)OR, —N(R)C(O)R, or —N(R)S(O)₂R.

In some embodiments, R^{2A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain substituted by r² instances of R^{2C}. In some embodiments, R^{2A} is phenyl substituted by r² instances of R^{2C}. In some embodiments, R^{2A} is naphthyl substituted by r² instances of R^{2C}. In some embodiments, R^{2A} is cubanyl substituted by r² instances of R^{2C}. In some embodiments, R^{2A} is adamantyl substituted by r² instances of R^{2C}. In some embodiments, R^{2A} is a 5-6 membered monocyclic heteroaryl ring having 1-4 heteroatoms independently selected from nitrogen, oxygen, and sulfur; wherein said ring is substituted by r² instances of R^{2C}. In some embodiments, R^{2A} is an 8-10 membered bicyclic heteroaryl ring having 1-4 heteroatoms independently selected from nitrogen, oxygen, and sulfur; wherein said ring is substituted by r² instances of R^{2C}. In some embodiments, R^{2A} is a 3-7 membered saturated or partially unsaturated monocyclic carbocyclic ring substituted by r² instances of R^{2C}. In some embodiments, R^{2A} is a 5-12 membered saturated or partially unsaturated bicyclic carbocyclic ring substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; wherein said ring is substituted by r² instances of R^{2C}. In some embodiments, R^{2A} is a 7-12 membered saturated or partially unsaturated bicyclic heterocyclic ring having 1-4 heteroatoms independently selected from nitrogen, oxygen, and sulfur; wherein said ring is substituted by r² instances of R^{2C}.

In some embodiments, R^{2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or naphthyl; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 5-12 membered saturated or partially unsaturated bicyclic carbocyclic ring; each of which is substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 5-6 membered monocyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 3-7 membered saturated or partially unsaturated monocyclic carbocyclic ring; each of which is substituted by r² instances of R^{2C}In some embodiments, R^{2A} is naphthyl; cubanyl; adamantyl; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}. In some embodiments, R^{2A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}.

In some embodiments, R^{2A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by r² instances of R^{2C}. In some embodiments, R^{2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is a C₁₋₆ aliphatic chain, phenyl, or a 3-7 membered saturated or partially unsaturated monocyclic carbocyclic ring; each of which is substituted by r² instances of R^{2C}.

In some embodiments, R^{2A} is selected from the groups depicted in the compounds in Table 1.

As defined generally above, each instance of R^{EA} is independently R^{A} or R^{B} substituted by r³ instances of R^{EC}. In some embodiments, each instance of R^{EA} is independently R^{A}. In some embodiments, each instance of R^{EA} is independently R^{B} substituted by r³ instances of R^{EC}.

In some embodiments, each instance of R^{EA} is independently C₁₋₆ aliphatic substituted by r³ instances of R^{EC}. In some embodiments, each instance of R^{EA} is independently C₁₋₃ aliphatic substituted by r³ instances of R^{EC}. In some embodiments, each instance of R^{EA} is independently C₁₋₃ aliphatic substituted by r³ instances of halogen. In some embodiments, each instance of R^{EA} is independently C₁₋₃ aliphatic. In some embodiments, each instance of R^{EA} is independently —CH₃, —CH₂F, —CHF₂—, or —CF₃. In some embodiments, R^{EA} is —CH₃.

In some embodiments, R^{EA} is selected from the groups depicted in the compounds in Table 1.

As defined generally above, R^{GA} is R^{A} or R substituted by r⁴ instances of R^{GC}. In some embodiments, R^{GA} is R^{A}. In some embodiments, R^{GA} is R^{B} substituted by r⁴ instances of R^{EC}.

In some embodiments, each instance of R^{GA} is independently C₁₋₆ aliphatic substituted by r⁴ instances of R^{GC}. In some embodiments, each instance of R^{GA} is independently C₁₋₃ aliphatic substituted by r⁴ instances of R^{GC}. In some embodiments, each instance of R^{GA} is independently C₁₋₃ aliphatic substituted by r⁴ instances of halogen. In some embodiments, each instance of R^{GA} is independently C₁₋₃ aliphatic. In some embodiments, each instance of R^{GA} is independently —CH₃, —CH₂F, —CHF₂—, or —CF₃. In some embodiments, R^{GA} is —CH₃.

In some embodiments, each instance of R^{GA} is independently a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; wherein said ring is substituted by r⁴ instances of R^{E}C.

In some embodiments, each instance of R^{GA} is independently a 5-6 membered monocyclic heteroaryl ring having 1-4 heteroatoms independently selected from nitrogen, oxygen, and sulfur; wherein said ring is substituted by r⁴ instances of R^{EC}. In some embodiments, each instance of R^{GA} is independently a 5-6 membered monocyclic heteroaryl ring having 1-3 nitrogen atoms; wherein said ring is substituted by r⁴ instances of R^{EC}. In some embodiments, each instance of R^{GA} is independently a 5-membered monocyclic heteroaryl ring having 1-3 nitrogen atoms.

In some embodiments, each instance of R^{GA} is independently a 3-7 membered saturated or partially unsaturated monocyclic heterocyclic ring having 1-2 heteroatoms independently selected from nitrogen, oxygen, and sulfur; wherein said ring is substituted by r⁴ instances of R^{EC}. In some embodiments, each instance of R^{GA} is independently a 4-6 membered saturated monocyclic heterocyclic ring having one nitrogen atom and optionally one additional heteroatom selected from nitrogen, oxygen, and sulfur; wherein said ring is substituted by r⁴ instances of R^{EC}. In some embodiments, each instance of R^{GA} is independently a 4-6 membered saturated monocyclic heterocyclic ring having one nitrogen atom and optionally one additional heteroatom selected from nitrogen, oxygen, and sulfur. In some embodiments, each instance of R^{GA} is independently pyrrolidin-1-yl, piperidin-1-yl, morpholin-4-yl, or piperazin-1-yl; each of which is substituted by r⁴ instances of R^{EC}. In some embodiments, each instance of R^{GA} is independently pyrrolidin-1-yl, piperidin-1-yl, morpholin-4-yl, or piperazin-1-yl.

In some embodiments, R^{GA} is selected from the groups depicted in the compounds in Table 1.

As defined generally above, R^{QA} is R^{A} or R substituted by r⁵ instances of R^{QC}. In some embodiments, R^{QA} is R^{A}. In some embodiments, R^{QA} is R^{B} substituted by r⁵ instances of R^{QC}.

In some embodiments, R^{QA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{QA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{QA} is oxo. In some embodiments, R^{QA} is halogen. In some embodiments, R^{QA} is —CN. In some embodiments, R^{QA} is —NO₂. In some embodiments, R^{QA} is —OR. In some embodiments, R^{QA} is —SR. In some embodiments, R^{QA} is —NR₂. In some embodiments, R^{QA} is —S(O)₂R. In some embodiments, R^{QA} is —S(O)₂NR₂. In some embodiments, R^{QA} is —S(O)₂F. In some embodiments, R^{QA} is —S(O)R. In some embodiments, R^{QA} is —S(O)NR₂. In some embodiments, R^{QA} is —S(O)(NR)R. In some embodiments, R^{QA}is —C(O)R. In some embodiments, R^{QA} is —C(O)OR. In some embodiments, R^{QA} is —C(O)NR₂. In some embodiments, R^{QA} is —C(O)N(R)OR. In some embodiments, R^{QA} is —OC(O)R. In some embodiments, R^{QA} is —OC(O)NR₂. In some embodiments, R^{QA} is —N(R)C(O)OR. In some embodiments, R^{QA} is —N(R)C(O)R. In some embodiments, R^{QA} is —N(R)C(O)NR₂. In some embodiments, R^{QA} is —N(R)C(NR)NR₂. In some embodiments, R^{QA} is —N(R)S(O)₂NR₂. In some embodiments, R^{QA} is —N(R)S(O)₂R. In some embodiments, R^{QA} is —P(O)R₂. In some embodiments, R^{QA} is —P(O)(R)OR. In some embodiments, R^{QA} is —B(OR)₂. In some embodiments, R^{QA} is deuterium.

In some embodiments, R^{QA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{QA} is halogen, —CN, or —NO₂. In some embodiments, R^{QA} is —OR, —SR, or —NR₂. In some embodiments, R^{QA} is —S(O)₂R, —S(O)₂NR₂, —S(O)₂F, —S(O)R, —S(O)NR₂, or —S(O)(NR)R. In some embodiments, R^{QA} is —C(O)R, —C(O)OR, —C(O)NR₂, or —C(O)N(R)OR. In some embodiments, R^{QA} is —OC(O)R or —OC(O)NR₂. In some embodiments, R^{QA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{QA} is —P(O)R₂ or —P(O)(R)OR.

In some embodiments, R^{QA} is —OR, —OC(O)R, or —OC(O)NR₂. In some embodiments, R^{QA} is —SR, —S(O)₂R, —S(O)₂NR₂, —S(O)₂F, —S(O)R, —S(O)NR₂, or —S(O)(NR)R. In some embodiments, R^{QA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{QA} is —S(O)₂R, —S(O)₂NR₂, or —S(O)₂F. In some embodiments, R^{QA} is —S(O)R, —S(O)NR₂, or —S(O)(NR)R. In some embodiments, R^{QA} is —SR, —S(O)₂R, or —S(O)R. In some embodiments, R^{QA} is —S(O)₂NR₂, —S(O)NR₂, or —S(O)(NR)R. In some embodiments, R^{QA} is —S(O)₂NR₂ or —S(O)NR₂. In some embodiments, R^{QA} is —SR, —S(O)₂R, —S(O)₂NR₂, or —S(O)R.

In some embodiments, R^{QA} is —N(R)C(O)OR, —N(R)C(O)R, or —N(R)C(O)NR₂. In some embodiments, R^{QA} is —N(R)S(O)₂NR₂ or —N(R)S(O)₂R. In some embodiments, R^{QA} is —N(R)C(O)OR or —N(R)C(O)R. In some embodiments, R^{QA} is —N(R)C(O)NR₂ or —N(R)S(O)₂NR₂. In some embodiments, R^{QA} is —N(R)C(O)OR, —N(R)C(O)R, or —N(R)S(O)₂R.

In some embodiments, R^{QA} is —NR₂, —N(R)C(O)OR, —N(R)C(O)R, or —N(R)C(O)NR₂. In some embodiments, R^{QA} is —NR₂, —N(R)C(O)OR, or —N(R)C(O)R. In some embodiments, R^{QA} is —NR₂, —N(R)C(O)OR, —N(R)C(O)R, or —N(R)S(O)₂R.

In some embodiments, R^{QA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is a C₁₋₆ aliphatic chain substituted by r⁵ instances of R^{QC} In some embodiments, R^{QA} is phenyl substituted by r⁵ instances of R^{QC}. In some embodiments, R^{QA} is naphthyl substituted by r⁵ instances of R^{QC}. In some embodiments, R^{QA} is a 5-6 membered monocyclic heteroaryl ring having 1-4 heteroatoms independently selected from nitrogen, oxygen, and sulfur; wherein said ring is substituted by r⁵ instances of R^{QC}. In some embodiments, R^{QA} is an 8-10 membered bicyclic heteroaryl ring having 1-4 heteroatoms independently selected from nitrogen, oxygen, and sulfur; wherein said ring is substituted by r⁵ instances of R^{QC}. In some embodiments, R^{QA} is a 3-7 membered saturated or partially unsaturated monocyclic carbocyclic ring substituted by r⁵ instances of R^{QC}. In some embodiments, R^{QA} is a 5-12 membered saturated or partially unsaturated bicyclic carbocyclic ring substituted by r⁵ instances of R^{QC}. In some embodiments, R^{QA} is a 3-7 membered saturated or partially unsaturated monocyclic heterocyclic ring having 1-2 heteroatoms independently selected from nitrogen, oxygen, and sulfur; wherein said ring is substituted by r⁵ instances of R^{QC}. In some embodiments, R^{QA} is a 7-12 membered saturated or partially unsaturated bicyclic heterocyclic ring having 1-4 heteroatoms independently selected from nitrogen, oxygen, and sulfur; wherein said ring is substituted by r⁵ instances of R^{QC}.

In some embodiments, R^{QA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁵ instances of R^{QC}. In some embodiments, R^{QA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁵ instances of R^{QC}. In some embodiments, R^{QA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁵ instances of R^{QC}. In some embodiments, R^{QA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is phenyl or naphthyl; each of which is substituted by r⁵ instances of R^{QC}. In some embodiments, R^{QA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁵ instances of R^{QC}. In some embodiments, R^{QA} is a 3-7 membered saturated or partially unsaturated monocyclic carbocyclic ring or a 5-12 membered saturated or partially unsaturated bicyclic carbocyclic ring; each of which is substituted by r⁵ instances of R^{QC}. In some embodiments, R^{QA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is phenyl or a 5-6 membered monocyclic heteroaryl ring having 1-4 heteroatoms independently selected from nitrogen, oxygen, and sulfur; each of which is substituted by r⁵ instances of R^{QC}. In some embodiments, R^{QA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁵ instances of R^{QC}. In some embodiments, R^{QA} is naphthyl or an 8-10 membered bicyclic heteroaryl ring having 1-4 heteroatoms independently selected from nitrogen, oxygen, and sulfur; each of which is substituted by r⁵ instances of R^{QC}. In some embodiments, R^{QA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is phenyl or a 3-7 membered saturated or partially unsaturated monocyclic carbocyclic ring; each of which is substituted by r⁵ instances of R^{QC}. In some embodiments, R^{QA} is naphthyl or a 5-12 membered saturated or partially unsaturated bicyclic carbocyclic ring; each of which is substituted by r⁵ instances of R^{QC}. In some embodiments, R^{QA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁵ instances of R^{QC}. In some embodiments, R^{QA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}.

In some embodiments, R^{QA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁵ instances of R^{QC}. In some embodiments, R^{QA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁵ instances of R^{QC}. In some embodiments, R^{QA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁵ instances of R^{QC}.

In some embodiments, R^{QA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁵ instances of R^{QC}. In some embodiments, R^{QA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁵ instances of R^{QC}. In some embodiments, R^{QA} is a C₁₋₆ aliphatic chain, phenyl, or a 3-7 membered saturated or partially unsaturated monocyclic carbocyclic ring; each of which is substituted by r⁵ instances of R^{QC}.

In some embodiments, R^{QA} is selected from the groups depicted in the compounds in Table 1.

As defined generally above, R^{XA} is R^{A} or R^{B} substituted by r⁶ instances of R^{XC}. In some embodiments, R^{XA} is R^{A}. In some embodiments, R^{XA} is R^{B} substituted by r⁶ instances of R^{XC}.

In some embodiments, R^{XA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{XA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{XA} is oxo. In some embodiments, R^{XA} is halogen. In some embodiments, R^{XA} is —CN. In some embodiments, R^{XA} is —NO₂. In some embodiments, R^{XA} is —OR. In some embodiments, R^{XA} is —SR. In some embodiments, R^{XA} is —NR₂. In some embodiments, R^{XA} is —S(O)₂R. In some embodiments, R^{XA} is —S(O)₂NR₂. In some embodiments, R^{XA} is —S(O)₂F. In some embodiments, R^{XA} is —S(O)R. In some embodiments, R^{XA} is —S(O)NR₂. In some embodiments, R^{XA} is —S(O)(NR)R. In some embodiments, R^{XA} is —C(O)R. In some embodiments, R^{XA} is —C(O)OR. In some embodiments, R^{XA} is —C(O)NR₂. In some embodiments, R^{XA} is —C(O)N(R)OR. In some embodiments, R^{XA} is —OC(O)R. In some embodiments, R^{XA} is —OC(O)NR₂. In some embodiments, R^{XA} is —N(R)C(O)OR. In some embodiments, R^{XA} is —N(R)C(O)R. In some embodiments, R^{XA} is —N(R)C(O)NR₂. In some embodiments, R^{XA} is —N(R)C(NR)NR₂. In some embodiments, R^{XA} is —N(R)S(O)₂NR₂. In some embodiments, R^{XA} is —N(R)S(O)₂R. In some embodiments, R^{XA} is —P(O)R₂. In some embodiments, R^{XA} is —P(O)(R)OR. In some embodiments, R^{XA} is —B(OR)₂. In some embodiments, R^{XA} is deuterium.

In some embodiments, R^{XA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{XA} is halogen, —CN, or —NO₂. In some embodiments, R^{XA} is —OR, —SR, or —NR₂. In some embodiments, R^{XA} is —S(O)₂R, —S(O)₂NR₂, —S(O)₂F, —S(O)R, —S(O)NR₂, or —S(O)(NR)R. In some embodiments, R^{XA} is —C(O)R, —C(O)OR, —C(O)NR₂, or —C(O)N(R)OR. In some embodiments, R^{XA} is —OC(O)R or —OC(O)NR₂. In some embodiments, R^{XA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{XA} is —P(O)R₂ or —P(O)(R)OR.

In some embodiments, R^{XA} is —OR, —OC(O)R, or —OC(O)NR₂. In some embodiments, R^{XA} is —SR, —S(O)₂R, —S(O)₂NR₂, —S(O)₂F, —S(O)R, —S(O)NR₂, or —S(O)(NR)R. In some embodiments, R^{XA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{XA} is —S(O)₂R, —S(O)₂NR₂, or —S(O)₂F. In some embodiments, R^{XA} is —S(O)R, —S(O)NR₂, or —S(O)(NR)R. In some embodiments, R^{XA} is —SR, —S(O)₂R, or —S(O)R. In some embodiments, R^{XA} is —S(O)₂NR₂, —S(O)NR₂, or —S(O)(NR)R. In some embodiments, R^{XA} is —S(O)₂NR₂ or —S(O)NR₂. In some embodiments, R^{XA} is —SR, —S(O)₂R, —S(O)₂NR₂, or —S(O)R.

In some embodiments, R^{XA} is —N(R)C(O)OR, —N(R)C(O)R, or —N(R)C(O)NR₂. In some embodiments, R^{XA} is —N(R)S(O)₂NR₂ or —N(R)S(O)₂R. In some embodiments, R^{XA} is —N(R)C(O)OR or —N(R)C(O)R. In some embodiments, R^{XA} is —N(R)C(O)NR₂ or —N(R)S(O)₂NR₂. In some embodiments, R^{XA} is —N(R)C(O)OR, —N(R)C(O)R, or —N(R)S(O)₂R.

In some embodiments, R^{XA} is —NR₂, —N(R)C(O)OR, —N(R)C(O)R, or —N(R)C(O)NR₂. In some embodiments, R^{XA} is —NR₂, —N(R)C(O)OR, or —N(R)C(O)R. In some embodiments, R^{XA} is —NR₂, —N(R)C(O)OR, —N(R)C(O)R, or —N(R)S(O)₂R.

In some embodiments, R^{XA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is a C₁₋₆ aliphatic chain substituted by r⁶ instances of R^{XC} In some embodiments, R^{XA} is phenyl substituted by r⁶ instances of R^{XC}. In some embodiments, R^{XA} is naphthyl substituted by r⁶ instances of R^{XC}. In some embodiments, R^{XA} is a 5-6 membered monocyclic heteroaryl ring having 1-4 heteroatoms independently selected from nitrogen, oxygen, and sulfur; wherein said ring is substituted by r⁶ instances of R^{XC}. In some embodiments, R^{XA} is an 8-10 membered bicyclic heteroaryl ring having 1-4 heteroatoms independently selected from nitrogen, oxygen, and sulfur; wherein said ring is substituted by r⁶ instances of R^{XC}. In some embodiments, R^{XA} is a 3-7 membered saturated or partially unsaturated monocyclic carbocyclic ring substituted by r⁶ instances of R^{XC}. In some embodiments, R^{XA} is a 5-12 membered saturated or partially unsaturated bicyclic carbocyclic ring substituted by r⁶ instances of R^{XC}. In some embodiments, R^{XA} is a 3-7 membered saturated or partially unsaturated monocyclic heterocyclic ring having 1-2 heteroatoms independently selected from nitrogen, oxygen, and sulfur; wherein said ring is substituted by r⁶ instances of R^{XC}. In some embodiments, R^{XA} is a 7-12 membered saturated or partially unsaturated bicyclic heterocyclic ring having 1-4 heteroatoms independently selected from nitrogen, oxygen, and sulfur; wherein said ring is substituted by r⁶ instances of R^{XC}.

In some embodiments, R^{XA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁶ instances of R^{XC}. In some embodiments, R^{XA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁶ instances of R^{XC}. In some embodiments, R^{XA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁶ instances of R^{XC}. In some embodiments, R^{XA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is phenyl or naphthyl; each of which is substituted by r⁶ instances of R^{XC}. In some embodiments, R^{XA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁶ instances of R^{XC}. In some embodiments, R^{XA} is a 3-7 membered saturated or partially unsaturated monocyclic carbocyclic ring or a 5-12 membered saturated or partially unsaturated bicyclic carbocyclic ring; each of which is substituted by r⁶ instances of R^{XC}. In some embodiments, R^{XA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is phenyl or a 5-6 membered monocyclic heteroaryl ring having 1-4 heteroatoms independently selected from nitrogen, oxygen, and sulfur; each of which is substituted by r⁶ instances of R^{XC}. In some embodiments, R^{XA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁶ instances of R^{XC}. In some embodiments, R^{XA} is naphthyl or an 8-10 membered bicyclic heteroaryl ring having 1-4 heteroatoms independently selected from nitrogen, oxygen, and sulfur; each of which is substituted by r⁶ instances of R^{XC}. In some embodiments, R^{XA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is phenyl or a 3-7 membered saturated or partially unsaturated monocyclic carbocyclic ring; each of which is substituted by r⁶ instances of R^{XC} In some embodiments, R^{XA} is naphthyl or a 5-12 membered saturated or partially unsaturated bicyclic carbocyclic ring; each of which is substituted by r⁶ instances of R^{XC}. In some embodiments, R^{XA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁶ instances of R^{XC}. In some embodiments, R^{XA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}.

In some embodiments, R^{XA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁶ instances of R^{XC}. In some embodiments, R^{XA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁶ instances of R^{XC}. In some embodiments, R^{XA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁶ instances of R^{XC}.

In some embodiments, R^{XA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁶ instances of R^{XC}. In some embodiments, R^{XA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁶ instances of R^{XC}. In some embodiments, R^{XA} is a C₁₋₆ aliphatic chain, phenyl, or a 3-7 membered saturated or partially unsaturated monocyclic carbocyclic ring; each of which is substituted by r⁶ instances of R^{XC}.

In some embodiments, R^{XA} is selected from the groups depicted in the compounds in Table 1.

As defined generally above, R^{YA} is R^{A} or R^{B} substituted by r instances of R^{YC}. In some embodiments, R^{YA} is R^{A}. In some embodiments, R^{YA} is R^{B} substituted by r⁷ instances of R^{YC}.

In some embodiments, R^{YA} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁷ instances of R^{YC}.

In some embodiments, R^{YA} is a C₁₋₆ aliphatic chain substituted by r⁷ instances of R^{YC}In some embodiments, R^{YA} is phenyl substituted by r⁷ instances of R^{YC}. In some embodiments, R^{YA} is naphthyl substituted by r⁷ instances of R^{YC}. In some embodiments, R^{YA} is cubanyl substituted by r⁷ instances of R^{YC}. In some embodiments, R^{YA} is adamantyl substituted by r⁷ instances of R^{YC}. In some embodiments, R^{YA} is a 5-6 membered monocyclic heteroaryl ring having 1-4 heteroatoms independently selected from nitrogen, oxygen, and sulfur; wherein said ring is substituted by r⁷ instances of R^{YC}. In some embodiments, R^{YA} is an 8-10 membered bicyclic heteroaryl ring having 1-4 heteroatoms independently selected from nitrogen, oxygen, and sulfur; wherein said ring is substituted by r⁷ instances of R^{YC}. In some embodiments, R^{YA} is a 3-7 membered saturated or partially unsaturated monocyclic carbocyclic ring; wherein said ring is substituted by r⁷ instances of R^{YC}. In some embodiments, R^{YA} is a 5-12 membered saturated or partially unsaturated bicyclic carbocyclic ring; wherein said ring is substituted by r⁷ instances of R^{YC}. In some embodiments, R^{YA} is a 3-7 membered saturated or partially unsaturated monocyclic heterocyclic ring having 1-2 heteroatoms independently selected from nitrogen, oxygen, and sulfur; wherein said ring is substituted by r⁷ instances of R^{YC}. In some embodiments, R^{YA} is a 7-12 membered saturated or partially unsaturated bicyclic heterocyclic ring having 1-4 heteroatoms independently selected from nitrogen, oxygen, and sulfur; wherein said ring is substituted by r⁷ instances of R^{YC}.

In some embodiments, R^{YA} is a C₁₋₆ aliphatic chain; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁷ instances of R^{YC}. In some embodiments, R^{YA} is a C₁₋₆ aliphatic chain or a 3-7 membered saturated or partially unsaturated monocyclic carbocyclic ring; each of which is substituted by r⁷ instances of R^{YC}. In some embodiments, R^{YA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁷ instances of R^{YC}.

In some embodiments, R^{YA} is a 5-6 membered monocyclic heteroaryl ring having 1-4 heteroatoms independently selected from nitrogen, oxygen, and sulfur; wherein said ring is substituted by r⁷ instances of R^{YC}.

In some embodiments, R^{YA} is a 6-membered monocyclic heteroaryl ring having 1-4 heteroatoms independently selected from nitrogen, oxygen, and sulfur; wherein said ring is substituted by r⁷ instances of R^{YC}. In some embodiments, R^{YA} is a 6-membered monocyclic heteroaryl ring having 1 or 2 nitrogen atoms; wherein said ring is substituted by r⁷ instances of R^{YC}. In some embodiments, R^{YA} is pyridinyl substituted by r⁷ instances of R^{YC}. In some embodiments, R^{YA} is pyridin-3-yl substituted by r⁷ instances of R^{YC}. In some embodiments, R^{YA} is pyridin-3-yl substituted by one R^{YC}. In some embodiments, R^{YA} is pyridin-2-yl substituted by r⁷ instances of R^{YC}. In some embodiments, R^{YA} is pyridin-2-yl substituted by one R^{YC}. In some embodiments, R^{YA} is pyrimidinyl or pyridazinyl substituted by r⁷ instances of R^{YC}. In some embodiments, R^{YA} is pyrimidin-2-yl. In some embodiments, R^{YA} is pyridazine-3-yl.

In some embodiments, R^{YA} is a 5-membered monocyclic heteroaryl ring having 1-4 heteroatoms independently selected from nitrogen, oxygen, and sulfur; wherein said ring is substituted by r⁷ instances of R^{YC}. In some embodiments, R^{YA} is a 5-membered monocyclic heteroaryl ring having 1-2 heteroatoms independently selected from nitrogen and oxygen; wherein said ring is substituted by r⁷ instances of R^{YC}. In some embodiments, R^{YA} is isoxazolyl, oxazolyl, pyrazolyl, imidazolyl, or triazolyl; each of which is substituted by r⁷ instances of R^{YC}. In some embodiments, R^{YA} is isoxazolyl, oxazolyl, pyrazolyl, imidazolyl, or triazolyl; each of which is substituted by one R^{YC}. In some embodiments, R^{YA} is isoxazolyl, oxazolyl, pyrazolyl, imidazolyl, or triazolyl. In some embodiments, R^{YA} is isoxazolyl, oxazolyl, pyrazolyl, or imidazolyl; each of which is substituted by r⁷ instances of R^{YC}.

In some embodiments, R^{YA} is isoxazolyl or oxazolyl; each of which is substituted by r⁷ instances of R^{YC}. In some embodiments, R^{Y}A is isoxazolyl substituted by r⁷ instances of R^{YC}. In some embodiments, R^{YA} is isoxazolyl. In some embodiments, R^{YA} is oxazolyl substituted by r⁷ instances of R^{YC}. In some embodiments, R^{YA} is oxazolyl. In some embodiments, R^{YA} is pyrazolyl or imidazolyl; each of which is substituted by r instances of R^{YC}. In some embodiments, R^{YA} is pyrazolyl substituted by r⁷ instances of R^{YC}. In some embodiments, R^{YA} is pyrazolyl. In some embodiments, R^{YA} is imidazolyl substituted by r⁷ instances of R^{YC}. In some embodiments, R^{YA} is imidazolyl. In some embodiments, R^{YA} is triazolyl substituted by r⁷ instances of R^{YC}. In some embodiments, R^{YA} is triazolyl.

In some embodiments, R^{YA} is

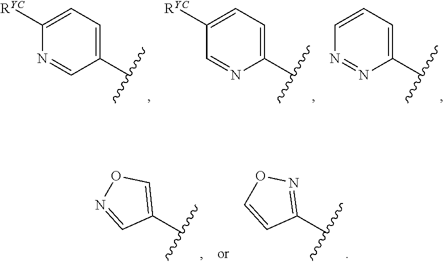

In some embodiments, R^{YA} is

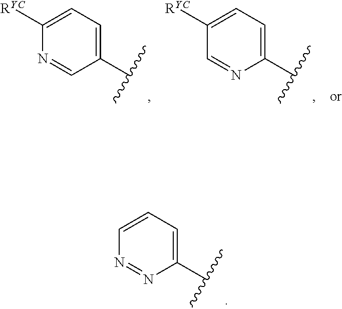

In some embodiments, R^{YA} is

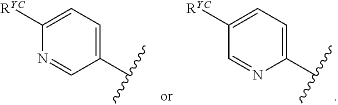

In some embodiments, R^{YA} is

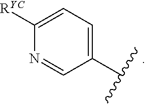

In some embodiments R^{YA} is

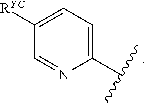

In some embodiments, R^{YA} is

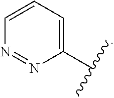 [SMILES: *c1cccnn1]

In some embodiments, R^{YA} is

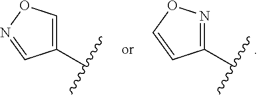 [SMILES: *c1cnoc1 ; *c1ccon1]

In some embodiments, R^{YA} is

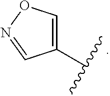 [SMILES: *c1cnoc1]

In some embodiments, R^{YA} is

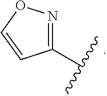 [SMILES: *c1ccon1]

In some embodiments, R^{YA} is a 3-7 membered saturated or partially unsaturated monocyclic heterocyclic ring having 1-2 heteroatoms independently selected from nitrogen, oxygen, and sulfur; wherein said ring is substituted by r⁷ instances of R^{YC}. In some embodiments, R^{YA} is a 4-6 membered saturated or partially unsaturated monocyclic heterocyclic ring having 1-2 heteroatoms independently selected from nitrogen, oxygen, and sulfur; wherein said ring is substituted by r⁷ instances of R^{YC}. In some embodiments, R^{YA} is a 4-6 membered saturated or partially unsaturated monocyclic heterocyclic ring having 1-2 heteroatoms independently selected from nitrogen, oxygen, and sulfur.

In some embodiments, R^{YA} is a 4-6 membered saturated monocyclic heterocyclic ring having 1-2 oxygen atoms; wherein said ring is substituted by r⁷ instances of R^{YC}. In some embodiments, R^{YA} is a 4-6 membered saturated monocyclic heterocyclic ring having 1-2 oxygen atoms. In some embodiments, R^{YA} is oxetanyl, tetrahydrofuranyl, tetrahydropyranyl, or 1,4-dioxan-2-yl, each of which is substituted by r⁷ instances of R^{YC}. In some embodiments, R^{YA} is oxetanyl, tetrahydrofuranyl, tetrahydropyranyl, or 1,4-dioxan-2-yl. In some embodiments, R^{YA} is oxetanyl, tetrahydrofuranyl, or tetrahydropyranyl, each of which is substituted by r⁷ instances of R^{YC}. In some embodiments, R^{YA} is oxetanyl, tetrahydrofuranyl, or tetrahydropyranyl. In some embodiments, R^{YA} is oxetanyl. In some embodiments, R^{YA} is tetrahydrofuranyl. In some embodiments, R^{YA} is tetrahydropyranyl. In some embodiments, R^{YA} is 1,4-dioxan-2-yl.

In some embodiments, R^{YA} is

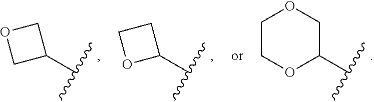 [SMILES: *C1COC1 ; *C1CCO1 ; *C1COCCO1]

In some embodiments, R^{YA} is

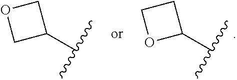 [SMILES: *C1COC1 ; *C1CCO1]

In some embodiments, R^{YA} is

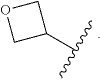 [SMILES: *C1COC1]

In some embodiments, R^{YA} is

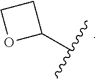 [SMILES: *C1CCO1]

In some embodiments, R^{YA} is

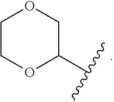 [SMILES: *C1COCCO1]

In some embodiments, R^{YA} is a C₁₋₆ aliphatic substituted by r⁷ instances of R^{YC}. In some embodiments, R^{YA} is a C₁₋₆ aliphatic chain substituted by r⁷ instances of R^{YC}. In some embodiments, R^{YA} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, R^{YA} is a C₁₋₆ aliphatic that is (i) substituted with 1 or 2 groups independently selected from —O—(C₁₋₆ aliphatic), —OH, —N(C₁₋₆ aliphatic)₂, and —CN; and (ii) optionally substituted with 1, 2, or 3 halogen atoms. In some embodiments, R^{YA} is a C₁₋₆ aliphatic substituted with one —OH and 1, 2, or 3 halogen atoms. In some embodiments, R^{YA} is

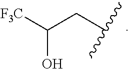 [SMILES: *CC(O)C(F)(F)F]

In some embodiments, R^{YA} is a C₁₋₆ aliphatic substituted with one —CN. In some embodiments, R^{YA} is —(CH₂)₂CN. In some embodiments, R^{YA} is a C₁₋₆ aliphatic substituted with 1, 2, or 3 halogen atoms. In some embodiments, R^{YA} is —CH₂CHF₂. In some embodiments, R^{YA} is a C₁₋₆ aliphatic substituted with 1, 2, or 3 deuterium atoms. In some embodiments, R^{YA} is a C₁₋₆ aliphatic. In some embodiments, R^{YA} is a C₁₋₄ alkyl. In some embodiments, R^{YA} is —CH₃.

In some embodiments, R^{YA} is

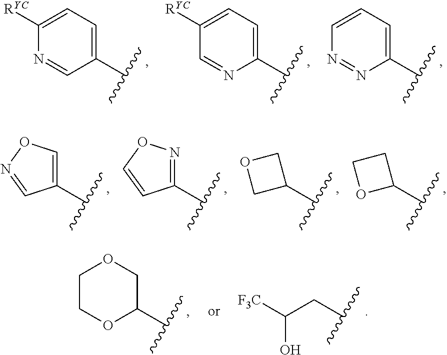

In some embodiments, R^{YA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, R^{YA} is halogen, —CN, —S(O)₂R, —S(O)₂NR₂, —S(O)₂F, —S(O)R, —S(O)NR₂, —S(O)(NR)R, —C(O)R, —C(O)OR, —C(O)NR₂, or —C(O)N(R)OR. In some embodiments, R^{YA} is halogen or —CN. In some embodiments,R^{A} is selected from the groups depicted in the compounds in Table 1.

As defined generally above, R^{L} is R^{A} or R^{B} substituted by r⁸ instances of R^{LC}. In some embodiments, R^{L} is R^{A}. In some embodiments, R^{L} is R^{B} substituted by r⁸ instances of R^{LC}.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is oxo. In some embodiments, R^{L} is halogen. In some embodiments, R^{L} is —CN. In some embodiments, R^{L} is —NO₂. In some embodiments, R^{L} is —OR. In some embodiments, R^{L} is —SR. In some embodiments, R^{L} is —NR₂. In some embodiments, R^{L} is —S(O)₂R. In some embodiments, R^{L} is —S(O)₂NR₂. In some embodiments, R^{L} is —S(O)₂F. In some embodiments, R^{L} is —S(O)R. In some embodiments, R^{L} is —S(O)NR₂. In some embodiments, R^{L} is —S(O)(NR)R. In some embodiments, R^{L} is —C(O)R. In some embodiments, R^{L} is —C(O)OR. In some embodiments, R^{L} is —C(O)NR₂. In some embodiments, R^{L} is —C(O)N(R)OR. In some embodiments, R^{L} is —OC(O)R. In some embodiments, R^{L} is —OC(O)NR₂. In some embodiments, R^{L} is —N(R)C(O)OR. In some embodiments, R^{L} is —N(R)C(O)R. In some embodiments, R^{L} is —N(R)C(O)NR₂. In some embodiments, R^{L} is —N(R)C(NR)NR₂. In some embodiments, R^{L} is —N(R)S(O)₂NR₂. In some embodiments, R^{L} is —N(R)S(O)₂R. In some embodiments, R^{L} is —P(O)R₂. In some embodiments, R^{L} is —P(O)(R)OR. In some embodiments, R^{L} is —B(OR)₂. In some embodiments, R^{L} is deuterium.

In some embodiments, R^{L} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is halogen, —CN, or —NO₂. In some embodiments, R^{L} is —OR, —SR, or —NR₂. In some embodiments, R^{L} is —S(O)₂R, —S(O)₂NR₂, —S(O)₂F, —S(O)R, —S(O)NR₂, or —S(O)(NR)R. In some embodiments, R^{L} is —C(O)R, —C(O)OR, —C(O)NR₂, or —C(O)N(R)OR. In some embodiments, R^{L} is —OC(O)R or —OC(O)NR₂. In some embodiments, R^{L} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L} is —P(O)R₂ or —P(O)(R)OR.

In some embodiments, R^{L} is —OR, —OC(O)R, or —OC(O)NR₂. In some embodiments, R^{L} is —SR, —S(O)₂R, —S(O)₂NR₂, —S(O)₂F, —S(O)R, —S(O)NR₂, or —S(O)(NR)R. In some embodiments, R^{L} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L} is —S(O)₂R, —S(O)₂NR₂, or —S(O)₂F. In some embodiments, R^{L} is —S(O)R, —S(O)NR₂, or —S(O)(NR)R. In some embodiments, R^{L} is —SR, —S(O)₂R, or —S(O)R. In some embodiments, R^{L} is —S(O)₂NR₂, —S(O)NR₂, or —S(O)(NR)R. In some embodiments, R^{L} is —S(O)₂NR₂ or —S(O)NR₂. In some embodiments, R^{L} is —SR, —S(O)₂R, —S(O)₂NR₂, or —S(O)R.

In some embodiments, R^{L} is —N(R)C(O)OR, —N(R)C(O)R, or —N(R)C(O)NR₂. In some embodiments, R^{L} is —N(R)S(O)₂NR₂ or —N(R)S(O)₂R. In some embodiments, R^{L} is —N(R)C(O)OR or —N(R)C(O)R. In some embodiments, R^{L} is —N(R)C(O)NR₂ or —N(R)S(O)₂NR₂. In some embodiments, R^{L} is —N(R)C(O)OR, —N(R)C(O)R, or —N(R)S(O)₂R.

In some embodiments, R^{L} is —NR₂, —N(R)C(O)OR, —N(R)C(O)R, or —N(R)C(O)NR₂. In some embodiments, R^{L} is —NR₂, —N(R)C(O)OR, or —N(R)C(O)R. In some embodiments, R^{L} is —NR₂, —N(R)C(O)OR, —N(R)C(O)R, or —N(R)S(O)₂R.

In some embodiments, R^{L} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is a C₁₋₆ aliphatic chain substituted by r⁸ instances of R^{LC} In some embodiments, R^{L} is phenyl substituted by r⁸ instances of R^{LC}. In some embodiments, R^{L} is naphthyl substituted by r⁸ instances of R^{LC}. In some embodiments, R^{L} is a 5-6 membered monocyclic heteroaryl ring having 1-4 heteroatoms independently selected from nitrogen, oxygen, and sulfur; wherein said ring is substituted by r⁸ instances of R^{LC}. In some embodiments, R^{L} is an 8-10 membered bicyclic heteroaryl ring having 1-4 heteroatoms independently selected from nitrogen, oxygen, and sulfur; wherein said ring is substituted by r⁸ instances of R^{LC}. In some embodiments, R^{L} is a 3-7 membered saturated or partially unsaturated monocyclic carbocyclic ring substituted by r⁸ instances of R^{LC}. In some embodiments, R^{L} is a 5-12 membered saturated or partially unsaturated bicyclic carbocyclic ring substituted by r⁸ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; wherein said ring is substituted by r⁸ instances of R^{LC}. In some embodiments, R^{L} is a 7-12 membered saturated or partially unsaturated bicyclic heterocyclic ring having 1-4 heteroatoms independently selected from nitrogen, oxygen, and sulfur; wherein said ring is substituted by r⁸ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁸ instances of R^{LC}. In some embodiments, R^{L} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁸ instances of R^{LC}. In some embodiments, R^{L} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁸ instances of R^{LC}. In some embodiments, R^{L} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is phenyl or naphthyl; each of which is substituted by r⁸ instances of R^{LC}. In some embodiments, R^{L} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁸ instances of R^{LC}. In some embodiments, R^{L} is a 3-7 membered saturated or partially unsaturated monocyclic carbocyclic ring or a 5-12 membered saturated or partially unsaturated bicyclic carbocyclic ring; each of which is substituted by r⁸ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is phenyl or a 5-6 membered monocyclic heteroaryl ring having 1-4 heteroatoms independently selected from nitrogen, oxygen, and sulfur; each of which is substituted by r⁸ instances of R^{LC}. In some embodiments, R^{L} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁸ instances of R^{LC}. In some embodiments, R^{L} is naphthyl or an 8-10 membered bicyclic heteroaryl ring having 1-4 heteroatoms independently selected from nitrogen, oxygen, and sulfur; each of which is substituted by r⁸ instances of R^{LC}. In some embodiments, R^{L} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is phenyl or a 3-7 membered saturated or partially unsaturated monocyclic carbocyclic ring; each of which is substituted by r⁸ instances of R^{LC} In some embodiments, R^{L} is naphthyl or a 5-12 membered saturated or partially unsaturated bicyclic carbocyclic ring; each of which is substituted by r⁸ instances of R^{LC}. In some embodiments, R^{L} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁸ instances of R^{LC}. In some embodiments, R^{L} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}.

In some embodiments, R^{L} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁸ instances of R^{LC}. In some embodiments, R^{L} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁸ instances of R^{LC}. In some embodiments, R^{L} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁸ instances of R^{LC}.

In some embodiments, R^{L} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁸ instances of R^{LC}. In some embodiments, R^{L} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁸ instances of R^{LC}. In some embodiments, R^{L} is a C₁₋₆ aliphatic chain, phenyl, or a 3-7 membered saturated or partially unsaturated monocyclic carbocyclic ring; each of which is substituted by r⁸ instances of R^{LC}.

In some embodiments, R^{L} is selected from the groups depicted in the compounds in Table 1.

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{A} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is oxo. In some embodiments, R^{A} is halogen. In some embodiments, R^{A} is —CN. In some embodiments, R^{A} is —NO₂. In some embodiments, R^{A} is —OR. In some embodiments, R^{A} is —SF₅. In some embodiments, R^{A} is —SR. In some embodiments, R^{A} is —NR₂. In some embodiments, R^{A} is —S(O)₂R. In some embodiments, R^{A} is —S(O)₂NR₂. In some embodiments, R^{A} is —S(O)₂F. In some embodiments, R^{A} is —S(O)R. In some embodiments, R^{A} is —S(O)NR₂. In some embodiments, R^{A} is —S(O)(NR)R. In some embodiments, R^{A} is —C(O)R. In some embodiments, R^{A} is —C(O)OR. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)C(O)OR. In some embodiments, R^{A} is —N(R)C(O)R. In some embodiments, R^{A} is —N(R)C(O)NR₂. In some embodiments, R^{A} is —N(R)C(NR)NR₂. In some embodiments, R^{A} is —N(R)S(O)₂NR₂. In some embodiments, R^{A} is —N(R)S(O)₂R. In some embodiments, R^{A} is —P(O)R₂. In some embodiments, R^{A} is —P(O)(R)OR. In some embodiments, R^{A} is —B(OR)₂. In some embodiments, R^{A} is deuterium.

In some embodiments, R^{A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is halogen, —CN, or —NO₂. In some embodiments, R^{A} is —OR, —SR, or —NR₂. In some embodiments, R^{A} is —S(O)₂R, —S(O)₂NR₂, —S(O)₂F, —S(O)R, —S(O)NR₂, or —S(O)(NR)R. In some embodiments, R^{A} is —C(O)R, —C(O)OR, —C(O)NR₂, or —C(O)N(R)OR. In some embodiments, R^{A} is —OC(O)R or —OC(O)NR₂. In some embodiments, R^{A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{A} is —P(O)R₂ or —P(O)(R)OR.

In some embodiments, R^{A} is —OR, —OC(O)R, or —OC(O)NR₂. In some embodiments, R^{A} is —SR, —S(O)₂R, —S(O)₂NR₂, —S(O)₂F, —S(O)R, —S(O)NR₂, or —S(O)(NR)R. In some embodiments, R^{A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{A} is —S(O)₂R, —S(O)₂NR₂, or —S(O)₂F. In some embodiments, R^{A} is —S(O)R, —S(O)NR₂, or —S(O)(NR)R. In some embodiments, R^{A} is —SR, —S(O)₂R, or —S(O)R. In some embodiments, R^{A} is —S(O)₂NR₂, —S(O)NR₂, or —S(O)(NR)R. In some embodiments, R^{A} is —S(O)₂NR₂ or —S(O)NR₂. In some embodiments, R^{A} is —SR, —S(O)₂R, —S(O)₂NR₂, or —S(O)R.

In some embodiments, R^{A} is —N(R)C(O)OR, —N(R)C(O)R, or —N(R)C(O)NR₂. In some embodiments, R^{A} is —N(R)S(O)₂NR₂ or —N(R)S(O)₂R. In some embodiments, R^{A} is —N(R)C(O)OR or —N(R)C(O)R. In some embodiments, R^{A} is —N(R)C(O)NR₂ or —N(R)S(O)₂NR₂. In some embodiments, R^{A} is —N(R)C(O)OR, —N(R)C(O)R, or —N(R)S(O)₂R.

In some embodiments, R^{A} is —NR₂, —N(R)C(O)OR, —N(R)C(O)R, or —N(R)C(O)NR₂. In some embodiments, R^{A} is —NR₂, —N(R)C(O)OR, or —N(R)C(O)R. In some embodiments, R^{A} is —NR₂, —N(R)C(O)OR, —N(R)C(O)R, or —N(R)S(O)₂R.

In some embodiments, R^{A} is selected from the groups depicted in the compounds in Table 1.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain. In some embodiments, R^{B} is phenyl. In some embodiments, R^{B} is naphthyl. In some embodiments, R^{B} is cubanyl. In some embodiments, R^{B} is adamantyl. In some embodiments, R^{B} is a 5-6 membered monocyclic heteroaryl ring having 1-4 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring. In some embodiments, R^{B} is a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur. In some embodiments, R^{B} is a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{B} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{B} is phenyl or naphthyl. In some embodiments, R^{B} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl or a 5-6 membered monocyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{B} is naphthyl or an 8-10 membered bicyclic heteroaryl ring having 1-4 heteroatoms independently selected from nitrogen, oxygen, and sulfur. In some embodiments, R^{B} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{B} is phenyl or a 3-7 membered saturated or partially unsaturated monocyclic carbocyclic ring. In some embodiments, R^{B} is naphthyl or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{B} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{B} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{B} is a C₁₋₆ aliphatic chain, phenyl, or a 3-7 membered saturated or partially unsaturated monocyclic carbocyclic ring.

In some embodiments, R^{B} is selected from the groups depicted in the compounds in Table 1.

As defined generally above, each instance of R^{1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{1C} is oxo. In some embodiments, R^{1C} is deuterium. In some embodiments, each instance of R^{1C} is independently halogen. In some embodiments, R^{1C} is —CN. In some embodiments, R^{1C} is —NO₂. In some embodiments, R^{1C} is —OR. In some embodiments, R^{1C} is —SR. In some embodiments, R^{1C} is —NR₂. In some embodiments, R^{1C} is —S(O)₂R. In some embodiments, R^{1C} is —S(O)₂NR₂. In some embodiments, R^{1C} is —S(O)₂F. In some embodiments, R^{1C} is —S(O)R. In some embodiments, R^{1C} is —S(O)NR₂. In some embodiments, R^{1C} is —S(O)(NR)R. In some embodiments, R^{1C} is —C(O)R. In some embodiments, R^{1C} is —C(O)OR. In some embodiments, R^{1C} is —C(O)NR₂. In some embodiments, R^{1C} is —C(O)N(R)OR. In some embodiments, R^{1C} is —OC(O)R. In some embodiments, R^{1C} is —OC(O)NR₂. In some embodiments, R^{1C} is —N(R)C(O)OR. In some embodiments, R^{1C} is —N(R)C(O)R. In some embodiments, R^{1C} is —N(R)C(O)NR₂. In some embodiments, R^{1C} is —N(R)C(NR)NR₂. In some embodiments, R^{1C} is —N(R)S(O)₂NR₂. In some embodiments, R^{1C} is —N(R)S(O)₂R. In some embodiments, R^{1C} is —P(O)R₂. In some embodiments, R^{1C} is —P(O)(R)OR. In some embodiments, R^{1C} is —B(OR)₂.

In some embodiments, each instance of R^{1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{1C} is independently halogen, —CN, or —NO₂. In some embodiments, each instance of R^{1C} is independently —OR, —SR, or —NR₂. In some embodiments, each instance of R^{1C} is independently —S(O)₂R, —S(O)₂NR₂, —S(O)₂F, —S(O)R, —S(O)NR₂, or —S(O)(NR)R. In some embodiments, each instance of R^{1C} is independently —C(O)R, —C(O)OR, —C(O)NR₂, or —C(O)N(R)OR. In some embodiments, each instance of R^{1C} is independently —OC(O)R or —OC(O)NR₂. In some embodiments, each instance of R^{1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{1C} is independently —P(O)R₂ or —P(O)(R)OR.

In some embodiments, each instance of R^{1C} is independently —OR, —OC(O)R, or —OC(O)NR₂. In some embodiments, each instance of R^{1C} is independently —SR, —S(O)₂R, —S(O)₂NR₂, —S(O)₂F, —S(O)R, —S(O)NR₂, or —S(O)(NR)R. In some embodiments, each instance of R^{1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{1C} is independently —S(O)₂R, —S(O)₂NR₂, or —S(O)₂F. In some embodiments, each instance of R^{1C} is independently —S(O)R, —S(O)NR₂, or —S(O)(NR)R. In some embodiments, each instance of R^{1C} is independently —SR, —S(O)₂R, or —S(O)R. In some embodiments, each instance of R^{1C} is independently —S(O)₂NR₂, —S(O)NR₂, or —S(O)(NR)R. In some embodiments, each instance of R^{1C} is independently —S(O)₂NR₂ or —S(O)NR₂. In some embodiments, each instance of R^{1C} is independently —SR, —S(O)₂R, —S(O)₂NR₂, or —S(O)R.

In some embodiments, each instance of R^{1C} is independently —N(R)C(O)OR, —N(R)C(O)R, or —N(R)C(O)NR₂. In some embodiments, each instance of R^{1C} is independently —N(R)S(O)₂NR₂ or —N(R)S(O)₂R. In some embodiments, each instance of R^{1C} is independently —N(R)C(O)OR or —N(R)C(O)R. In some embodiments, each instance of R^{1C} is independently —N(R)C(O)NR₂ or —N(R)S(O)₂NR₂. In some embodiments, each instance of R^{1C} is independently —N(R)C(O)OR, —N(R)C(O)R, or —N(R)S(O)₂R.

In some embodiments, each instance of R^{1C} is independently —NR₂, —N(R)C(O)OR, —N(R)C(O)R, or —N(R)C(O)NR₂. In some embodiments, each instance of R^{1C} is independently —NR₂, —N(R)C(O)OR, or —N(R)C(O)R. In some embodiments, each instance of R^{1C} is independently —NR₂, —N(R)C(O)OR, —N(R)C(O)R, or —N(R)S(O)₂R.

In some embodiments, each instance of R^{1C} is independently an optionally substituted C₁₋₆ aliphatic. In some embodiments, each instance of R^{1C} is independently an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic. In some embodiments, R^{1C} is phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur. In some embodiments, each instance of R^{1C} is independently a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{1C} is independently phenyl or a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently halogen, —CN, —O-(optionally substituted C₁₋₆ aliphatic), or an optionally substituted C₁₋₆ aliphatic. In some embodiments, each instance of R^{1C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{1C} is independently halogen or C₁₋₃ aliphatic optionally substituted with 1-3 halogen. In some embodiments, each instance of R^{1C} is independently fluorine, chlorine, —CH₃, —CHF₂, or —CF₃.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{1C} is independently selected from the groups depicted in the compounds in Table 1.

As defined generally above, each instance of R^{2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{2C} is oxo. In some embodiments, R^{2C} is deuterium. In some embodiments, each instance of R^{2C} is independently halogen. In some embodiments, R^{2C} is —CN. In some embodiments, R^{2C} is —NO₂. In some embodiments, R^{2C} is —OR. In some embodiments, R^{2C} is —SR. In some embodiments, R^{2C} is —NR₂. In some embodiments, R^{2C} is —S(O)₂R. In some embodiments, R^{2C} is —S(O)₂NR₂. In some embodiments, R^{2C} is —S(O)₂F. In some embodiments, R^{2C} is —S(O)R. In some embodiments, R^{2C} is —S(O)NR₂. In some embodiments, R^{2C} is —S(O)(NR)R. In some embodiments, R^{2C} is —C(O)R. In some embodiments, R^{2C} is —C(O)OR. In some embodiments, R^{2C} is —C(O)NR₂. In some embodiments, R^{2C} is —C(O)N(R)OR. In some embodiments, R^{2C} is —OC(O)R. In some embodiments, R^{2C} is —OC(O)NR₂. In some embodiments, R^{2C} is —N(R)C(O)OR. In some embodiments, R^{2C} is —N(R)C(O)R. In some embodiments, R^{2C} is —N(R)C(O)NR₂. In some embodiments, R^{2C} is —N(R)C(NR)NR₂. In some embodiments, R^{2C} is —N(R)S(O)₂NR₂. In some embodiments, R^{2C} is —N(R)S(O)₂R. In some embodiments, R^{2C} is —P(O)R₂. In some embodiments, R^{2C} is —P(O)(R)OR. In some embodiments, R^{2C} is —B(OR)₂.

In some embodiments, each instance of R^{2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{2C} is independently halogen, —CN, or —NO₂. In some embodiments, each instance of R^{2C} is independently —OR, —SR, or —NR₂. In some embodiments, each instance of R^{2C} is independently —S(O)₂R, —S(O)₂NR₂, —S(O)₂F, —S(O)R, —S(O)NR₂, or —S(O)(NR)R. In some embodiments, each instance of R^{2C} is independently —C(O)R, —C(O)OR, —C(O)NR₂, or —C(O)N(R)OR. In some embodiments, each instance of R^{2C} is independently —OC(O)R or —OC(O)NR₂. In some embodiments, each instance of R^{2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{2C} is independently —P(O)R₂ or —P(O)(R)OR.

In some embodiments, each instance of R^{2C} is independently —OR, —OC(O)R, or —OC(O)NR₂. In some embodiments, each instance of R^{2C} is independently —SR, —S(O)₂R, —S(O)₂NR₂, —S(O)₂F, —S(O)R, —S(O)NR₂, or —S(O)(NR)R. In some embodiments, each instance of R^{2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{2C} is independently —S(O)₂R, —S(O)₂NR₂, or —S(O)₂F. In some embodiments, each instance of R^{2C} is independently —S(O)R, —S(O)NR₂, or —S(O)(NR)R. In some embodiments, each instance of R^{2C} is independently —SR, —S(O)₂R, or —S(O)R. In some embodiments, each instance of R^{2C} is independently —S(O)₂NR₂, —S(O)NR₂, or —S(O)(NR)R. In some embodiments, each instance of R^{2C} is independently —S(O)₂NR₂ or —S(O)NR₂. In some embodiments, each instance of R^{2C} is independently —SR, —S(O)₂R, —S(O)₂NR₂, or —S(O)R.

In some embodiments, each instance of R^{2C} is independently —N(R)C(O)OR, —N(R)C(O)R, or —N(R)C(O)NR₂. In some embodiments, each instance of R^{2C} is independently —N(R)S(O)₂NR₂ or —N(R)S(O)₂R. In some embodiments, each instance of R^{2C} is independently —N(R)C(O)OR or —N(R)C(O)R. In some embodiments, each instance of R^{2C} is independently —N(R)C(O)NR₂ or —N(R)S(O)₂NR₂. In some embodiments, each instance of R^{2C} is independently —N(R)C(O)OR, —N(R)C(O)R, or —N(R)S(O)₂R.

In some embodiments, each instance of R^{2C} is independently —NR₂, —N(R)C(O)OR, —N(R)C(O)R, or —N(R)C(O)NR₂. In some embodiments, each instance of R^{2C} is independently —NR₂, —N(R)C(O)OR, or —N(R)C(O)R. In some embodiments, each instance of R^{2C} is independently —NR₂, —N(R)C(O)OR, —N(R)C(O)R, or —N(R)S(O)₂R.

In some embodiments, each instance of R^{2C} is independently an optionally substituted C₁₋₆ aliphatic. In some embodiments, each instance of R^{2C} is independently an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic. In some embodiments, R^{2C} is phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur. In some embodiments, each instance of R^{2C} is independently a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{2C} is independently phenyl or a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently halogen, —CN, —O-(optionally substituted C₁₋₆ aliphatic), or an optionally substituted C₁₋₆ aliphatic. In some embodiments, each instance of R^{2C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{2C} is independently halogen or C₁₋₃ aliphatic optionally substituted with 1-3 halogen. In some embodiments, each instance of R^{2C} is independently fluorine, chlorine, —CH₃, —CHF₂, or —CF₃.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{2C} is independently selected from the groups depicted in the compounds in Table 1.

As defined generally above, each instance of R^{EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R) C(O)R, —N(R)C(O)NR₂, —N(R)C(NR)NR₂, —N(R)S(O)₂NR₂, —N(R)S(O)₂R, —P(O)R₂, —P(O)(R) OR, or —B(OR)₂. In some embodiments, each instance of R^{EC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{EC} is independently deuterium, halogen, —CN, —OR, or —NR₂. In some embodiments, each instance of R^{EC} is independently deuterium or halogen. In some embodiments, each instance of R^{EC} is independently halogen.

In some embodiments, each instance of R^{EC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EC} is independently selected from the groups depicted in the compounds in Table 1.

As defined generally above, each instance of R^{GC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{GC} is independently C₁₋₆ aliphatic, oxo, deuterium, halogen, —CN, —OR, or —NR₂. In some embodiments, each instance of R^{GC} is independently C₁₋₃ aliphatic, oxo, deuterium, or halogen. In some embodiments, each instance of R^{GC} is oxo.

In some embodiments, each instance of R^{GC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{GC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{GC} is independently deuterium, halogen, —CN, —OR, or —NR₂. In some embodiments, each instance of R^{GC} is independently deuterium or halogen. In some embodiments, each instance of R^{GC} is independently halogen.

In some embodiments, each instance of R^{GC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{GC} is independently selected from the groups depicted in the compounds in Table 1.

As defined generally above, each instance of R^{QC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{QC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{QC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{QC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{QC} is oxo. In some embodiments, R^{QC} is deuterium. In some embodiments, each instance of R^{QC} is independently halogen. In some embodiments, R^{QC} is —CN. In some embodiments, R^{QC} is —NO₂. In some embodiments, R^{QC} is —OR. In some embodiments, R^{QC} is —SR. In some embodiments, R^{QC} is —NR₂. In some embodiments, R^{QC} is —S(O)₂R. In some embodiments, R^{QC} is —S(O)₂NR₂. In some embodiments, R^{QC} is —S(O)₂F. In some embodiments, R^{QC} is —S(O)R. In some embodiments, R^{QC} is —S(O)NR₂. In some embodiments, R^{QC} is —S(O)(NR)R. In some embodiments, R^{QC} is —C(O)R. In some embodiments, R^{QC} is —C(O)OR. In some embodiments, R^{QC} is —C(O)NR₂. In some embodiments, R^{QC} is —C(O)N(R)OR. In some embodiments, R^{QC} is —OC(O)R. In some embodiments, R^{QC} is —OC(O)NR₂. In some embodiments, R^{QC} is —N(R)C(O)OR. In some embodiments, R^{QC} is —N(R)C(O)R. In some embodiments, R^{QC} is —N(R)C(O)NR₂. In some embodiments, R^{QC} is —N(R)C(NR)NR₂. In some embodiments, R^{QC} is —N(R)S(O)₂NR₂. In some embodiments, R^{QC} is —N(R)S(O)₂R. In some embodiments, R^{QC} is —P(O)R₂. In some embodiments, R^{QC} is —P(O)(R)OR. In some embodiments, R^{QC} is —B(OR)₂.

In some embodiments, each instance of R^{QC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{QC} is independently halogen, —CN, or —NO₂. In some embodiments, each instance of R^{QC} is independently —OR, —SR, or —NR₂. In some embodiments, each instance of R^{QC} is independently —S(O)₂R, —S(O)₂NR₂, —S(O)₂F, —S(O)R, —S(O)NR₂, or —S(O)(NR)R. In some embodiments, each instance of R^{QC} is independently —C(O)R, —C(O)OR, —C(O)NR₂, or —C(O)N(R)OR. In some embodiments, each instance of R^{QC} is independently —OC(O)R or —OC(O)NR₂. In some embodiments, each instance of R^{QC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{QC} is independently —P(O)R₂ or —P(O)(R)OR.

In some embodiments, each instance of R^{QC} is independently —OR, —OC(O)R, or —OC(O)NR₂. In some embodiments, each instance of R^{QC} is independently —SR, —S(O)₂R, —S(O)₂NR₂, —S(O)₂F, —S(O)R, —S(O)NR₂, or —S(O)(NR)R. In some embodiments, each instance of R^{QC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{QC} is independently —S(O)₂R, —S(O)₂NR₂, or —S(O)₂F. In some embodiments, each instance of R^{QC} is independently —S(O)R, —S(O)NR₂, or —S(O)(NR)R. In some embodiments, each instance of R^{QC} is independently —SR, —S(O)₂R, or —S(O)R. In some embodiments, each instance of R^{QC} is independently —S(O)₂NR₂, —S(O)NR₂, or —S(O)(NR)R. In some embodiments, each instance of R^{QC} is independently —S(O)₂NR₂ or —S(O)NR₂. In some embodiments, each instance of R^{QC} is independently —SR, —S(O)₂R, —S(O)₂NR₂, or —S(O)R.

In some embodiments, each instance of R^{QC} is independently —N(R)C(O)OR, —N(R)C(O)R, or —N(R)C(O)NR₂. In some embodiments, each instance of R^{QC} is independently —N(R)S(O)₂NR₂ or —N(R)S(O)₂R. In some embodiments, each instance of R^{QC} is independently —N(R)C(O)OR or —N(R)C(O)R. In some embodiments, each instance of R^{QC} is independently —N(R)C(O)NR₂ or —N(R)S(O)₂NR₂. In some embodiments, each instance of R^{QC} is independently —N(R)C(O)OR, —N(R)C(O)R, or —N(R)S(O)₂R.

In some embodiments, each instance of R^{QC} is independently —NR₂, —N(R)C(O)OR, —N(R)C(O)R, or —N(R)C(O)NR₂. In some embodiments, each instance of R^{QC} is independently —NR₂, —N(R)C(O)OR, or —N(R)C(O)R. In some embodiments, each instance of R^{QC} is independently —NR₂, —N(R)C(O)OR, —N(R)C(O)R, or —N(R)S(O)₂R.

In some embodiments, each instance of R^{QC} is independently an optionally substituted C₁₋₆ aliphatic. In some embodiments, each instance of R^{QC} is independently an optionally substituted phenyl. In some embodiments, each instance of R^{QC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{QC} is independently an optionally substituted 5-6 membered monocyclic heteroaryl ring having 1-4 heteroatoms independently selected from nitrogen, oxygen, and sulfur.

In some embodiments, each instance of R^{QC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{QC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{QC} is independently an optionally substituted C₁₋₆ aliphatic or an optionally substituted phenyl. In some embodiments, each instance of R^{QC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{QC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{QC} is independently a C₁₋₆ aliphatic. In some embodiments, R^{QC} is phenyl. In some embodiments, each instance of R^{QC} is independently a 3-7 membered saturated or partially unsaturated monocyclic heterocyclic ring having 1-2 heteroatoms independently selected from nitrogen, oxygen, and sulfur. In some embodiments, each instance of R^{QC} is independently a 5-6 membered monocyclic heteroaryl ring having 1-4 heteroatoms independently selected from nitrogen, oxygen, and sulfur.

In some embodiments, each instance of R^{QC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{QC} is independently phenyl or a 5-6 membered monocyclic heteroaryl ring having 1-4 heteroatoms independently selected from nitrogen, oxygen, and sulfur.

In some embodiments, each instance of R^{QC} is independently a C₁₋₆ aliphatic or phenyl. In some embodiments, each instance of R^{QC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{QC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{QC} is independently selected from the groups depicted in the compounds in Table 1.

As defined generally above, each instance of R^{XC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{XC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{XC} is oxo. In some embodiments, R^{XC} is deuterium. In some embodiments, each instance of R^{XC} is independently halogen. In some embodiments, R^{XC} is —CN. In some embodiments, R^{XC} is —NO₂. In some embodiments, R^{XC} is —OR. In some embodiments, R^{XC} is —SR. In some embodiments, R^{XC} is —NR₂. In some embodiments, R^{XC} is —S(O)₂R. In some embodiments, R^{XC} is —S(O)₂NR₂. In some embodiments, R^{XC} is —S(O)₂F. In some embodiments, R^{XC} is —S(O)R. In some embodiments, R^{XC} is —S(O)NR₂. In some embodiments, R^{XC} is —S(O)(NR)R. In some embodiments, R^{XC} is —C(O)R. In some embodiments, R^{XC} is —C(O)OR. In some embodiments, R^{XC} is —C(O)NR₂. In some embodiments, R^{XC} is —C(O)N(R)OR. In some embodiments, R^{XC} is —OC(O)R. In some embodiments, R^{XC} is —OC(O)NR₂. In some embodiments, R^{XC} is —N(R)C(O)OR. In some embodiments, R^{XC} is —N(R)C(O)R. In some embodiments, R^{XC} is —N(R)C(O)NR₂. In some embodiments, R^{XC} is —N(R)C(NR)NR₂. In some embodiments, R^{XC} is —N(R)S(O)₂NR₂. In some embodiments, R^{XC} is —N(R)S(O)₂R. In some embodiments, R^{XC} is —P(O)R₂. In some embodiments, R^{XC} is —P(O)(R)OR. In some embodiments, R^{XC} is —B(OR)₂.

In some embodiments, each instance of R^{XC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{XC} is independently halogen, —CN, or —NO₂. In some embodiments, each instance of R^{XC} is independently —OR, —SR, or —NR₂. In some embodiments, each instance of R^{XC} is independently —S(O)₂R, —S(O)₂NR₂, —S(O)₂F, —S(O)R, —S(O)NR₂, or —S(O)(NR)R. In some embodiments, each instance of R^{XC} is independently —C(O)R, —C(O)OR, —C(O)NR₂, or —C(O)N(R)OR. In some embodiments, each instance of R^{XC} is independently —OC(O)R or —OC(O)NR₂. In some embodiments, each instance of R^{XC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{XC} is independently —P(O)R₂ or —P(O)(R)OR.

In some embodiments, each instance of R^{XC} is independently —OR, —OC(O)R, or —OC(O)NR₂. In some embodiments, each instance of R^{XC} is independently —SR, —S(O)₂R, —S(O)₂NR₂, —S(O)₂F, —S(O)R, —S(O)NR₂, or —S(O)(NR)R. In some embodiments, each instance of R^{XC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{XC} is independently —S(O)₂R, —S(O)₂NR₂, or —S(O)₂F. In some embodiments, each instance of R^{XC} is independently —S(O)R, —S(O)NR₂, or —S(O)(NR)R. In some embodiments, each instance of R^{XC} is independently —SR, —S(O)₂R, or —S(O)R. In some embodiments, each instance of R^{XC} is independently —S(O)₂NR₂, —S(O)NR₂, or —S(O)(NR)R. In some embodiments, each instance of R^{XC} is independently —S(O)₂NR₂ or —S(O)NR₂. In some embodiments, each instance of R^{XC} is independently —SR, —S(O)₂R, —S(O)₂NR₂, or —S(O)R.

In some embodiments, each instance of R^{XC} is independently —N(R)C(O)OR, —N(R)C(O)R, or —N(R)C(O)NR₂. In some embodiments, each instance of R^{XC} is independently —N(R)S(O)₂NR₂ or —N(R)S(O)₂R. In some embodiments, each instance of R^{X}c is independently —N(R)C(O)OR or —N(R)C(O)R. In some embodiments, each instance of R^{X}c is independently —N(R)C(O)NR₂ or —N(R)S(O)₂NR₂. In some embodiments, each instance of R^{XC} is independently —N(R)C(O)OR, —N(R)C(O)R, or —N(R)S(O)₂R.

In some embodiments, each instance of R^{XC} is independently —NR₂, —N(R)C(O)OR, —N(R)C(O)R, or —N(R)C(O)NR₂. In some embodiments, each instance of R^{XC} is independently —NR₂, —N(R)C(O)OR, or —N(R)C(O)R. In some embodiments, each instance of R^{XC} is independently —NR₂, —N(R)C(O)OR, —N(R)C(O)R, or —N(R)S(O)₂R.

In some embodiments, each instance of R^{XC} is independently an optionally substituted C₁₋₆ aliphatic. In some embodiments, each instance of R^{XC} is independently an optionally substituted phenyl. In some embodiments, each instance of R^{XC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{XC} is independently an optionally substituted 5-6 membered monocyclic heteroaryl ring having 1-4 heteroatoms independently selected from nitrogen, oxygen, and sulfur.

In some embodiments, each instance of R^{XC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{XC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{XC} is independently an optionally substituted C₁₋₆ aliphatic or an optionally substituted phenyl. In some embodiments, each instance of R^{XC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{XC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic. In some embodiments, R^{XC} is phenyl. In some embodiments, each instance of R^{XC} is independently a 3-7 membered saturated or partially unsaturated monocyclic heterocyclic ring having 1-2 heteroatoms independently selected from nitrogen, oxygen, and sulfur. In some embodiments, each instance of R^{XC} is independently a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{XC} is independently phenyl or a 5-6 membered monocyclic heteroaryl ring having 1-4 heteroatoms independently selected from nitrogen, oxygen, and sulfur.

In some embodiments, each instance of R^{XC} is independently a C₁₋₆ aliphatic or phenyl. In some embodiments, each instance of R^{XC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{XC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{XC} is independently selected from the groups depicted in the compounds in Table 1.

As defined generally above, each instance of R^{YC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{YC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{YC} is oxo. In some embodiments, R^{YC} is deuterium. In some embodiments, each instance of R^{YC} is independently halogen. In some embodiments, R^{YC} is —CN. In some embodiments, R^{YC} is —NO₂. In some embodiments, R^{YC} is —OR. In some embodiments, R^{YC} is —SR. In some embodiments, R^{YC} is —NR₂. In some embodiments, R^{YC} is —S(O)₂R. In some embodiments, R^{YC} is —S(O)₂NR₂. In some embodiments, R^{YC} is —S(O)₂F. In some embodiments, R^{YC} is —S(O)R. In some embodiments, R^{YC} is —S(O)NR₂. In some embodiments, R^{YC} is —S(O)(NR)R. In some embodiments, R^{YC} is —C(O)R. In some embodiments, R^{YC} is —C(O)OR. In some embodiments, R^{YC} is —C(O)NR₂. In some embodiments, R^{YC} is —C(O)N(R)OR. In some embodiments, R^{YC} is —OC(O)R. In some embodiments, R^{YC} is —OC(O)NR₂. In some embodiments, R^{YC} is —N(R)C(O)OR. In some embodiments, R^{YC} is —N(R)C(O)R. In some embodiments, R^{YC} is —N(R)C(O)NR₂. In some embodiments, R^{Y}C is —N(R)C(NR)NR₂. In some embodiments, R^{Y}C is —N(R)S(O)₂NR₂. In some embodiments, R^{YC} is —N(R)S(O)₂R. In some embodiments, R^{YC} is —P(O)R₂. In some embodiments, R^{YC} is —P(O)(R)OR. In some embodiments, R^{YC} is —B(OR)₂.

In some embodiments, each instance of R^{YC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{YC} is independently halogen, —CN, or —NO₂. In some embodiments, each instance of R^{YC} is independently —OR, —SR, or —NR₂. In some embodiments, each instance of R^{YC} is independently —S(O)₂R, —S(O)₂NR₂, —S(O)₂F, —S(O)R, —S(O)NR₂, or —S(O)(NR)R. In some embodiments, each instance of R^{YC} is independently —C(O)R, —C(O)OR, —C(O)NR₂, or —C(O)N(R)OR. In some embodiments, each instance of R^{YC} is independently —OC(O)R or —OC(O)NR₂. In some embodiments, each instance of R^{YC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{YC} is independently —P(O)R₂ or —P(O)(R)OR.

In some embodiments, each instance of R^{YC} is independently —OR, —OC(O)R, or —OC(O)NR₂. In some embodiments, each instance of R^{YC} is independently —SR, —S(O)₂R, —S(O)₂NR₂, —S(O)₂F, —S(O)R, —S(O)NR₂, or —S(O)(NR)R. In some embodiments, each instance of R^{YC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{YC} is independently —S(O)₂R, —S(O)₂NR₂, or —S(O)₂F. In some embodiments, each instance of R^{YC} is independently —S(O)R, —S(O)NR₂, or —S(O)(NR)R. In some embodiments, each instance of R^{YC} is independently —SR, —S(O)₂R, or —S(O)R. In some embodiments, each instance of R^{YC} is independently —S(O)₂NR₂, —S(O)NR₂, or —S(O)(NR)R. In some embodiments, each instance of R^{YC} is independently —S(O)₂NR₂ or —S(O)NR₂. In some embodiments, each instance of R^{YC} is independently —SR, —S(O)₂R, —S(O)₂NR₂, or —S(O)R.

In some embodiments, each instance of R^{YC} is independently —N(R)C(O)OR, —N(R)C(O)R, or —N(R)C(O)NR₂. In some embodiments, each instance of R^{YC} is independently —N(R)S(O)₂NR₂ or —N(R)S(O)₂R. In some embodiments, each instance of R^{YC} is independently —N(R)C(O)OR or —N(R)C(O)R. In some embodiments, each instance of R^{YC} is independently —N(R)C(O)NR₂ or —N(R)S(O)₂NR₂. In some embodiments, each instance of R^{YC} is independently —N(R)C(O)OR, —N(R)C(O)R, or —N(R)S(O)₂R.

In some embodiments, each instance of R^{YC} is independently —NR₂, —N(R)C(O)OR, —N(R)C(O)R, or —N(R)C(O)NR₂. In some embodiments, each instance of R^{YC} is independently —NR₂, —N(R)C(O)OR, or —N(R)C(O)R. In some embodiments, each instance of R^{Y}C is independently —NR₂, —N(R)C(O)OR, —N(R)C(O)R, or —N(R)S(O)₂R.

In some embodiments, each instance of R^{YC} is independently an optionally substituted C₁₋₆ aliphatic. In some embodiments, each instance of R^{YC} is independently an optionally substituted phenyl. In some embodiments, each instance of R^{YC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{YC} is independently an optionally substituted 5-6 membered monocyclic heteroaryl ring having 1-4 heteroatoms independently selected from nitrogen, oxygen, and sulfur.

In some embodiments, each instance of R^{YC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{YC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{YC} is independently an optionally substituted C₁₋₆ aliphatic or an optionally substituted phenyl. In some embodiments, each instance of R^{YC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{YC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic. In some embodiments, R^{YC} is phenyl. In some embodiments, each instance of R^{YC} is independently a 3-7 membered saturated or partially unsaturated monocyclic heterocyclic ring having 1-2 heteroatoms independently selected from nitrogen, oxygen, and sulfur. In some embodiments, each instance of R^{YC} is independently a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{YC} is independently phenyl or a 5-6 membered monocyclic heteroaryl ring having 1-4 heteroatoms independently selected from nitrogen, oxygen, and sulfur.

In some embodiments, each instance of R^{YC} is independently a C₁₋₆ aliphatic or phenyl. In some embodiments, each instance of R^{YC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{YC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{YC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{YC} is independently halogen, —CN, —OH, —O-(optionally substituted C₁₋₃ aliphatic), or an optionally substituted C₁₋₃ aliphatic. In some embodiments, each instance of R^{YC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{YC} is independently fluorine, chlorine, —OH, —OCH₃, —OCF₃, —CH₃, —CHF₂, or —CF₃. In some embodiments, each instance of R^{Y}c is independently fluorine or —OH.

In some embodiments, each instance of R^{Y}c is independently oxo, deuterium, halogen, —CN, —OH, —O-(optionally substituted C₁₋₃ aliphatic), or an optionally substituted C₁₋₃ aliphatic. In some embodiments, each instance of R^{Y}c is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{Y}c is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{Y}c is independently oxo, deuterium, fluorine, chlorine, —CN, —OH, —OCH₃, —OCF₃, —CH₃, —CHF₂, or —CF₃. In some embodiments, each instance of R^{YC} is independently oxo, deuterium, —CN, fluorine, or —OH. In some embodiments, each instance of R^{Y}c is independently oxo, deuterium, —CN, —CH₃, or —CHF₂. In some embodiments, each instance of R^{Y}c is independently deuterium, —CN, —CH₃, or —CHF₂.

In some embodiments, each instance of R^{Y}c is independently oxo, halogen, —CN, —OH, —O-(optionally substituted C₁₋₃ aliphatic), or an optionally substituted C₁₋₃ aliphatic. In some embodiments, each instance of R^{Y}c is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{Y}c is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{Y}c is independently oxo, fluorine, chlorine, —CN, —OH, —OCH₃, —OCF₃, —CH₃, —CHF₂, or —CF₃. In some embodiments, each instance of R^{Y}c is independently oxo, —CN, fluorine, or —OH. In some embodiments, each instance of R^{Y}c is independently oxo, —CN, —CH₃, or —CHF₂. In some embodiments, each instance of R^{Y}c is independently —CN, —CH₃, or —CHF₂.

In some embodiments, each instance of R^{Y}c is independently selected from the groups depicted in the compounds in Table 1.

As defined generally above, each instance of R^{LC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{LC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{LC} is oxo. In some embodiments, R^{LC} is deuterium. In some embodiments, each instance of R^{LC} is independently halogen. In some embodiments, R^{LC} is —CN. In some embodiments, R^{LC} is —NO₂. In some embodiments, R^{LC} is —OR. In some embodiments, R^{LC} is —SR. In some embodiments, R^{LC} is —NR₂. In some embodiments, R^{LC} is —S(O)₂R. In some embodiments, R^{LC} is —S(O)₂NR₂. In some embodiments, R^{LC} is —S(O)₂F. In some embodiments, R^{LC} is —S(O)R. In some embodiments, R^{LC} is —S(O)NR₂. In some embodiments, R^{LC} is —S(O)(NR)R. In some embodiments, R^{LC} is —C(O)R. In some embodiments, R^{LC} is —C(O)OR. In some embodiments, R^{LC} is —C(O)NR₂. In some embodiments, R^{LC} is —C(O)N(R)OR. In some embodiments, R^{LC} is —OC(O)R. In some embodiments, R^{LC} is —OC(O)NR₂. In some embodiments, R^{LC} is —N(R)C(O)OR. In some embodiments, R^{LC} is —N(R)C(O)R. In some embodiments, R^{LC} is —N(R)C(O)NR₂. In some embodiments, R^{LC} is —N(R)C(NR)NR₂. In some embodiments, R^{LC} is —N(R)S(O)₂NR₂. In some embodiments, R^{LC} is —N(R)S(O)₂R. In some embodiments, R^{LC} is —P(O)R₂. In some embodiments, R^{LC} is —P(O)(R)OR. In some embodiments, R^{LC} is —B(OR)₂.

In some embodiments, each instance of R^{LC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{LC} is independently halogen, —CN, or —NO₂. In some embodiments, each instance of R^{LC} is independently —OR, —SR, or —NR₂. In some embodiments, each instance of R^{LC} is independently —S(O)₂R, —S(O)₂NR₂, —S(O)₂F, —S(O)R, —S(O)NR₂, or —S(O)(NR)R. In some embodiments, each instance of R^{LC} is independently —C(O)R, —C(O)OR, —C(O)NR₂, or —C(O)N(R)OR. In some embodiments, each instance of R^{LC} is independently —OC(O)R or —OC(O)NR₂. In some embodiments, each instance of R^{LC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{LC} is independently —P(O)R₂ or —P(O)(R)OR.

In some embodiments, each instance of R^{LC} is independently —OR, —OC(O)R, or —OC(O)NR₂. In some embodiments, each instance of R^{LC} is independently —SR, —S(O)₂R, —S(O)₂NR₂, —S(O)₂F, —S(O)R, —S(O)NR₂, or —S(O)(NR)R. In some embodiments, each instance of R^{LC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{LC} is independently —S(O)₂R, —S(O)₂NR₂, or —S(O)₂F. In some embodiments, each instance of R^{LC} is independently —S(O)R, —S(O)NR₂, or —S(O)(NR)R. In some embodiments, each instance of R^{LC} is independently —SR, —S(O)₂R, or —S(O)R. In some embodiments, each instance of R^{LC} is independently —S(O)₂NR₂, —S(O)NR₂, or —S(O)(NR)R. In some embodiments, each instance of R^{LC} is independently —S(O)₂NR₂ or —S(O)NR₂. In some embodiments, each instance of R^{LC} is independently —SR, —S(O)₂R, —S(O)₂NR₂, or —S(O)R.

In some embodiments, each instance of R^{LC} is independently —N(R)C(O)OR, —N(R)C(O)R, or —N(R)C(O)NR₂. In some embodiments, each instance of R^{LC} is independently —N(R)S(O)₂NR₂ or —N(R)S(O)₂R. In some embodiments, each instance of R^{LC} is independently —N(R)C(O)OR or —N(R)C(O)R. In some embodiments, each instance of R^{LC} is independently —N(R)C(O)NR₂ or —N(R)S(O)₂NR₂. In some embodiments, each instance of R^{LC} is independently —N(R)C(O)OR, —N(R)C(O)R, or —N(R)S(O)₂R.

In some embodiments, each instance of R^{LC} is independently —NR₂, —N(R)C(O)OR, —N(R)C(O)R, or —N(R)C(O)NR₂. In some embodiments, each instance of R^{LC} is independently —NR₂, —N(R)C(O)OR, or —N(R)C(O)R. In some embodiments, each instance of R^{LC} is independently —NR₂, —N(R)C(O)OR, —N(R)C(O)R, or —N(R)S(O)₂R.

In some embodiments, each instance of R^{LC} is independently an optionally substituted C₁₋₆ aliphatic. In some embodiments, each instance of R^{LC} is independently an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic. In some embodiments, R^{LC} is phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur. In some embodiments, each instance of R^{LC} is independently a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{LC} is independently phenyl or a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently selected from the groups depicted in the compounds in Table 1.

As defined generally above, each instance of R^{EEC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EEC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{EEC} is independently oxo, deuterium, halogen, —CN, —NO₂, —OR, —NR₂, —C(O)R, —C(O)OR, —C(O)NR₂, —OC(O)R, —N(R)C(O)R, or —N(R)S(O)₂R. In some embodiments, each instance of R^{EEC} is independently deuterium, halogen, —CN, —OR, or —NR₂. In some embodiments, each instance of R^{EEC} is independently deuterium or halogen. In some embodiments, each instance of R^{EEC} is independently halogen.

In some embodiments, each instance of R^{EEC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{EEC} is independently selected from the groups depicted in the compounds in Table 1.

As defined generally above, each instance of R^{Q1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{Q1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{Q1C} is oxo. In some embodiments, R^{Q1C} is deuterium. In some embodiments, each instance of R^{Q1C} is independently halogen. In some embodiments, R^{Q1C} is —CN. In some embodiments, R^{Q1C} is —NO₂. In some embodiments, R^{Q1C} is —OR. In some embodiments, R^{Q1C} is —SR. In some embodiments, R^{Q1C} is —NR₂. In some embodiments, R^{Q1C} is —S(O)₂R. In some embodiments, R^{Q1C} is —S(O)₂NR₂. In some embodiments, R^{Q1C} is —S(O)₂F. In some embodiments, R^{Q1C} is —S(O)R. In some embodiments, R^{Q1C} is —S(O)NR₂. In some embodiments, R^{Q1C} is —S(O)(NR)R. In some embodiments, R^{Q1C} is —C(O)R. In some embodiments, R^{Q1C} is —C(O)OR. In some embodiments, R^{Q1C} is —C(O)NR₂. In some embodiments, R^{Q1C} is —C(O)N(R)OR. In some embodiments, R^{Q1C} is —OC(O)R. In some embodiments, R^{Q1C} is —OC(O)NR₂. In some embodiments, R^{Q1C} is —N(R)C(O)OR. In some embodiments, R^{Q1C} is —N(R)C(O)R. In some embodiments, R^{Q1C} is —N(R)C(O)NR₂. In some embodiments, R^{Q1C} is —N(R)C(NR)NR₂. In some embodiments, R^{Q1C} is —N(R)S(O)₂NR₂. In some embodiments, R^{Q1C} is —N(R)S(O)₂R. In some embodiments, R^{Q1C} is —P(O)R₂. In some embodiments, R^{Q1C} is —P(O)(R)OR. In some embodiments, R^{Q1C} is —B(OR)₂.

In some embodiments, each instance of R^{Q1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{Q1C} is independently halogen, —CN, or —NO₂. In some embodiments, each instance of R^{Q1C} is independently —OR, —SR, or —NR₂. In some embodiments, each instance of R^{Q1C} is independently —S(O)₂R, —S(O)₂NR₂, —S(O)₂F, —S(O)R, —S(O)NR₂, or —S(O)(NR)R. In some embodiments, each instance of R^{Q1C} is independently —C(O)R, —C(O)OR, —C(O)NR₂, or —C(O)N(R)OR. In some embodiments, each instance of R^{Q1C} is independently —OC(O)R or —OC(O)NR₂. In some embodiments, each instance of R^{Q1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{Q1C} is independently —P(O)R₂ or —P(O)(R)OR.

In some embodiments, each instance of R^{Q1C} is independently —OR, —OC(O)R, or —OC(O)NR₂. In some embodiments, each instance of R^{Q1C} is independently —SR, —S(O)₂R, —S(O)₂NR₂, —S(O)₂F, —S(O)R, —S(O)NR₂, or —S(O)(NR)R. In some embodiments, each instance of R^{Q1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{Q1C} is independently —S(O)₂R, —S(O)₂NR₂, or —S(O)₂F. In some embodiments, each instance of R^{Q1C} is independently —S(O)R, —S(O)NR₂, or —S(O)(NR)R. In some embodiments, each instance of R^{Q1C} is independently —SR, —S(O)₂R, or —S(O)R. In some embodiments, each instance of R^{Q1C} is independently —S(O)₂NR₂, —S(O)NR₂, or —S(O)(NR)R. In some embodiments, each instance of R^{Q1C} is independently —S(O)₂NR₂ or —S(O)NR₂. In some embodiments, each instance of R^{Q1C} is independently —SR, —S(O)₂R, —S(O)₂NR₂, or —S(O)R.

In some embodiments, each instance of R^{Q1C} is independently —N(R)C(O)OR, —N(R)C(O)R, or —N(R)C(O)NR₂. In some embodiments, each instance of R^{Q1C} is independently —N(R)S(O)₂NR₂ or —N(R)S(O)₂R. In some embodiments, each instance of R^{Q1C} is independently —N(R)C(O)OR or —N(R)C(O)R. In some embodiments, each instance of R^{Q1C} is independently —N(R)C(O)NR₂ or —N(R)S(O)₂NR₂. In some embodiments, each instance of R^{Q1C} is independently —N(R)C(O)OR, —N(R)C(O)R, or —N(R)S(O)₂R.

In some embodiments, each instance of R^{Q1C} is independently —NR₂, —N(R)C(O)OR, —N(R)C(O)R, or —N(R)C(O)NR₂. In some embodiments, each instance of R^{Q1C} is independently —NR₂, —N(R)C(O)OR, or —N(R)C(O)R. In some embodiments, each instance of R^{Q1C} is independently —NR₂, —N(R)C(O)OR, —N(R)C(O)R, or —N(R)S(O)₂R.

In some embodiments, each instance of R^{Q1C} is independently an optionally substituted C₁₋₆ aliphatic. In some embodiments, each instance of R^{Q1C} is independently an optionally substituted phenyl. In some embodiments, each instance of R^{Q1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{Q1C} is independently an optionally substituted 5-6 membered monocyclic heteroaryl ring having 1-4 heteroatoms independently selected from nitrogen, oxygen, and sulfur.

In some embodiments, each instance of R^{Q1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{Q1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{Q1C} is independently an optionally substituted C₁₋₆ aliphatic or an optionally substituted phenyl. In some embodiments, each instance of R^{Q1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{Q1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{Q1C} is independently a C₁₋₆ aliphatic. In some embodiments, R^{Q1C} is phenyl. In some embodiments, each instance of R^{Q1C} is independently a 3-7 membered saturated or partially unsaturated monocyclic heterocyclic ring having 1-2 heteroatoms independently selected from nitrogen, oxygen, and sulfur. In some embodiments, each instance of R^{Q1C} is independently a 5-6 membered monocyclic heteroaryl ring having 1-4 heteroatoms independently selected from nitrogen, oxygen, and sulfur.

In some embodiments, each instance of R^{Q1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{Q1C} is independently phenyl or a 5-6 membered monocyclic heteroaryl ring having 1-4 heteroatoms independently selected from nitrogen, oxygen, and sulfur.

In some embodiments, each instance of R^{Q1C} is independently a C₁₋₆ aliphatic or phenyl. In some embodiments, each instance of R^{Q1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{Q1C} is independently oxo, halogen, —CN, —O-(optionally substituted C₁₋₆ aliphatic), or an optionally substituted C₁₋₆ aliphatic. In some embodiments, each instance of R^{Q1C} is independently oxo, halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{Q1C} is independently oxo, halogen, or C₁₋₃ aliphatic optionally substituted with 1-3 halogen. In some embodiments, each instance of R^{Q1C} is independently oxo, fluorine, chlorine, —CH₃, —CHF₂, or —CF₃.

In some embodiments, each instance of R^{Q1C} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0 or 1. In some embodiments, n is 0, 1, or 2. In some embodiments, n is 0, 1, 2, or 3. In some embodiments, n is 1 or 2. In some embodiments, n is 1, 2, or 3. In some embodiments, n is 1, 2, 3, or 4. In some embodiments, n is 2 or 3. In some embodiments, n is 2, 3, or 4. In some embodiments, n is 3 or 4. In some embodiments, n is selected from the values represented in the compounds in Table 1.

As defined generally above, p is 0, 1, 2, 3, or 4. In some embodiments, p is 0. In some embodiments, p is 1. In some embodiments, p is 2. In some embodiments, p is 3. In some embodiments, p is 4. In some embodiments, p is 0 or 1. In some embodiments, p is 0, 1, or 2. In some embodiments, p is 0, 1, 2, or 3. In some embodiments, p is 1 or 2. In some embodiments, p is 1, 2, or 3. In some embodiments, p is 1, 2, 3, or 4. In some embodiments, p is 2 or 3. In some embodiments, p is 2, 3, or 4. In some embodiments, p is 3 or 4. In some embodiments, p is selected from the values represented in the compounds in Table 1.

As defined generally above, r¹ is 0, 1, 2, 3, or 4. In some embodiments, r¹ is 0. In some embodiments, r¹ is 1. In some embodiments, r¹ is 2. In some embodiments, r¹ is 3. In some embodiments, r¹ is 4. In some embodiments, r¹ is 0 or 1. In some embodiments, r¹ is 0, 1, or 2. In some embodiments, r¹ is 0, 1, 2, or 3. In some embodiments, r¹ is 1 or 2. In some embodiments, r¹ is 1, 2, or 3. In some embodiments, r¹ is 1, 2, 3, or 4. In some embodiments, r¹ is 2 or 3. In some embodiments, r¹ is 2, 3, or 4. In some embodiments, r¹ is 3 or 4. In some embodiments, r¹ is selected from the values represented in the compounds in Table 1.

As defined generally above, r² is 0, 1, 2, 3, or 4. In some embodiments, r² is 0. In some embodiments, r² is 1. In some embodiments, r² is 2. In some embodiments, r² is 3. In some embodiments, r² is 4. In some embodiments, r² is 0 or 1. In some embodiments, r² is 0, 1, or 2. In some embodiments, r² is 0, 1, 2, or 3. In some embodiments, r² is 1 or 2. In some embodiments, r² is 1, 2, or 3. In some embodiments, r² is 1, 2, 3, or 4. In some embodiments, r² is 2 or 3. In some embodiments, r² is 2, 3, or 4. In some embodiments, r² is 3 or 4. In some embodiments, r² is selected from the values represented in the compounds in Table 1.

As defined generally above, r³ is 0, 1, 2, 3, or 4. In some embodiments, r³ is 0. In some embodiments, r³ is 1. In some embodiments, r³ is 2. In some embodiments, r³ is 3. In some embodiments, r³ is 4. In some embodiments, r³ is 0 or 1. In some embodiments, r³ is 0, 1, or 2. In some embodiments, r³ is 0, 1, 2, or 3. In some embodiments, r³ is 1 or 2. In some embodiments, r³ is 1, 2, or 3. In some embodiments, r³ is 1, 2, 3, or 4. In some embodiments, r³ is 2 or 3. In some embodiments, r³ is 2, 3, or 4. In some embodiments, r³ is 3 or 4. In some embodiments, r³ is selected from the values represented in the compounds in Table 1.

As defined generally above, r⁴ is 0, 1, 2, 3, or 4. In some embodiments, r⁴ is 0. In some embodiments, r⁴ is 1. In some embodiments, r⁴ is 2. In some embodiments, r⁴ is 3. In some embodiments, r⁴ is 4. In some embodiments, r⁴ is 0 or 1. In some embodiments, r⁴ is 0, 1, or 2. In some embodiments, r⁴ is 0, 1, 2, or 3. In some embodiments, r⁴ is 1 or 2. In some embodiments, r⁴ is 1, 2, or 3. In some embodiments, r⁴ is 1, 2, 3, or 4. In some embodiments, r⁴ is 2 or 3. In some embodiments, r⁴ is 2, 3, or 4. In some embodiments, r⁴ is 3 or 4. In some embodiments, r⁴ is selected from the values represented in the compounds in Table 1.

As defined generally above, r⁵ is 0, 1, 2, 3, or 4. In some embodiments, r⁵ is 0. In some embodiments, r⁵ is 1. In some embodiments, r⁵ is 2. In some embodiments, r⁵ is 3. In some embodiments, r⁵ is 4. In some embodiments, r⁵ is 0 or 1. In some embodiments, r⁵ is 0, 1, or 2. In some embodiments, r⁵ is 0, 1, 2, or 3. In some embodiments, r⁵ is 1 or 2. In some embodiments, r⁵ is 1, 2, or 3. In some embodiments, r⁵ is 1, 2, 3, or 4. In some embodiments, r⁵ is 2 or 3. In some embodiments, r⁵ is 2, 3, or 4. In some embodiments, r⁵ is 3 or 4. In some embodiments, r⁵ is selected from the values represented in the compounds in Table 1.

As defined generally above, r⁶ is 0, 1, 2, 3, or 4. In some embodiments, r⁶ is 0. In some embodiments, r⁶ is 1. In some embodiments, r⁶ is 2. In some embodiments, r⁶ is 3. In some embodiments, r⁶ is 4. In some embodiments, r⁶ is 0 or 1. In some embodiments, r⁶ is 0, 1, or 2. In some embodiments, r⁶ is 0, 1, 2, or 3. In some embodiments, r⁶ is 1 or 2. In some embodiments, r⁶ is 1, 2, or 3. In some embodiments, r⁶ is 1, 2, 3, or 4. In some embodiments, r⁶ is 2 or 3. In some embodiments, r⁶ is 2, 3, or 4. In some embodiments, r⁶ is 3 or 4. In some embodiments, r⁶ is selected from the values represented in the compounds in Table 1.

As defined generally above, r⁷ is 0, 1, 2, 3, or 4. In some embodiments, r⁷ is 0. In some embodiments, r⁷ is 1. In some embodiments, r⁷ is 2. In some embodiments, r⁷ is 3. In some embodiments, r⁷ is 4. In some embodiments, r⁷ is 0 or 1. In some embodiments, r⁷ is 0, 1, or 2. In some embodiments, r⁷ is 0, 1, 2, or 3. In some embodiments, r⁷ is 1 or 2. In some embodiments, r⁷ is 1, 2, or 3. In some embodiments, r⁷ is 1, 2, 3, or 4. In some embodiments, r⁷ is 2 or 3. In some embodiments, r⁷ is 2, 3, or 4. In some embodiments, r⁷ is 3 or 4. In some embodiments, r⁷ is selected from the values represented in the compounds in Table 1.

As defined generally above, r⁸ is 0, 1, 2, 3, or 4. In some embodiments, r⁸ is 0. In some embodiments, r⁸ is 1. In some embodiments, r⁸ is 2. In some embodiments, r⁸ is 3. In some embodiments, r⁸ is 4. In some embodiments, r⁸ is 0 or 1. In some embodiments, r⁸ is 0, 1, or 2. In some embodiments, r⁸ is 0, 1, 2, or 3. In some embodiments, r⁸ is 1 or 2. In some embodiments, r⁸ is 1, 2, or 3. In some embodiments, r⁸ is 1, 2, 3, or 4. In some embodiments, r⁸ is 2 or 3. In some embodiments, r⁸ is 2, 3, or 4. In some embodiments, r⁸ is 3 or 4. In some embodiments, r⁸ is selected from the values represented in the compounds in Table 1.

In some embodiments, the present disclosure provides a compound of formula I wherein E is —C(O)—, thereby forming a compound of formula II:

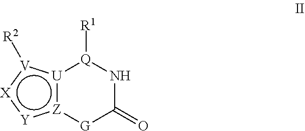

or a pharmaceutically acceptable salt thereof, wherein each of R¹, R², Q, G, U, V, X, Y, and Z is as defined in embodiments and classes and subclasses herein.

In some embodiments, the present disclosure provides a compound of formula II wherein U and V are C, thereby forming a compound of formula III:

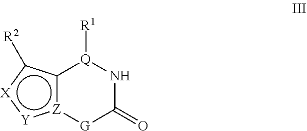

or a pharmaceutically acceptable salt thereof, wherein each of R¹, R², Q, G, X, Y, and Z is as defined in embodiments and classes and subclasses herein.

In some embodiments, the present disclosure provides a compound of formula III wherein Q is CH, thereby forming a compound of formula IV:

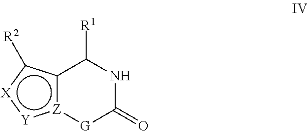

or a pharmaceutically acceptable salt thereof, wherein each of R¹, R², G, X, Y, and Z is as defined in embodiments and classes and subclasses herein.

In some embodiments, the present disclosure provides a compound of formula IV wherein G is CH₂, thereby forming a compound of formula V:

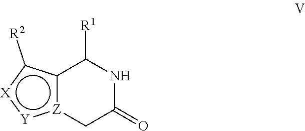

or a pharmaceutically acceptable salt thereof, wherein each of R¹, R², X, Y, and Z is as defined in embodiments and classes and subclasses herein.

In some embodiments, the present disclosure provides a compound of formula IV wherein Z is N, thereby forming a compound of formula VI:

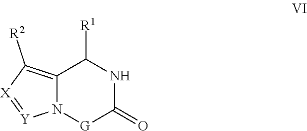

or a pharmaceutically acceptable salt thereof, wherein each of R¹, R², G, X, and Y is as defined in embodiments and classes and subclasses herein.

In some embodiments, the present disclosure provides a compound of formula IV, V, or VI, or a pharmaceutically acceptable salt thereof.

In some embodiments, the present disclosure provides a compound of formula III wherein Z is N, and X or Y is CH, thereby forming a compound of formula VII or VIII:

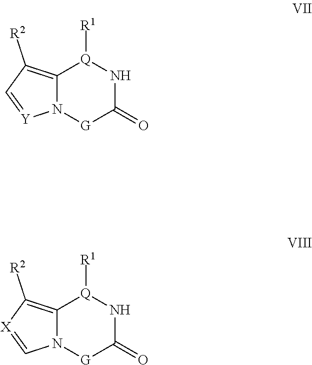

or a pharmaceutically acceptable salt thereof, wherein each of R¹, R², Q, G, X, and Y is as defined in embodiments and classes and subclasses herein.

In some embodiments, the present disclosure provides a compound of formula III wherein Z is N, and X or Y is N, thereby forming a compound of formula IX or X:

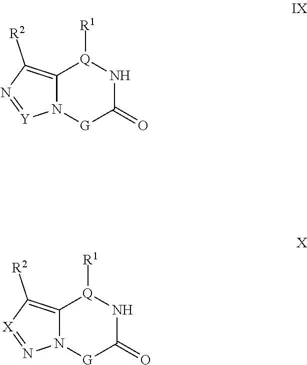

or a pharmaceutically acceptable salt thereof, wherein each of R¹, R², Q, G, X, and Y is as defined in embodiments and classes and subclasses herein.

In some embodiments, the present disclosure provides a compound of formula V wherein X or Y is CH, thereby forming a compound of formula XI or XII:

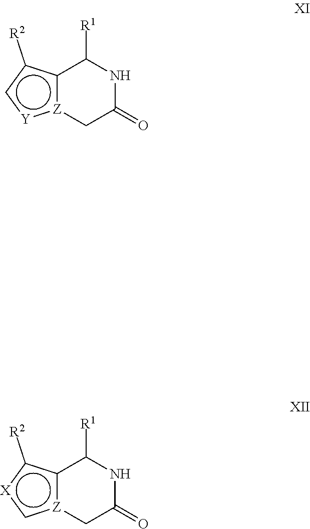

or a pharmaceutically acceptable salt thereof, wherein each of R¹, R², X, Y, and Z is as defined in embodiments and classes and subclasses herein.

In some embodiments, the present disclosure provides a compound of formula V wherein X or Y is N, thereby forming a compound of formula XIII or XIV:

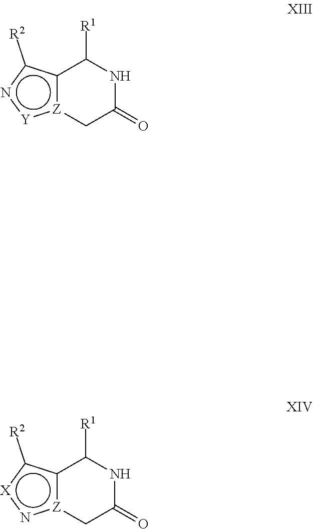

or a pharmaceutically acceptable salt thereof, wherein each of R¹, R², X, Y, and Z is as defined in embodiments and classes and subclasses herein.

In some embodiments, the present disclosure provides a compound of formula V wherein Z is N, thereby forming a compound of formula XV:

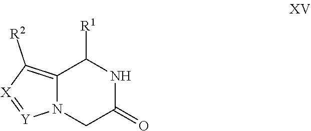

or a pharmaceutically acceptable salt thereof, wherein each of R¹, R², X, and Y is as defined in embodiments and classes and subclasses herein.

In some embodiments, the present disclosure provides a compound of formula XV, wherein X is CH or N, and Y is C(R^{Y}). In some embodiments, the present disclosure provides a compound of formula XV, wherein X is CH, and Y is C(R^{Y}). In some embodiments, the present disclosure provides a compound of formula XV, wherein X is N, and Y is C(R^{Y}).

In some embodiments, the present disclosure provides a compound of formula XI, XII, XIII, XIV, or XV, or a pharmaceutically acceptable salt thereof. In some embodiments, the present disclosure provides a compound of formula XI, XII, XIII, or XIV, or a pharmaceutically acceptable salt thereof.

In some embodiments, the present disclosure provides a compound of formula XV wherein X or Y is CH, thereby forming a compound of formula XVI or XVII:

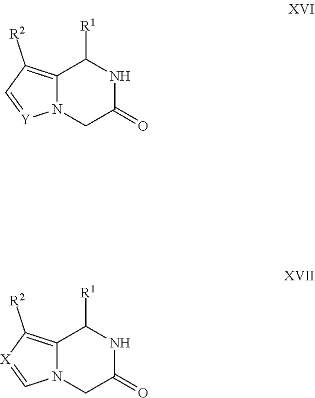

or a pharmaceutically acceptable salt thereof, wherein each of R¹, R², X, and Y is as defined in embodiments and classes and subclasses herein.

In some embodiments, the present disclosure provides a compound of formula II, wherein Z is C, and wherein the compound is of formula XVIII, XIX, or XX:

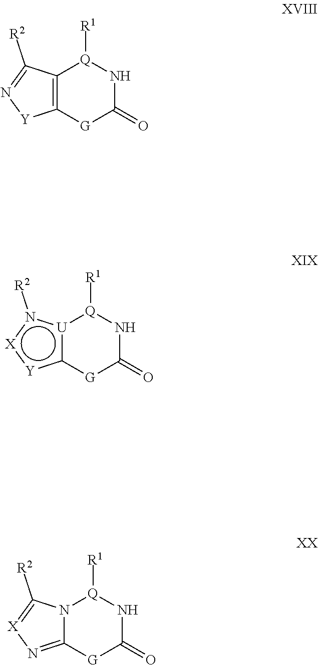

or a pharmaceutically acceptable salt thereof, wherein each of R¹, R², Q, G, U, X, and Y is as defined in embodiments and classes and subclasses herein.

In some embodiments, the present disclosure provides a compound of formula XVIII, XIX, or XX wherein G is a covalent bond, thereby forming a compound of formula XXI, XXII, or XXIII, respectively:

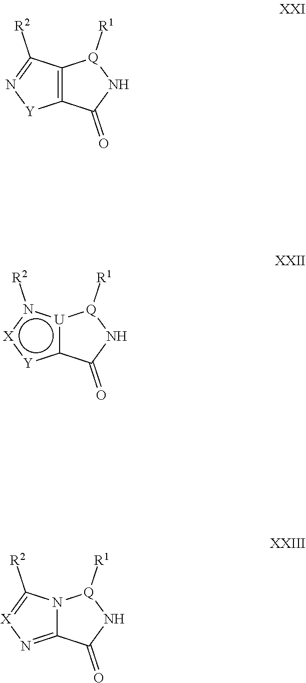

or a pharmaceutically acceptable salt thereof, wherein each of R¹, R², Q, U, X, and Y is as defined in embodiments and classes and subclasses herein.

In some embodiments, the present disclosure provides a compound of formula XXI, XXII, or XXIII, wherein Q is CH, thereby forming a compound of formula XXIV, XXV, or XXVI, respectively:

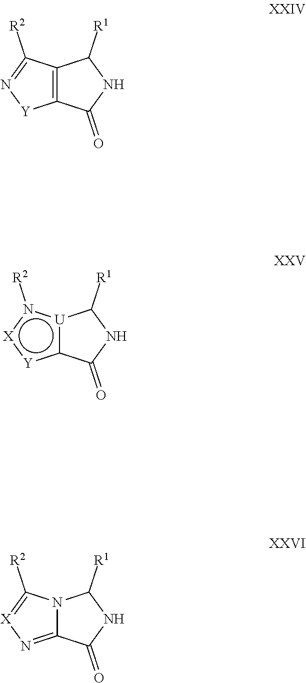

or a pharmaceutically acceptable salt thereof, wherein each of R¹, R², U, X, and Y is as defined in embodiments and classes and subclasses herein.

In some embodiments, the present disclosure provides a compound of formula I wherein E is —OC(O)—, —N(R^{E})C(O)—, or —C(R^{E})₂C(O)—, thereby forming a compound of formula XXVII, XXVIII, or XXIX, respectively:

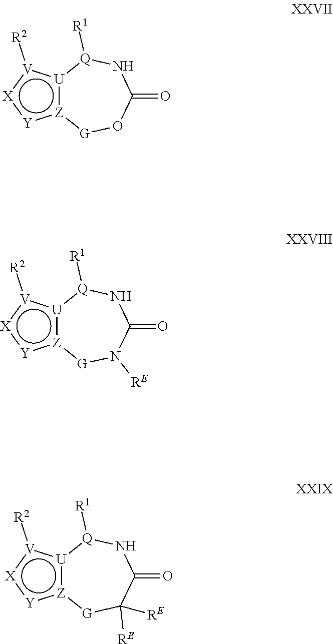

or a pharmaceutically acceptable salt thereof, wherein each of Q, R¹, R², R^{E}, G, U, V, X, Y, and Z is as defined in embodiments and classes and subclasses herein.

In some embodiments, the present disclosure provides a compound of formula XXVII, XXVIII, or XXIX wherein Q is CH, thereby forming a compound of formula XXX, XXXI, or XXXII, respectively:

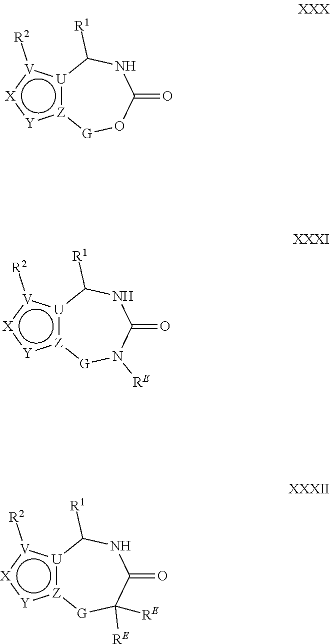

or a pharmaceutically acceptable salt thereof, wherein each of R¹, R², R^{E}, G, U, V, X, Y, and Z is as defined in embodiments and classes and subclasses herein.

In some embodiments, the present disclosure provides a compound of formula IV, V, VI, XI, XII, XIII, XIV, XV, XVI, XVII, XXIV, XXV, XXVI, XXX, XXXI, or XXXII, having the depicted stereochemistry at Q when Q is CH, thereby forming a compound of formula XXXIII, XXXIV, XXXV, XXXVI, XXXVII, XXXVIII, XXXIX, XL, XLI, XLII, XLIII, XLIV, XLV, XLVI, XLVII, or XLVIII respectively:

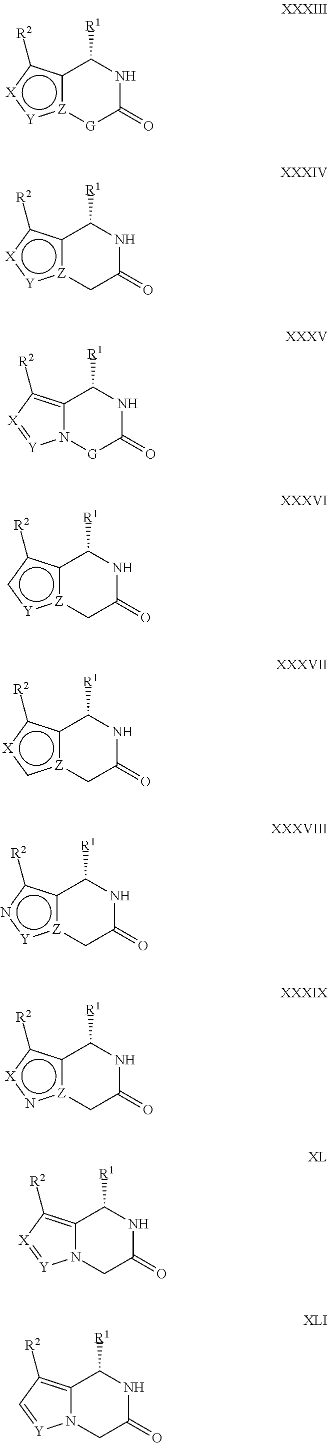

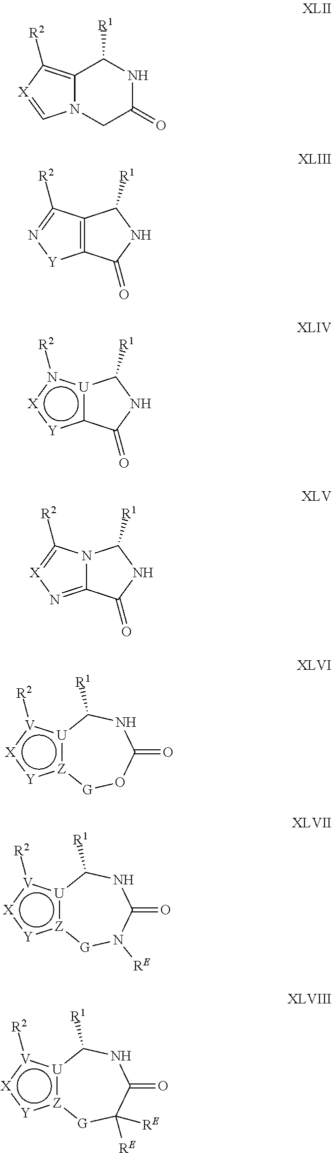

or a pharmaceutically acceptable salt thereof, wherein each of R¹, R², R^{E}, G, U, V, X, Y, and Z is as defined in embodiments and classes and subclasses herein.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —N(R)C(O)—R^{2A}, —N(R)—R^{2A}, or —R^{2A}.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —N(R)C(O)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —N(R)—R^{2A}. In some embodiments, the present disclosure provides a compound of formula II, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —R^{A}.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —N(H)C(O)—R^{2A}, —N(H)—R^{2A}, or —R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —N(H)C(O)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond, and R² is —N(H)—R^{2A}.

In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}), L¹ is a covalent bond, and R² is —N(R)C(O)—R^{2A}. In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}), L¹ is a covalent bond, and R² is —N(R)—R^{2A}. In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}), L¹ is a covalent bond, and R² is —R^{2A}.

In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}), L¹ is a covalent bond, and R² is —N(H)C(O)—R^{2A}, —N(H)—R^{2A}, or —R^{2A}. In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}), L¹ is a covalent bond, and R² is —N(H)C(O)—R^{2A}. In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}), L¹ is a covalent bond, and R² is —N(H)—R^{2A}.

In some embodiments, the present disclosure provides a compound of formula I, II, III, IV, V, VI, VII, IX, XI, XIII, XV, XVI, XVIII, XIX, XXI, XXII, XXIV, XXV, XXVII, XXVIII, XXIX, XXX, XXXI, XXXII, XXXIII, XXXIV, XXXV, XXXVI, XXXVIII, XL, XLI, XLIII, XLIV, XLVI, XLVII, or XLVIII, wherein Y is C(R^{Y}).

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein L¹ is a covalent bond (i.e. R¹ is —R^{1A}).

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —N(R)C(O)—R^{2A}, —N(R)—R^{2A}, or —R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —N(R)C(O)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —N(R)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —R^{2A}.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —N(H)C(O)—R^{2A}, —N(H)—R^{2A}, or —R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —N(H)C(O)—R^{2A}. In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, wherein R² is —N(H)—R^{2A}.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

Lengthy table referenced here
US20250019380A1-20250116-T00001
Please refer to the end of the specification for access instructions.

In chemical structures in Table 1, above, and the Examples, below, stereogenic centers are described according to the Enhanced Stereo Representation format (MDL/Biovia, e.g. using labels “or1”, “or2”, “abs”, “and1”). (See, for example, the structures of Compounds I-7, I-8, I-10, and I-11.)

In some embodiments, the present disclosure provides a compound in Table 1, above, wherein the compound is denoted as having an ADP-Glo IC₅₀ of “A”. In some embodiments, the present disclosure provides a compound in Table 1, above, wherein the compound is denoted as having an ADP-Glo IC₅₀ of “A” or “B”. In some embodiments, the present disclosure provides a compound in Table 1, above, wherein the compound is denoted as having an ADP-Glo IC₅₀ of “A” or “B” or “C”. In some embodiments, the present disclosure provides a compound in Table 1, above, wherein the compound is denoted as having an ADP-Glo IC₅₀ of “A” or “B” or “C” or “D”.

In some embodiments, the present disclosure provides a compound in Table 1, above, wherein the compound is denoted as having an MCF10A IC₅₀ of “A”. In some embodiments, the present disclosure provides a compound in Table 1, above, wherein the compound is denoted as having an MCF10A IC₅₀ of “A” or “B”. In some embodiments, the present disclosure provides a compound in Table 1, above, wherein the compound is denoted as having an MCF10A IC₅₀ of “A” or “B” or “C”. In some embodiments, the present disclosure provides a compound in Table 1, above, wherein the compound is denoted as having an MCF10A IC₅₀ of “A” or “B” or “C” or “D”.

In some embodiments, the present disclosure comprises a compound of formula I selected from those depicted in Table 1, above, or a pharmaceutically acceptable salt, stereoisomer, or mixture of stereoisomers thereof. In some embodiments, the present disclosure provides a compound of formula I selected from those depicted in Table 1, above, or a pharmaceutically acceptable salt thereof. In some embodiments, the present disclosure provides a compound of formula I selected from those depicted in Table 1, above.

In some embodiments, the present disclosure comprises a compound of formula II selected from those depicted in Table 1, above, or a pharmaceutically acceptable salt, stereoisomer, or mixture of stereoisomers thereof. In some embodiments, the present disclosure provides a compound of formula II selected from those depicted in Table 1, above, or a pharmaceutically acceptable salt thereof. In some embodiments, the present disclosure provides a compound of formula II selected from those depicted in Table 1, above.

In some embodiments, the present disclosure comprises a compound of formula III selected from those depicted in Table 1, above, or a pharmaceutically acceptable salt, stereoisomer, or mixture of stereoisomers thereof. In some embodiments, the present disclosure provides a compound of formula III selected from those depicted in Table 1, above, or a pharmaceutically acceptable salt thereof. In some embodiments, the present disclosure provides a compound of formula III selected from those depicted in Table 1, above.

In some embodiments, the present disclosure comprises a compound of formula IV selected from those depicted in Table 1, above, or a pharmaceutically acceptable salt, stereoisomer, or mixture of stereoisomers thereof. In some embodiments, the present disclosure provides a compound of formula IV selected from those depicted in Table 1, above, or a pharmaceutically acceptable salt thereof. In some embodiments, the present disclosure provides a compound of formula IV selected from those depicted in Table 1, above.

In some embodiments, the present disclosure comprises a compound of formula V selected from those depicted in Table 1, above, or a pharmaceutically acceptable salt, stereoisomer, or mixture of stereoisomers thereof. In some embodiments, the present disclosure provides a compound of formula V selected from those depicted in Table 1, above, or a pharmaceutically acceptable salt thereof. In some embodiments, the present disclosure provides a compound of formula V selected from those depicted in Table 1, above.

In some embodiments, the present disclosure comprises a compound of formula VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, selected from those depicted in Table 1, above, or a pharmaceutically acceptable salt, stereoisomer, or mixture of stereoisomers thereof. In some embodiments, the present disclosure provides a compound of formula VVI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, selected from those depicted in Table 1, above, or a pharmaceutically acceptable salt thereof. In some embodiments, the present disclosure provides a compound of formula VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII, selected from those depicted in Table 1, above.

### 4. Uses, Formulation, and Administration Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that is effective to measurably inhibit a PI3Kα protein kinase, or a mutant thereof, in a biological sample or in a patient. In certain embodiments, the amount of compound in compositions of this disclosure is such that it is effective to measurably inhibit a PI3Kα protein kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of a PI3Kα protein kinase, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y—, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is a phosphatidylinositol 3-kinase (PI3K). In some embodiments, the kinase inhibited by the compounds and compositions described herein is one or more of a PI3Kα, PI3Kδ, and PI3Kγ. In some embodiments, the kinase inhibited by the compounds and compositions described herein is a PI3Kα. In some embodiments, the kinase inhibited by the compounds and compositions described herein is a PI3Kα containing at least one of the following mutations: H1047R, E542K, and E545K.

Compounds or compositions of the disclosure can be useful in applications that benefit from inhibition of PI3K enzymes. For example, PI3K inhibitors of the present disclosure are useful for the treatment of cellular proliferative diseases generally. Compounds or compositions of the disclosure can be useful in applications that benefit from inhibition of PI3Kα enzymes. For example, PI3Kα inhibitors of the present disclosure are useful for the treatment of cellular proliferative diseases generally.

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

The activity of a compound utilized in this disclosure as an inhibitor of a PI3K kinase, for example, a PI3Kα, or a mutant thereof, may be assayed in vitro, in vivo or in a cell line. In vitro assays include assays that determine inhibition of either the phosphorylation activity and/or the subsequent functional consequences, or ATPase activity of an activated PI3Kα, or a mutant thereof. Alternative in vitro assays quantitate the ability of the inhibitor to bind to a a PI3Kα. Inhibitor binding may be measured by radiolabeling the inhibitor prior to binding, isolating the inhibitor/PI3Kα complex and determining the amount of radiolabel bound. Alternatively, inhibitor binding may be determined by running a competition experiment where new inhibitors are incubated with a PI3Kα bound to known radioligands. Representative in vitro and in vivo assays useful in assaying a PI3Kα inhibitor include those described and disclosed in the patent and scientific publications described herein. Detailed conditions for assaying a compound utilized in this disclosure as an inhibitor of a PI3Kα, or a mutant thereof, are set forth in the Examples below.

### Treatment of Disorders

Provided compounds are inhibitors of PI3Kα and are therefore useful for treating one or more disorders associated with activity of PI3Kα or mutants thereof. Thus, in certain embodiments, the present disclosure provides a method of treating a PI3Kα-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a PI3Kα-mediated disorder in a subject comprising administering a therapeutically effective amount of a compound of the present disclosure, or a pharmaceutically acceptable composition thereof, to a subject in need thereof. In some embodiments, the subject has a mutant PI3Kα. In some embodiments, the subject has PI3Kα containing at least one of the following mutations: H1047R, E542K, and E545K.

As used herein, the term “PI3Kα-mediated” disorders, diseases, and/or conditions means any disease or other deleterious condition in which PI3Kα or a mutant thereof is known to play a role. Accordingly, another embodiment of the present disclosure relates to treating or lessening the severity of one or more diseases in which PI3Kα, or a mutant thereof, is known to play a role. Such PI3Kα-mediated disorders include, but are not limited to, cellular proliferative disorders (e.g. cancer). In some embodiments, the PI3Kα-mediated disorder is a disorder mediated by a mutant PI3Kα. In some embodiments, the PI3Kα-mediated disorder is a disorder mediated by a PI3Kα containing at least one of the following mutations: H1047R, E542K, and E545K.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof, and (iii) administering said provided compound in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

In some embodiments, the method of treatment comprises the steps of: i) identifying a subject in need of such treatment; (ii) providing a composition comprising a disclosed compound, or a pharmaceutically acceptable salt thereof, and (iii) administering said composition in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cancer is ovarian cancer. In some embodiments, the ovarian cancer is clear cell ovarian cancer.

In some embodiments, the cellular proliferative disease has mutant PI3Kα. In some embodiments, the cancer has mutant PI3Kα. In some embodiments, the breast cancer has mutant PI3Kα. In some embodiments, the ovarian cancer has mutant PI3Kα.

In some embodiments, the cellular proliferative disease has PI3Kα containing at least one of the following mutations: H1047R, E542K, and E545K. In some embodiments, the cancer has PI3Kα containing at least one of the following mutations: H1047R, E542K, and E545K. In some embodiments, the breast cancer has PI3Kα containing at least one of the following mutations: H1047R, E542K, and E545K. In some embodiments, the ovarian cancer has PI3Kα containing at least one of the following mutations: H1047R, E542K, and E545K.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of PI3Kα, somatic mutation of PIK3CA, germline mutations or somatic mutation of PTEN, or mutations and translocation of p85a that serve to up-regulate the p85-p110 complex. In some embodiments, the cellular proliferative disease displays overexpression or amplification of PI3Kα. In some embodiments, the cellular proliferative disease displays somatic mutation of PIK3CA. In some embodiments, the cellular proliferative disease displays germline mutations or somatic mutation of PTEN. In some embodiments, the cellular proliferative disease displays mutations and translocation of p85a that serve to up-regulate the p85-p110 complex.

### Additional Disorders

In some embodiments, the PI3Kα-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the PI3Kα-mediated disorder is polycythemia vera, essential thrombocythemia, or myelofibrosis with myeloid metaplasia. In some embodiments, the PI3Kα-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the PI3Kα-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, or scleroderma. In some embodiments, the PI3Kα-mediated disorder is vitiligo, hypersensitivity angiitis, urticaria, bullous pemphigoid, lupus erythematosus, pemphisus, epidermolysis bullosa acquisita, or autoimmune haematogical disorders (e.g. haemolytic anaemia, aplastic anaemia, pure red cell anaemia and idiopathic thrombocytopenia). In some embodiments, the PI3Kα-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g. ulcerative colitis and Crohn's disease).

In some embodiments, the PI3Kα-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the PI3Kα-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the PI3Kα-mediated disorder is retinopathy, such as diabetic retinopathy or hyperbaric oxygen-induced retinopathy, and conditions characterized by elevated intraocular pressure or secretion of ocular aqueous humor, such as glaucoma.

### Routes of Administration and Dosage Forms

The compounds and compositions, according to the methods of the present disclosure, may be administered using any amount and any route of administration effective for treating or lessening the severity of the disorder (e.g. a proliferative disorder). The exact amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar--agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the disclosure relates to a method of inhibiting protein kinase activity in a biological sample comprising the step of contacting said biological sample with a compound of this disclosure, or a composition comprising said compound. According to another embodiment, the disclosure relates to a method of inhibiting activity of a PI3K, or a mutant thereof, in a biological sample comprising the step of contacting said biological sample with a compound of this disclosure, or a composition comprising said compound. According to another embodiment, the disclosure relates to a method of inhibiting activity of PI3Kα, or a mutant thereof, in a biological sample comprising the step of contacting said biological sample with a compound of this disclosure, or a composition comprising said compound. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting PI3Kα over one or both of PI3Kδ and PI3Kγ. In some embodiments, a compound of the present disclosure is more than 5-fold selective over PI3Kδ and PI3Kγ. In some embodiments, a compound of the present disclosure is more than 10-fold selective over PI3Kδ and PI3Kγ. In some embodiments, a compound of the present disclosure is more than 50-fold selective over PI3Kδ and PI3Kγ. In some embodiments, a compound of the present disclosure is more than 100-fold selective over PI3Kδ and PI3Kγ. In some embodiments, a compound of the present disclosure is more than 200-fold selective over PI3Kδ and PI3Kγ. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting a mutant PI3Kα over a wild-type PI3Kα. In some embodiments, a compound of the present disclosure is more than 5-fold selective for mutant PI3Kα over wild-type PI3Kα. In some embodiments, a compound of the present disclosure is more than 10-fold selective for mutant PI3Kα over wild-type PI3Kα. In some embodiments, a compound of the present disclosure is more than 50-fold selective for mutant PI3Kα over wild-type PI3Kα. In some embodiments, a compound of the present disclosure is more than 100-fold selective for mutant PI3Kα over wild-type PI3Kα. In some embodiments, a compound of the present disclosure is more than 200-fold selective for mutant PI3Kα over wild-type PI3Kα. In some embodiments, the mutant PI3Kα contains at least one of the following mutations: H1047R, E542K, and E545K.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound of the present disclosure, or a composition comprising said compound.

According to another embodiment, the disclosure relates to a method of inhibiting activity of a PI3K, or a mutant thereof, in a patient comprising the step of administering to said patient a compound of the present disclosure, or a composition comprising said compound. In some embodiments, the disclosure relates to a method of inhibiting activity of PI3Kα, or a mutant thereof, in a patient comprising the step of administering to said patient a compound of the present disclosure, or a composition comprising said compound. In some embodiments, the PI3Kα is a mutant PI3Kα. In some embodiments, the PI3Kα contains at least one of the following mutations: H1047R, E542K, and E545K.

According to another embodiment, the present disclosure provides a method for treating a disorder mediated by a PI3K, or a mutant thereof, in a patient in need thereof, comprising the step of administering to said patient a compound according to the present disclosure or pharmaceutically acceptable composition thereof. In some embodiments, the present disclosure provides a method for treating a disorder mediated by PI3Kα, or a mutant thereof, in a patient in need thereof, comprising the step of administering to said patient a compound according to the present disclosure or pharmaceutically acceptable composition thereof. In some embodiments, the PI3Kα is a mutant PI3Kα. In some embodiments, the PI3Kα contains at least one of the following mutations: H1047R, E542K, and E545K.

According to another embodiment, the present disclosure provides a method of inhibiting signaling activity of PI3Kα, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting PI3Kα signaling activity in a subject, comprising administering a therapeutically effective amount of a compound according to the present disclosure, or a pharmaceutically acceptable composition thereof, to a subject in need thereof. In some embodiments, the PI3Kα is a mutant PI3Kα. In some embodiments, the PI3Kα contains at least one of the following mutations: H1047R, E542K, and E545K. In some embodiments, the subject has a mutant PI3Kα. In some embodiments, the subject has PI3Kα containing at least one of the following mutations: H1047R, E542K, and E545K.

The compounds described herein can also inhibit PI3Kα function through incorporation into agents that catalyze the destruction of PI3Kα. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of PI3Kα to the E3 ligase will thus result in the destruction of the PI3Kα protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Additionally, PI3K serves as a second messenger node that integrates parallel signaling pathways, and evidence is emerging that the combination of a PI3K inhibitor with inhibitors of other pathways will be useful in treating cancer and cellular proliferative diseases.

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI13K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)). Accordingly, in certain embodiments, the method of treatment comprises administering the compound or composition of the disclosure in combination with trastuzumab. In certain embodiments, the cancer is a human breast cancer that overexpresses Her-2/neu-ErbB2.

A variety of human malignancies express activating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway.

For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Accordingly, in certain embodiments, the method of treatment comprises administering the compound or composition of the disclosure in combination with an inhibitor of Her1/EGFR. In certain embodiments, the method of treatment comprises administering the compound or composition of the disclosure in combination with one or more of tarceva, gefitinib, and erbitux. In certain embodiments, the method of treatment comprises administering the compound or composition of the disclosure in combination with gefitinib. In certain embodiments, the cancer expresses activating mutations or increased levels of Her1/EGFR.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Aid also abrogates its role to arrest the cell cycle (Viglietto et al., Nat. Med. 8:1145 (2002)).

Accordingly, in certain embodiments, the method of treatment comprises administering the compound or composition of the disclosure in combination with a treatment for a hormone-dependent cancer. In certain embodiments, the method of treatment comprises administering the compound or composition of the disclosure in combination with tamoxifen. In certain embodiments, the cancer is a hormone dependent cancer, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Abl employs the Ras-Raf kinase pathway to elicit its effects. In addition, inhibiting more than one kinase in the same pathway provides additional protection against resistance-conferring mutations.

Accordingly, in another aspect, the compounds and compositions of the disclosure are used in combination with at least one additional agent selected from the group of kinase inhibitors, such as imatinib, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In some embodiments, the one or more additional therapeutic agents is selected from antibodies, antibody-drug conjugates, kinase inhibitors, immunomodulators, and histone deacetylase inhibitors. Synergistic combinations with PIK3CA inhibitors and other therapeutic agents are described in, for example, Castel et al., Mol. Cell Oncol. (2014)1(3) e963447.

In some embodiments, the one or more additional therapeutic agent is selected from the following agents, or a pharmaceutically acceptable salt thereof: BCR-ABL inhibitors (see e.g. Ultimo et al. Oncotarget (2017) 8 (14) 23213-23227.): e.g. imatinib, inilotinib, nilotinib, dasatinib, bosutinib, ponatinib, bafetinib, danusertib, saracatinib, PF03814735; ALK inhibitors (see e.g. Yang et al. Tumour Biol. (2014) 35 (10) 9759-67): e.g. crizotinib, NVP-TAE684, ceritinib, alectinib, brigatinib, entrecinib, lorlatinib; BRAF inhibitors (see e.g. Silva et al. Mol. Cancer Res. (2014) 12, 447-463): e.g. vemurafenib, dabrafenib; FGFR inhibitors (see e.g. Packer et al. Mol. Cancer Ther. (2017) 16(4) 637-648): e.g. infigratinib, dovitinib, erdafitinib, TAS-120, pemigatinib, BLU-554, AZD4547; FLT3 inhibitors: e.g. sunitinib, midostaurin, tanutinib, sorafenib, lestaurtinib, quizartinib, and crenolanib; MEK Inhibitors (see e.g. Jokinen et al. Ther. Adv. Med. Oncol. (2015) 7(3) 170-180): e.g. trametinib, cobimetinib, binimetinib, selumetinib; ERK inhibitors: e.g. ulixertinib, MK 8353, LY 3214996; KRAS inhibitors: e.g. AMG-510, MRTX849, ARS-3248; Tyrosine kinase inhibitors (see e.g. Makhov et al. Mol. Cancer. Ther. (2012) 11(7) 1510-1517): e.g. erlotinib, linifanib, sunitinib, pazopanib; Epidermal growth factor receptor (EGFR) inhibitors (see e.g. She et al. BMC Cancer (2016) 16, 587): gefitnib, osimertinib, cetuximab, panitumumab; HER2 receptor inhibitors (see e.g. Lopez et al. Mol. Cancer Ther. (2015) 14(11) 2519-2526): e.g. trastuzumab, pertuzumab, neratinib, lapatinib, lapatinib; MET inhibitors (see e.g. Hervieu et al. Front. Mol. Biosci. (2018) 5, 86): e.g. crizotinib, cabozantinib; CD20 antibodies: e.g. rituximab, tositumomab, ofatumumab; DNA Synthesis inhibitors: e.g. capecitabine, gemcitabine, nelarabine, hydroxycarbamide; Antineoplastic agents (see e.g. Wang et al. Cell Death & Disease (2018) 9, 739): e.g. oxaliplatin, carboplatin, cisplatin; Immunomodulators: e.g. afutuzumab, lenalidomide, thalidomide, pomalidomide; CD40 inhibitors: e.g. dacetuzumab; Pro-apoptotic receptor agonists (PARAs): e.g. dulanermin; Heat Shock Protein (HSP) inhibitors (see e.g. Chen et al. Oncotarget (2014) 5 (9). 2372-2389): e.g. tanespimycin; Hedgehog antagonists (see e.g. Chaturvedi et al. Oncotarget (2018) 9 (24), 16619-16633): e.g. vismodegib; Proteasome inhibitors (see e.g. Lin et al. Int. J. Oncol. (2014) 44 (2), 557-562): e.g. bortezomib; PI3K inhibitors: e.g. pictilisib, dactolisib, alpelisib, buparlisib, taselisib, idelalisib, duvelisib, umbralisib; SHP2 inhibitors (see e.g. Sun et al. Am. J. Cancer Res. (2019) 9 (1), 149-159: e.g. SHP099, RMC-4550, RMC-4630); BCL-2 inhibitors (see e.g. Bojarczuk et al. Blood (2018) 133 (1), 70-80): e.g. venetoclax; Aromatase inhibitors (see e.g. Mayer et al. Clin. Cancer Res. (2019) 25 (10), 2975-2987): exemestane, letrozole, anastrozole, fulvestrant, tamoxifen; mTOR inhibitors (see e.g. Woo et al. Oncogenesis (2017) 6, e385): e.g. temsirolimus, ridaforolimus, everolimus, sirolimus; CTLA-4 inhibitors (see e.g. O'Donnell et al. (2018) 48, 91-103): e.g. tremelimumab, ipilimumab; PD1 inhibitors (see O'Donnell, supra): e.g. nivolumab, pembrolizumab; an immunoadhesin; Other immune checkpoint inhibitors (see e.g. Zappasodi et al. Cancer Cell (2018) 33, 581-598, where the term “immune checkpoint” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta): e.g. pidilizumab, AMP-224; PDL1 inhibitors (see e.g. O'Donnell supra): e.g. MSB0010718C; YW243.55.570, MPDL3280A; MEDI-4736, MSB-0010718C, or MDX-1105; Histone deacetylase inhibitors (HDI, see e.g. Rahmani et al. Clin. Cancer Res. (2014) 20(18), 4849-4860): e.g. vorinostat; Androgen Receptor inhibitors (see e.g. Thomas et al. Mol. Cancer Ther. (2013) 12(11), 2342-2355): e.g. enzalutamide, abiraterone acetate, orteronel, galeterone, seviteronel, bicalutamide, flutamide; Androgens: e.g. fluoxymesterone; CDK4/6 inhibitors (see e.g. Gul et al. Am. J. Cancer Res. (2018) 8(12), 2359-2376): e.g. alvocidib, palbociclib, ribociclib, trilaciclib, abemaciclib.

In some embodiments, the one or more additional therapeutic agent is selected from the following agents: anti-FGFR antibodies; FGFR inhibitors, cytotoxic agents; Estrogen Receptor-targeted or other endocrine therapies, immune-checkpoint inhibitors, CDK inhibitors, Receptor Tyrosine Kinase inhibitors, BRAF inhibitors, MEK inhibitors, other PI3K inhibitors, SHP2 inhibitors, and SRC inhibitors. (See Katoh, Nat. Rev. Clin. Oncol. (2019), 16:105-122; Chae, et al. Oncotarget (2017), 8:16052-16074; Formisano et al., Nat. Comm. (2019), 10:1373-1386; and references cited therein.)

The structure of the active compounds identified by code numbers, generic or trade names may be taken from the actual edition of the standard compendium “The Merck Index” or from databases, e.g. Patents International (e.g. IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### List of Abbreviations

aq: aqueous
Ac: acetyl
ACN or MeCN: acetonitrile
AmF: ammonium formate
anhyd.: anhydrous
BINAP: (±)-2,2′-Bis(diphenylphosphino)-1,1′-binaphthalene
Bn: Benzyl
conc.: concentrated
DBU: 1,8-Diazabicyclo[5.4.0]undec-7-ene
DCE: Dichloroethane
DCM: Dichloromethane
DIPEA: Diisopropylamine
DMF: N,N-dimethylformamide
DMP: Dess-Martin periodinane
DMPU: N,N′-Dimethylpropyleneurea
DMSO: dimethylsulfoxide
DIPEA: diisopropylethylamine
EA or EtOAc: ethyl acetate
EDCI, EDC, or EDAC: 1-Ethyl-3-(3-dimethylaminopropyl)carbodiimide
equiv or eq: molar equivalents
Et: ethyl
HATU: 1-[Bis(dimethylamino)methylene]-1H-1,2,3-triazolo[4,5-b]pyridinium 3-oxid Hexafluorophosphate
HPLC: high pressure liquid chromatography
LCMS or LC-MS: liquid chromatography-mass spectrometry
Ms: methanesulfonyl
NBS: N-bromosuccinimide
NMR: nuclear magnetic resonance
PE: petroleum ether
PMB: p-methoxybenzyl
rt or RT: room temperature
sat: saturated
TBS: tert-butyldimethylsilyl
TEA: triethylamine
Tf: trifluoromethanesulfonate
TFA: trifluoroacetic acid
THF: tetrahydrofuran
TLC: thin layer chromatography
Tol: toluene
UV: ultra violet

### LC-MS Methods

The following methods were used for LC-MS analysis:
Method A: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.3 m/min of a binary gradient consisting of water+0.05% trifluoroacetic acid (A) and acetonitrile+0.05% trifluoroacetic acid (B). The retention times (RT) are expressed in minutes based on the UV-trace at 254 nm. Gradient: 0.01 min 5% B, 1.20 min 100% B, 1.80 min 100% B, 1.82 min 5% B. Total run time: 2.0 min.
Method B: The analytical LC-MS system is equipped with Shimadzu LCMS-2020, PDA detector (operating at 220 nm), ELSD detector, and ESI-source operating in positive ion mode. LC-conditions: The column is HALO C18 30*3.0 mm, 2 μm, operating at 40° C. with 1.3 m/min of a binary gradient consisting of water+0.05% trifluoroacetic acid (A) and acetonitrile+0.05% trifluoroacetic acid (B). The retention times (RT) are expressed in minutes based on UV-trace at 220 nm. Gradient: 0.01 min 5% B, 1.20 min 100% B, 1.80 min 100% B, 1.82 min 5% B. Total run time: 2.0 min.
Method C: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.3 m/min of a binary gradient consisting of water+0.05% trifluoroacetic acid (A) and acetonitrile+0.05% trifluoroacetic acid (B). The retention times (RT) are expressed in minutes based on UV-trace at 254 nm. Gradient: 0.01 min 5% B, 0.70 min 100% B, 1.10 min 100% B, 1.12 min 5% B. Total run time: 1.2 min.
Method D: Waters Acquity UPLC CSH C18, 1.8 μm, 2.1×30 mm at 40° C.; 5% to 100% B in 2.0 minutes; hold 100% B for 0.7 minute, total run time=2.7 min; Eluents: A=Milli-Q H₂O+10 mM ammonium formate (pH 3.8); B=acetonitrile. Waters Acquity H-Class UPLC system. UV Detector=Waters Acquity PDA, 195-360 nm. MS Detector=Acquity QDa Performance ESI.
Method E: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.5 m/min of a binary gradient consisting of water+0.05% trifluoroacetic acid (A) and acetonitrile+0.05% trifluoroacetic acid (B). The retention times (RT) are expressed in minutes based on UV-trace at 254 nm. Gradient: 0.01 min 5% B, 1.20 min 100% B, 1.80 min 100% B, 1.82 min 5% B. Total run time: 2.0 min.
Method F: The analytical LC-MS system is equipped with Shimadzu LCMS-2020, PDA detector (operating at 254 nm), ELSD detector, and ESI-source operating in positive ion mode. LC-conditions: The column is Kinetex EVO C18 50*3.0 mm, 2.6 μm, operating at 40° C. with 1.2 mL/min of a binary gradient consisting of water+6.5 mM NH₄HCO₃+ammonia (pH=10) (A) and acetonitrile (B). The retention times (RT) are expressed in minutes based on UV-trace at 254 nm. Gradient: 0.01 min 10% B, 2.20 min 50% B, 2.70 min 95% B, 3.20 min 95% B, 3.30 min 10% B. Total run time: 3.5 min.
Method G: Column: Acquity CSH C18, 2.1×30 mm, 1.7 μm particles; Solvent A=0.1% formic acid in water. Solvent B=0.1% formic acid in acetonitrile. Flow rate=0.8 mL/min. Column temp: 40° C. Gradient: B=5% to 95%. Gradient Time=1.7 min, then a 0.2 min hold at 95% B. Wavelength=215 and 254 nm. ESI+ Range: 150 to 1500 Dalton. System: Agilent 1290 Infinity II LCMS.
Method H: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.2 m/min of a binary gradient consisting of water+0.1% formic acid (A) and acetonitrile+0.1% formic acid (B). The retention times (RT) are expressed in minutes based on UV-trace at 254 nm. Gradient: 0.01 min 5% B, 1.70 min 50% B, 2.30 min 100% B, 2.80 min 100% B, 2.83 min 5% B. Total run time: 3.0 min.
Method I: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.5 m/min of a binary gradient consisting of water+0.1% formic acid (A) and acetonitrile+0.1% formic acid (B). The retention times (RT) are expressed in minutes based on UV-trace at 254 nm. Gradient: 0.01 min 5% B, 1.20 min 100% B, 1.80 min 100% B, 1.82 min 5% B. Total run time: 2.0 min.
Method J: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.5 m/min of a binary gradient consisting of water+0.1% formic acid (A) and acetonitrile+0.1% formic acid (B). The retention times (RT) are expressed in minutes based on UV-trace at 254 nm. Gradient: 0.01 min 5% B, 0.70 min 100% B, 1.10 min 100% B, 1.12 min 5% B. Total run time: 1.2 min.
Method K: The analytical LC-MS system is equipped with Shimadzu LCMS-2020, PDA detector (operating at 254 nm), ELSD detector, and ESI-source operating in positive ion mode. LC-conditions: The column is HALO C18 30*3.0 mm, 2 μm, operating at 40° C. with 1.2 m/min of a binary gradient consisting of water+0.1% formic acid (A) and acetonitrile+0.1% formic acid (B). The retention times (RT) are expressed in minutes based on UV-trace at 254 nm. Gradient: 0.01 min 5% B, 1.20 min 100% B, 1.80 min 100% B, 1.82 min 5% B. Total run time: 2.0 min.
Method L: Waters Acquity UPLC CSH C18, 1.8 μm, 2.1×30 mm at 40° C.; 5% to 100% B in 5.2 minutes; hold 100% B for 1.8 minute, total run time=7.0 min; Eluents: A=Milli-Q H₂O+10 mM ammonium formate (pH=3.8); B=acetonitrile. Waters Acquity H-Class UPLC system. UV Detector=Waters Acquity PDA, 195-360 nm. MS Detector=Acquity QDa Performance ESI.
Method M: The analytical LC-MS system is equipped with Shimadzu LCMS-2020, PDA detector (operating at 254 nm), ELSD detector, and ESI-source operating in positive ion mode. LC-conditions: The column is Kinetex EVO C18 50*3.0 mm, 2.6 μm, operating at 40° C. with 1.2 mL/min of a binary gradient consisting of water+6.5 mM NH₄HCO₃+ammonia (pH=10) (A) and acetonitrile (B). The retention times (RT) are expressed in minutes based on UV-trace at 254 nm. Gradient: 0.01 min 10% B, 2.00 min 95% B, 2.70 min 95% B, 2.75 min 10% B. Total run time: 3.0 min.
Method N: The analytical LC-MS system is equipped with Shimadzu LCMS-2020, PDA detector (operating at 254 nm), ELSD detector, and ESI-source operating in positive ion mode. LC-conditions: The column is Shim-pack Scepter C18-120, 33*3.0 mm, 3 μm, operating at 30° C. with 1.5 mL/min of a binary gradient consisting of water+5 mM NH₄HCO₃ (A) and acetonitrile (B). The retention times (RT) are expressed in minutes based on UV-trace at 254 nm. Gradient: 0.01 min 10% B, 1.70 min 70% B, 2.30 min 95% B, 2.80 min 95% B, 2.83 min 10% B. Total run time: 3.0 min.
Method O: The analytical LC-MS system is equipped with Shimadzu LCMS-2020, PDA detector (operating at 254 nm), ELSD detector, and ESI-source operating in positive ion mode. LC-conditions: The column is Shim-pack Scepter C18-120, 33*3.0 mm, 3 μm, operating at 30° C. with 1.5 mL/min of a binary gradient consisting of water+5 mM NH₄HCO₃ (A) and acetonitrile (B). The retention times (RT) are expressed in minutes based on UV-trace at 220 nm. Gradient: 0.01 min 10% B, 0.70 min 95% B, 1.10 min 95% B, 1.12 min 10% B. Total run time: 1.2 min.
Method P: The analytical LC-MS system is equipped with Shimadzu LCMS-2020, PDA detector (operating at 254 nm), ELSD detector, and ESI-source operating in positive ion mode. LC-conditions: The column is Shim-pack Scepter C18-120, 33*3.0 mm, 3 μm, operating at 30° C. with 1.5 mL/min of a binary gradient consisting of water+5 mM NH₄HCO₃ (A) and acetonitrile (B). The retention times (RT) are expressed in minutes based on UV-trace at 254 nm. Gradient: 0.01 min 10% B, 1.20 min 95% B, 1.80 min 95% B, 1.82 min 10% B. Total run time: 2.0 min.
Method Q: Waters Alliance UPLC CSH C18, 3.5 μm, 4.6×30 mm at 40° C.; 5% B for 0.2 min, 5% to 100% B in 1.8 minutes; hold 100% B for 1 minute, total run time=3.0 min, flow 3 mL/min; Eluents: A=Milli-Q H₂O+10 mM ammonium formate (pH 3.8); B=acetonitrile. Waters Alliance HPLC system. UV Detector=Waters 2996 PDA, 198-360 nm. MS Detector=Waters ZQ 2000.
Method R: Waters Alliance UPLC CSH C18, 3.5 μm, 4.6×30 mm at 40° C.; 5% B for 0.5 min, 5% to 100% B in 5.0 minutes; hold 100% B for 1.5 minute, total run time=7.0 min, flow 3 mL/min; Eluents: A=Milli-Q H₂O+10 mM ammonium formate (pH 3.8); B=acetonitrile. Waters Alliance HPLC system. UV Detector=Waters 2996 PDA, 198-360 nm. MS Detector=Waters ZQ 2000.
Method S: Column: Waters Acquity UPLC CSH C18, 1.8 μm, 2.1×30 mm at 40° C.; Gradient: 5% to 100% B in 2.0 minutes; hold 100% B for 0.7 minute; total run time: 2.7 min; flow 0.9 mL/min; Eluents: A=Milli-Q H₂O+10 mM ammonium formate (pH 3.8); Eluent B: acetonitrile; Waters UPLC system equipped with: UV Detector=Waters Acquity PDA (198-360 nm). MS Detector=Waters 3100, ESI (ES+/ES−, 120-1200 amu).
Method T: Column: Waters Acquity UPLC CSH C18, 1.8 μm, 2.1×30 mm at 40° C.; Gradient: 5% B for 0.2 min, 5 to 100% B in 5.0 minutes; hold 100% B for 1.8 minute; total run time: 7.0 min; flow 0.9 mL/min; Eluents: Milli-Q H₂O+10 mM ammonium formate (pH 3.8); Eluent B: acetonitrile; Waters UPLC system equipped with: UV Detector=Waters Acquity PDA (198-360 nm). MS Detector=Waters 3100, ESI (ES+/ES−, 120-1200 amu).
Method U: Column: Waters Acquity UPLC CSH C18, 1.8 μm, 2.1×30 mm at 40° C.; Gradient: 5% to 100% B in 2.0 minutes; hold 100% B for 0.7 minute; runtime: 2.7 min; flow 0.9 mL/min; Eluents: A=Milli-Q H₂O+10 mM ammonium formate (pH 3.8); Eluent B: acetonitrile; Waters UPLC system equipped with: UV Detector=Waters Acquity PDA (198-360 nm), 220 and 254 nm. MS Detector Waters SQD, ESI (ES+/ES−, 120-1200 amu).
Method V: Column: Waters Acquity UPLC CSH C18, 1.8 μm, 2.1×30 mm at 40° C.; Gradient: 5% to 100% B in 5.2 minutes; hold 100% B for 1.8 minutes, total run time=7.0 min, flow 0.9 mL/min; Eluents: A=Milli-Q H₂O+10 mM ammonium formate (pH 3.8); Eluent B: acetonitrile; Waters HPLC system equipped with Waters Acquity UPLC. UV Detector=Waters Acquity PDA (198-360 nm). MS Detector=Waters SQD, ESI (ES+/ES−, 120-1200 amu).
Method W: Column: Waters Acquity UPLC CSH C18, 1.8 μm, 2.1×30 mm at 40° C.; Gradient: 5% to 100% B in 5.2 minutes; hold 100% B for 1.8 minutes, total run time=7.0 min, flow 0.9 mL/min; Eluents: A=Milli-Q H₂O+10 mM ammonium bicarbonate (pH 10); Eluent B: Acetonitrile (no additive); Waters HPLC system equipped with Waters Acquity UPLC. UV Detector=Waters Acquity PDA (198-360 nm). MS Detector=Waters SQD, ESI (ES+/ES−, 120-1200 amu).
Method X: Column: Kinetex EVO C18 30*2.1 mm, 5 μm C18 at 50° C.; Gradient: 0% to 60% B in 0.8 minute; hold 60% B for 0.4 minute, then 0% B, total run time=1.6 min, flow rate 1.5 mL/min; Eluents: A=H₂O+0.0375% TFA; Eluent B: Acetonitrile+0.01875% TFA; SHIMADZU LCMS-2020. UV Detector=PDA (220 & 254 nm). ESI (ES+, 100-1000 amu).
Method Y: Column: Kinetex EVO C18 30*2.1 mm, 5 μm C18 at 50° C.; Gradient: 5% to 95% B in 0.8 minute; hold 95% B for 0.4 minute, then 5% B, total run time=1.6 min, flow rate 1.5 mL/min; Eluents: A=H₂O+0.0375% TFA; Eluent B: Acetonitrile+0.01875% TFA; SHIMADZU LCMS-2020. UV Detector=PDA (220 & 254 nm). ESI (ES+, 100-1000 amu).
Method Z: Column: Kinetex EVO C18 30*2.1 mm, 5 μm C18 at 40° C.; Gradient: 90% B isocratic, flow rate 1.5 mL/min; Eluents: A=H₂O+0.025% TFA; Eluent B: Acetonitrile+0.01875% TFA; SHIMADZU LCMS-2020. UV Detector=PDA (220 & 254 nm). ESI (ES+, 100-1000 amu).

### Example 1

### Ethyl (S)-1-amino-8-(2-chloro-5-fluorophenyl)-6-oxo-5,6,7,8-tetrahydroimidazo[1,5-a]pyrazine-3-carboxylate (Intermediate I)

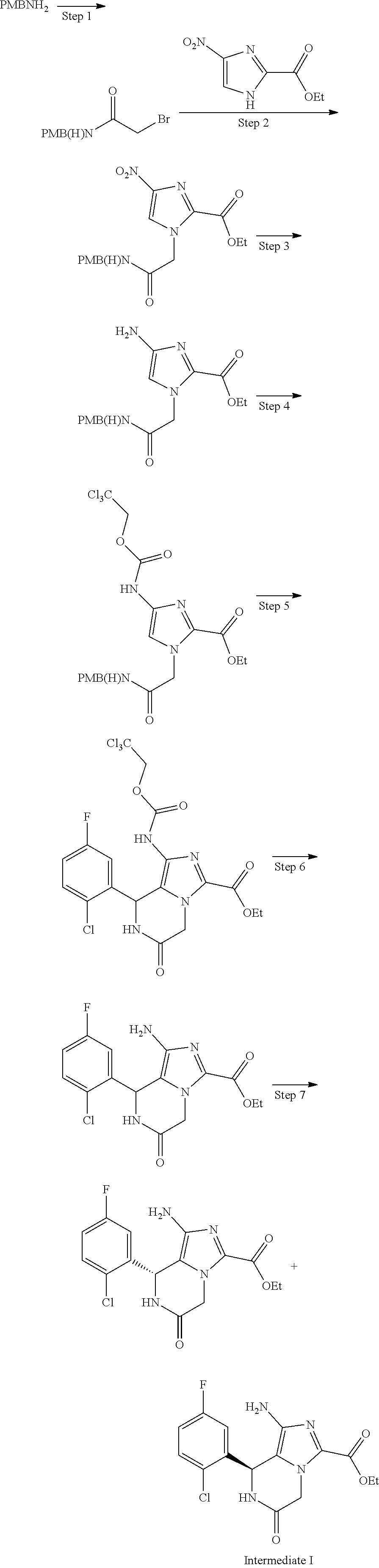

### Step 1. 2-bromo-N-(4-methoxybenzyl)acetamide

Three batches were run in parallel. To a solution of PMBNH₂ (180 g) in dichloromethane (1.80 L) was added Et₃N (239 g) at 25° C. The mixture was cooled to 0° C. To the mixture bromoacetyl bromide (291 g) was added dropwise at −10 to 0° C. The mixture was stirred at −10 to 0° C. for 0.5 hour. The three batches were combined. The mixture was poured into water (10.0 L) and extracted with dichloromethane (2.00 L*2). The combined organic phase was washed with 1 N HCl (2.00 L), saturated NaHCO₃ (3.00 L), brine (3.00 L), dried over Na₂SO₄, filtered, and concentrated to give 2-bromo-N-(4-methoxybenzyl)acetamide as a brown solid (911 g). The crude product was used in the next step without purification. ¹H NMR: (400 MHz CDCl₃) δ 7.23 (d, J=8.8 Hz, 2H), 6.89 (d, J=8.8 Hz, 2H), 9.71 (s, 1H), 4.42 (d, J=5.6 Hz, 2H), 3.92 (s, 2H), 3.81 (s, 3H).

### Step 2. ethyl 1-(2-((4-methoxybenzyl)amino)-2-oxoethyl)-4-nitro-1H-imidazole-2-carboxylate

Three batches were run in parallel. To a solution of ethyl 4-nitro-1H-imidazole-2-carboxylate (100 g) in acetonitrile (1.20 L) was added 2-tert-butyl-1,1,3,3-tetramethylguanidine (106 g). The mixture was stirred at 25° C. for 10 minutes. 2-Bromo-N-(4-methoxybenzyl)acetamide (223 g) was added to the mixture. The reaction mixture was stirred at 25° C. for 30 hours. Three batches of the mixture were combined and poured into water (18.0 L). The suspension was filtered, and the filter cake was washed with water (1.00 L) and petroleum ether (1.00 L). The filter cake was triturated with acetonitrile (600 mL). The suspension was filtered, and the filter cake was washed with acetonitrile (200 mL) and petroleum ether (1.00 L). The filter cake was dried under vacuum to give the desired product as an off-white solid (528 g, purity: 42.9% at 220 nm). The crude product was used in the next step without further purification. ¹H NMR: (400 MHz DMSO-d₆) δ 8.73 (t, J=5.6 Hz, 1H), 8.63 (s, 1H), 7.21 (d, J=8.4 Hz, 2H), 6.90 (d, J=8.4 Hz, 2H), 5.17 (s, 2H), 4.34-4.28 (m, 2H), 4.25 (d, J=5.6 Hz, 2H), 3.74 (s, 3H), 1.29 (t, J=7.2 Hz, 3H).

### Step 3. ethyl 4-amino-1-(2-((4-methoxybenzyl)amino)-2-oxoethyl)-1H-imidazole-2-carboxylate

Four batches were run in parallel. To a solution of ethyl 1-(2-((4-methoxybenzyl) amino)-2-oxoethyl)-4-nitro-1H-imidazole-2-carboxylate (100 g) in MeOH (1.30 L) was added 10% Pd on carbon (10.0 g) under a N₂ atmosphere. Then the mixture was stirred at 25° C. for 15 hours under H₂ (50 psi). The four batches of reaction mixture were combined. The mixture was diluted with methanol (18.0 L) and dichloromethane (18.0 L) and filtered. The mother liquor was concentrated to give the desired product as a green solid (326 g), which was used in the next step without purification. LCMS: RT 0.820 min, [M+H]⁺ 333.1, LCMS method X. ¹H NMR: (400 MHz DMSO-d₆) δ 8.44 (t, J=6.0 Hz, 1H), 7.19 (d, J=8.8 Hz, 2H), 6.87 (d, J=8.8 Hz, 2H), 6.47 (s, 1H), 4.92 (s, 2H), 4.59 (s, 2H), 4.20 (d, J=5.6 Hz, 2H), 4.19-4.13 (m, 2H), 3.72 (s, 3H), 1.23 (t, J=7.2 Hz, 3H).

### Step 4. ethyl 1-(2-((4-methoxybenzyl)amino)-2-oxoethyl)-4-(((2,2,2-trichloroethoxy)carbonyl)amino)-1H-imidazole-2-carboxylate

Four batches were run in parallel. To a solution of ethyl 4-amino-1-(2-((4-methoxybenzyl)amino)-2-oxoethyl)-1H-imidazole-2-carboxylate (125 g, 93.0% purity) and DIEA (136 g) in THF (1.25 L) was added 2,2,2-trichloroethyl carbonochloridate (96.3 g) at 0 to 10° C. The mixture was stirred at 0° C. for 0.5 hour. Then the mixture was warmed to 25° C. and stirred at 25° C. for 2 hours. The four batches of reaction mixture were combined. The combined mixture was poured into water (10.0 L), diluted with tetrahydrofuran (8.00 L) and extracted with ethyl acetate (8.00 L*2). The combined organic phase was washed with brine (4.00 L), dried over Na₂SO₄, and concentrated. The residue was diluted with petroleum ether:ethyl acetate=10:1 (1.50 L) and filtered. The filter cake was washed with petroleum ether (1.00 L) and dried under vacuum to give the desired product as an off-white solid (630 g), which was used in the next step without purification. LCMS: RT=0.905 min, [M+H]⁺ 507.1, LCMS method Y. ¹H NMR: (400 MHz DMSO-d₆) δ 10.6 (s, 1H), 8.56 (t, J=6.0 Hz, 1H), 7.40 (s, 1H), 7.20 (d, J=8.8 Hz, 2H), 6.87 (d, J=8.4 Hz, 2H), 5.07 (s, 2H), 4.93 (s, 2H), 4.25-4.19 (m, 4H), 3.73 (s, 3H), 1.25 (t, J=7.2 Hz, 3H).

### Step 5. ethyl 8-(2-chloro-5-fluorophenyl)-6-oxo-1-(((2,2,2-trichloroethoxy)carbonyl)amino)-5,6,7,8-tetrahydroimidazo[1,5-a]pyrazine-3-carboxylate

Three batches were run in parallel. To a solution of ethyl 1-(2-((4-methoxybenzyl)amino)-2-oxoethyl)-4-(((2,2,2-trichloroethoxy)carbonyl)amino)-1H-imidazole-2-carboxylate (220 g) in Eaton's reagent (3.04 kg) was added 2-chloro-5-fluorobenzaldehyde (132 g) at 25° C. The mixture was heated at 80° C. for 5 hours. The mixture was cooled to 25° C. Then the three batches of mixture were combined. The mixture was poured into cold ice water (15.0 L) and tetrahydrofuran:ethyl acetate (1:1, 15.0 L). Then the aqueous phase was extracted with ethyl acetate (10.0 L). The combined organic phase was washed with saturated NaHCO₃ solution (5.00 L), brine (5.00 L), dried over Na₂SO₄, filtered, and concentrated. The residue was purified by column chromatography (SiO₂, petroleum ether: ethyl acetate=10:1 to 4:1) to give the desired product (420 g) as a brown solid. LCMS: RT 1.066 min, [M+H]⁺ 528.8, LCMS method X. ¹HNMR: (400 MHz DMSO-d₆) δ 9.65 (s, 1H), 8.96 (s, 1H), 7.49-7.45 (m, 1H), 7.26-7.16 (m, 2H), 6.07 (s, 1H), 5.14-5.05 (m, 2H), 4.81-4.72 (m, 2H), 4.35-4.29 (m, 2H), 1.32 (t, J=7.6 Hz, 3H).

### Step 6. ethyl 1-amino-8-(2-chloro-5-fluorophenyl)-6-oxo-5,6,7,8-tetrahydroimidazo[1,5-a]pyrazine-3-carboxylate

Three batches were run in parallel. To a mixture of ethyl 8-(2-chloro-5-fluorophenyl)-6-oxo-1-(((2,2,2-trichloroethoxy)carbonyl)amino)-5,6,7,8-tetrahydroimidazo[1,5-a]pyrazine-3-carboxylate (140 g) in acetic acid (1.40 L) was added zinc powder (109 g) at 25° C. Then the mixture was stirred at 25° C. for 18 hours. Three batches of the reaction mixture were combined. The mixture was diluted with THF (35.0 L) and filtered. The pH of the filtrate was adjusted to 6-7 with saturated NaHCO₃ solution. The aqueous phase was extracted with ethyl acetate (5.00 L*2). The combined organic phase was washed with brine (5.00 L), dried over Na₂SO₄, and filtered through a Celite pad twice. The filtrate was concentrated to 1.20 L to give a suspension. The suspension was filtered, and the filter cake was washed with ethyl acetate (200 mL). The filter cake was dried under reduced pressure to give the desired product (120 g) as an off-white solid. LCMS: RT 0.736 min, [M+H]⁺ 353.0, LCMS method Y. ¹H NMR: (400 MHz DMSO-d₆) δ 8.90 (d, J=2.4 Hz, 1H), 7.51-7.47 (m, 1H), 7.39-7.36 (m, 1H), 7.27-7.22 (m, 1H), 5.92 (d, J=2.0 Hz, 1H), 4.96 (s, 2H), 4.30-4.22 (m, 4H), 1.28 (t, J=7.2 Hz, 3H).

### Step 7. ethyl (S)-1-amino-8-(2-chloro-5-fluorophenyl)-6-oxo-5,6,7,8-tetrahydroimidazo[1,5-a]pyrazine-3-carboxylate and ethyl (R)-1-amino-8-(2-chloro-5-fluorophenyl)-6-oxo-5,6,7,8-tetrahydroimidazo[1,5-a]pyrazine-3-carboxylate

Ethyl 1-amino-8-(2-chloro-5-fluorophenyl)-6-oxo-5,6,7,8-tetrahydroimidazo[1,5-a]pyrazine-3-carboxylate (120 g) was chirally resolved by chiral SFC (column: DAICEL CHIRALPAK IC, 250 mm*50 mm, 10 μm, at 35° C.; Waters SFC prep 350; mobile phase: 40% of EtOH/acetonitrile (ratio 3:1) in supercritical CO₂; flow rate 250 g/min) to give peak 1 (RT 1.485 min, 52.2 g) and peak 2 (RT 2.139 min, 52.2 g), both as a yellow amorphous solid.

Peak 1. ethyl (R)-1-amino-8-(2-chloro-5-fluorophenyl)-6-oxo-5,6,7,8-tetrahydroimidazo[1,5-a]pyrazine-3-carboxylate. LCMS: RT 1.455 min, [M+H]⁺ 353.0, LCMS method Z. ¹H NMR: (400 MHz DMSO-d₆) δ 8.90 (d, J=2.4 Hz, 1H), 7.51-7.47 (m, 1H), 7.39-7.36 (m, 1H), 7.27-7.22 (m, 1H), 5.92 (d, J=2.0 Hz, 1H), 4.96 (s, 2H), 4.30-4.22 (m, 4H), 1.28 (t, J=7.2 Hz, 3H). ¹⁹F NMR: (400 MHz DMSO-d₆) δ −114.6.

Peak 2. ethyl (S)-1-amino-8-(2-chloro-5-fluorophenyl)-6-oxo-5,6,7,8-tetrahydroimidazo[1,5-a]pyrazine-3-carboxylate. LCMS: RT 1.453 min, [M+H]⁺ 353.1, LCMS method Z. ¹H NMR: (400 MHz DMSO-d₆) δ 8.90 (d, J=2.4 Hz, 1H), 7.51-7.47 (m, 1H), 7.39-7.36 (m, 1H), 7.27-7.22 (m, 1H), 5.92 (d, J=2.0 Hz, 1H), 4.96 (s, 2H), 4.30-4.22 (m, 4H), 1.28 (t, J=7.2 Hz, 3H). ¹⁹F NMR: (400 MHz DMSO-d₆) δ −114.6.

### Example 2

### Ethyl (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (Intermediate II)

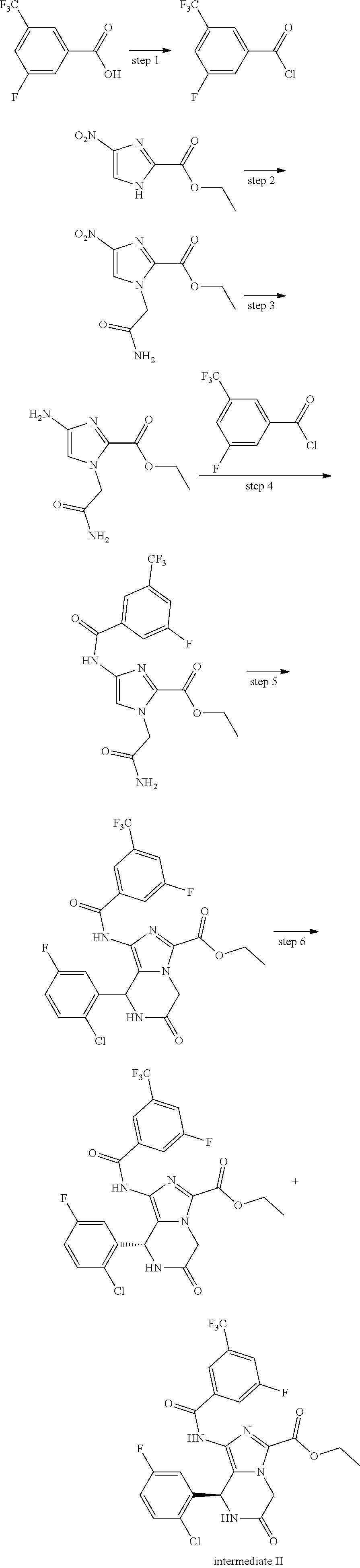

### Step 1. 3-fluoro-5-(trifluoromethyl)benzoyl chloride

Five batches were run in parallel. To a solution of 3-fluoro-5-(trifluoromethyl)benzoic acid (124 g) in dichloromethane (720 mL) was added DMF (2.29 mL) at 25° C. Oxalyl chloride (121 g) was added dropwise at 15-25° C. The resulting solution was stirred at 15-25° C. for 2 hours. All five batches of the mixture were combined and concentrated to give the desired product (700 g, crude) as a yellow oil, which was used in Step 4 without purification.

### Step 2. ethyl 1-(2-amino-2-oxoethyl)-4-nitro-1H-imidazole-2-carboxylate

Four batches were run in parallel. To a solution of ethyl 4-nitro-1H-imidazole-2-carboxylate (143 g) in acetonitrile (1.43 L) was added 2-tert-butyl-1,1,3,3-tetramethylguanidine (152 g). The mixture was stirred at 25° C. for 10 minutes. 2-Bromoacetamide (171 g) was added. The reaction mixture was stirred at 25° C. for 4 hours. All four batches of the reaction mixture were poured into H₂O (6.00 L) and filtered. The filter cake was washed with H₂O (1.50 L) and acetonitrile (1.50 L), and dried to give the desired product (635 g) as an off-white solid. ¹H NMR (400 MHz, DMSO-d₆) δ 8.60 (s, 1H), 7.76 (s, 1H), 7.38 (s, 1H), 5.08 (s, 2H), 4.31 (dd, J=7.2, 14.4 Hz, 2H), 1.31 (t, J=7.6 Hz, 3H).

### Step 3. ethyl 4-amino-1-(2-amino-2-oxoethyl)-1H-imidazole-2-carboxylate

Five batches were run in parallel. To a solution of ethyl 1-(2-amino-2-oxoethyl)-4-nitro-1H-imidazole-2-carboxylate (127 g) in MeOH (1.27 L) was added Pd/C (10% by weight, 12.7 g) at 25° C. The mixture was degassed and purged with H₂ for 3 times, and then the mixture was stirred at 25° C. under H₂ (50 psi) for 24 hours. Five batches of the reaction mixture were combined, and MeOH (75.0 L) was added. The suspension was filtered, and the filtrate was concentrated to remove most of the solvent. It was then filtered again, and the filter cake was dried to give the desired product (485 g) as a light-yellow solid. LCMS: RT 0.128 min, [M+H]⁺ 213.1, LCMS method X. ¹H NMR (400 MHz, DMSO-d₆) δ 7.45 (s, 1H), 7.10 (s, 1H), 6.44 (s, 1H), 4.84 (s, 2H), 4.56 (s, 2H), 4.16 (dd, J=7.2, 14.4 Hz, 2H), 1.24 (t, J=7.2 Hz, 3H).

### Step 4. ethyl 1-(2-amino-2-oxoethyl)-4-(3-fluoro-5-(trifluoromethyl)benzamido)-1H-imidazole-2-carboxylate

Five batches were run in parallel. To a suspension of ethyl 4-amino-1-(2-amino-2-oxoethyl)-1H-imidazole-2-carboxylate (97.0 g) in dichloromethane (1.00 L) was added pyridine (108 g) at 25° C. The mixture was cooled to 0° C., then a solution of 3-fluoro-5-(trifluoromethyl)benzoyl chloride (135 g) in dichloromethane (110 mL) was added. The mixture was warmed to 25° C. and stirred at 25° C. for 1 hour. Five batches of the reaction were combined and filtered. The filter cake was triturated with H₂O (10.0 L*3) at 25° C. for 3 hours to give the desired product (1020 g, crude) as a brown solid. LCMS: RT 0.728 min, [M+H]⁺ 403.1, LCMS method X. ¹H NMR (400 MHz, DMSO-d₆) δ 11.6 (s, 1H), 8.31 (s, 1H), 8.17 (d, J=9.2 Hz, 1H), 7.93 (d, J=8.4 Hz, 1H), 7.77 (s, 1H), 7.63 (s, 1H), 7.21 (s, 1H), 5.06 (s, 2H), 4.25 (dd, J=7.2, 14.0 Hz, 2H), 1.28 (t, J=6.8 Hz, 3H).

### Step 5. ethyl 8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate

Four batches were run in parallel. To a mixture of ethyl 1-(2-amino-2-oxoethyl)-4-(3-fluoro-5-(trifluoromethyl)benzamido)-1H-imidazole-2-carboxylate (200 g,) and 2-chloro-5-fluorobenzaldehyde (118 g) was added Eaton's reagent (2.13 kg) at 25° C. It was then heated at 80° C. for 12 hours. Four batches of the mixture were combined, poured into H₂O (6.00 L) and extracted with ethyl acetate (5.00 L*2). The organic layer was washed with saturated NaHCO₃ solution (6.00 L) and brine (4.00 L), dried over Na₂SO₄, filtered and concentrated. The residue was triturated with petroleum ether:ethyl acetate 3:1 (1.50 L) for 0.5 hour and filtered. The filter cake was washed with petroleum ether:ethyl acetate 3:1 (400 mL), then dried to give the desired product (280 g) as an off-white solid. LCMS: RT 0.724 min, [M+H]⁺ 543.0, LCMS method X. ¹H NMR (400 MHz, DMSO-d₆) δ 10.6 (s, 1H), 8.97 (s, 1H), 7.92 (d, J=8.4 Hz, 1H), 7.84-7.81 (m, 2H), 7.37-7.33 (m, 1H), 7.12-7.07 (m, 2H), 6.06 (s, 1H), 5.17-5.04 (m, 2H), 4.36-4.31 (m, 2H), 1.33 (t, J=7.2 Hz, 3H).

### Step 6. ethyl (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate and ethyl (R)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate

Ethyl 8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (80.0 g) was chirally resolved by chiral SFC (column: REGIS (S,S) WHELK-O1 (250 mm*50 mm, 10 μm); mobile phase: 50% EtOH in CO₂) to give the two enantiomers, both as a yellow solid.

Peak 1: ethyl (R)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate, 31.0 g. MS: [M+H]⁺ 543.1. Analytical chiral SFC: RT=1.139 min, 100% ee under 220 nm. Column: (S,S) Whelk-O1 100×4.6 mm I.D., 3.5 μm; mobile phase A: CO₂, phase B: EtOH with 0.05% diethylamine; gradient: isocratic, 40% EtOH (0.05% DEA) in CO₂; flow rate: 3 mL/min; column temperature: 35° C.; back pressure: 100 bar. ¹H NMR (400 MHz, DMSO-d₆) δ 10.6 (s, 1H), 8.98 (s, 1H), 7.93 (d, J=8.4 Hz, 1H), 7.83-7.81 (m, 2H), 7.37-7.33 (m, 1H), 7.11-7.07 (m, 2H), 6.05 (s, 1H), 5.16-5.08 (m, 2H), 4.36-4.30 (m, 2H), 1.32 (t, J=6.8 Hz, 3H).

Peak 2: ethyl (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate, 31.0 g. MS: [M+H]⁺ 543.1. Analytical chiral SFC: RT=1.977 min, 100% ee under 220 nm. Column: (S,S)-Whelk-O1 100×4.6 mm I.D., 3.5 μm; mobile phase A: CO₂, phase B: EtOH with 0.05% diethylamine; gradient: isocratic, 40% EtOH (0.05% DEA) in CO₂; flow rate: 3 mL/min; column temperature: 35° C.; back pressure: 100 bar. ¹H NMR (400 MHz, DMSO-d₆) δ 10.6 (s, 1H), 8.97 (s, 1H), 7.91 (d, J=8.0 Hz, 1H), 7.84-7.81 (m, 2H), 7.37-7.33 (m, 1H), 7.12-7.07 (m, 2H), 6.05 (s, 1H), 5.16-5.03 (m, 2H), 4.36-4.31 (m, 2H), 1.32 (t, J=6.8 Hz, 3H).

Additional compounds prepared according to the methods of Example 2 are listed in Table 2 below. Corresponding ¹H NMR and mass spectrometry characterization for these compounds are described in Table 1. Certain compounds in Table 2 below were prepared with other compounds whose preparation is described further below in the Examples.

TABLE 2
Additional Exemplary Compounds
Compound

I-438
I-439
I-690
I-734
I-735

### Example 3

### ethyl 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylate (Intermediate III)

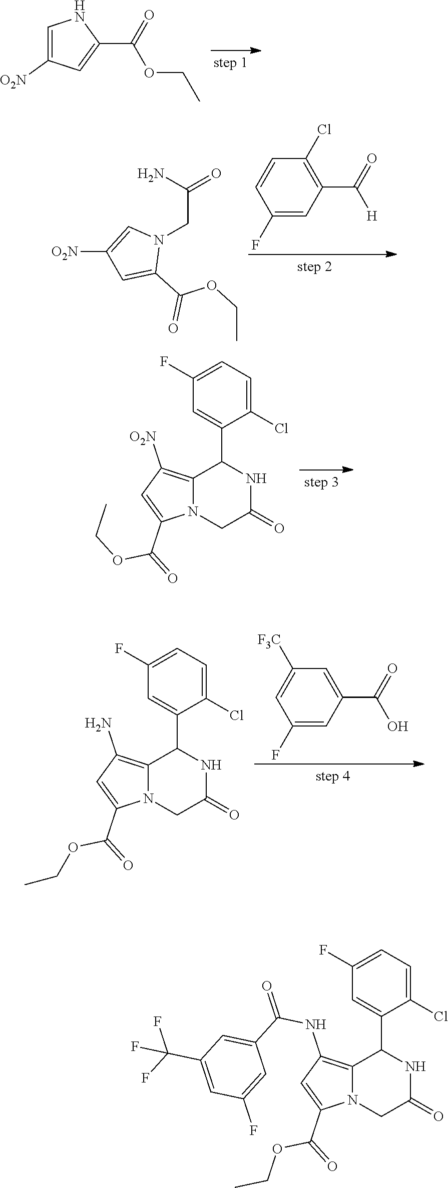

### Step 1. ethyl 1-(2-amino-2-oxoethyl)-4-nitro-1H-pyrrole-2-carboxylate

A round bottom flask was charged with ethyl 4-nitro-1H-pyrrole-2-carboxylate (8.3 g) dissolved in acetonitrile (150 mL). 2-(tert-butyl)-1,1,3,3-tetramethylguanidine (8.9 g) was added, and the solution was stirred at RT for 5 min. 2-Bromoacetamide (7.70 g) was added and the solution was stirred at room temperature for 1 h. The reaction was quenched with water and extracted with ethyl acetate. The organic layer was washed with brine, dried over Na₂SO₄ and concentrated. The residue was dissolved in acetonitrile and concentrated. A precipitate formed and was collected by filtration. After drying the desired product (9.3 g) was obtained as an off-white solid. LCMS: RT 0.543 min, [M+H]⁺ 242.05, LCMS method J.

### Step 2. ethyl 1-(2-chloro-5-fluorophenyl)-8-nitro-3-oxo-1,2,3,4-tetrahydropyrrolo[1,2-a]pyrazine-6-carboxylate

A round bottom flask was charged with ethyl 1-(carbamoylmethyl)-4-nitro-1H-pyrrole-2-carboxylate (2 g), 2-chloro-5-fluorobenzaldehyde (1.6 g) and a stir bar. Eaton's reagent (50 mL) was added, and the solution was stirred at 80° C. for 50 minutes. The reaction mixture was diluted with ethyl acetate, cooled to 0° C., then quenched with aqueous sodium bicarbonate solution and extracted with ethyl acetate. The organic phase was dried over Na₂SO₄ and concentrated. A precipitate formed and was collected by filtration to give the desired product (2.7 g) as an off-white solid. LCMS: RT 0.859 min, [M+H]⁺=381.95, LCMS method C.

### Step 3. ethyl 8-amino-1-(2-chloro-5-fluorophenyl)-3-oxo-1,2,3,4-tetrahydropyrrolo[1,2-a]pyrazine-6-carboxylate

A round bottom flask was charged with ethyl 1-(2-chloro-5-fluorophenyl)-8-nitro-3-oxo-1H,2H,3H,4H-pyrrolo[1,2-a]pyrazine-6-carboxylate (5 g), ammonium chloride (2.8 g), iron powder (3.75 g) and a stir bar. EtOH/H₂O (3:2, 67 mL) was added, and the solution was stirred at 90° C. for 2 hours. After filtration, the filtrate was concentrated and purified by silica gel chromatography (10 g column, eluting with dichloromethane:methanol 10:1) to the desired product (2.8 g) as a white solid. LCMS: RT 0.662 min, [M+H]⁺=352.15, LCMS method C.

### Step 4. ethyl 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylate

A round bottom flask was charged with (1-chloro-2-methylprop-1-en-1-yl)dimethylamine (1.25 g), 3-fluoro-5-(trifluoromethyl)benzoic acid (2.15 g) and a stir bar. Dichloromethane (30 mL) was added, and the solution was stirred at room temperature for 1 hour. Triethylamine (173 mg) and ethyl 8-amino-1-(2-chloro-5-fluorophenyl)-3-oxo-1,2,3,4-tetrahydropyrrolo[1,2-a]pyrazine-6-carboxylate (3 g) were added, and the solution was stirred at room temperature for 1 hour. The reaction was quenched with water and extracted with ethyl acetate. The organic layer was washed with brine, dried over Na₂SO₄ and concentrated to give the desired product (3.2 g) as a white solid. LCMS: RT 0.919 min, [M+H]⁺=542.20, LCMS method C.

### Example 4

### N-(4-(2-chloro-5-fluorophenyl)-6-oxo-4,5,6,7-tetrahydropyrazolo[1,5-a]pyrazin-3-yl)-3-fluoro-5-(trifluoromethyl)benzamide (I-1)

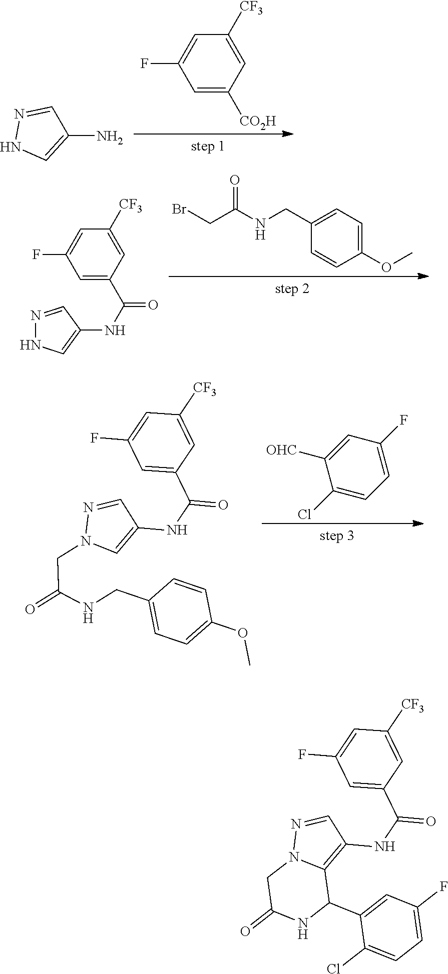

### Step 1. 3-fluoro-N-(1H-pyrazol-4-yl)-5-(trifluoromethyl)benzamide

To a solution of 3-fluoro-5-(trifluoromethyl)benzoic acid (5.20 g) in dichloromethane (20 mL) was added Ghosez's reagent (3.34 g). After stirring at room temperature for 0.5 hour, Et₃N (6.90 g) and 1H-pyrazol-4-amine (2.08 g) were added, and the resulting mixture was stirred at room temperature for 1.5 hour. The reaction was quenched with water and extracted with ethyl acetate. The organic layer was washed with brine and dried over Na₂SO₄. The resulting mixture was filtered and concentrated under reduced pressure. The residue was dissolved in DMF. The resulting solution was purified using reverse phase chromatography with the following conditions (C18 column; mobile phase A: water, mobile phase B: acetonitrile; flow rate: 60 mL/min; Gradient: 0% B to 100% B in 40 min; UV wavelength 254/220 nm), which gave the desired product (2 g) as a yellow oil. LCMS: RT 1.199 min, [M+H]⁺ 274.10 (LCMS method A).

### Step 2. 3-fluoro-N-(1-(2-((4-methoxybenzyl)amino)-2-oxoethyl)-1H-pyrazol-4-yl)-5-(trifluoromethyl)benzamide

To a stirred solution of 3-fluoro-N-(1H-pyrazol-4-yl)-5-(trifluoromethyl)benzamide (700 mg) and 2-bromo-N-[(4-methoxyphenyl)methyl]acetamide (991 mg) in DMF (7.0 mL) was added Cs₂CO₃ (2.50 g) at room temperature. The resulting mixture was stirred at 50° C. for 5 hours. The reaction was quenched with water and extracted with ethyl acetate. The organic layer was washed with brine, dried over Na₂SO₄, filtered and concentrated to give the desired product (500 mg) as a light yellow solid. LCMS: RT 1.278 min, [M+H]⁺ 451.30 (LCMS method E).

### Step 3. N-(4-(2-chloro-5-fluorophenyl)-6-oxo-4,5,6,7-tetrahydropyrazolo[1,5-a]pyrazin-3-yl)-3-fluoro-5-(trifluoromethyl)benzamide

To a stirred solution of 3-fluoro-N-(1-(2-((4-methoxybenzyl)amino)-2-oxoethyl)-1H-pyrazol-4-yl)-5-(trifluoromethyl)benzamide (300 mg) in Eaton's reagent (3 mL) was added 2-chloro-5-fluorobenzaldehyde (126 mg) at room temperature. The resulting mixture was stirred at 120° C. for 1 hour. Once cooled, the reaction mixture was poured into saturated NaHCO₃ solution (20 ml) and extracted with ethyl acetate (2×15 ml). The organic phase was washed with saturated NaHCO₃ solution (10 ml), dried over sodium sulfate, filtered and concentrated. The residue was dissolved in DMF and purified using prep-HPLC (Column: XBridge Prep OBD C18 Column, 30×150 mm, 5 μm; mobile phase A: 10 mM NH₄HCO₃ solution, mobile phase B: acetonitrile; flow rate: 60 mL/min; Gradient: 30% B to 60% B in 8 minutes; wavelength: 254/220 nm; RT: 7.48 min) to give the desired product (19.2 mg) as a light yellow oil. LCMS: RT=0.856 min, [M+H]⁺ 471.15 (LCMS method E).

Additional compounds prepared according to the methods of Examples 1-4 are listed in Table 3 below. Corresponding ¹H NMR and mass spectrometry characterization for these compounds are described in Table 1. Certain compounds in Table 3 below were prepared with other compounds whose preparation is described further below in the Examples.

TABLE 3
Additional Exemplary Compounds
Compound

I-2
I-3
I-357

### Example 5

### N-(8-(2-chloro-5-fluorophenyl)-6-oxo-5,6,7,8-tetrahydroimidazo[1,5-a]pyrazin-1-yl)-3-fluoro-5-(trifluoromethyl)benzamide (I-4)

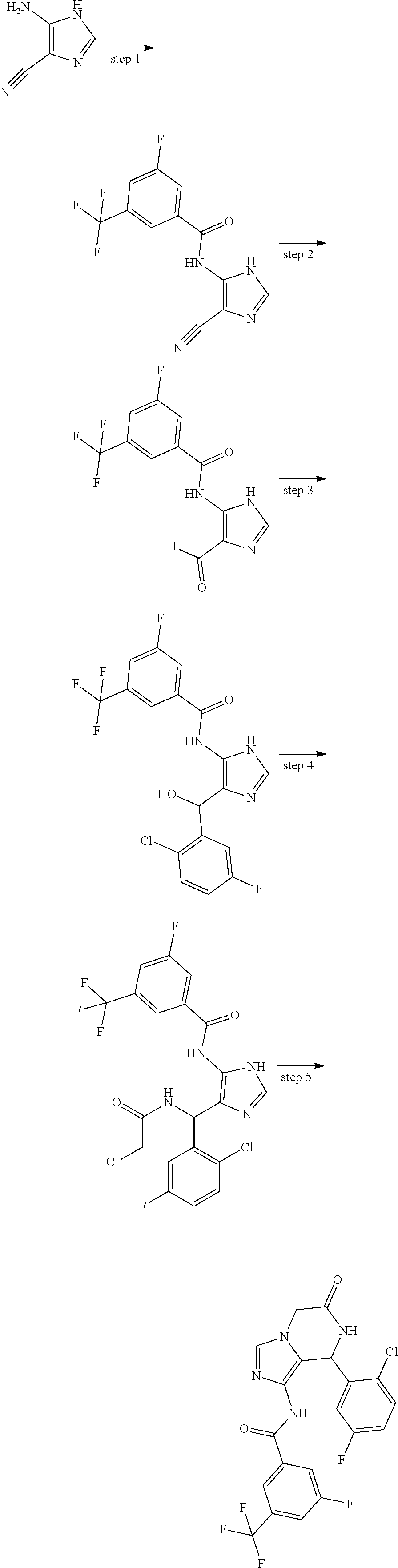

### Step 1. N-(4-cyano-1H-imidazol-5-yl)-3-fluoro-5-(trifluoromethyl)benzamide

A flame-dried microwave vial was charged with 5-amino-1H-imidazole-4-carbonitrile (400 mg). Dichloromethane (4 mL) was added, followed by pyridine (1.19 mL) and 3-fluoro-5-(trifluoromethyl)benzoyl chloride (844 μL) dropwise under nitrogen. The reaction media was stirred overnight. A beige precipitate formed. MeOH was added and the solution was concentrated to 1-2 mL of solution in MeOH. The crude mixture was cooled to 0° C. and dichloromethane (5 mL) was added. The formed beige precipitate was collected by filtration, washed with cold dichloromethane and dried to give the desired product (583 mg) as a pale beige powder. LCMS: RT 1.37 min, [M+H]⁺ 299.0, LCMS method Q.

### Step 2. 3-fluoro-N-(4-formyl-1H-imidazol-5-yl)-5-(trifluoromethyl)benzamide

A flame-dried microwave vial was charged with N-(4-cyano-1H-imidazol-5-yl)-3-fluoro-5-(trifluoromethyl)benzamide (1.5 g). THF (30 mL) was added, and the solution was cooled to −78° C. under N₂. A solution of DIBAL (1 M in toluene, 16 mL) was added dropwise at −78° C. and the mixture was stirred at this temperature for 30 minutes. After 30 minutes of stirring more DIBAL (1 M in toluene, 5 mL) was added at −78° C. and the reaction mixture was slowly warmed up to −30° C. and stirred for 2 hours. The reaction was quenched with MeOH (20 mL) and stirred for 30 minutes at −30° C. The solution was then concentrated under reduced pressure and taken up in ethyl acetate (40 mL). Saturated Rochelle salt solution was added (40 mL). The mixture was stirred vigorously for 30 minutes and the aqueous phase was extracted 4 times with ethyl acetate. The combined organic layers were dried over MgSO₄ and concentrated under reduced pressure to afford a yellow/orange solid. The solid was triturated in cold Et₂O/hexane and collected by filtration. After drying the desired product (1.25 g) was obtained as an orange solid. LCMS: RT 1.35 min, [M+H]⁺ 302.0, LCMS method Q. ¹H NMR (400 MHz, DMSO-d6) δ 13.08 (br. s, 1H), 11.32 (br. s, 1H), 9.95 (s, 1H), 8.23 (s, 1H), 8.12 (d, J=9.0 Hz, 1H), 7.95 (d, J=6.9 Hz, 1H), 7.89 (br. s, 1H).

### Step 3. N-(4-((2-chloro-5-fluorophenyl)(hydroxy)methyl)-1H-imidazol-5-yl)-3-fluoro-5-(trifluoromethyl)benzamide

A flame-dried microwave vial was charged with a freshly prepared solution of bromo(2-chloro-5-fluorophenyl)magnesium in THF (˜0.5 M, 15.7 mL). The solution was cooled to 0° C. under N₂ before a freshly prepared solution of 3-fluoro-N-(4-formyl-1H-imidazol-5-yl)-5-(trifluoromethyl)benzamide (320 mg) in THF (10 mL) was added dropwise over 40 minutes at 0° C. The reaction mixture was warmed up to room temperature. After 1 hour the solution was cooled to 0° C. and quenched by addition of saturated NH₄Cl solution (15 mL). The aqueous layer was then extracted with ethyl acetate (3×10 mL). The organic layers were combined, dried over MgSO₄ and concentrated under reduced pressure. The crude material was purified on reverse phase column chromatography (80 g column, 10 mM ammonium formate solution:acetonitrile 95:5 to 10:90) to give the desired product (311 mg) as a white solid. LCMS: RT 1.50 min, [M+H]⁺ 432.2, LCMS method Q. ¹H NMR (400 MHz, DMSO-d6) δ 12.26 (br. s, 1H), 10.26 (br. s, 1H), 8.06 (s, 1H), 7.98 (d, J=9.3 Hz, 1H), 7.94 (d, J=8.3 Hz, 1H), 7.51 (s, 1H), 7.48 (dd, J=9.8, 2.7 Hz, 1H), 7.34 (dd, J=8.8, 5.1 Hz, 1H), 7.04 (td, J=8.4, 3.0 Hz, 1H), 6.16 (submerged br. s, 1H), 6.09 (s, 1H).

### Step 4. N-(4-((2-chloro-5-fluorophenyl)(2-chloroacetamido)methyl)-1H-imidazol-5-yl)-3-fluoro-5-(trifluoromethyl)benzamide

To a microwave vial was added N-(4-((2-chloro-5-fluorophenyl)(hydroxy)methyl)-1H-imidazol-5-yl)-3-fluoro-5-(trifluoromethyl)benzamide (60 mg). 2-Chloroacetonitrile (0.4 mL) was added, followed by sulfuric acid (0.1 mL) dropwise. After 5 minutes the reaction was cooled to −20° C. and diluted with dichloromethane. It was then carefully quenched with saturated NaHCO₃ solution until no more gas evolution was observed. The aqueous phase was then extracted with dichloromethane (2×4 mL). The organic layers were combined, dried over MgSO₄ and concentrated under reduced pressure to give the desired product (69 mg) as a yellow viscous oil. LCMS: RT 1.55 min, [M+H]⁺ 507.1, LCMS method Q.

### Step 5. N-(8-(2-chloro-5-fluorophenyl)-6-oxo-5,6,7,8-tetrahydroimidazo[1,5-a]pyrazin-1-yl)-3-fluoro-5-(trifluoromethyl)benzamide

A flame-dried microwave vial was charged with N-(4-((2-chloro-5-fluorophenyl)(2-chloroacetamido)methyl)-1H-imidazol-5-yl)-3-fluoro-5-(trifluoromethyl)benzamide (80 mg) and potassium carbonate (54.4 mg). DMF (2 mL) was added, and the reaction vial was purged with nitrogen for 2 minutes. The reaction was irradiated at 100° C. under microwave for 1 hour. The reaction media was filtered through a syringe filter and loaded on a reverse phase column for purification (12 g column, 10 mM ammonium formate solution/acetonitrile 95:5 to 60:40 in 16 minutes). The product was further purified on an XSELECT HSS PFP OBD, 5 μm, 30×75 mm column; mobile phase A: 10 mM ammonium formate solution; mobile phase B: MeOH; gradient: 30% B for 1 min, 30% B to 50% B over 11 minutes, 50% B to 100% B for 0.1 minute, hold 100% B for 2.9 minutes; flow: 45 mL/min) to give the desired product (4.7 mg). LCMS: RT 2.67 min, [M+H]⁺ 471.1, LCMS method R. ¹H NMR (400 MHz, DSMO-d6) δ 10.36 (br. s, 1H), 8.87 (d, J=1.4 Hz, 1H), 7.91 (d, J=8.6 Hz, 1H), 7.83 (s, 1H), 7.82 (submerged d, J=9.7 Hz, 1H), 7.65 (s, 1H), 7.34 (dd, J=8.7, 5.2 Hz, 1H), 7.08 (td, J=8.4, 3.0 Hz, 1H), 7.01 (dd, J=9.2, 3.1 Hz, 1H), 5.98 (s, 1H), 4.92 (d with roof effect, J=17.8 Hz, 1H), 4.85 (d with roof effect, J=17.7 Hz, 1H).

Additional compounds prepared according to the methods of Example 5 are listed in Table 4 below. Corresponding ¹H NMR and mass spectrometry characterization for these compounds are described in Table 1. Certain compounds in Table 4 below were prepared with other compounds whose preparation is described in the Examples herein.

TABLE 4
Additional Exemplary Compound
Compound

I-6

### Example 6

### N-(4-(2-chloro-5-fluorophenyl)-1-methyl-6-oxo-1,4,5,6-tetrahydropyrrolo[3,4-c]pyrazol-3-yl)-3-fluoro-5-(trifluoromethyl)benzamide (I-5)

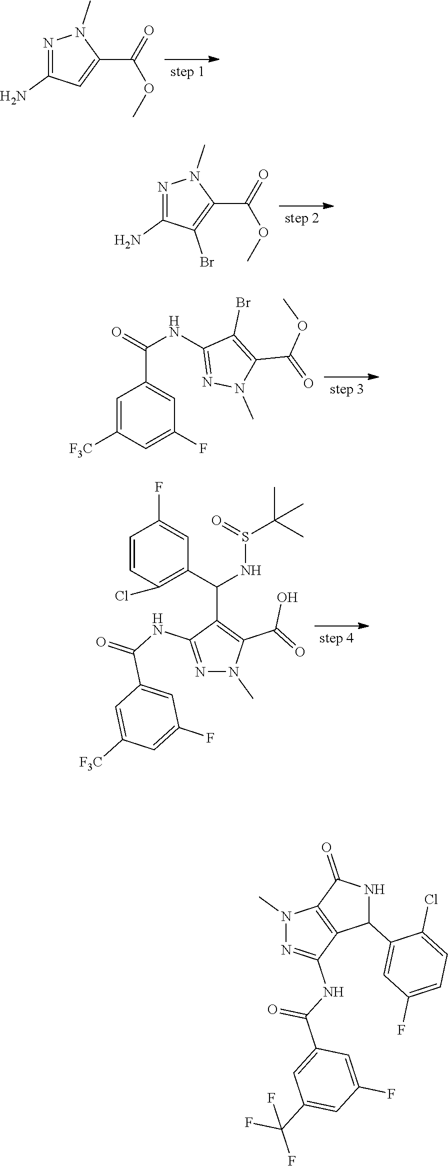

### Step 1. methyl 3-amino-4-bromo-1-methyl-1H-pyrazole-5-carboxylate

A 50 mL round bottom flask was charged with methyl 3-amino-1-methyl-1H-pyrazole-5-carboxylate (500 mg). THF (12 mL) was added, followed by NBS (630 mg) in one single portion. After 1h 45 minutes, the solution was concentrated under reduced pressure. Dichloromethane (10 mL) and water (10 mL) were added. The aqueous phase was extracted twice with dichloromethane. The organic layers were combined, washed once with water, dried over MgSO₄ and concentrated under reduced pressure to afford the desired product (720 mg) as an orange solid. LCMS: RT 1.09 min, [M+H]⁺ 234.0/236.0, LCMS method Q.

### Step 2. methyl 4-bromo-3-(3-fluoro-5-(trifluoromethyl)benzamido)-1-methyl-1H-pyrazole-5-carboxylate

A flame-dried microwave vial was charged with methyl 3-amino-4-bromo-1-methyl-1H-pyrazole-5-carboxylate (480 mg). Dichloromethane (4 mL) was added, followed by pyridine (659 μL) and 3-fluoro-5-(trifluoromethyl)benzoyl chloride (343 μL) dropwise under nitrogen. After 1 hour the solution was concentrated under reduced pressure. The residue was dissolved in DMF (1 mL) and loaded on a reverse phase column for purification (30 g column, 10 mM ammonium formate solution/acetonitrile 95:5 to 35:65 in 16 minutes) to give the desired product (530 mg) as a white powder. LCMS: RT 1.67 min, [M+H]⁺ 424.0/426.0, LCMS method Q. ¹H NMR (400 MHz, DMSO-d6) δ 10.72 (s, 1H), 8.20 (s, 1H), 8.11 (d, J=9.0 Hz, 1H), 8.02 (d, J=8.4 Hz, 1H), 4.10 (s, 3H), 3.90 (s, 3H).

### Step 3. 4-(((tert-butylsulfinyl)amino)(2-chloro-5-fluorophenyl)methyl)-3-(3-fluoro-5-(trifluoromethyl)benzamido)-1-methyl-1H-pyrazole-5-carboxylic acid

To a 25 mL round bottom flask containing a solution of methyl 4-bromo-3-(3-fluoro-5-(trifluoromethyl)benzamido)-1-methyl-1H-pyrazole-5-carboxylate (150 mg) in dry THF (4 mL) under nitrogen at 0° C. was added sodium hydride (18.5 mg) and the resulting yellow mixture was stirred at 0° C. for 1 h. The mixture was cooled to −78° C. and treated with a solution of n-BuLi (2.5 M in hexanes, 148 μL). After 20 min at −78° C., a solution of N-(2-chloro-5-fluorobenzylidene)-2-methylpropane-2-sulfinamide (101 mg) in THF (1 mL) was added. The reaction mixture was stirred at −78° C. and allowed to gradually warm to room temperature. After 16 hours, the reaction was quenched with saturated NH₄Cl solution. The aqueous phase was extracted with ethyl acetate. The organic layers were combined, washed with saturated NaHCO₃ solution, dried over sodium sulfate, filtered and concentrated to obtain a yellow residue which was loaded directly onto a C18 column and purified (30 g column, 10 mM ammonium formate solution:acetonitrile 95:5 to 50:50 in 16 minutes) to give the desired product (41 mg). LCMS: RT 1.50 min, [M+H]⁺ 593.2, LCMS method Q.

### Step 4. N-(4-(2-chloro-5-fluorophenyl)-1-methyl-6-oxo-1,4,5,6-tetrahydropyrrolo[3,4-c]pyrazol-3-yl)-3-fluoro-5-(trifluoromethyl)benzamide

A flame-dried microwave vial was charged with 4-(((tert-butylsulfinyl)amino)(2-chloro-5-fluorophenyl)methyl)-3-(3-fluoro-5-(trifluoromethyl)benzamido)-1-methyl-1H-pyrazole-5-carboxylic acid (40 mg). MeOH (1 mL) was added, followed by dropwise addition of hydrogen chloride (4 M in dioxane, 136 μL). After 1 hour LCMS showed deprotection of the starting material into the corresponding amine (LCMS: RT 1.32 min, [M+H]⁺ 489.1, LCMS method Q). The reaction mixture was concentrated under reduced pressure and taken up in DMF (0.4 mL). DIPEA (31.6 μL) was added, followed by HATU (37.3 mg). After 30 minutes at room temperature the crude material was purified by reverse phase column chromatography (30 g column, 10 mM ammonium formate solution:acetonitrile 95:5 to 35:65 in 16 minutes) to give the desired product (6.9 mg). LCMS: RT 1.67 min, [M+H]⁺ 471.1, LCMS method Q. ¹H NMR (400 MHz, DMSO-d6) δ 11.40 (br. s, 1H), 9.00 (s, 1H), 8.03 (s, 1H), 7.92 (app. d, J=8.8 Hz, 2H), 7.46 (dd, J=8.9, 5.2 Hz, 1H), 7.12 (ddd, J=8.7, 8.1, 3.1 Hz, 1H), 6.82 (dd, J=9.5, 3.1 Hz, 1H), 6.05 (s, 1H), 3.94 (s, 3H). Partial formate salt at 8.44.

### Example 7

### N-(5-(2-chloro-5-fluorophenyl)-7-oxo-6,7-dihydro-5H-imidazo[1,5-a]imidazol-3-yl)-3-fluoro-5-(trifluoromethyl)benzamide (I-9)

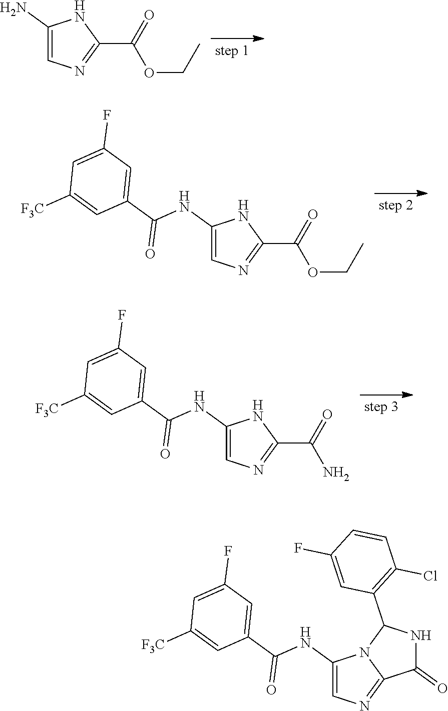 [SMILES: CCOC(=O)c1ncc(N)[nH]1 ; CCOC(=O)c1ncc(NC(=O)c2cc(F)cc(C(F)(F)F)c2)[nH]1 ; NC(=O)c1ncc(NC(=O)c2cc(F)cc(C(F)(F)F)c2)[nH]1 ; O=C(Nc1cnc2n1C(c1cc(F)ccc1Cl)NC2=O)c1cc(F)cc(C(F)(F)F)c1]

### Step 1. ethyl 5-(3-fluoro-5-(trifluoromethyl)benzamido)-1H-imidazole-2-carboxylate

A flame-dried 25 mL round bottom flask was charged with ethyl 5-amino-1H-imidazole-2-carboxylate (200 mg). Dichloromethane (4 mL) was added, followed by pyridine (519 μL) and 3-fluoro-5-(trifluoromethyl)benzoyl chloride (197 μL) under nitrogen. After 20 minutes the white precipitate formed was collected by filtration and washed once with cold dichloromethane. After drying the desired product (423 mg) was obtained. LCMS: RT 1.66 min; [M+H]⁺ 346.1, LCMS method Q.

### Step 2. 5-(3-fluoro-5-(trifluoromethyl)benzamido)-1H-imidazole-2-carboxamide

A microwave vial was charged with ethyl 5-(3-fluoro-5-(trifluoromethyl)benzamido)-1H-imidazole-2-carboxylate (500 mg) and sodium cyanide (10.5 mg). Ammonia (7 N in MeOH, 7.18 mL) was added and the reaction media was stirred at 55° C. for 48 hours. The precipitate was collected by filtration and washed with MeOH. The filtrate was concentrated under reduced pressure to afford the desired product (376 mg) as a pale yellow solid. LCMS: RT 1.33 min, [M+H]⁺ 317.1, LCMS method Q.

### Step 3. N-(5-(2-chloro-5-fluorophenyl)-7-oxo-6,7-dihydro-5H-imidazo[1,5-a]imidazol-3-yl)-3-fluoro-5-(trifluoromethyl)benzamide

A microwave vial was charged with 5-(3-fluoro-5-(trifluoromethyl)benzamido)-1H-imidazole-2-carboxamide (50 mg) and 2-chloro-5-fluorobenzaldehyde (25.0 mg). Eaton's reagent (0.5 mL) was added, and the reaction was heated at 100° C. for 20 minutes. The reaction mixture was cooled to 0° C., diluted with ethyl acetate (5 mL) and saturated NaHCO₃ solution until pH was 9-10. The aqueous phase was extracted twice with ethyl acetate. The organic layers were combined, dried over MgSO₄ and concentrated under reduced pressure. The crude material was dissolved in DMF (1 mL) and directly loaded on a reverse phase column for purification (10 mM ammonium formate solution:acetonitrile 95:5 to 35:65 in 16 minutes, 30 g column) to give the desired product (23.2 mg) as a white powder. LCMS: RT 3.36 min, [M+H]⁺ 457.2, LCMS method R. ¹H NMR (400 MHz, DMSO-d6) δ 11.65 (br. s, 1H), 9.85 (br. s, 1H), 8.26 (s, 1H), 8.14 (d, J=9.1 Hz, 1H), 7.95 (d, J=8.3 Hz, 1H), 7.69 (submerged dd, J=8.7, 5.3 Hz, 1H), 7.68 (overlapping s, 1H), 7.429 (ddd, J=8.8, 8.1, 3.1 Hz, 1H), 7.24 (dd, J=9.0, 3.0 Hz, 1H), 6.99 (s, 1H).

### Example 8

### N-(1-(2-chloro-5-fluorophenyl)-6-(hydroxymethyl)-3-oxo-1,2,3,4-tetrahydropyrrolo[1,2-a]pyrazin-8-yl)-3-fluoro-5-(trifluoromethyl)benzamide (I-12)

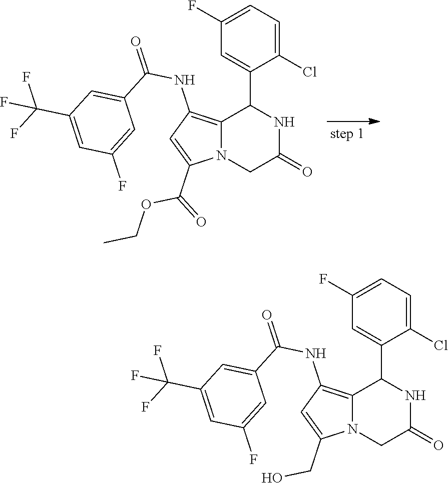 [SMILES: CCOC(=O)c1cc(NC(=O)c2cc(F)cc(C(F)(F)F)c2)c2n1CC(=O)NC2c1cc(F)ccc1Cl ; O=C1Cn2c(CO)cc(NC(=O)c3cc(F)cc(C(F)(F)F)c3)c2C(c2cc(F)ccc2Cl)N1]

### Step 1. N-(1-(2-chloro-5-fluorophenyl)-6-(hydroxymethyl)-3-oxo-1,2,3,4-tetrahydropyrrolo[1,2-a]pyrazin-8-yl)-3-fluoro-5-(trifluoromethyl)benzamide

A microwave vial under nitrogen was charged with ethyl 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylate (Intermediate III, 25 mg) and dry tetrahydrofuran (0.5 mL). The mixture was cooled to 0° C. in an ice bath. LiAlH₄ (2 M in THF, 23.0 μL) was added dropwise and the mixture was allowed to warm to room temperature. More LiAlH₄ (2 M in THF, 23.0 μL) was added dropwise and the reaction mixture was stirred for 2.5 hours. The reaction mixture was cooled to −20° C. and quenched with Rochelle's salt solution (5 mL) and ethyl acetate (4 mL). The aqueous layer was extracted 3 times with ethyl acetate (3×3 mL). The organic layers were combined, dried over Na₂SO₄ and concentrated under reduced pressure. The crude material was purified on a C18 column (10 mM ammonium formate solution: acetonitrile 90:10 to 60:40, 14 min run, 30 g column) to give the desired product (3.1 mg). LCMS: RT 2.85 min, [M+H]⁺ 500.2, LCMS method R. ¹H NMR (400 MHz, DMSO-d6) δ 9.76 (s, 1H), 8.84 (d, J=2.4 Hz, 1H), 7.90 (d, J=8.5 Hz, 1H), 7.81 (overlapping s, 1H), 7.80 (submerged d, J=8.1 Hz, 1H), 7.34 (dd, J=8.8, 5.1 Hz, 1H), 7.08 (td, J=8.4, 3.0 Hz, 1H), 6.96 (dd, J=9.1, 3.0 Hz, 1H), 6.01 (submerged br. s, 1H), 6.01 (s, 1H), 5.06 (br. s, 1H), 4.81 (d, J=18.0 Hz, 1H), 4.71 (d, J=17.9 Hz, 1H), 4.46 (d, J=13.8 Hz, 1H), 4.42 (d, J=12.7 Hz, 1H).

### Example 9

### 7-(2-chloro-5-fluorophenyl)-1-(7-fluoro-5-(trifluoromethyl)-1H-benzo[d]imidazol-2-yl)-3-methyl-1,4,6,7-tetrahydro-5H-pyrazolo[3,4-c]pyridin-5-one (I-13)

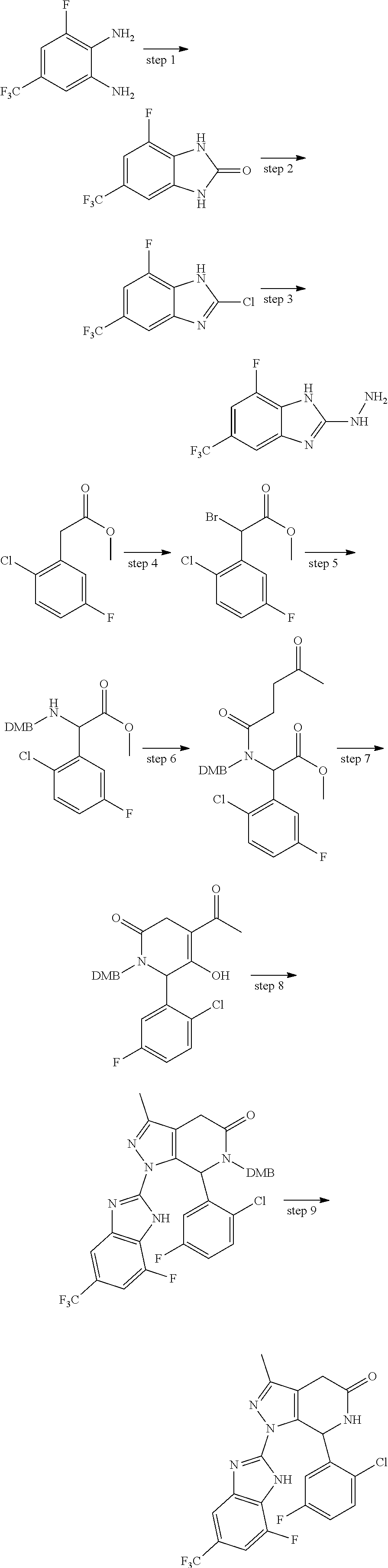

### Step 1. 4-fluoro-6-(trifluoromethyl)-1,3-dihydro-2H-benzo[d]imidazol-2-one

To a 50 mL round bottom flask was added 3-fluoro-5-(trifluoromethyl) benzene-1,2-diamine (1.40 g). DMF (15.6 mL) was added, followed by 1,1′-carbonylimidazole (3.11 g) in several portions. The flask was purged with N₂ and stirred overnight. Water was added, leading to the formation of a yellow precipitate which was collected by filtration, washed with water, and dried to afford the desired product (1.38 g). LCMS: RT 1.07 min, (M−H)−=219.0, LCMS method D.

### Step 2. 2-chloro-7-fluoro-5-(trifluoromethyl)-1H-benzo[d]imidazole

A 20 mL microwave vial was charged with 4-fluoro-6-(trifluoromethyl)-1,3-dihydro-2H-benzo[d]imidazol-2-one (200 mg). Phosphorus oxychloride (5.26 mL) was added, and the vial was flushed with nitrogen for 30 seconds. The vial was heated at 100° C. for 20 hours. The reaction mixture was cooled to room temperature and carefully poured into a vigorously stirred ice-water mixture. NaOH (3 N) solution was added to adjust the pH to 9, and the aqueous layer was extracted twice with ethyl acetate. The organic layers were combined, dried over Na₂SO₄, and concentrated under reduced pressure to afford the desired product (217 mg) as a white solid. LCMS: RT 1.32 min, [M+H]⁺ 239.0, LCMS method D.

### Step 3. 7-fluoro-2-hydrazinyl-5-(trifluoromethyl)-1H-benzo[d]imidazole

A 20 mL microwave vial was charged with 2-chloro-7-fluoro-5-(trifluoromethyl)-1H-benzo[d]imidazole (217 mg). Hydrazine (1.0 M solution in THF, 10.8 mL) was added, and the vial was flushed with nitrogen for 30 seconds. The reaction was heated at 100° C. for 20 hours. The reaction mixture was cooled to room temperature and concentrated under reduced pressure to afford the crude product (213 mg) as a beige solid. The material was used in the next step without further purification. LCMS: RT 0.95 min, [M+H]⁺ 235.0, LCMS method D. ¹H NMR (400 MHz, DMSO-d6) δ 8.39 (s, 1H), 7.23 (d, J=0.9 Hz, 1H), 7.07 (d, J=11.1 Hz, 1H), 4.66 (s, 2H), 3.30 (s, 11).

### Step 4. methyl 2-bromo-2-(2-chloro-5-fluorophenyl)acetate

Methyl 2-(2-chloro-5-fluorophenyl)acetate (1.40 g), NBS (1.48 g) and AIBN (56.2 mg) were added to a flask under N₂. CCl₄ (29.7 mL) was added. The reaction mixture was heated to reflux with an oil bath. After 6 hours the reaction was quenched with water, and the aqueous phase was extracted with dichloromethane (30 mL). The organic layers were combined and dried over sodium sulfate, filtered and concentrated to afford the desired product (1.93 g) as a yellow oil, which was used in the next step without further purification. LCMS: RT 1.53 min, not ionizable, LCMS method D. ¹H NMR (400 MHz, CDCl₃) δ 7.53 (dd, J=9.2, 3.0 Hz, 1H), 7.34 (dd, J=8.9, 5.1 Hz, 1H), 7.01 (ddd, J=8.8, 7.5, 3.0 Hz, 1H), 5.82 (d, J=1.0 Hz, 1H), 3.82 (s, 3H).

### Step 5. methyl 2-(2-chloro-5-fluorophenyl)-2-((2,4-dimethoxybenzyl)amino)acetate

To a microwave vial (20 mL) was added 2,4-dimethoxybenzylamine (164 μL) and DIPEA (281 μL). Acetonitrile (10.0 mL) was added under N₂, followed by methyl 2-bromo-2-(2-chloro-5-fluorophenyl)acetate (301 mg). The colorless solution was stirred at room temperature for 4 hours. Volatiles were removed under reduced pressure and the residue was extracted by dichloromethane (50 mL×2). The organic layers were combined, washed with water (50 mL×2) and brine, then dried over sodium sulfate, filtered and concentrated to afford the desired product (357 mg, 91%) as a brown oil, which was used in the next step without further purification. LCMS: RT 1.52 min, [M+H]⁺ 368.1, LCMS method D. ¹H NMR (400 MHz, CDCl₃) δ 7.31 (ddd, J=13.3, 9.1, 4.1 Hz, 2H), 7.07 (d, J=7.9 Hz, 1H), 7.01-6.87 (m, 1H), 6.47-6.30 (m, 2H), 4.85 (s, 1H), 3.80 (s, 3H), 3.79 (s, 3H), 3.66 (s, 3H). (M+H)=368.1.

### Step 6. methyl 2-(2-chloro-5-fluorophenyl)-2-(N-(2,4-dimethoxybenzyl)-4-oxopentanamido)acetate

A 10 mL round bottom flask was charged with methyl 2-(2-chloro-5-fluorophenyl)-2-((2,4-dimethoxybenzyl)amino)acetate (339 mg). Acetonitrile (18.2 mL) was added, and the reaction mixture was purged with N₂ for 30 seconds. DIPEA (161 μL) was added, followed by 4-oxopentanoyl chloride (126 mg). The mixture was stirred at room temperature for 24 hours under N₂. The solution was concentrated under reduced pressure and the residue was extracted with dichloromethane (100 mL×2). The organic phase was washed with water (100 mL×2) and brine, dried over sodium sulfate, filtered and concentrated to afford the desired product as a brown oil. The crude material was purified by normal phase flash chromatography (heptane:ethyl acetate 70:30 to 0:100) to give the desired product (358 mg) as a yellow oil. LCMS: RT 2.67 min, [M+H]⁺ 466.1, LCMS method L.

### Step 7. 4-acetyl-6-(2-chloro-5-fluorophenyl)-1-(2,4-dimethoxybenzyl)-5-hydroxy-3,6-dihydropyridin-2(1H)-one

To a flame-dried round bottom flask containing a solution of methyl 2-(2-chloro-5-fluorophenyl)-2-(N-(2,4-dimethoxybenzyl)-4-oxopentanamido)acetate (358 mg) in THF (10 mL) was added LiHMDS (1 M in THF, 2.31 mL) dropwise at room temperature under N₂. The reaction mixture was refluxed overnight. After cooling to room temperature, the solution was washed with 1 M HCl, H₂O and brine, and dried over sodium sulfate. The organic solution was filtered and concentrated under vacuum to afford the crude product, which was purified by normal phase flash chromatography (dichloromethane:MeOH=95:5) to give the desired product (185 mg) as an orange gum. LCMS: RT 1.56 min, [M+H]⁺ 434.2, LCMS method D. ¹H NMR (400 MHz, CDCl₃) δ 7.36 (dd, J=8.8, 5.1 Hz, 1H), 7.19 (d, J=8.3 Hz, 1H), 6.99 (ddd, J=8.8, 7.6, 3.0 Hz, 1H), 6.89 (dd, J=8.8, 2.9 Hz, 1H), 6.42 (dd, J=8.3, 2.4 Hz, 1H), 6.37 (d, J=2.4 Hz, 1H), 5.62 (s, 1H), 5.00 (d, J=14.5 Hz, 1H), 3.78 (s, 3H), 3.76 (d, J=14.7 Hz, 2H), 3.74 (s, 3H), 3.50 (qd, J=20.2, 1.8 Hz, 2H), 2.18 (s, 3H).

### Step 8. 7-(2-chloro-5-fluorophenyl)-6-(2,4-dimethoxybenzyl)-1-(7-fluoro-5-(trifluoromethyl)-1H-benzo[d]imidazol-2-yl)-3-methyl-1,4,6,7-tetrahydro-5H-pyrazolo[3,4-c]pyridin-5-one

To a 20 mL microwave vial were added 4-acetyl-6-(2-chloro-5-fluorophenyl)-1-(2,4-dimethoxybenzyl)-5-hydroxy-3,6-dihydropyridin-2(1H)-one (80.0 mg) and 4-fluoro-2-hydrazinyl-6-(trifluoromethyl)-1H-benzo[d]imidazole (86.4 mg). EtOH (4.45 mL) was added and the reaction vial was flushed with nitrogen for 30 seconds, then sealed and heated at 100° C. for 20 hours. The reaction mixture was concentrated under reduced pressure and the crude product was purified by flash chromatography (dichloromethane:ethyl acetate=50:50) to give the desired product (48.0 mg) as a white solid. LCMS: RT 1.95 min, [M+H]⁺ 632.1, LCMS method D.

### Step 9. 7-(2-chloro-5-fluorophenyl)-1-(7-fluoro-5-(trifluoromethyl)-1H-benzo[d]imidazol-2-yl)-3-methyl-1,4,6,7-tetrahydro-5H-pyrazolo[3,4-c]pyridin-5-one

To a 2 mL microwave vial was added 7-(2-chloro-5-fluorophenyl)-6-(2,4-dimethoxybenzyl)-1-(7-fluoro-5-(trifluoromethyl)-1H-benzo[d]imidazol-2-yl)-3-methyl-1,4,6,7-tetrahydro-5H-pyrazolo[3,4-c]pyridin-5-one (95.0 mg), trifluoroacetic acid (500 μL) and anisole (500 μL). The vial was sealed and heated at 150° C. for 10 minutes in a microwave reactor, then cooled to room temperature. The pH of the reaction mixture was adjusted to 8-9 by careful addition of saturated NaHCO₃ solution at room temperature. The aqueous phase was extracted with ethyl acetate. The organic layers were combined, dried over sodium sulfate, filtered, and concentrated under reduced pressure. The residue was triturated with heptane. The solid was collected by filtration and dried to afford the crude product as a beige powder, which was purified by flash chromatography (dichloromethane:ethyl acetate=40:60) to give the desired product (16 mg) as a white solid. LCMS: RT 3.05 min; [M+H]⁺ 482.1, LCMS method L. ¹H NMR (400 MHz, DMSO-d6) δ 13.61 (s, 1H), 8.53 (s, 1H), 7.56-7.38 (m, 2H), 7.33 (d, J=10.5 Hz, 1H), 7.06 (td, J=8.4, 3.0 Hz, 1H), 6.95 (d, J=6.4 Hz, 1H), 6.62 (s, 1H), 3.63 (d, J=20.8 Hz, 1H), 3.42 (dd, J=20.7, 2.4 Hz, 1H), 2.32 (s, 3H).

### Example 10

### 7-(2-chloro-5-fluorophenyl)-1-(7-fluoro-5-(trifluoromethyl)-1H-benzo[d]imidazol-2-yl)-N-methyl-5-oxo-4,5,6,7-tetrahydro-1H-pyrrolo[2,3-c]pyridine-3-carboxamide (I-31) and methyl 7-(2-chloro-5-fluorophenyl)-1-(7-fluoro-5-(trifluoromethyl)-1H-benzo[d]imidazol-2-yl)-5-oxo-4,5,6,7-tetrahydro-1H-pyrrolo[2,3-c]pyridine-3-carboxylate (I-45)

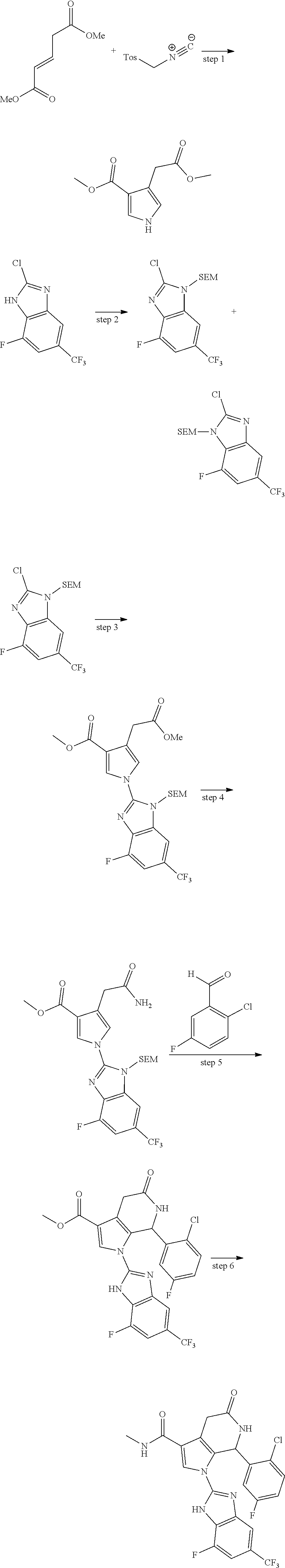

### Step 1. Methyl 4-(2-methoxy-2-oxoethyl)-1H-pyrrole-3-carboxylate

To a stirred 1 M solution of lithium bis(trimethylsilyl)amide solution (1.0 M, 10.0 mL) in THF cooled to −78° C. under a nitrogen atmosphere was added a solution of p-toluenesulfonylmethyl isocyanide (2.00 g) in dry THF (42.5 mL) dropwise over 30 min via a syringe. After stirring for 30 minutes at −78° C., a solution of dimethyl pent-2-enedioate (1.57 g) in dry THF (14.2 mL) was added over 10 minutes at −78° C. When the addition was completed, the cold bath was removed, and the reaction was warmed to room temperature and stirred overnight. The dark red suspension was concentrated in vacuo, and the residue was partitioned between H₂O (150 mL) and CH₂Cl₂ (150 mL). The aqueous layer was extracted with CH₂Cl₂ (5×150 mL). The combined organic layers were dried over Na₂SO₄, filtered and concentrated in vacuo to give a red oil, which was purified by flash chromatography (2:1 hexane:ethyl acetate) to afford the desired product (1.95 g) as a white solid. LCMS: RT 0.82 min, [M+H]⁺ 198.1, LCMS method D. ¹H NMR (400 MHz, CDCl₃) δ 8.72 (s, 1H), 7.43-7.30 (m, 1H), 6.76-6.49 (m, 1H), 3.77 (m, 5H), 3.70 (d, J=1.3 Hz, 3H).

### Step 2. 2-Chloro-4-fluoro-6-(trifluoromethyl)-1-((2-(trimethylsilyl)ethoxy)methyl)-1H-benzo[d]imidazole

To a solution of 2-chloro-4-fluoro-6-(trifluoromethyl)-1H-benzo[d]imidazole (70 mg) in THF (4 mL) at 0° C. was added sodium hydride (60% dispersion in mineral oil, 14.1 mg). The reaction mixture was stirred for 20 min at 0° C. before addition of (2-chloromethoxyethyl) trimethylsilane (58.3 uL). The solution was stirred at room temperature for 16 hours. Water was added, and the organic layer was extracted three times with EtOAc. The combined organic layers were dried over sodium sulfate and concentrated in vacuo. Purification by normal phase flash chromatography (25 g silica cartridge) eluting with a gradient of 0-60% EtOAc in heptanes afforded 2-chloro-7-fluoro-5-(trifluoromethyl)-1-((2-(trimethylsilyl) ethoxy)methyl)-1H-benzo[d]imidazole (25.5 mg) and 2-chloro-4-fluoro-6-(trifluoromethyl)-1-((2-(trimethylsilyl)ethoxy)methyl)-1H-benzo[d]imidazole (65.5 mg), both as clear liquids.

2-chloro-4-fluoro-6-(trifluoromethyl)-1-((2-(trimethylsilyl)ethoxy)methyl)-1H-benzo[d]imidazole; LCMS: RT 2.00 min, [M+H]⁺ not observed, LCMS method D. ¹H NMR (400 MHz, CDCl₃) δ 7.82-7.72 (m, 1H), 7.32-7.18 (m, 1H), 5.68 (s, 2H), 3.72-3.53 (m, 2H), 0.90 (d, J=8.1 Hz, 2H), −0.06 (s, 9H).

2-chloro-7-fluoro-5-(trifluoromethyl)-1-((2-(trimethylsilyl)ethoxy)methyl)-1H-benzo[d]imidazole: LCMS: RT 1.96 min, [M+H]⁺ not observed, LCMS method D. ¹H NMR (400 MHz, CDCl₃) δ 7.57-7.50 (m, 1H), 7.26 (dd, J=6.8, 5.0 Hz, 1H), 5.60 (s, 2H), 3.72-3.50 (m, 2H), 0.98-0.73 (m, 2H), −0.05 (s, 9H).

### Step 3. Methyl 1-(4-fluoro-6-(trifluoromethyl)-1-((2-(trimethylsilyl)ethoxy)methyl)-1H-benzo[d]imidazol-2-yl)-4-(2-methoxy-2-oxoethyl)-1H-pyrrole-3-carboxylate

To a microwave vial were added methyl 4-(2-methoxy-2-oxoethyl)-1H-pyrrole-3-carboxylate (10.2 mg), 2-chloro-4-fluoro-6-(trifluoromethyl)-1-((2-(trimethylsilyl)ethoxy) methyl)-1H-benzo[d]imidazole (12.7 mg), sodium tert-butoxide (10.3 mg) and toluene (300 uL). The vial was purged with nitrogen gas for 30 seconds, then heated at 150° C. under microwave for 60 minutes. After cooling to room temperature, the reaction residue was purified by flash chromatography (heptane:EtOAc=80:20) to give the desired product (10.0 mg) as a colorless oil. LCMS RT 2.04 min, [M+Na]⁺ 552.3, LCMS method D. ¹H NMR (400 MHz, CDCl₃) δ 8.10 (d, J=2.4 Hz, 1H), 7.82 (s, 1H), 7.47 (d, J=2.4 Hz, 1H), 7.33 (d, J=12.7 Hz, 1H), 5.65 (s, 2H), 3.84 (s, 2H), 3.82 (s, 3H), 3.79-3.75 (m, 2H), 3.73 (s, 3H), 1.04-0.96 (m, 2H), 0.01 (s, 9H).

### Step 4. Methyl 4-(2-amino-2-oxoethyl)-1-(4-fluoro-6-(trifluoromethyl)-1-((2-(trimethylsilyl)ethoxy)methyl)-1H-benzo[d]imidazol-2-yl)-1H-pyrrole-3-carboxylate

A stirred suspension of ammonium chloride (41.2 mg) in toluene (2 mL) at 5° C. was treated with trimethylaluminum (337 uL) and stirred at room temperature for 2 hours. A solution of methyl 1-(4-fluoro-6-(trifluoromethyl)-1-((2-(trimethylsilyl)ethoxy)methyl)-1H-benzo[d]imidazol-2-yl)-4-(2-methoxy-2-oxoethyl)-1H-pyrrole-3-carboxylate (150 mg) in toluene (5.5 mL) was added and the reaction was heated at 60° C. for 20 hours. After cooling to room temperature, the reaction was quenched with water and extracted with EtOAc. The organic layers were combined, dried over Na₂SO₄ and concentrated in vacuo to give the crude product, which was purified by flash chromatography (ethyl acetate 100%) to give the desired product (80.0 mg) as a white solid. LCMS RT 1.76 min, [M+Na]⁺ 537.3, LCMS method D. ¹H NMR (400 MHz, CDCl₃) δ 8.10 (d, J=2.3 Hz, 1H), 7.81 (s, 1H), 7.49 (d, J=2.1 Hz, 1H), 7.32 (d, J=11.4 Hz, 1H), 6.53 (s, 1H), 5.63 (s, 2H), 5.51 (s, 1H), 3.84 (s, 3H), 3.77 (t, J=8.0 Hz, 2H), 3.70 (s, 2H), 1.07-0.88 (m, 2H), −0.01 (s, 9H).

### Step 5. Methyl 7-(2-chloro-5-fluorophenyl)-1-(7-fluoro-5-(trifluoromethyl)-1H-benzo[d]imidazol-2-yl)-5-oxo-4,5,6,7-tetrahydro-1H-pyrrolo[2,3-c]pyridine-3-carboxylate

A microwave vial was charged with methyl 4-(2-amino-2-oxoethyl)-1-(7-fluoro-5-(trifluoromethyl)-1-((2-(trimethylsilyl)ethoxy)methyl)-1H-benzo[d]imidazol-2-yl)-1H-pyrrole-3-carboxylate (50 mg) and 2-chloro-5-fluorobenzaldehyde (40 mg). Eaton's reagent (2 mL) was added, and the reaction media was heated at 100° C. under microwave for 15 minutes. After cooling to room temperature, the material was diluted with ethyl acetate (5 mL) and cold saturated NaHCO₃ solution until no more bubbling was observed. The aqueous phase was extracted with ethyl acetate twice. The organic layers were combined, dried over Na₂SO₄, and concentrated to afford the crude product as a brownish oil. The crude material was dissolved in DMF (1 mL) and purified on a 12 g reverse phase column (10 mM ammonium formate solution:acetonitrile 95:5 to 20:80) to give the desired product (16.0 mg) as a white solid. LCMS: RT 1.31 min, [M+H]⁺ 525.4, LCMS method S.

### Step 6. 7-(2-chloro-5-fluorophenyl)-1-(7-fluoro-5-(trifluoromethyl)-1H-benzo[d]imidazol-2-yl)-N-methyl-5-oxo-4,5,6,7-tetrahydro-1H-pyrrolo[2,3-c]pyridine-3-carboxamide

A microwave vial was charged with methyl 7-(2-chloro-5-fluorophenyl)-1-(7-fluoro-5-(trifluoromethyl)-1H-benzo[d]imidazol-2-yl)-5-oxo-4,5,6,7-tetrahydro-1H-pyrrolo[2,3-c]pyridine-3-carboxylate (15 mg). THF (1.2 mL) and MeOH (0.6 mL) were added, followed by methylamine in water (40% by weight, 0.25 mL). The reaction vial was sealed and heated at 50° C. for 5 days. The reaction media was concentrated under reduced pressure. The crude material was dissolved in DMF (1 mL) and purified on a 12 g reverse phase column (10 mM ammonium formate solution:acetonitrile 95:5 to 20:80) to give the desired product (4.0 mg) as a white solid. LCMS: RT 1.40 min, [M+H]⁺ 524.2, LCMS method S. ¹H NMR (400 MHz, DMSO-d6) δ 8.44 (d, J=2.1 Hz, 1H), 8.06 (s, 1H), 7.97 (q, J=4.8 Hz, 1H), 7.57 (s, 1H), 7.31 (dd, J=8.8, 5.2 Hz, 2H), 6.99 (ddd, J=8.7, 8.0, 3.1 Hz, 1H), 6.71 (dd, J=9.4, 3.1 Hz, 1H), 6.68 (overlapping br s, 1H), 3.76 (dd, J=21.5, 2.6 Hz, 1H), 3.68 (dd, J=21.4, 2.5 Hz, 1H), 2.76 (d, J=4.5 Hz, 3H).

### Example 11

### 1-(2-chloro-5-fluorophenyl)-8-(3-fluoro-5-(trifluoromethyl)benzamido)-3-oxo-N-(pyridin-2-ylmethyl)-1,2,3,4-tetrahydropyrrolo[1,2-a]pyrazine-6-carboxamide (I-34)

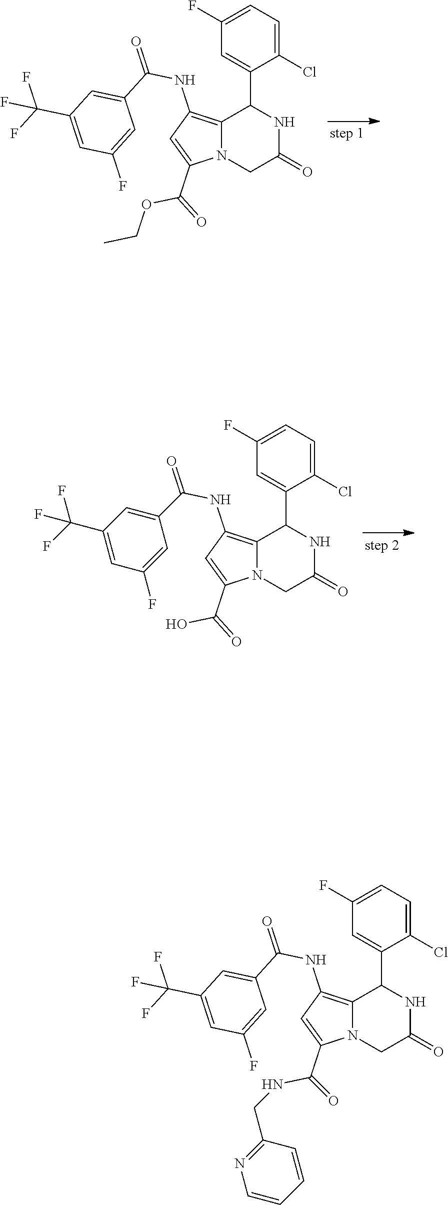 [SMILES: CCOC(=O)c1cc(NC(=O)c2cc(F)cc(C(F)(F)F)c2)c2n1CC(=O)NC2c1cc(F)ccc1Cl ; O=C1Cn2c(C(=O)O)cc(NC(=O)c3cc(F)cc(C(F)(F)F)c3)c2C(c2cc(F)ccc2Cl)N1 ; O=C1Cn2c(C(=O)NCc3ccccn3)cc(NC(=O)c3cc(F)cc(C(F)(F)F)c3)c2C(c2cc(F)ccc2Cl)N1]

### Step 1. 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylic acid

To a solution of ethyl 1-(2-chloro-5-fluorophenyl)-8-[3-fluoro-5-(trifluoromethyl)benzamido]-3-oxo-1H,2H,3H,4H-pyrrolo[1,2-a]pyrazine-6-carboxylate (Intermediate III, 4.0 g) in THF (18.48 mL) was added 2 M LiOH in water (18.48 mL). The mixture was stirred at 40° C. overnight. HCl (5 M) was added at 0° C., and the mixture was stirred at room temperature for 10 hours. The reaction was quenched with water and extracted with ethyl acetate. The organic layer was washed with brine, dried over Na₂SO₄, and concentrated to give the desired product (3.2 g) as a white solid. LCMS: RT 0.811 min, [M+H]⁺ 514.10, LCMS method P.

### Step 2. 1-(2-chloro-5-fluorophenyl)-8-(3-fluoro-5-(trifluoromethyl)benzamido)-3-oxo-N-(pyridin-2-ylmethyl)-1,2,3,4-tetrahydropyrrolo[1,2-a]pyrazine-6-carboxamide

To a stirred solution of 1-(2-chloro-5-fluorophenyl)-8-[3-fluoro-5-(trifluoromethyl)benzamido]-3-oxo-1H,2H,3H,4H-pyrrolo[1,2-a]pyrazine-6-carboxylic acid (50 mg), 1-(pyridin-2-yl)methanamine (15.6 mg), and NaHCO₃ (25 mg) in DMF (1.0 mL) was added HATU (55.55 mg) at room temperature. The resulting mixture was stirred at room temperature for 1 hour. The reaction was quenched with water and extracted with ethyl acetate. The organic layer was washed with brine, dried over Na₂SO₄ and concentrated. The resulting crude material was purified using prep-HPLC (Column: XBridge Prep OBD C18 Column, 30×150 mm, 5 μm; mobile phase A: water (10 mM NH₄HCO₃+0.1% NH₃·H₂O), mobile phase B: acetonitrile; flow rate: 60 mL/min; Gradient: 25% B to 60% B in 8 min; RT 7.23 min) to give the desired product (20.9 mg) as a white amorphous solid. LCMS: RT 1.016 min, [M+H]⁺ 604.30, LCMS method P.

Additional compounds prepared according to the methods of Example 11 are listed in Table 5 below. Corresponding ¹H NMR and mass spectrometry characterization for these compounds are described in Table 1. Certain compounds in Table 5 below were prepared with other compounds whose preparation is described in the Examples herein.

TABLE 5
Additional Exemplary Compounds
Compound

| | | |
|---|---|---|
| I-10 | I-38 | I-66 |
| I-11 | I-39 | I-67 |
| I-14 | I-40 | I-68 |
| I-16 | I-41 | I-69 |
| I-18 | I-42 | I-70 |
| I-19 | I-43 | I-71 |
| I-20 | I-47 | I-74 |
| I-21 | I-48 | I-75 |
| I-22 | I-49 | I-76 |
| I-23 | I-58 | I-78 |
| I-26 | I-59 | I-79 |
| I-27 | I-61 | I-87 |
| I-28 | I-62 | I-88 |
| I-29 | I-63 | I-89 |
| I-33 | I-64 | I-93 |
| I-37 | I-65 | I-94 |
| I-95 | I-172 | I-218 |
| I-96 | I-173 | I-219 |
| I-97 | I-174 | I-220 |
| I-98 | I-175 | I-221 |
| I-99 | I-176 | I-222 |
| I-101 | I-177 | I-231 |
| I-102 | I-178 | I-232 |
| I-117 | I-179 | I-237 |
| I-118 | I-180 | I-252 |
| I-119 | I-181 | I-253 |
| I-123 | I-182 | I-260 |
| I-124 | I-188 | I-261 |
| I-125 | I-190 | I-282 |
| I-126 | I-191 | I-283 |
| I-127 | I-192 | I-286 |
| I-129 | I-193 | I-287 |
| I-130 | I-194 | I-300 |
| I-140 | I-195 | I-301 |
| I-141 | I-196 | I-313 |
| I-142 | I-197 | I-345 |
| I-143 | I-198 | I-346 |
| I-144 | I-199 | I-409 |
| I-145 | I-213 | I-410 |
| I-166 | I-214 | I-442 |
| I-167 | I-215 | I-443 |
| I-170 | I-216 | I-484 |
| I-171 | I-217 | |

### Example 12

### (R)-1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylic acid (I-35 or I-36) and (S)-1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylic acid (I-35 or I-36)

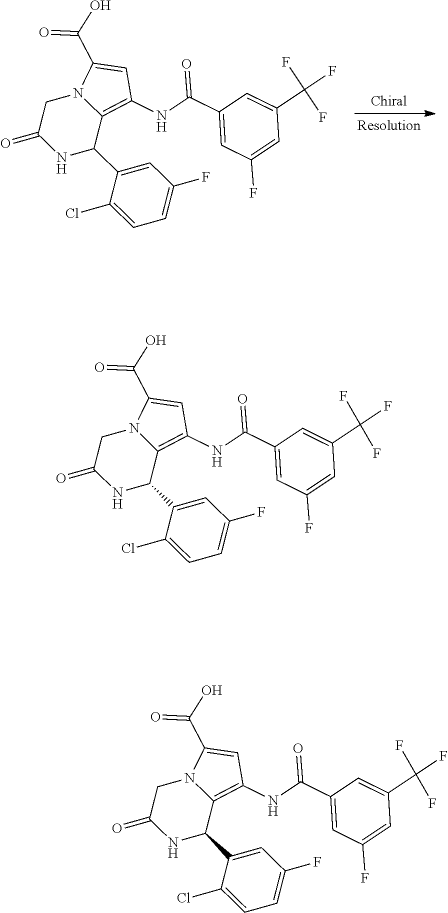 [SMILES: O=C1Cn2c(C(=O)O)cc(NC(=O)c3cc(F)cc(C(F)(F)F)c3)c2C(c2cc(F)ccc2Cl)N1 ; O=C1Cn2c(C(=O)O)cc(NC(=O)c3cc(F)cc(C(F)(F)F)c3)c2[C@H](c2cc(F)ccc2Cl)N1 ; O=C1Cn2c(C(=O)O)cc(NC(=O)c3cc(F)cc(C(F)(F)F)c3)c2[C@@H](c2cc(F)ccc2Cl)N1]

1-(2-Chloro-5-fluorophenyl)-8-(3-fluoro-5-(trifluoromethyl)benzamido)-3-oxo-1,2,3,4-tetrahydropyrrolo[1,2-a]pyrazine-6-carboxylic acid (120 mg) was chirally resolved using prep-chiral-HPLC with the following conditions: Column: CHIRALPAK IH, 3*25 cm, 5 μm; mobile phase A: hexane (with 0.2% formic acid), mobile phase B: EtOH:dichloromethane 1:1; flow rate: 18 mL/min; gradient:60% B isocratic; wavelength: 220/254 nm; peak 1 RT 3.263 min; peak 2 RT 8.203 min; injection volume: 3 mL; number of runs: 3) to give both enantiomers as an off-white amorphous solid.

Compound I-35, Peak 1. 49.7 mg. LCMS: RT 0.790 min, [M+H]⁺ 514.05, LCMS method P. ¹HNMR (400 MHz, DMSO-d6) δ 9.87 (1H, s), 8.89 (1H, d, J=2.4 Hz), 7.92 (1H, d, J=8.3 Hz), 7.80 (2H, d, J=6.5 Hz), 7.36 (1H, dd, J=8.7, 5.1 Hz), 7.09 (2H, ddt, J=12.2, 6.3, 3.1 Hz), 6.78 (1H, s), 6.07-6.02 (1H, m), 5.02 (2H, s).

Compound I-36, Peak 2. 46.5 mg. LCMS: RT 1.121 min, [M+H]⁺ 514.10, LCMS method N. ¹HNMR (400 MHz, DMSO-d6) δ 12.56 (1H, s), 9.87 (1H, s), 8.90 (1H, d, J=2.5 Hz), 7.92 (1H, d, J=8.5 Hz), 7.80 (2H, d, J=7.4 Hz), 7.36 (1H, dd, J=8.5, 5.1 Hz), 7.15-7.04 (2H, m), 6.80 (1H, s), 6.04 (1H, d, J=2.3 Hz), 5.02 (2H, s).

### Example 13

### (R)—N-(1-(2-chloro-5-fluorophenyl)-6-cyano-3-oxo-1,2,3,4-tetrahydropyrrolo[1,2-a]pyrazin-8-yl)-3-fluoro-5-(trifluoromethyl)benzamide (I-72 or I-73) and (S)—N-(1-(2-chloro-5-fluorophenyl)-6-cyano-3-oxo-1,2,3,4-tetrahydropyrrolo[1,2-a]pyrazin-8-yl)-3-fluoro-5-(trifluoromethyl)benzamide (I-72 or I-73)

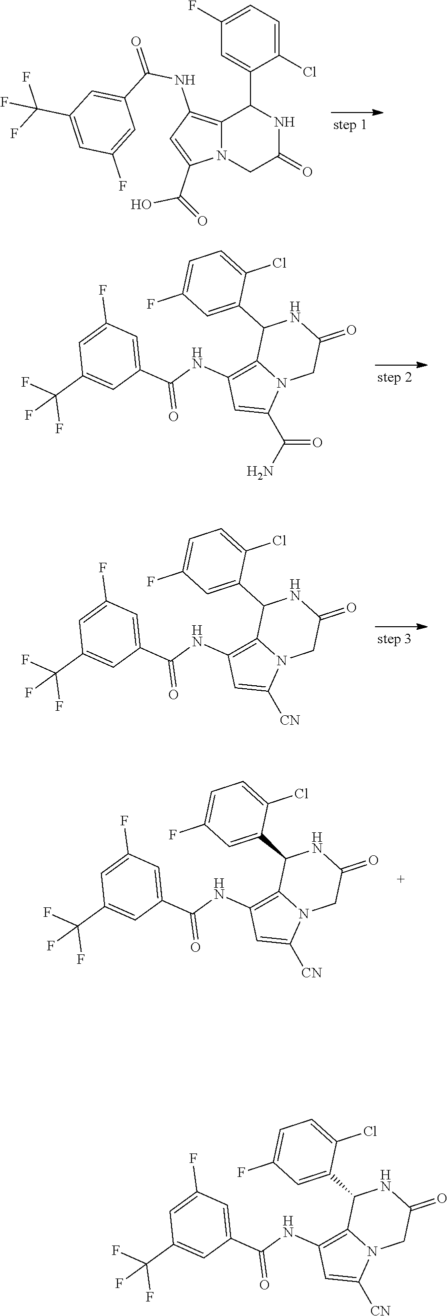

### Step 1. 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxamide

To a stirred solution of 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxylic acid (100 mg), NH₃ in THF (584 μL, 0.5 molar) and NaHCO₃ (49 mg) in DMF (1.0 mL) was added HATU (111 mg) at room temperature. The resulting mixture was stirred at room temperature for 1 hour and purified using C18 flash chromatography with the following conditions (mobile phase A: water, mobile phase B: acetonitrile; flow rate: 60 mL/min; gradient: 0% B to 100% B in 40 min; wavelength: 254/220 nm) to give the desired product (80 mg) as a white solid. LCMS: RT 0.97 min, [M+H]⁺=513.3, LCMS method O.

### Step 2. N-(1-(2-chloro-5-fluorophenyl)-6-cyano-3-oxo-1,2,3,4-tetrahydropyrrolo[1,2-a]pyrazin-8-yl)-3-fluoro-5-(trifluoromethyl)benzamide

To a solution of 1-(2-chloro-5-fluorophenyl)-8-(3-fluoro-5-(trifluoromethyl)benzamido)-3-oxo-1,2,3,4-tetrahydropyrrolo[1,2-a]pyrazine-6-carboxamide (120 mg) and pyridine (147 mg) in 1,4-dioxane (3 mL) at 0° C. was added trifluoroacetyl anhydride (196 mg) dropwise. After 1 hour at room temperature, the reaction was quenched with water and extracted with ethyl acetate. The organic layer was washed with brine, dried over Na₂SO₄ and concentrated. The crude residue was purified using prep-HPLC with the following conditions: Column: YMC-Actus Triart C18 ExRS, 30 mm×150 mm, 5 μm; mobile phase A: 10 mM NH₄HCO₃ solution, mobile phase B: acetonitrile; flow rate: 60 mL/min; gradient: 35% B to 60% B in 7 min then 60% B; wavelength: 220 nm; RT 6.42 min, which gave the desired product (33.5 mg) as an amorphous solid. LCMS: RT 1.62 min, [M+H]⁺ 495.0. LCMS method E.

### Step 3. (R)—N-(1-(2-chloro-5-fluorophenyl)-6-cyano-3-oxo-1,2,3,4-tetrahydropyrrolo[1,2-a]pyrazin-8-yl)-3-fluoro-5-(trifluoromethyl)benzamide and (S)—N-(1-(2-chloro-5-fluorophenyl)-6-cyano-3-oxo-1,2,3,4-tetrahydropyrrolo[1,2-a]pyrazin-8-yl)-3-fluoro-5-(trifluoromethyl)benzamide

N-(1-(2-chloro-5-fluorophenyl)-6-cyano-3-oxo-1,2,3,4-tetrahydropyrrolo[1,2-a]pyrazin-8-yl)-3-fluoro-5-(trifluoromethyl)benzamide (20 mg) was chirally resolved using chiral HPLC with the following conditions: Column: CHIRALPAK IH, 2*25 cm, 5 μm; mobile phase A: hexane (0.2% triethylamine), mobile phase B: EtOH:dichloromethane 1:1; flow rate: 20 mL/min; gradient: 60% B isocratic; wavelength 220/254 nm; peak 1 RT 3.473 min; peak 2 RT 11.196 min; injection volume: 1.65 mL. Both enantiomers were obtained as a white amorphous solid.

Compound I-73, Peak 1. 6.6 mg. LCMS: RT 1.08 min, [M+H]⁺=495.1, LCMS method E. ¹H NMR (400 MHz, DMSO-d6) δ 9.97 (1H, s), 9.00 (1H, d, J=2.3 Hz), 7.93 (1H, dt, J=8.7, 2.0 Hz), 7.78 (2H, dd, J=8.5, 2.1 Hz), 7.41-7.32 (1H, m), 7.11 (2H, t, J=8.0 Hz), 7.03 (1H, s), 6.03 (1H, d, J=2.4 Hz), 5.09-4.74 (2H, m).

Compound I-72, Peak 2. 4.3 mg. LCMS: RT 1.08 min, [M+H]⁺=495.0, LCMS method E. ¹H NMR (400 MHz, DMSO-d6) δ 9.97 (1H, s), 9.00 (1H, d, J=2.3 Hz), 7.93 (1H, dt, J=8.7, 2.0 Hz), 7.78 (2H, dd, J=8.5, 2.1 Hz), 7.41-7.32 (1H, m), 7.16-7.06 (2H, m), 7.03 (1H, s), 6.03 (1H, q, J=1.8 Hz), 5.12-4.76 (2H, m).

Additional compounds prepared according to the methods of Example 13 are listed in Table 6 below. Corresponding ¹H NMR and mass spectrometry characterization for these compounds are described in Table 1. Certain compounds in Table 6 below were prepared with other compounds whose preparation is described in the Examples herein.

TABLE 6
Additional Exemplary Compounds
Compound

I-30
I-209
I-210

### Example 14

### ethyl 1-(2-chloro-5-fluorophenyl)-8-(3-fluoro-5-(trifluoromethyl)benzamido)-4,4-dimethyl-3-oxo-1,2,3,4-tetrahydropyrrolo[1,2-a]pyrazine-6-carboxylate (I-46) and 1-(2-chloro-5-fluorophenyl)-8-(3-fluoro-5-(trifluoromethyl)benzamido)-N,4,4-trimethyl-3-oxo-1,2,3,4-tetrahydropyrrolo[1,2-a]pyrazine-6-carboxamide (I-77)

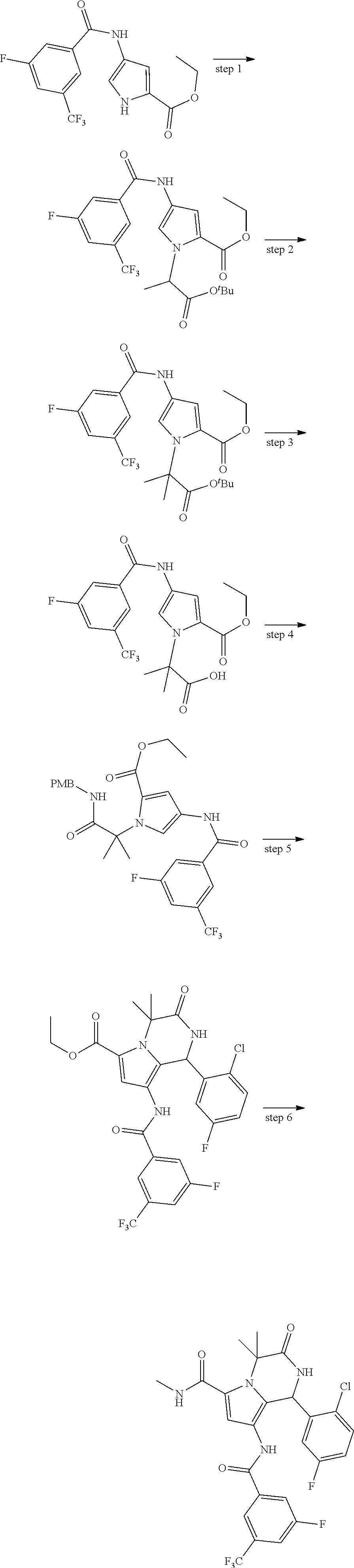

### Step 1. ethyl 1-(1-(tert-butoxy)-1-oxopropan-2-yl)-4-(3-fluoro-5-(trifluoromethyl)benzamido)-1H-pyrrole-2-carboxylate

A 10 mL round bottom flask was charged with ethyl 4-(3-fluoro-5-(trifluoromethyl)benzamido)-1H-pyrrole-2-carboxylate (0.5 g). Acetonitrile (2 mL) was added, followed by 2-(tert-butyl)-1,1,3,3-tetramethylguanidine (441 μL) dropwise. The resulting yellow solution was stirred for 5 minutes prior to addition of tert-butyl 2-bromopropanoate (1.21 g). After 30 minutes the solution was concentrated under vacuum and the crude material was dissolved in DMF (1 mL) and loaded on a 30 g reverse phase column for purification (10 mM ammonium formate solution:acetonitrile 95:5 to 35:65) to give the desired product (600 mg) as a white solid. LCMS: RT 1.62 min, [M−H]⁻ 471.5, LCMS method U.

### Step 2. ethyl 1-(1-(tert-butoxy)-2-methyl-1-oxopropan-2-yl)-4-(3-fluoro-5-(trifluoromethyl)benzamido)-1H-pyrrole-2-carboxylate

To a flame-dried 10 mL round bottom flask was added ethyl 1-(1-(tert-butoxy)-1-oxopropan-2-yl)-4-(3-fluoro-5-(trifluoromethyl)benzamido)-1H-pyrrole-2-carboxylate (600 mg). THF (10 ml) was added under nitrogen and the reaction mixture was cooled to 0° C. Sodium hydride (33.3 mg) was added, the reaction mixture was stirred for 15 min and then cooled to −78° C. KHMDS (1 M in THF, 6.35 mL) was added dropwise. The reaction mixture was stirred for 30 minutes at −78° C., followed by addition of methyl iodide (276 μL). The reaction mixture was stirred at −78° C. for 90 minutes. The reaction was quenched at −78° C. with saturated ammonium chloride solution. The solution was extracted with ethyl acetate, dried over Na₂SO₄, filtered and concentrated under reduced pressure to give the crude product (230 mg), which was used in the next step without purification. LCMS: RT 1.65 min, [M−H]⁻ 485.5, LCMS method U.

### Step 3. 2-(2-(ethoxycarbonyl)-4-(3-fluoro-5-(trifluoromethyl)benzamido)-1H-pyrrol-1-yl)-2-methylpropanoic acid

To a flame-dried 25 mL round bottom flask was added ethyl 1-(1-(tert-butoxy)-2-methyl-1-oxopropan-2-yl)-4-(3-fluoro-5-(trifluoromethyl)benzamido)-1H-pyrrole-2-carboxylate (0.23 g). TFA (1.5 mL) was added and the reaction mixture was stirred at 90° C. for 1 hour. The reaction mixture was concentrated under reduced pressure to give the desired product (203 mg), which was used in the next step without purification. LCMS: RT 1.25 min, [M−H]⁻ 429.4, LCMS method U.

### Step 4. ethyl 4-(3-fluoro-5-(trifluoromethyl)benzamido)-1-(1-((4-methoxybenzyl)amino)-2-methyl-1-oxopropan-2-yl)-1H-pyrrole-2-carboxylate

To a 20 mL microwave vial was added 2-(2-(ethoxycarbonyl)-4-(3-fluoro-5-(trifluoromethyl)benzamido)-1H-pyrrol-1-yl)-2-methylpropanoic acid (140 mg), 1-(4-methoxyphenyl)methanamine (89.1 mg), chloro-N,N,N′,N′-tetramethylformamidinium hexafluorophosphate (182 mg) and N-methylimidazole (80 mg). Acetonitrile (15 mL) was added, and the reaction mixture was heated under microwave at 100° C. for 20 minutes. The solution was concentrated, and the residue was dissolved in ethyl acetate. The organic solution was washed with water, saturated NH₄Cl solution and brine, dried over Na₂SO₄ and concentrated to afford the crude product (160 mg), which was used in the next step without further purification. LCMS: RT 1.43 min, [M+H]⁺ 550.5, LCMS method U.

### Step 5. ethyl 1-(2-chloro-5-fluorophenyl)-8-(3-fluoro-5-(trifluoromethyl)benzamido)-4,4-dimethyl-3-oxo-1,2,3,4-tetrahydropyrrolo[1,2-a]pyrazine-6-carboxylate

A microwave vial was charged with ethyl 4-(3-fluoro-5-(trifluoromethyl)benzamido)-1-(1-((4-methoxybenzyl)amino)-2-methyl-1-oxopropan-2-yl)-1H-pyrrole-2-carboxylate (120 mg) and 2-chloro-5-fluorobenzaldehyde (47.6 mg). Eaton's reagent (4 mL) was added, and the reaction was heated under microwave at 100° C. for 15 minutes. After cooling to room temperature ethyl acetate (5 mL) was added, followed by cold saturated NaHCO₃ solution until no more bubbling was observed. The aqueous phase was extracted with ethyl acetate twice. The organic layers were combined, dried over Na₂SO₄ and concentrated under vacuum to afford the crude product as a brownish oil. The crude material was dissolved in DMF (1 mL) and loaded on a reverse phase column (30 g column, 10 mM ammonium formate solution:acetonitrile 95:5 to 35:65 in 16 minutes) to give the desired product (15 mg) as a white solid. LCMS: RT 1.72 min, [M+H]⁺ 570.2, LCMS method Q. ¹H NMR (400 MHz, DMSO-d6) δ 8.00 (br. s, 1H), 7.62 (br. d, J=8.4 Hz, 1H), 7.57 (d, J=9.1 Hz, 1H), 7.54 (br s, 1H), 7.29 (dd, J=8.8, 5.1 Hz, 1H), 7.07 (s, 1H), 7.00-6.94 (m, 1H), 6.89 (dd, J=9.1, 3.0 Hz, 2H), 6.11 (s, 1H), 4.36-4.20 (m, 2H), 2.03 (s, 3H), 2.00 (s, 3H), 1.33 (t, J=7.1 Hz, 3H).

### Step 6. 1-(2-chloro-5-fluorophenyl)-8-(3-fluoro-5-(trifluoromethyl)benzamido)-N,4,4-trimethyl-3-oxo-1,2,3,4-tetrahydropyrrolo[1,2-a]pyrazine-6-carboxamide

To a flame-dried microwave vial was added ethyl 1-(2-chloro-5-fluorophenyl)-8-(3-fluoro-5-(trifluoromethyl)benzamido)-4,4-dimethyl-3-oxo-1,2,3,4-tetrahydropyrrolo[1,2-a]pyrazine-6-carboxylate (5 mg). Methylamine (2 M in THF, 438 μL) was added, followed by trimethyl aluminum (219 μL) dropwise under nitrogen. The reaction was heated at 55° C. for 72 hours. The reaction mixture was quenched with saturated NaHCO₃ solution, and the crude reaction mixture was extracted with ethyl acetate. The organic layer was dried over Na₂SO₄ and concentrated under reduced pressure. The crude material was dissolved in DMF (1 mL) and loaded on a reverse phase column for purification (30 g column. 10 mM ammonium formate solution:acetonitrile 95:5 to 50:50 in 16 minutes) to give the desired product (3 mg) as a white solid. LCMS: RT 2.24 min, [M+H]⁺ 555.3, LCMS method V. ¹H NMR (400 MHz, acetonitrile-d3) δ 7.98 (br s, 1H), 7.61 (d, J=8.4 Hz, 1H), 7.56 (submerged br d, J=7.6 Hz, 1H), 7.55 (overlapping s, 1H), 7.28 (dd, J=8.8, 5.1 Hz, 1H), 6.96 (td, J=8.4, 3.1 Hz, 1H), 6.88 (dd, J=9.2, 3.1 Hz, 1H), 6.83 (br s, 1H), 6.78 (br s, 1H), 6.56 (s, 1H), 6.10 (d, J=1.2 Hz, 1H), 2.82 (d, J=4.8 Hz, 3H), 1.99 (s, 3H), 1.96 (s, 3H).

### Example 15

### 1-(2-chloro-5-fluorophenyl)-8-(7-fluoro-5-(trifluoromethyl)-1H-benzo[d]imidazol-2-yl)-N-methyl-3-oxo-1,2,3,4-tetrahydropyrrolo[1,2-a]pyrazine-6-carboxamide (I-84)

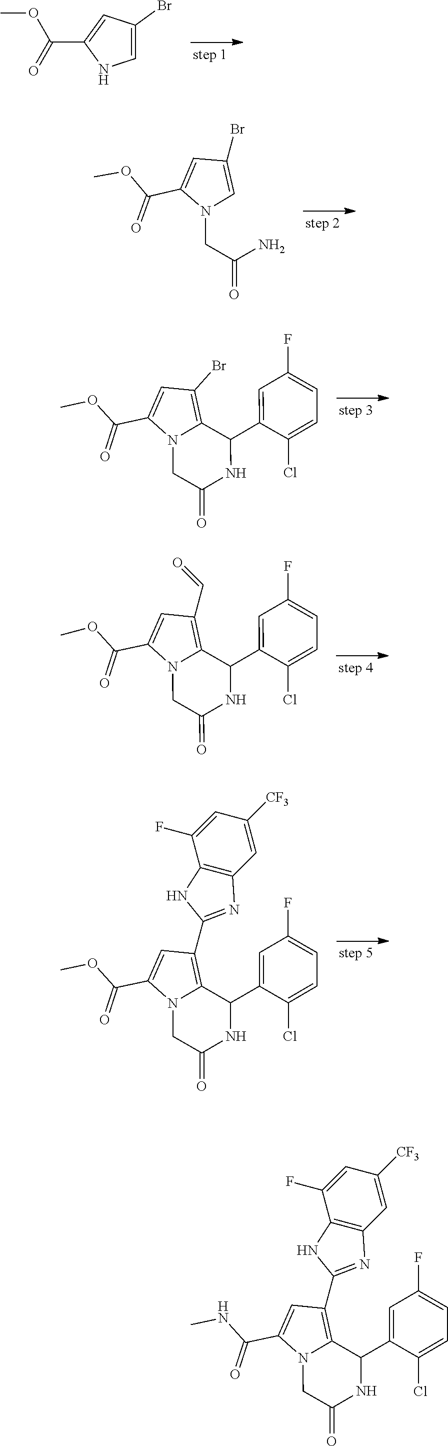

### Step 1. methyl 1-(2-amino-2-oxoethyl)-4-bromo-1H-pyrrole-2-carboxylate

A round bottom flask was charged with methyl 4-bromo-1H-pyrrole-2-carboxylate (2 g) dissolved in acetonitrile (30 mL). 2-(tert-butyl)-1,1,3,3-tetramethylguanidine (1.8 g) was added in the solution, and the solution was stirred at room temperature for 5 minutes. Then 2-bromoacetamide (2.02 g) was added and the solution was stirred at room temperature for 1 hour. The reaction was quenched with water and extracted with ethyl acetate. The organic layer was washed with brine, dried over Na₂SO₄ and concentrated. The residue was dissolved in acetonitrile and concentrated when a precipitate formed. It was collected by filtration to give the desired product (2.2 g) as an off-white solid. LCMS: RT 0.954 min, [M+H]⁺ 260.95. LCMS method E.

### Step 2. methyl 8-bromo-1-(2-chloro-5-fluorophenyl)-3-oxo-1,2,3,4-tetrahydropyrrolo[1,2-a]pyrazine-6-carboxylate

A round bottom flask was charged with methyl 1-(2-aminoacetyl)-4-bromo-1H-pyrrole-2-carboxylate (100 mg) and 2-chloro-5-fluorobenzaldehyde (74 mg). Eaton's reagent (2.5 mL) was added, and the solution was stirred at 85° C. for 50 minutes. The reaction mixture was diluted with ethyl acetate, cooled to 0° C., then quenched with saturated NaHCO₃ solution and extracted with ethyl acetate. The organic phase was dried over Na₂SO₄ and concentrated. A precipitate formed and was collected by filtration to give the desired product (108 mg) as an off-white solid. LCMS: RT 0.954 min, [M+H]⁺ 402.90, LCMS method E.

### Step 3. methyl 1-(2-chloro-5-fluorophenyl)-8-formyl-3-oxo-1,2,3,4-tetrahydropyrrolo[1,2-a]pyrazine-6-carboxylate

A round bottom flask was charged with methyl 8-bromo-1-(2-chloro-5-fluorophenyl)-3-oxo-1,2,3,4-tetrahydropyrrolo[1,2-a]pyrazine-6-carboxylate (300 mg) dissolved in THF (10 mL) and a stir bar. At −78° C., butyllithium (1.7 M THF solution, 1.09 mL) was added dropwise under N₂ and the solution was stirred at room temperature for 25 min. N,N-dimethylformamide (276 mg) was added and the solution was stirred at room temperature for 1 hour. The reaction mixture was slowly transferred into a stirred NH₄Cl solution and extracted with ethyl acetate. The organic layer was washed with brine, dried over Na₂SO₄ and concentrated to give the desired product (186 mg) as a white solid. LCMS: RT 0.915 min, [M+H]⁺ 350.95, LCMS method A.

### Step 4. methyl 1-(2-chloro-5-fluorophenyl)-8-(7-fluoro-5-(trifluoromethyl)-1H-benzo[d]imidazol-2-yl)-3-oxo-1,2,3,4-tetrahydropyrrolo[1,2-a]pyrazine-6-carboxylate

To a solution of methyl 1-(2-chloro-5-fluorophenyl)-8-formyl-3-oxo-1,2,3,4-tetrahydropyrrolo[1,2-a]pyrazine-6-carboxylate (50 mg, 142 μmol) in DMF/H₂O (1.1 mL, 10:1) chilled in an ice-water bath were added 3-fluoro-5-(trifluoromethyl)benzene-1,2-diamine (28 mg) and [(hydroperoxysulfonyl)oxy]potassium (14 mg). The mixture was stirred overnight at room temperature. The reaction was quenched with water and extracted with ethyl acetate. The organic layer was washed with brine, dried over Na₂SO₄ and concentrated to give the desired product (30 mg), which was used in the next step without purification. LCMS: RT 1.023 min, [M+H]⁺ 525.0, LCMS method E.

### Step 5. 1-(2-chloro-5-fluorophenyl)-8-(7-fluoro-5-(trifluoromethyl)-1H-benzo[d]imidazol-2-yl)-N-methyl-3-oxo-1,2,3,4-tetrahydropyrrolo[1,2-a]pyrazine-6-carboxamide

A round bottom flask was charged with methyl 1-(2-chloro-5-fluorophenyl)-8-[7-fluoro-5-(trifluoromethyl)-1H-1,3-benzodiazol-2-yl]-3-oxo-1H,2H,3H,4H-pyrrolo[1,2-a]pyrazine-6-carboxylate (50 mg), methylamine (2 M in methanol, 10 mL) and a stir bar. Methanol (10 mL) was added, and the solution was stirred at room temperature for 2 days. The reaction was quenched with water and extracted with ethyl acetate. The organic layer was washed with brine, dried over Na₂SO₄ and concentrated. The resulting crude material was purified using prep-HPLC with the following conditions: Column: XBridge Shield RP18 OBD Column, 30*150 mm, 5 μm; mobile phase A: 10 mM NH₄HCO₃, mobile phase B: acetonitrile; flow rate: 60 mL/min; Gradient: 35% B to 60% B in 7 min; 220 nm; RT 6.47 min. This gave the desired product (8.7 mg) as a white amorphous solid. LCMS: RT 1.082 min, [M+H]⁺ 524.30, LCMS method P. ¹H NMR (400 MHz, DMSO-d6) δ 9.12 (d, J=3.7 Hz, 1H), 8.37 (s, 1H), 7.57 (s, 1H), 7.47-7.39 (m, 2H), 7.31 (d, J=10.4 Hz, 1H), 7.09 (td, J=8.4, 3.1 Hz, 1H), 6.99-6.93 (m, 1H), 6.82 (d, J=3.6 Hz, 1H), 5.27 (s, 1H), 5.06 (s, 1H), 2.80 (d, J=4.6 Hz, 3H).

### Example 16

### (S)-1-(2-chloro-5-fluorophenyl)-8-(indoline-1-carboxamido)-N-methyl-3-oxo-1,2,3,4-tetrahydropyrrolo[1,2-a]pyrazine-6-carboxamide (I-85 or I-86) and (R)-1-(2-chloro-5-fluorophenyl)-8-(indoline-1-carboxamido)-N-methyl-3-oxo-1,2,3,4-tetrahydropyrrolo[1,2-a]pyrazine-6-carboxamide (I-85 or I-86)

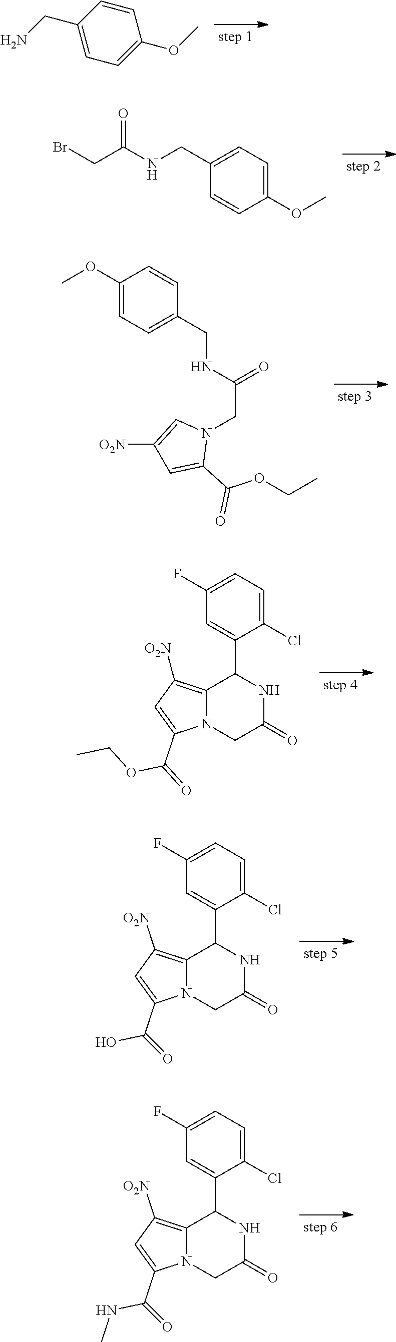

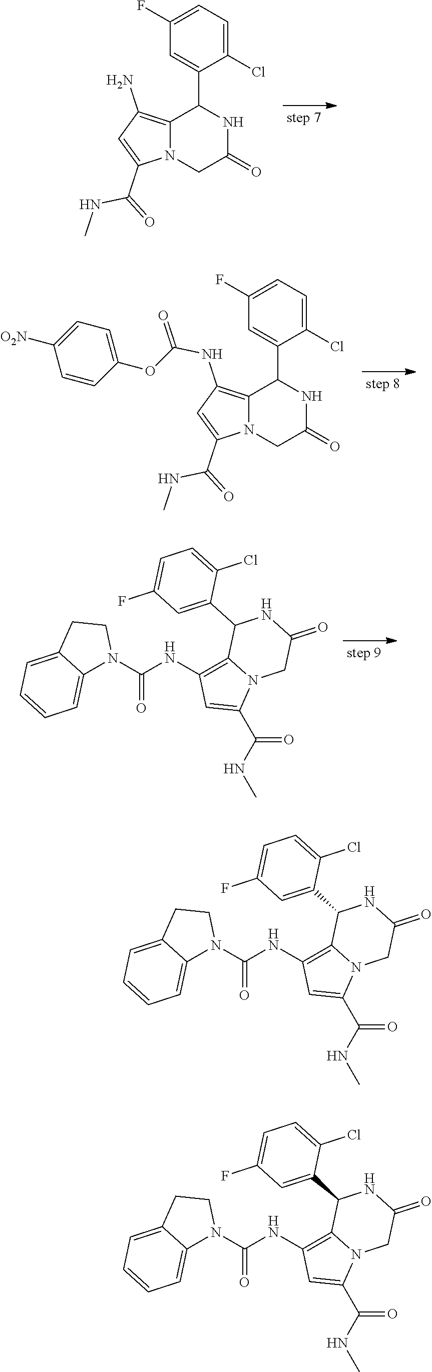 [SMILES: CNC(=O)c1cc(N)c2n1CC(=O)NC2c1cc(F)ccc1Cl ; CNC(=O)c1cc(NC(=O)Oc2ccc([N+](=O)[O-])cc2)c2n1CC(=O)NC2c1cc(F)ccc1Cl ; CNC(=O)c1cc(NC(=O)N2CCc3ccccc32)c2n1CC(=O)NC2c1cc(F)ccc1Cl ; CNC(=O)c1cc(NC(=O)N2CCc3ccccc32)c2n1CC(=O)N[C@H]2c1cc(F)ccc1Cl ; CNC(=O)c1cc(NC(=O)N2CCc3ccccc32)c2n1CC(=O)N[C@@H]2c1cc(F)ccc1Cl]

### Step 1. 2-bromo-N-(4-methoxybenzyl)acetamide

A round bottom flask was charged with 1-(4-methoxyphenyl)methanamine (12 g), triethylamine (8.84 g) and a stir bar. Tetrahydrofuran (120 mL) was added, and the solution was stirred at −70° C. 2-Bromoacetyl chloride (13.7 g) was added slowly, and the solution was stirred at room temperature overnight. The reaction was quenched with water and extracted with ethyl acetate. The organic layer was washed with brine, dried over Na₂SO₄ and concentrated to give the desired product (20 g), which was used in the next step without purification. LCMS: RT 1.077 min, [M+H]⁺ 258.05, LCMS method A.

### Step 2. ethyl 1-(2-((4-methoxybenzyl)amino)-2-oxoethyl)-4-nitro-1H-pyrrole-2-carboxylate

A round bottom flask was charged with ethyl 4-nitro-1H-pyrrole-2-carboxylate (10 g) dissolved in acetonitrile (150 mL) and a stir bar. 2-(tert-butyl)-1,1,3,3-tetramethylguanidine (9.30 g) was added and the solution was stirred at room temperature for 5 minutes. Then 2-bromo-N-[(4-methoxyphenyl)methyl]acetamide (21.0 g) was added and the solution was stirred at room temperature for 2 hours. The reaction was quenched with water and extracted with ethyl acetate. The organic layer was washed with brine, dried over Na₂SO₄ and concentrated. A precipitate formed and was collected by filtration to give the product (10 g) as an off-white solid. CMS: RT 1.065 min, [M+H]⁺ 362.05, LCMS method A.

### Step 3. ethyl 1-(2-chloro-5-fluorophenyl)-8-nitro-3-oxo-1,2,3,4-tetrahydropyrrolo[1,2-a]pyrazine-6-carboxylate

A round bottom flask was charged with ethyl 1-({[(4-methoxyphenyl)methyl]carbamoyl}methyl)-4-nitro-1H-pyrrole-2-carboxylate (260 mg), 2-chloro-5-fluorobenzaldehyde (136 mg) and a stir bar. Eaton's reagent (6.5 mL) was added, and the solution was stirred at 80° C. for 50 minutes. The reaction mixture was diluted with ethyl acetate, cooled to −20° C., quenched with saturated NaHCO₃ solution and extracted with ethyl acetate. The organic phase was dried over Na₂SO₄ and concentrated. The residue was purified by prep-TLC (petroleum ether:ethyl acetate 1:1) to the desired product (130 mg) as a yellow solid. LCMS: RT 1.098 min, [M+H]⁺ 381.95, LCMS method A.

### Step 4. 1-(2-chloro-5-fluorophenyl)-8-nitro-3-oxo-1,2,3,4-tetrahydropyrrolo[1,2-a]pyrazine-6-carboxylic acid

A round bottom flask was charged with ethyl 1-(2-chloro-5-fluorophenyl)-8-nitro-3-oxo-1,2,3,4-tetrahydropyrrolo[1,2-a]pyrazine-6-carboxylate (1.5 g), trimethylstannanol (3.52 g) and a stir bar. Dichloroethane (7.5 mL) was added, and the solution was stirred at 80° C. overnight. The solution was concentrated and purified using C18 flash chromatography with the following conditions (mobile phase A: water, mobile phase B: acetonitrile; flow rate: 60 mL/min; Gradient: 0% B to 100% B in 40 minutes; wavelength: 254/220 nm) to give the desired product (300 mg) as a brown solid. LCMS: RT 0.749 min, [M−H]⁻ 351.80, LCMS method I.

### Step 5. 1-(2-chloro-5-fluorophenyl)-N-methyl-8-nitro-3-oxo-1,2,3,4-tetrahydropyrrolo[1,2-a]pyrazine-6-carboxamide

A round bottom flask was charged with 1-(2-chloro-5-fluorophenyl)-8-nitro-3-oxo-1,2,3,4-tetrahydropyrrolo[1,2-a]pyrazine-6-carboxylic acid (200 mg) and methylamine hydrochloride (49.5 mg) dissolved in dimethylformamide (3 mL). HATU (322 mg) and sodium bicarbonate (283 mg) were added, and the solution was stirred at room temperature for 1 hour. The solution was concentrated, and the residue was purified using C18 flash chromatography with the following conditions (mobile phase A: water, mobile phase B: acetonitrile; flow rate: 60 mL/min; Gradient: 0% B to 100% B in 40 minutes; wavelength 254/220 nm) to give the desired product (100 mg) as a yellow solid. LCMS: RT 0.907 min, [M+H]⁺ 366.95, LCMS method A.

### Step 6. 8-amino-1-(2-chloro-5-fluorophenyl)-N-methyl-3-oxo-1,2,3,4-tetrahydropyrrolo[1,2-a]pyrazine-6-carboxamide

A round bottom flask was charged with 1-(2-chloro-5-fluorophenyl)-N-methyl-8-nitro-3-oxo-1H,2H,3H,4H-pyrrolo[1,2-a]pyrazine-6-carboxamide (100 mg) dissolved in dioxane (2.2 mL). A solution of sodium hydrosulfite (217 mg) and sodium bicarbonate (157 mg) dissolved in water (2.2 mL) was added, and the solution was stirred at room temperature for 1 hour. The reaction was diluted with water and extracted with ethyl acetate. The organic layer was washed with brine, dried over Na₂SO₄ and concentrated to give the desired product (50 mg) as a yellow solid. LCMS: RT 0.615 min, [M+H]⁺ 337.00, LCMS method A.

### Step 7. 4-nitrophenyl (1-(2-chloro-5-fluorophenyl)-6-(methylcarbamoyl)-3-oxo-1,2,3,4-tetrahydropyrrolo[1,2-a]pyrazin-8-yl)carbamate

A round bottom flask was charged with 8-amino-1-(2-chloro-5-fluorophenyl)-N-methyl-3-oxo-1H,2H,3H,4H-pyrrolo[1,2-a]pyrazine-6-carboxamide (60 mg) and 4-nitrophenyl carbonochloridate (53.8 mg). Tetrahydrofuran (2 mL) was added, the solution was stirred at 70° C. for 1 hour and was used directly in the next step. LCMS: RT 0.965 min, [M+H]⁺=501.95, LCMS method A.

### Step 8. 1-(2-chloro-5-fluorophenyl)-8-(indoline-1-carboxamido)-N-methyl-3-oxo-1,2,3,4-tetrahydropyrrolo[1,2-a]pyrazine-6-carboxamide

A round bottom flask was charged with 2,3-dihydro-1H-indole (59.3 mg), triethylamine (30.1 mg) and a stir bar. Tetrahydrofuran (1 mL) was added, and the solution was poured into 4-nitrophenyl (1-(2-chloro-5-fluorophenyl)-6-(methylcarbamoyl)-3-oxo-1,2,3,4-tetrahydropyrrolo[1,2-a]pyrazin-8-yl)carbamate (50 mg, 99.6 μmol) in tetrahydrofuran (1 mL). The mixture was stirred at 70° C. for 1 hour. The reaction was quenched with water and extracted with ethyl acetate. The organic layer was washed with brine, dried over Na₂SO₄ and concentrated. The resulting crude material was purified by HPLC (Column: XBridge Prep OBD C18 Column, 30×150 mm, 5 μm; mobile phase A: 10 mM NH₄HCO₃ solution, mobile phase B: acetonitrile (containing 0.1% diethylamine); flow rate: 60 mL/min; Gradient: 12% B to 30% B in 8 min; wavelength 254/220 nm; RT: 7.8 min) to give the desired product (20.6 mg) as a white amorphous solid. LCMS: RT 0.795 min, [M+H]⁺ 482.1, LCMS method E.

### Step 9. (S)-1-(2-chloro-5-fluorophenyl)-8-(indoline-1-carboxamido)-N-methyl-3-oxo-1,2,3,4-tetrahydropyrrolo[1,2-a]pyrazine-6-carboxamide and (R)-1-(2-chloro-5-fluorophenyl)-8-(indoline-1-carboxamido)-N-methyl-3-oxo-1,2,3,4-tetrahydropyrrolo[1,2-a]pyrazine-6-carboxamide

1-(2-Chloro-5-fluorophenyl)-8-(indoline-1-carboxamido)-N-methyl-3-oxo-1,2,3,4-tetrahydropyrrolo[1,2-a]pyrazine-6-carboxamide (17 mg) was chirally resolved using prep-chiral-HPLC with the following conditions: Column: CHIRALPAK IH, 2*25 cm, 5 μm; mobile phase A: hexane (containing 0.2% triethylamine), mobile phase B: EtOH:dichloromethane 1:1; flow rate: 20 mL/min; Gradient: 50% B isocratic for 11 min; wavelength: 220/254 nm; injection volume: 3.8 mL. Lyophilization yielded the two enantiomers, both as an off-white amorphous solid.

Compound I-85, Peak 1, 6.3 mg. Chiral HPLC RT: 2.86 min. LCMS: RT 1.413 min, [M+H]⁺ 482.1, LCMS method H. ¹H NMR (400 MHz, DMSO-d6) δ 8.83 (1H, d, J=2.7 Hz), 8.01 (1H, q, J=4.5 Hz), 7.77 (2H, d, J=7.9 Hz), 7.40 (1H, dd, J=8.8, 5.1 Hz), 7.15 (2H, td, J=8.5, 2.6 Hz), 7.13-7.04 (1H, m), 7.03 (1H, dd, J=9.2, 3.1 Hz), 6.86 (1H, td, J=7.4, 1.1 Hz), 6.77 (1H, s), 6.10-6.04 (1H, m), 5.06 (2H, s), 3.85-3.74 (1H, m), 3.56-3.45 (1H, m), 3.07 (2H, t, J=8.7 Hz), 2.73 (3H, d, J=4.5 Hz).

Compound I-86, Peak 2, 7.1 mg. Chiral HPLC RT: 9.90 min. LCMS: RT 0.744 min, [M+H]⁺ 482.1, LCMS method I. ¹H NMR (400 MHz, DMSO-d6) 8.83 (1H, d, J=2.7 Hz), 8.01 (1H, q, J=4.5 Hz), 7.77 (2H, d, J=7.9 Hz), 7.40 (1H, dd, J=8.8, 5.1 Hz), 7.15 (2H, td, J=8.5, 2.6 Hz), 7.11-6.99 (2H, m), 6.86 (1H, td, J=7.4, 1.1 Hz), 6.77 (1H, s), 6.07 (1H, d, J=2.5 Hz), 5.06 (2H, s), 3.85-3.74 (1H, m), 3.56-3.45 (1H, m), 3.07 (2H, t, J=8.7 Hz), 2.73 (3H, d, J=4.5 Hz).

Additional compounds prepared according to the methods of Example 16 are listed in Table 7 below. Corresponding ¹H NMR and mass spectrometry characterization for these compounds are described in Table 1. Certain compounds in Table 7 below were prepared with other compounds whose preparation is described in the Examples herein.

TABLE 7
Additional Exemplary Compounds
Compound

I-15
I-17
I-50
I-51
I-52
I-53
I-54
I-55
I-56
I-57
I-340
I-341
I-342
I-343
I-344
I-440
I-441

### Example 17

### 1-(2-chloro-5-fluorophenyl)-9-(3-fluoro-5-(trifluoromethyl)benzamido)-N-methyl-3-oxo-2,3,4,5-tetrahydro-1H-pyrrolo[1,2-a][1,4]diazepine-7-carboxamide (I-90)

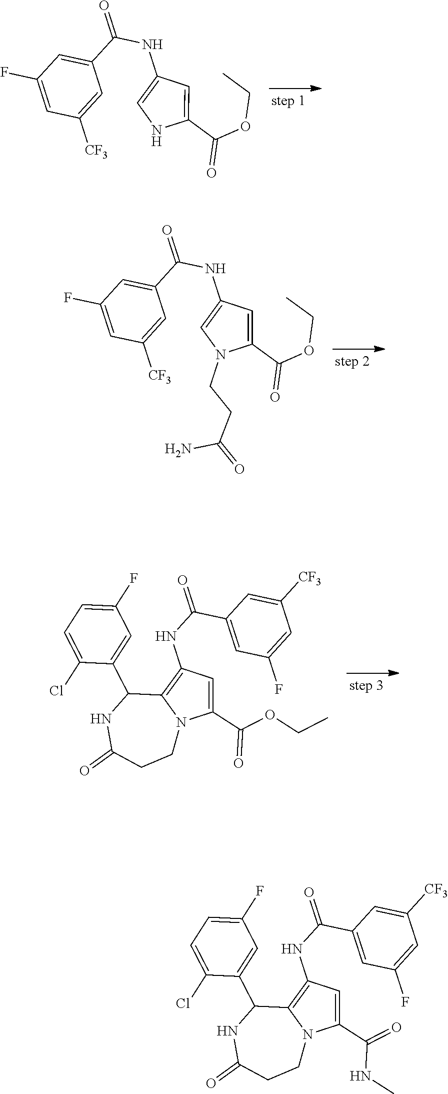 [SMILES: CCOC(=O)c1cc(NC(=O)c2cc(F)cc(C(F)(F)F)c2)c[nH]1 ; CCOC(=O)c1cc(NC(=O)c2cc(F)cc(C(F)(F)F)c2)cn1CCC(N)=O ; CCOC(=O)c1cc(NC(=O)c2cc(F)cc(C(F)(F)F)c2)c2n1CCC(=O)NC2c1cc(F)ccc1Cl ; CNC(=O)c1cc(NC(=O)c2cc(F)cc(C(F)(F)F)c2)c2n1CCC(=O)NC2c1cc(F)ccc1Cl]

### Step 1. ethyl 1-(3-amino-3-oxopropyl)-4-(3-fluoro-5-(trifluoromethyl)benzamido)-1H-pyrrole-2-carboxylate

A 25 mL round bottom flask was charged with ethyl 4-(3-fluoro-5-(trifluoromethyl)benzamido)-1H-pyrrole-2-carboxylate (0.4 g). Acetonitrile (4 mL) was added, followed by 2-(tert-butyl)-1,1,3,3-tetramethylguanidine (235 μL). The resulting yellow solution was stirred for 5 minutes prior to addition of prop-2-enamide (82 mg). The reaction was stirred for 72 hours. The solution was concentrated under vacuum and taken up in cold dichloromethane for trituration by sonication. The white solid was collected by filtration and washed with cold dichloromethane. After drying the desired product (381 mg) was obtained as a white solid. LCMS: RT 1.13 min, [M+H]⁺ 416.3, LCMS method U.

### Step 2. ethyl 1-(2-chloro-5-fluorophenyl)-9-(3-fluoro-5-(trifluoromethyl)benzamido)-3-oxo-2,3,4,5-tetrahydro-1H-pyrrolo[1,2-a][1,4]diazepine-7-carboxylate

A microwave vial was charged with ethyl 1-(3-amino-3-oxopropyl)-4-(3-fluoro-5-(trifluoromethyl)benzamido)-1H-pyrrole-2-carboxylate (200 mg) and 2-chloro-5-fluorobenzaldehyde (84 mg). Eaton's reagent (4.5 mL) was added, and the reaction was heated at 100° C. under microwave for 20 minutes. The material was diluted with ethyl acetate (5 mL) and quenched with cold saturated NaHCO₃ solution until no more bubbling was observed. The aqueous phase was extracted with ethyl acetate twice. The organic layers were combined and concentrated under reduced pressure to afford the crude product as a brownish oil. The crude material was dissolved in DMF (1 mL) and loaded on a 30 g normal phase column for purification (dichloromethane:ethyl acetate 100:0 to 70:30) to give the desired product (132 mg). LCMS: RT 1.35 min, [M+H]⁺ 556.3, LCMS method Q.

### Step 3. 1-(2-chloro-5-fluorophenyl)-9-(3-fluoro-5-(trifluoromethyl)benzamido)-N-methyl-3-oxo-2,3,4,5-tetrahydro-1H-pyrrolo[1,2-a][1,4]diazepine-7-carboxamide

To a suspension of methylamine hydrochloride (0.70 g) in 10 mL of dry toluene at 5° C. was slowly added trimethylaluminum (5 mL). The mixture was allowed to warm to room temperature and stirred for 1 hour until gas evolution ceased. To a solution of ethyl 1-(2-chloro-5-fluorophenyl)-9-(3-fluoro-5-(trifluoromethyl)benzamido)-3-oxo-2,3,4,5-tetrahydro-1H-pyrrolo[1,2-a][1,4]diazepine-7-carboxylate (85 mg) in 1.5 mL of dry toluene was added the prepared solution above (683 μL). The solution was heated under nitrogen overnight at 90° C. The reaction mixture was cooled to room temperature and was carefully quenched with 1 N HCl at 0° C. The organic layer was separated, and the aqueous layer was extracted three times with ethyl acetate. The organic layers were combined, dried over MgSO₄ and concentrated under vacuum. The crude material was dissolved in DMF (2 mL) and loaded on a 30 g reverse phase column for purification (10 mM ammonium formate solution:acetonitrile 95:5 to 50:50) to give the desired product (20.3 mg). LCMS: RT 2.94 min, [M+H]⁺ 541.1, LCMS method R. ¹H NMR (400 MHz, DMSO-d6) δ 10.23 (s, 1H), 8.40 (d, J=6.6 Hz, 1H), 8.16 (q, J=4.3 Hz, 1H), 8.08 (s, 1H), 7.99 (dd, J=16.7, 8.8 Hz, 2H), 7.45 (dd, J=8.8, 5.2 Hz, 1H), 7.29 (dd, J=9.8, 3H).

### Example 18

### N-(8-(2-chloro-5-fluorophenyl)-3-(hydroxymethyl)-6-oxo-5,6,7,8-tetrahydroimidazo[1,5-a]pyrazin-1-yl)-3-fluoro-5-(trifluoromethyl)benzamide (I-92)

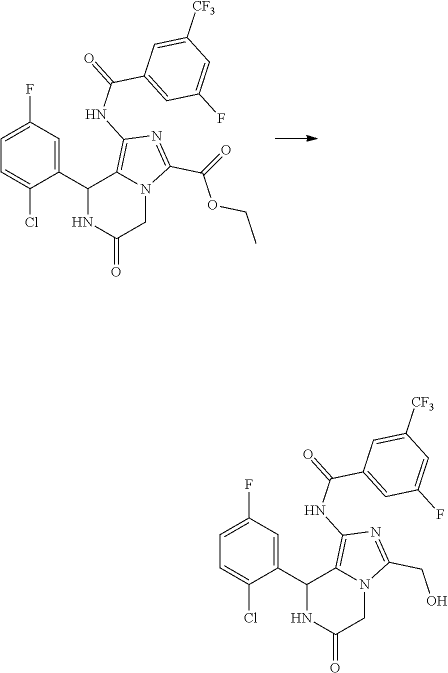 [SMILES: CCOC(=O)c1nc(NC(=O)c2cc(F)cc(C(F)(F)F)c2)c2n1CC(=O)NC2c1cc(F)ccc1Cl ; O=C1Cn2c(CO)nc(NC(=O)c3cc(F)cc(C(F)(F)F)c3)c2C(c2cc(F)ccc2Cl)N1]

In a flame-dried round bottom flask, a solution of ethyl 8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (586 mg) in THF (7 mL) was cooled to −15° C. A 2 M solution of LiAlH₄ in THF (1.62 mL) was added and the solution was stirred at −15° C. for 1 hour. More LiAlH₄ (2 M solution in THF, 1.62 mL) was added and stirring was continued for another 1 hour. The reaction mixture was quenched first with ethyl acetate (10 mL) at −15° C. then with saturated aqueous solution of NH₄Cl, followed by saturated aqueous solution of Rochelle's salt. The solution was stirred for 20 minutes. The solution was concentrated under reduced pressure to remove volatiles. The aqueous mixture was extracted with ethyl acetate (2×30 mL). The combined organic layers were washed with brine, dried over anhydrous Na₂SO₄ and concentrated in vacuo to afford the desired product (540 mg) as a light brown gum, which was used in the next step without purification. LC-MS RT 0.94 min; [M+H]⁺ 501.3, LCMS method U.

### Example 19

### (S)—N-(8-(2-chloro-5-fluorophenyl)-3-(2-hydroxypropan-2-yl)-6-oxo-5,6,7,8-tetrahydroimidazo[1,5-a]pyrazin-1-yl)-3-fluoro-5-(trifluoromethyl)benzamide (I-103 or I-104) and (R)—N-(8-(2-chloro-5-fluorophenyl)-3-(2-hydroxypropan-2-yl)-6-oxo-5,6,7,8-tetrahydroimidazo[1,5-a]pyrazin-1-yl)-3-fluoro-5-(trifluoromethyl)benzamide (I-103 or I-104)

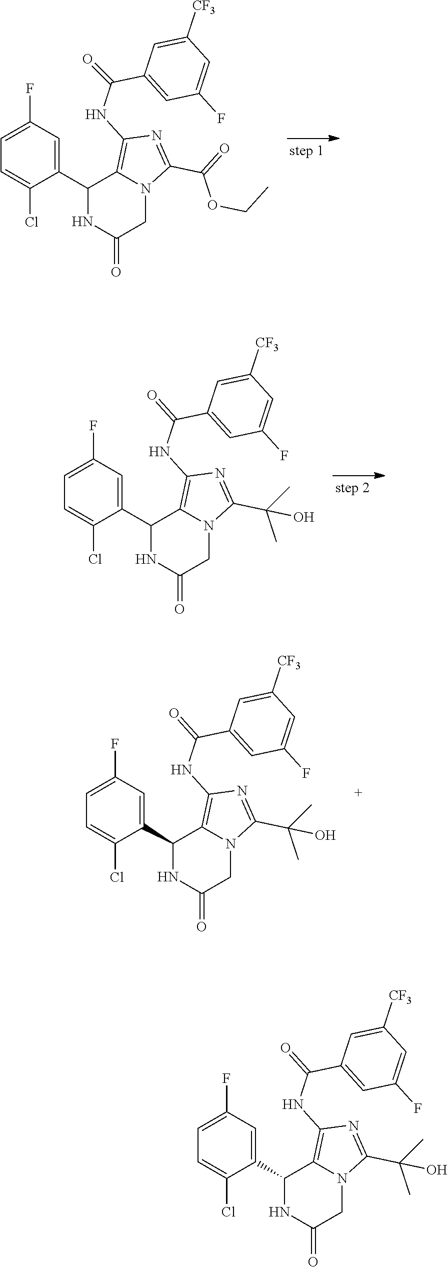 [SMILES: CCOC(=O)c1nc(NC(=O)c2cc(F)cc(C(F)(F)F)c2)c2n1CC(=O)NC2c1cc(F)ccc1Cl ; CC(C)(O)c1nc(NC(=O)c2cc(F)cc(C(F)(F)F)c2)c2n1CC(=O)NC2c1cc(F)ccc1Cl ; CC(C)(O)c1nc(NC(=O)c2cc(F)cc(C(F)(F)F)c2)c2n1CC(=O)N[C@H]2c1cc(F)ccc1Cl ; CC(C)(O)c1nc(NC(=O)c2cc(F)cc(C(F)(F)F)c2)c2n1CC(=O)N[C@@H]2c1cc(F)ccc1Cl]

### Step 1. N-(8-(2-chloro-5-fluorophenyl)-3-(2-hydroxypropan-2-yl)-6-oxo-5,6,7,8-tetrahydroimidazo[1,5-a]pyrazin-1-yl)-3-fluoro-5-(trifluoromethyl)benzamide

In a flame-dried microwave vial, to a stirred solution of ethyl 8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (50 mg) in THF (0.7 mL) at 0° C. was added 3 M solution of CH₃MgBr in Et₂O (153 μL) and the resulting solution was stirred at room temperature for 90 minutes. The reaction mixture was quenched with water at 0° C. and extracted with ethyl acetate (2×15 mL). The combined organic layers were washed with brine, dried over anhydrous Na₂SO₄ and concentrated in vacuo. The crude residue was purified on a reverse C18 column (12 g); eluent A: 10 mM ammonium formate solution; B: acetonitrile; gradient: 0-100% B to afford the desired product (26 mg) as a white solid. LC-MS: RT 1.96 min, [M+H]⁺ 529.3, LCMS method V. ¹H NMR (400 MHz, DMSO-d6) δ 10.33 (s, 1H), 8.88 (d, J=2.3 Hz, 1H), 7.90 (br. d, J=8.5 Hz, 1H), 7.85 (br. s, 1H), 7.81 (d, J=9.0 Hz, 1H), 7.35 (dd, J=8.8, 5.2 Hz, 1H), 7.08 (ddd, J=8.7, 8.1, 3.1 Hz, 1H), 6.98 (dd, J=9.3, 3.1 Hz, 1H), 5.97 (s, 1H), 5.52 (s, 1H), 5.07 (d, J=18.2 Hz, 1H), 4.97 (dd, J=18.2, 1.0 Hz, 1H), 1.52 (s, 6H).

### Step 2. (S)—N-(8-(2-chloro-5-fluorophenyl)-3-(2-hydroxypropan-2-yl)-6-oxo-5,6,7,8-tetrahydroimidazo[1,5-a]pyrazin-1-yl)-3-fluoro-5-(trifluoromethyl)benzamide and (R)—N-(8-(2-chloro-5-fluorophenyl)-3-(2-hydroxypropan-2-yl)-6-oxo-5,6,7,8-tetrahydroimidazo[1,5-a]pyrazin-1-yl)-3-fluoro-5-(trifluoromethyl)benzamide

N-(8-(2-chloro-5-fluorophenyl)-3-(2-hydroxypropan-2-yl)-6-oxo-5,6,7,8-tetrahydroimidazo[1,5-a]pyrazin-1-yl)-3-fluoro-5-(trifluoromethyl)benzamide (16.1 mg) was chirally resolved using the SFC condition below to give the two enantiomers: Column: ChiralPak AD-H 21×250 mm; Mobile Phase: 25% Ethanol in CO₂; flow Rate: 70 mL/min; Sample: 16.1 mg of sample was dissolved in 10 mL Methanol+10 mL Dichloromethane; Injection: 2 mL; Detection: 220 nm.

Compound I-103, Peak 1: 7.1 mg. Chiral SFC RT 0.72 min. LCMS: RT 1.027 min, [M+H]⁺ 529.0, LCMS method G.

Compound I-104, Peak 2: 8.4 mg. Chiral SFC RT 1.64 min. LCMS: RT 1.028 min, [M−H]⁻ 527.2, LCMS method G.

### Example 20

### N-(8-(2-chloro-5-fluorophenyl)-3-(1-hydroxyethyl)-6-oxo-5,6,7,8-tetrahydroimidazo[1,5-a]pyrazin-1-yl)-3-fluoro-5-(trifluoromethyl)benzamide (I-111)

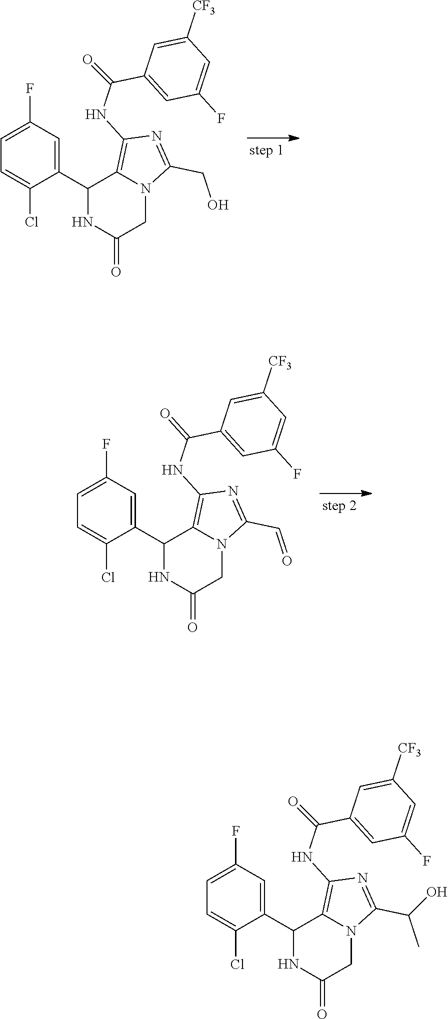 [SMILES: O=C1Cn2c(CO)nc(NC(=O)c3cc(F)cc(C(F)(F)F)c3)c2C(c2cc(F)ccc2Cl)N1 ; O=Cc1nc(NC(=O)c2cc(F)cc(C(F)(F)F)c2)c2n1CC(=O)NC2c1cc(F)ccc1Cl ; CC(O)c1nc(NC(=O)c2cc(F)cc(C(F)(F)F)c2)c2n1CC(=O)NC2c1cc(F)ccc1Cl]

### Step 1. N-(8-(2-chloro-5-fluorophenyl)-3-formyl-6-oxo-5,6,7,8-tetrahydroimidazo[1,5-a]pyrazin-1-yl)-3-fluoro-5-(trifluoromethyl)benzamide

In a flame-dried round bottom flask, to a solution of N-(8-(2-chloro-5-fluorophenyl)-3-(hydroxymethyl)-6-oxo-5,6,7,8-tetrahydroimidazo[1,5-a]pyrazin-1-yl)-3-fluoro-5-(trifluoromethyl)benzamide (520 mg) in dichloromethane (8 mL) at 0° C. was added Dess-Martin periodinane (484 mg) in a single portion. The solution was stirred at room temperature for 30 minutes. The reaction mixture was cooled to 0° C. and treated with saturated aqueous solution of Na₂S₂O₃, followed by saturated aqueous solution of NaHCO₃ and water (˜6 mL each). The resulting mixture was stirred at room temperature for 10 minutes. The resulting solution was diluted with water (20 mL) and extracted with dichloromethane (3×30 mL). The combined organic layers were dried over anhydrous Na₂SO₄, absorbed onto silica and purified by flash column chromatography (SiO₂ 25 g; eluent: 0-7% MeOH in dichloromethane) to afford the desired product (450 mg) as a brown film. LC-MS: RT 1.06 min, [M+H]⁺ 499.2, LCMS method U.

### Step 2. N-(8-(2-chloro-5-fluorophenyl)-3-(1-hydroxyethyl)-6-oxo-5,6,7,8-tetrahydroimidazo[1,5-a]pyrazin-1-yl)-3-fluoro-5-(trifluoromethyl)benzamide

In a flame-dried microwave vial, to a stirred solution of N-(8-(2-chloro-5-fluorophenyl)-3-formyl-6-oxo-5,6,7,8-tetrahydroimidazo[1,5-a]pyrazin-1-yl)-3-fluoro-5-(trifluoromethyl)benzamide (145 mg) in THF (2 mL) at 0° C. was added a 3 M solution of CH₃MgBr in Et₂O (678 μL). The resulting solution was stirred at room temperature for 30 minutes. The reaction mixture was quenched with water at 0° C. and extracted with ethyl acetate (2×30 mL). The combined organic layers were washed with brine, dried over anhydrous Na₂SO₄ and concentrated in vacuo. The crude residue was first purified by flash column chromatography (SiO₂ 12 g; eluent: 0-8% MeOH in dichloromethane), then re-purified on a reverse C18 column (12 g, eluent A: 10 mM ammonium formate solution; B: acetonitrile; gradient: 0-60% B) to afford the desired product (40 mg) as a white solid. LCMS: RT 2.31 min, [M+H]⁺ 515.1, LCMS method T. ¹HNMR (400 MHz, DMSO-d6) δ 10.36 (s, 0.4H), 10.34 (s, 0.6H), 8.89 (d, J=2.2 Hz, 0.4H), 8.88 (d, J=1.7 Hz, 0.6H), 7.96-7.74 (m, 3H), 7.35 (td, J=8.6, 2.1 Hz, 1H), 7.08 (qd, J=8.2, 3.2 Hz, 1H), 6.99 (dd, J=9.2, 2.9 Hz, 1H), 5.99 (br. s, 1H), 5.47 (br. s, 0.4H), 5.45 (br. s, 0.6H), 5.04-4.71 (m, 3H), 1.49 (d, J=2.4 Hz, 1.8H), 1.47 (d, J=2.3 Hz, 1.2H); The ratio of diastereomers is 1.5:1.

### Example 21

### 1-(2-chloro-5-fluorophenyl)-7-fluoro-8-(3-fluoro-5-(trifluoromethyl)benzamido)-N-methyl-3-oxo-1,2,3,4-tetrahydropyrrolo[1,2-a]pyrazine-6-carboxamide (I-112)

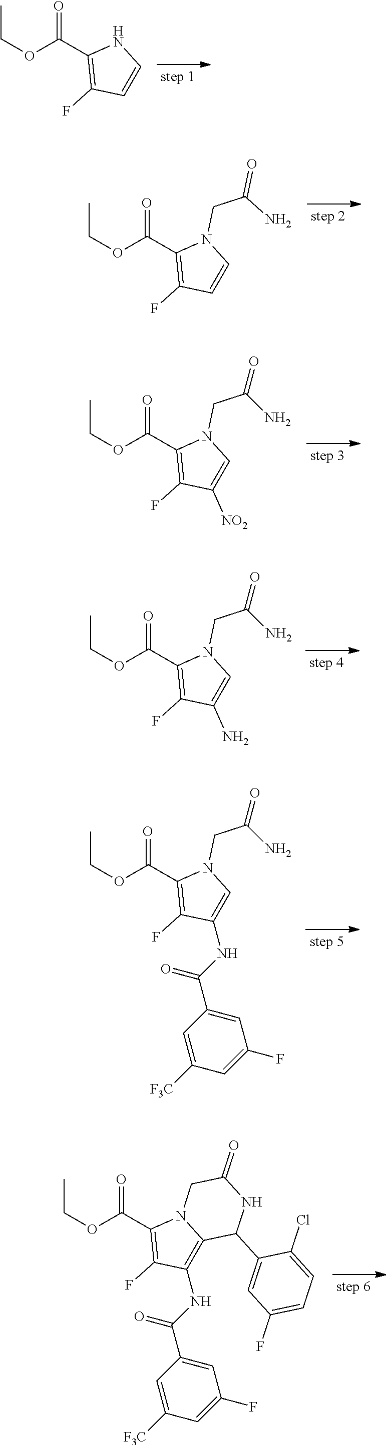 [SMILES: CCOC(=O)c1[nH]ccc1F ; CCOC(=O)c1c(F)ccn1CC(N)=O ; CCOC(=O)c1c(F)c([N+](=O)[O-])cn1CC(N)=O ; CCOC(=O)c1c(F)c(N)cn1CC(N)=O ; CCOC(=O)c1c(F)c(NC(=O)c2cc(F)cc(C(F)(F)F)c2)cn1CC(N)=O ; CCOC(=O)c1c(F)c(NC(=O)c2cc(F)cc(C(F)(F)F)c2)c2n1CC(=O)NC2c1cc(F)ccc1Cl]

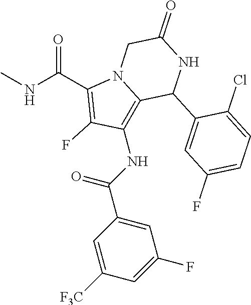 [SMILES: CNC(=O)c1c(F)c(NC(=O)c2cc(F)cc(C(F)(F)F)c2)c2n1CC(=O)NC2c1cc(F)ccc1Cl]

### Step 1. ethyl 1-(2-amino-2-oxoethyl)-3-fluoro-1H-pyrrole-2-carboxylate

A 50 mL round bottom flask was charged with ethyl 3-fluoro-1H-pyrrole-2-carboxylate (3.00 g). Acetonitrile (10 mL) was added, followed by dropwise addition of 2-(tert-butyl)-1,1,3,3-tetramethylguanidine (4.3 mL). The resulting yellow solution was stirred for 5 minutes prior to addition of 2-bromoacetamide (2.90 g, 21.0 mmol). A white precipitate formed. After 2 hours the reaction was cooled to 0° C. and filtered. The collected solid was washed once with cold dichloromethane and dried to give the desired product (3.69 g) as a white solid. LCMS: RT 1.07 min, [M+H]⁺ not observed, LCMS method Q.

### Step 2. ethyl 1-(2-amino-2-oxoethyl)-3-fluoro-4-nitro-1H-pyrrole-2-carboxylate

A 50 mL round bottom flask was charged with ethyl 1-(2-amino-2-oxoethyl)-3-fluoro-1H-pyrrole-2-carboxylate (1.50 g). H₂SO₄ (10 mL) was added. The reaction was cooled to 0° C. and stirred at this temperature for 5 minutes. Potassium nitrate (0.78 g) was added in portions and the reaction was kept under stirring for 1 hour at 0° C. The solution was poured into a cold saturated NaHCO₃ solution containing 10 mL of ethyl acetate. After bubbling ceased the aqueous layer was extracted with ethyl acetate twice. The organic phases were combined, dried over MgSO₄ and concentrated under reduced pressure to give a 2:1 mixture of ethyl 1-(2-amino-2-oxoethyl)-3-fluoro-4-nitro-1H-pyrrole-2-carboxylate and ethyl 1-(2-amino-2-oxoethyl)-3-fluoro-5-nitro-1H-pyrrole-2-carboxylate (250 mg). The mixture was used in the next step without purification. LCMS: RT of 0.96 min and 0.99 min, [M+H]⁺ not observed, LCMS method Q.

### Step 3. ethyl 4-amino-1-(2-amino-2-oxoethyl)-3-fluoro-1H-pyrrole-2-carboxylate

To a suspension of ethyl 1-(2-amino-2-oxoethyl)-3-fluoro-4-nitro-1H-pyrrole-2-carboxylate and ethyl 1-(2-amino-2-oxoethyl)-3-fluoro-5-nitro-1H-pyrrole-2-carboxylate (240 mg) in MeOH (5 mL) was added palladium on carbon (108 mg, 10 wt %). The mixture was degassed with hydrogen and stirred at room temperature under 1 atm of hydrogen. After 1 hour the reaction mixture was purged with N₂, filtered through a short pad of Celite and concentrated under reduced pressure to afford the desired product (200 mg, likely containing the byproduct from the reduction of ethyl 1-(2-amino-2-oxoethyl)-3-fluoro-5-nitro-1H-pyrrole-2-carboxylate) as a colorless solid. LCMS: RT 0.48 min. [M+H]⁺ not observed, LCMS method Q.

### Step 4. ethyl 1-(2-amino-2-oxoethyl)-3-fluoro-4-(3-fluoro-5-(trifluoromethyl) benzamido)-1H-pyrrole-2-carboxylate

A flame-dried microwave vial was charged with ethyl 4-amino-1-(2-amino-2-oxoethyl)-3-fluoro-1H-pyrrole-2-carboxylate (200 mg). Dichloromethane (3 mL) was added, followed by pyridine (281 μL) at room temperature and then 3-fluoro-5-(trifluoromethyl) benzoyl chloride (168 mg) dropwise under nitrogen. After 15 minutes the crude mixture was concentrated to about 1 mL by flushing N₂ through the solution while cooling to 0° C. The white precipitate was collected by filtration and washed with cold dichloromethane. After drying the desired regioisomer was obtained (120 mg) as a white powder. LCMS: RT 1.51 min, [M+H]⁺=420.1, LCMS method Q. ¹H NMR (400 MHz, DMSO-d6) δ 10.42 (s, 1H), 8.17 (s, 1H), 8.09 (d, J=9.1 Hz, 1H), 7.97 (d, J=8.5 Hz, 1H), 7.48 (br. s, 1H), 7.42 (d, J=5.0 Hz, 1H), 7.10 (br. s, 1H), 4.87 (s, 2H), 4.21 (q, J=7.1 Hz, 2H), 1.26 (t, J=7.1 Hz, 3H).

### Step 5. ethyl 1-(2-chloro-5-fluorophenyl)-7-fluoro-8-(3-fluoro-5-(trifluoromethyl) benzamido)-3-oxo-1,2,3,4-tetrahydropyrrolo[1,2-a]pyrazine-6-carboxylate

A microwave vial was charged with ethyl 1-(2-amino-2-oxoethyl)-3-fluoro-4-(3-fluoro-5-(trifluoromethyl)benzamido)-1H-pyrrole-2-carboxylate (120 mg) and 2-chloro-5-fluorobenzaldehyde (53 mg). Eaton's reagent (4 mL) was added, and the reaction was heated at 100° C. under microwave for 15 minutes. The material was diluted in ethyl acetate (5 mL) and quenched with cold saturate NaHCO₃ solution until no more bubbling was observed. The aqueous phase was extracted with ethyl acetate twice. The organic layers were combined, dried over Na₂SO₄ and concentrated under vacuum to afford the crude product as a brown oil. Purification by normal phase column chromatography (dichloromethane:ethyl acetate 100:0 to 70:30) afforded the desired product (120 mg) as a white powder. LCMS: RT 1.70 min, [M+H]⁺ 560.2, LCMS method U.

### Step 6. 1-(2-chloro-5-fluorophenyl)-7-fluoro-8-(3-fluoro-5-(trifluoromethyl)benzamido)-N-methyl-3-oxo-1,2,3,4-tetrahydropyrrolo[1,2-a]pyrazine-6-carboxamide

A microwave vial was charged with ethyl 1-(2-chloro-5-fluorophenyl)-7-fluoro-8-(3-fluoro-5-(trifluoromethyl)benzamido)-3-oxo-1,2,3,4-tetrahydropyrrolo[1,2-a]pyrazine-6-carboxylate (120 mg). THF (2.4 mL) and MeOH (1.2 mL) were added, followed by methylamine (1.66 g, 40% weight in water). The vial was sealed and heated at 45° C. for 36 hours. The solution was concentrated under reduced pressure. The crude material was dissolved in DMF (1 mL) and loaded on a 30 g reverse phase column for purification (10 mM ammonium formate solution:acetonitrile 95:5 to 35:65) to give the desired product (28.2 mg) as a white solid. LCMS: RT 2.98 min, [M+H]⁺ 545.1, LCMS method R. ¹H NMR (400 MHz, DMSO-d6) δ 9.80 (s, 1H), 8.91 (d, J=2.0 Hz, 1H), 7.95 (br. d, J=8.5 Hz, 1H), 7.81-7.76 (m, 2H), 7.61-7.54 (m, 1H), 7.34 (dd, J=8.6, 5.1 Hz, 1H), 7.13-7.04 (m, 2H), 5.92 (d, J=0.5 Hz, 1H), 4.99 (d, J=19.0 Hz, 1H), 4.94 (d, J=19.2 Hz, 1H), 2.76 (d, J=4.5 Hz, 3H). ¹⁹F NMR (376 MHz, DMSO-d6) δ −61.24 (s, 3F), −109.63 (t, J=9.0 Hz, 1F), −115.36 (d, J=5.1 Hz, 1F), −157.63 (s, 1F).

### Example 22

### (1R,4R)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide (I-131, I-132, I-133, or I-134), (1S,4S)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide (I-131, I-132, I-133, or I-134), (1S,4R)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide (I-131, I-132, I-133, or I-134), and (1R,4S)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide (I-131, I-132, I-133, or I-134)

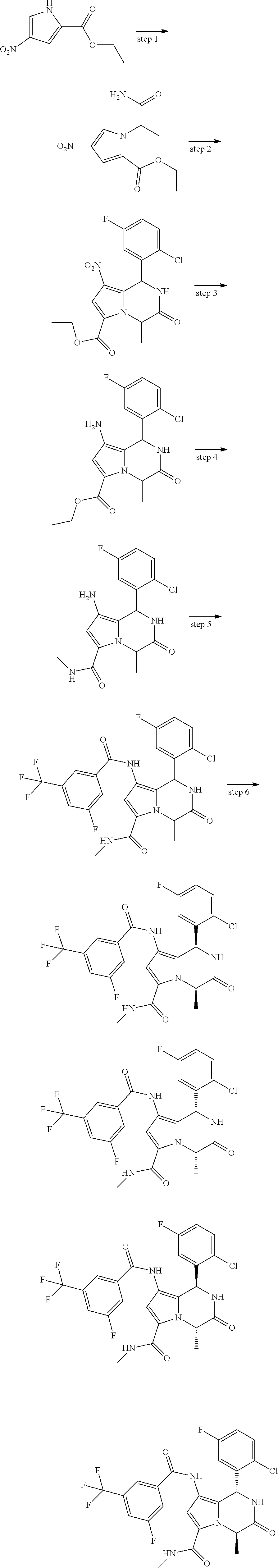

### Step 1. ethyl 1-(1-amino-1-oxopropan-2-yl)-4-nitro-1H-pyrrole-2-carboxylate

A reaction vial was charged with ethyl 4-nitro-1H-pyrrole-2-carboxylate (5 g), 2-(tert-butyl)-1,1,3,3-tetramethylguanidine (5 g, 0.03 mol), acetonitrile (50 mL) and a stir bar before being evacuated and purged with nitrogen three times, and the mixture was stirred at 25° C. for 5 minutes under nitrogen. 2-Bromopropanamide (5 g) was added, the vial was evacuated and purged with nitrogen three times, and the reaction mixture was stirred at 25° C. for 6 hours under nitrogen. The precipitate was collected by filtration, washed with ethyl acetate and dried to give the desired product (5 g) as an off-white amorphous solid. LCMS: RT 0.667 min, [M+H]⁺ 256.25, LCMS method K.

### Step 2. Ethyl 1-(2-chloro-5-fluorophenyl)-4-methyl-8-nitro-3-oxo-1,2,3,4-tetrahydropyrrolo[1,2-a]pyrazine-6-carboxylate

A reaction vial was charged with ethyl 1-(1-amino-1-oxopropan-2-yl)-4-nitro-1H-pyrrole-2-carboxylate (4.5 g) and 2-chloro-5-fluorobenzaldehyde (2.8 g) before being evacuated and purged with nitrogen three times. Eaton's reagent (80 mL) was added, and the mixture was stirred at 90° C. for 1 hour under N₂. The reaction was quenched with saturated NaHCO₃ solution. The reaction mixture was diluted with water (10 mL), and the aqueous phase was extracted with ethyl acetate (50 mL) three times. The combined organic layers were washed with brine, dried over sodium sulfate, filtered, and concentrated in vacuo. The resulting crude material was purified by prep-HPLC (mobile phase A: water with 0.1% formic acid, mobile phase B: acetonitrile with 0.10% formic acid) to give the desired product (4 g) as an off-white amorphous solid. LCMS: RT 1.128 min, [M+H]⁺ 396.05, LCMS method B.

### Step 3. ethyl 8-amino-1-(2-chloro-5-fluorophenyl)-4-methyl-3-oxo-1,2,3,4-tetrahydropyrrolo[1,2-a]pyrazine-6-carboxylate

A reaction vial was charged with ethyl 1-(2-chloro-5-fluorophenyl)-4-methyl-8-nitro-3-oxo-1,2,3,4-tetrahydropyrrolo[1,2-a]pyrazine-6-carboxylate (4 g), iron powder (3 g) and NH₄Cl (3 g) before being evacuated and purged with nitrogen three times. EtOH (30 mL) and H₂O (10 mL) were added, and the mixture was stirred at 90° C. for 1 hour under nitrogen. The mixture was filtered through a Celite pad. The filtrate was concentrated under vacuum. The resulting crude material was purified by prep-HPLC (mobile phase A: water, mobile phase B: acetonitrile) to give the desired product (3.5 g) as an off-white amorphous solid. LCMS: RT 0.793 min, [M+H]⁺=366.0, LCMS method B.

### Step 4. 8-amino-1-(2-chloro-5-fluorophenyl)-N,4-dimethyl-3-oxo-1,2,3,4-tetrahydropyrrolo[1,2-a]pyrazine-6-carboxamide

A reaction vial was charged with ethyl 8-amino-1-(2-chloro-5-fluorophenyl)-4-methyl-3-oxo-1,2,3,4-tetrahydropyrrolo[1,2-a]pyrazine-6-carboxylate (3.5 g) before being evacuated and purged with nitrogen three times. Methylamine aqueous solution (40% by weight, 80 mL) was added, and the mixture was stirred at 25° C. for 48 hours under nitrogen. The solution was concentrated under vacuum. The crude product was purified by silica gel chromatography (10 g column; eluting with petroleum ether:ethyl acetate 10:1) to give the desired product (1 g) as an off-white amorphous solid. LCMS: RT 0.710 min, [M+H]⁺ 350.95, LCMS method B.

### Step 5. 1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide

A reaction vial was charged with 8-amino-1-(2-chloro-5-fluorophenyl)-N,4-dimethyl-3-oxo-1,2,3,4-tetrahydropyrrolo[1,2-a]pyrazine-6-carboxamide (100 mg), 3-fluoro-5-(trifluoromethyl)benzoic acid (89.0 mg), HATU (162 mg) and DIEA (248 mg) before being evacuated and purged with nitrogen three times. DMF (5 mL) was added, and the mixture was stirred at 25° C. for 1 hour under nitrogen. The resulting crude material was purified by prep-HPLC (Column: XBridge Shield RP18 OBD Column, 30*150 mm, 5 μm; mobile phase A: Water (10 mM NH₄HCO₃ with 0.1% NH₃·H₂O), mobile phase B: acetonitrile; flow rate: 60 mL/min; gradient: 30% B to 55% B in 7 min, then 55% B; wavelength: 220 nm; RT: 6.5 min) to give the desired product (80 mg) as an off-white amorphous solid. LCMS: RT 0.941 min, [M+H]⁺ 541.15, LCMS method M.

### Step 6. (1R,4R)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide, (1S,4S)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide, (1S,4R)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide and (1R,4S)-1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide

1-(2-chloro-5-fluorophenyl)-8-(3-fluoro-5-(trifluoromethyl)benzamido)-N,4-dimethyl-3-oxo-1,2,3,4-tetrahydropyrrolo[1,2-a]pyrazine-6-carboxamide (77 mg) was chirally resolved by CHIRAL-HPLC (Column: DZ-CHIRALPAK IH-3, 4.6*50 mm, 3.0 μm; mobile phase A: hexane (with 0.2% isopropylamine); mobile phase B: EtOH:dichloromethane 1:1; gradient: 25% B isocratic; flow rate: 1 mL/min) to give 4 peaks, all as an off-white amorphous solid.

Compound I-134, Peak 1: 5.9 mg. LCMS: RT 2.011 min, [M+H]⁺ 541.15, LCMS method M. ¹H NMR (400 MHz, DMSO-d₆) δ 9.79 (s, 1H), 8.70 (s, 1H), 8.12 (q, J=4.5 Hz, 1H), 7.90 (d, J=8.3 Hz, 1H), 7.69-7.61 (m, 2H), 7.27 (dd, J=8.8, 5.3 Hz, 1H), 7.05 (dd, J=9.4, 3.1 Hz, 1H), 6.99 (td, J=8.3, 3.1 Hz, 1H), 6.83 (s, 1H), 5.92 (s, 1H), 5.70 (q, J=6.8 Hz, 1H), 2.75 (d, J=4.4 Hz, 3H), 1.52 (d, J=6.9 Hz, 3H).

Compound I-133, Peak 2: 3.3 mg. LCMS: RT 1.412 min, [M+H]⁺ 541, LCMS method M. ¹H NMR (400 MHz, DMSO-d₆) 9.79 (s, 1H), 8.70 (s, 1H), 8.11 (q, J=4.5 Hz, 1H), 7.90 (dt, J=8.6, 2.0 Hz, 1H), 7.69-7.61 (m, 2H), 7.27 (dd, J=8.9, 5.2 Hz, 1H), 7.05 (dd, J=9.3, 3.1 Hz, 1H), 6.99 (td, J=8.4, 3.1 Hz, 1H), 6.83 (s, 1H), 5.92 (s, 1H), 5.70 (q, J=6.8 Hz, 1H), 2.75 (d, J=4.5 Hz, 3H), 1.52 (d, J=6.8 Hz, 3H).

Compound I-132, Peak 3: 12 mg. LCMS: RT 2.101 min, [M+H]⁺=541.15, LCMS method M. ¹H NMR (400 MHz, DMSO-d₆) 9.88 (s, 1H), 8.95 (d, J=3.8 Hz, 1H), 8.21 (q, J=4.5 Hz, 1H), 7.92 (dt, J=8.7, 2.0 Hz, 1H), 7.79 (dd, J=8.5, 2.2 Hz, 2H), 7.43 (dd, J=8.8, 5.1 Hz, 1H), 7.22-7.13 (m, 1H), 6.98 (s, 1H), 6.64 (dd, J=9.3, 3.1 Hz, 1H), 6.18 (d, J=3.5 Hz, 1H), 5.59-5.49 (m, 1H), 2.76 (d, J=4.5 Hz, 3H), 1.58 (d, J=6.9 Hz, 3H).

Compound I-131, Peak 4:16 mg. LCMS: RT 2.091 min, [M+H]⁺ 541.15, LCMS method M. ¹H NMR (400 MHz, DMSO-d6) 9.89 (s, 1H), 8.95 (d, J=3.8 Hz, 1H), 8.21 (q, J=4.5 Hz, 1H), 7.92 (dt, J=8.7, 2.0 Hz, 1H), 7.79 (dd, J=8.5, 2.0 Hz, 2H), 7.43 (dd, J=8.9, 5.2 Hz, 1H), 7.17 (td, J=8.4, 3.1 Hz, 1H), 6.98 (s, 1H), 6.64 (dd, J=9.3, 3.1 Hz, 1H), 6.18 (d, J=3.6 Hz, 1H), 5.54 (q, J=6.8 Hz, 1H), 2.76 (d, J=4.5 Hz, 3H), 1.58 (d, J=6.8 Hz, 3H).

Additional compounds prepared according to the methods of Example 22 are listed in Table 8 below. Corresponding ¹H NMR and mass spectrometry characterization for these compounds are described in Table 1. Certain compounds in Table 8 below were prepared with other compounds whose preparation is described in the Examples herein.

TABLE 8
Additional Exemplary Compounds
Compound

I-24
I-25
I-60
I-150
I-151
I-152
I-153

### Example 23

### (S)—N-(8-(2-chloro-5-fluorophenyl)-3-(methylcarbamoyl)-6-oxo-5,6,7,8-tetrahydroimidazo[1,5-a]pyrazin-1-yl)benzo[d]isothiazole-3-carboxamide (I-158)

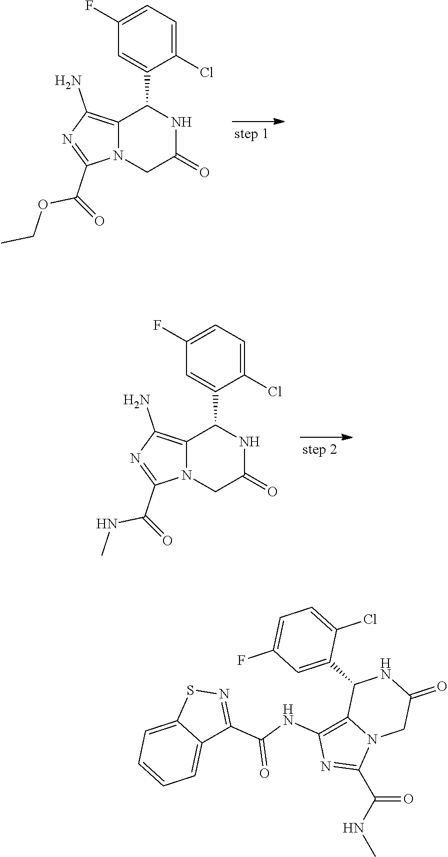 [SMILES: CCOC(=O)c1nc(N)c2n1CC(=O)N[C@H]2c1cc(F)ccc1Cl ; CNC(=O)c1nc(N)c2n1CC(=O)N[C@H]2c1cc(F)ccc1Cl ; CNC(=O)c1nc(NC(=O)c2nsc3ccccc23)c2n1CC(=O)N[C@H]2c1cc(F)ccc1Cl]

### Step 1. (S)-1-amino-8-(2-chloro-5-fluorophenyl)-N-methyl-6-oxo-5,6,7,8-tetrahydroimidazo[1,5-a]pyrazine-3-carboxamide

A round bottom flask was charged with ethyl (S)-1-amino-8-(2-chloro-5-fluorophenyl)-6-oxo-5,6,7,8-tetrahydroimidazo[1,5-a]pyrazine-3-carboxylate (intermediate I, 2.0 g) and dichloromethane (15 mL) at 0° C. under a nitrogen atmosphere. AlMe₃ (10 mL) was added slowly. The solution was stirred at 0° C. for 15 minutes under nitrogen. Then methylamine in THF (1.8 M, 10 mL) was added, and the solution was stirred at 50° C. for 16 hours. The reaction mixture was quenched with saturated NH₄Cl solution (50 mL), and the aqueous phase was extracted with ethyl acetate (50 mL) three times. The combined organic layers were washed with brine, dried over sodium sulfate, filtered, and concentrated in vacuo. The resulting crude material was purified by HPLC (mobile phase A: water with 0.1% formic acid, mobile phase B: acetonitrile with 0.1% formic acid) to give the desired product (1.3 g) as a white amorphous solid. LCMS: RT 0.68 min, [M+H]⁺ 338.15, LCMS method A.

### Step 2. (S)—N-(8-(2-chloro-5-fluorophenyl)-3-(methylcarbamoyl)-6-oxo-5,6,7,8-tetrahydroimidazo[1,5-a]pyrazin-1-yl)benzo[d]isothiazole-3-carboxamide

A round bottom flask was charged with (S)-1-amino-8-(2-chloro-5-fluorophenyl)-N-methyl-6-oxo-5,6,7,8-tetrahydroimidazo[1,5-a]pyrazine-3-carboxamide (1.1 g), benzo[d]isothiazole-3-carboxylic acid (0.88 g), HATU (1.5 g), DIEA (1.3 g), and DMF (20 mL). The solution was stirred at 25° C. for 1 hour. The reaction mixture was diluted with H₂O (20 mL), and the aqueous phase was extracted with ethyl acetate (15 mL) three times. The combined organic layers were washed with brine, dried over sodium sulfate, filtered, and concentrated in vacuo. The resulting crude material was purified by HPLC (mobile phase A: water, mobile phase B: acetonitrile) to give the desired product (1.2 g) as a white amorphous solid. LCMS: RT 1.324 min, [M+H]⁺ 499.15, LCMS method M. ¹H NMR (400 MHz, DMSO-d₆) δ 10.30 (s, 1H), 8.96 (d, J=2.4 Hz, 1H), 8.59 (dt, J=8.2, 1.1 Hz, 1H), 8.45 (q, J=4.7 Hz, 1H), 8.31 (dd, J=8.2, 1.1 Hz, 1H), 7.69 (ddd, J=8.2, 7.0, 1.2 Hz, 1H), 7.61 (ddd, J=8.1, 7.0, 1.1 Hz, 1H), 7.27 (dd, J=8.9, 5.1 Hz, 1H), 7.17 (dd, J=9.2, 3.1 Hz, 1H), 7.01 (td, J=8.4, 3.1 Hz, 1H), 6.18 (d, J=2.3 Hz, 1H), 5.30-5.21 (m, 1H), 5.12 (dd, J=18.8, 1.6 Hz, 1H), 2.77 (d, J=4.7 Hz, 3H).

### Example 24

### (S)—N-((6-(1H-pyrazol-1-yl)pyridin-3-yl)methyl)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxamide (I-238)

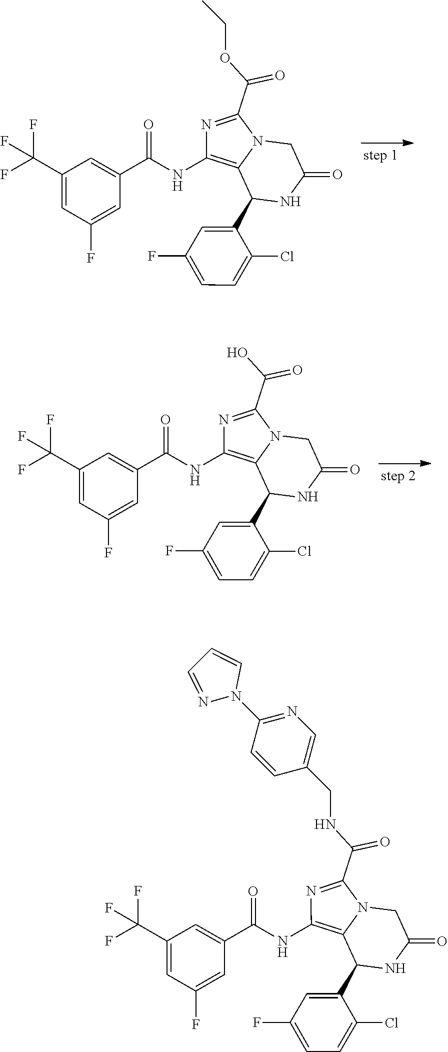 [SMILES: CCOC(=O)c1nc(NC(=O)c2cc(F)cc(C(F)(F)F)c2)c2n1CC(=O)N[C@H]2c1cc(F)ccc1Cl ; O=C1Cn2c(C(=O)O)nc(NC(=O)c3cc(F)cc(C(F)(F)F)c3)c2[C@H](c2cc(F)ccc2Cl)N1 ; O=C1Cn2c(C(=O)NCc3ccc(-n4cccn4)nc3)nc(NC(=O)c3cc(F)cc(C(F)(F)F)c3)c2[C@H](c2cc(F)ccc2Cl)N1]

### Step 1. (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylic acid

To a stirred solution of ethyl (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (intermediate II, 5.608 g) in methanol (55.9 mL) at 15° C. was added a solution of sodium hydroxide (1.240 g) in water (28.0 mL). The mixture was stirred at 15° C. for 2 hours. The pH was adjusted to 3 with 1 N HCl. The white precipitate was collected by filtration and washed with water (6×5 mL). The white solid was dried under high vacuum to yield the desired product (5.05 g). LCMS: RT 1.23 min, [M+H]⁺ 514.7, LCMS method U.

### Step 2. (S)—N-((6-(1H-pyrazol-1-yl)pyridin-3-yl)methyl)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxamide

To a stirred solution of (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylic acid (5.05), (6-(1H-pyrazol-1-yl)pyridin-3-yl)methanamine (2.11 g) and DIEA (3.61 g) in N,N-dimethylformamide (15 mL) was added HATU (5.3142 g). The mixture was briefly sonicated and then stirred 20 minutes. More HATU (1.2 g) was added, and the reaction was stirred for 15 minutes. The amber solution was poured into water (300 mL). The suspension was sonicated and then stirred for 5 minutes before allowing to stand for 10 minutes. An off-white solid was isolated by filtration, washed with 1 N aqueous NaOH (3×15 mL), water (4×20 mL), and then dried before loading as a silica gel slurry onto a 120 gram silica gel column, which was eluted with a dichloromethane (A) to 10:90:1 MeOH/dichloromethane/NH₄OH (B, gradient 0-100%) over 22 minutes. The collected product was concentrated and re-purified on an 80 g silica gel column, which was eluted with dichloromethane (A) and 10:90 MeOH:DCM (B, gradient 0=100%) over 25 minutes to give the desired product after drying (3.90 g) as a nearly white solid. LCMS: RT 1.43 min, [M+H]⁺ 671.4, LCMS method U. ¹H NMR (400 MHz, DMSO-d6) δ 10.36 (s, 1H), 9.16 (t, J=6.3 Hz, 1H), 8.91 (d, J=2.1 Hz, 1H), 8.59 (dd, J=2.6, 0.7 Hz, 1H), 8.43 (dd, J=2.3, 0.9 Hz, 1H), 7.95-7.74 (m, 6H), 7.33 (dd, J=8.8, 5.1 Hz, 1H), 7.14 (dd, J=9.2, 3.0 Hz, 1H), 7.08 (ddd, J=8.8, 8.0, 3.1 Hz, 1H), 6.55 (dd, J=2.6, 1.6 Hz, 1H), 6.04-5.95 (m, 1H), 5.21 (dd, J=18.8, 1.2 Hz, 1H), 5.06 (dd, J=18.6, 1.7 Hz, 1H), 4.47 (p, J=8.6 Hz, 2H).

Additional compounds prepared according to the methods of Examples 24 are listed in Table 9 below. Corresponding ¹H NMR and mass spectrometry characterization for these compounds are described in Table 1. Certain compounds in Table 9 below were prepared with other compounds whose preparation is described in the Examples herein.

TABLE 9
Additional Exemplary Compounds
Compound

I-32
I-44
I-80
I-81
I-82
I-100
I-105
I-106
I-107
I-108
I-109
I-110
I-136
I-138
I-139
I-159
I-168
I-169
I-184
I-185
I-186
I-187
I-189
I-201
I-202
I-203
I-204
I-205
I-206
I-207
I-208
I-225
I-226
I-230
I-233
I-234
I-235
I-236
I-239
I-240
I-243
I-244
I-245
I-246
I-247
I-248
I-254
I-255
I-256
I-257
I-258
I-259
I-270
I-273
I-278
I-279
I-284
I-285
I-298
I-299
I-306
I-307
I-308
I-309
I-311
I-312
I-314
I-318
I-319
I-320
I-321
I-322
I-323
I-324
I-325
I-326
I-327
I-328
I-329
I-330
I-331
I-332
I-333
I-334
I-335
I-336
I-337
I-338
I-339
I-347
I-348
I-349
I-350
I-351
I-360
I-361
I-362
I-363
I-364
I-365
I-366
I-367
I-368
I-369
I-370
I-371
I-372
I-373
I-374
I-375
I-376
I-377
I-378
I-379
I-380
I-381
I-382
I-383
I-384
I-385
I-386
I-387
I-388
I-389
I-391
I-392
I-393
I-394
I-395
I-398
I-399
I-400
I-401
I-402
I-403
I-404
I-405
I-406
I-407
I-408
I-416
I-417
I-418
I-419
I-420
I-421
I-422
I-423
I-424
I-426
I-427
I-428
I-429
I-430
I-431
I-432
I-433
I-434
I-435
I-436
I-437
I-444
I-445
I-447
I-448
I-449
I-450
I-451
I-452
I-453
I-454
I-455
I-456
I-457
I-458
I-459
I-460
I-461
I-462
I-463
I-464
I-465
I-466
I-467
I-468
I-473
I-474
I-475
I-476
I-478
I-480
I-481
I-482
I-483
I-487
I-488
I-489
I-491
I-492
I-493
I-494
I-495
I-496
I-498
I-499
I-500
I-501
I-502
I-503
I-504
I-505
I-506
I-507
I-508
I-509
I-510
I-511
I-512
I-513
I-514
I-515
I-516
I-517
I-518
I-519
I-520
I-521
I-522
I-523
I-524
I-525
I-526
I-527
I-528
I-529
I-534
I-535
I-536
I-537
I-540
I-541
I-542
I-543
I-544
I-545
I-546
I-547
I-548
I-549
I-550
I-551
I-552
I-553
I-554
I-555
I-556
I-558
I-559
I-560
I-561
I-562
I-563
I-564
I-565
I-566
I-576
I-577
I-578
I-579
I-580
I-581
I-582
I-583
I-584
I-585
I-586
I-587
I-588
I-598
I-601
I-602
I-605
I-606
I-607
I-608
I-609
I-610
I-611
I-612
I-613
I-614
I-615
I-616
I-627
I-639
I-640
I-641
I-642
I-643
I-644
I-645
I-646
I-647
I-648
I-651
I-660
I-661
I-662
I-663
I-666
I-667
I-669
I-678
I-680
I-681
I-682
I-684
I-686
I-687
I-688
I-703
I-704
I-706
I-707
I-708
I-709
I-710
I-711
I-712
I-713
I-714
I-715
I-716
I-717
I-718
I-719
I-720
I-721
I-722
I-723
I-724
I-733

### Example 25

### (S)—N-(3-(((1H-1,2,3-triazol-4-yl)methyl)carbamoyl)-8-(2-chloro-5-fluorophenyl)-6-oxo-5,6,7,8-tetrahydroimidazo[1,5-a]pyrazin-1-yl)benzo[d]isothiazole-3-carboxamide (I-603 or I-604) and (R)—N-(3-(((1H-1,2,3-triazol-4-yl)methyl)carbamoyl)-8-(2-chloro-5-fluorophenyl)-6-oxo-5,6,7,8-tetrahydroimidazo[1,5-a]pyrazin-1-yl)benzo[d]isothiazole-3-carboxamide (I-603 or I-604)

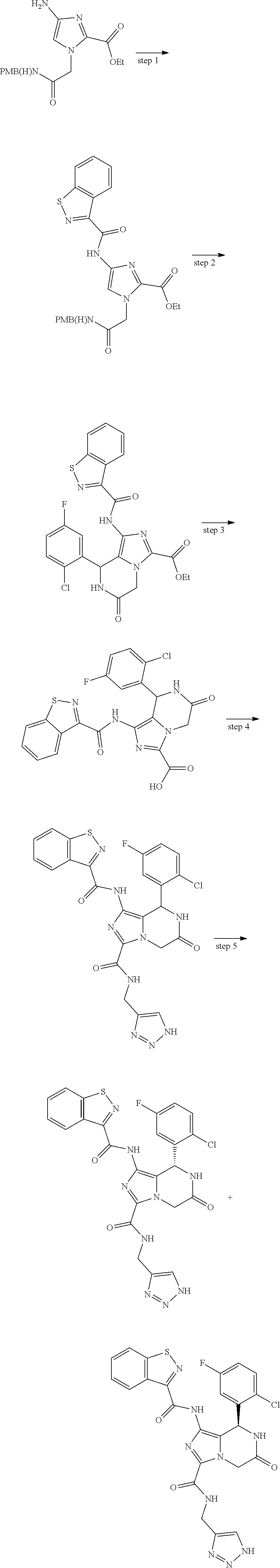

### Step 1. ethyl 4-(benzo[d]isothiazole-3-carboxamido)-1-(2-((4-methoxybenzyl)amino)-2-oxoethyl)-1H-imidazole-2-carboxylate

To a solution of ethyl 4-amino-1-(2-((4-methoxybenzyl)amino)-2-oxoethyl)-1H-imidazole-2-carboxylate (2.0 g) and benzo[d]isothiazole-3-carboxylic acid (1.078 g) in dichloromethane (50 mL) was added HOBt monohydrate (1.38 g), 1-(3-dimethylamino propyl)-3-ethylcarbodiimide hydrochloride (EDCI) (1.73 g) and DIEA (2.333 g) at room temperature under nitrogen. The reaction was stirred overnight at room temperature. The precipitate was collected by filtration, washed with cold Et₂O (2×15 mL) and dried to give the desired product (4.0 g, 33.5% Purity) as a brown solid, which was used in the next step without further purification. LCMS: RT 1.42 min, [M+H]⁺ 494.2, LCMS method D.

### Step 2. ethyl 1-(benzo[d]isothiazole-3-carboxamido)-8-(2-chloro-5-fluorophenyl)-6-oxo-5,6,7,8-tetrahydroimidazo[1,5-a]pyrazine-3-carboxylate

A microwave vial was charged with ethyl 4-(benzo[d]isothiazole-3-carboxamido)-1-(2-((4-methoxybenzyl)amino)-2-oxoethyl)-1H-imidazole-2-carboxylate (350 mg). 2-Chloro-5-fluorobenzaldehyde (179.9 mg) was added, followed by Eaton's reagent (7.09 g) in a single portion. The reaction was heated at 105° C. under microwave for 20 minutes. The reaction solution was cooled to 0° C., diluted with ethyl acetate (30 mL) and quenched with cold saturated NaHCO₃ solution. The aqueous phase was extracted with ethyl acetate (3×50 mL). The organic layers were combined, dried over Na₂SO₄ and concentrated under reduced pressure. The crude material was dissolved in DMF (1.5 mL) and purified by reverse phase chromatography [C18, 80 g cartridge, using a gradient of 5-100% of acetonitrile in 10 mM ammonium formate solution) to give the desired product (220 mg) as a brown solid. LCMS: RT 1.31 min, [M+H]⁺ 514.1, LCMS method D.

### Step 3. 1-(benzo[d]isothiazole-3-carboxamido)-8-(2-chloro-5-fluorophenyl)-6-oxo-5,6,7,8-tetrahydroimidazo[1,5-a]pyrazine-3-carboxylic acid

A 50 mL round bottom flask was charged with ethyl 1-(benzo[d]isothiazole-3-carboxamido)-8-(2-chloro-5-fluorophenyl)-6-oxo-5,6,7,8-tetrahydroimidazo[1,5-a]pyrazine-3-carboxylate (1.0 g). MeOH (7 mL) was added, followed by sodium hydroxide (233.5 mg) in H₂O (4 mL) at 15° C. The solution was stirred at 15° C. for 2 hours. After cooling to 0° C. HCl (1 N, 15 mL) was added. The formed precipitate was collected by filtration, washed with water (2×10 mL) and Et₂O (3×15 mL), and dried under reduced pressure to give the desired product (935 mg) as a brown solid, which was used in the next step without purification. LCMS: RT 1.04 min, [M+H]⁺ 486.1, LCMS method D.

### Step 4. N-(3-(((1H-1,2,3-triazol-4-yl)methyl)carbamoyl)-8-(2-chloro-5-fluorophenyl)-6-oxo-5,6,7,8-tetrahydroimidazo[1,5-a]pyrazin-1-yl)benzo[d]isothiazole-3-carboxamide

A 10 mL round bottom flask was charged with 1-(benzo[d]isothiazole-3-carboxamido)-8-(2-chloro-5-fluorophenyl)-6-oxo-5,6,7,8-tetrahydroimidazo[1,5-a]pyrazine-3-carboxylic acid (150 mg), HATU (293 mg), DMF (1 mL), triethylamine (156 mg) and (1H-1,2,3-triazol-4-yl)methanamine hydrochloride (103.9 mg). The reaction was stirred at room temperature for 72 hours. More (1H-1,2,3-triazol-4-yl)methanamine hydrochloride (50 mg) was added and the reaction was stirred at room temperature for 18 hours. The product was purified by reverse phase chromatography [C18, 30 g cartridge, using 5-100% of acetonitrile in 10 mM ammonium formate solution) to give the desired product (19.3 mg) as a white solid. LCMS: RT 2.03 min, [M+H]⁺ 566.2, LCMS method L. ¹H NMR (400 MHz, CD₃OD) δ 8.80 (d, J=8.2 Hz, 1H), 8.12 (d, J=8.3 Hz, 1H), 7.75 (br. s, 1H), 7.64 (td, J=7.2, 0.8 Hz, 1H), 7.58 (td, J=7.2, 0.7 Hz, 1H), 7.22 (dd, J=8.8, 5.0 Hz, 1H), 7.11 (dd, J=9.0, 3.0 Hz, 1H), 6.85 (td, J=5.1, 3.0 Hz, 1H), 6.36 (br. s, 1H), 5.33 (dd, J=19.0, 0.8 Hz, 1H), 5.24 (dd, J=19.0, 1.6 Hz, 1H), 4.68 (d, J=1.6 Hz, 2H).

### Step 5. (S)—N-(3-(((1H-1,2,3-triazol-4-yl)methyl)carbamoyl)-8-(2-chloro-5-fluorophenyl)-6-oxo-5,6,7,8-tetrahydroimidazo[1,5-a]pyrazin-1-yl)benzo[d]isothiazole-3-carboxamide and (R)—N-(3-(((1H-1,2,3-triazol-4-yl)methyl)carbamoyl)-8-(2-chloro-5-fluorophenyl)-6-oxo-5,6,7,8-tetrahydroimidazo[1,5-a]pyrazin-1-yl)benzo[d]isothiazole-3-carboxamide

N-(3-(((1H-1,2,3-triazol-4-yl)methyl)carbamoyl)-8-(2-chloro-5-fluorophenyl)-6-oxo-5,6,7,8-tetrahydroimidazo[1,5-a]pyrazin-1-yl)benzo[d]isothiazole-3-carboxamide (25 mg) was chirally resolved using the chiral SFC condition below to give both enantiomers as a white solid. Column: AS-H, 10×250 mm, 5 μm; mobile phase: 60% MeOH containing 10 mM ammonium formate, 40% supercritical CO₂; gradient: isocratic; flow rate: 10 m/min; backpressure: 120 bar; column temperature: 40 C; run time: 20 minutes.

Compound I-604, Peak 1: 11 mg. LCMS: RT 2.02 min, [M+H]⁺ 566.1, LCMS method L. ¹H NMR (400 MHz, DMSO-d6) δ 10.31 (br. s, 1H), 8.95 (d, J=2.2 Hz, 1H), 8.92 (t, J=6.2 Hz, 1H), 8.59 (dt, J=8.0, 1.2 Hz, 1H), 8.30 (dt, J=8.4, 0.8 Hz, 1H), 7.71 (br. s, 1H), 7.68 (td, J=7.2, 1.2 Hz, 1H), 7.60 (td, J=6.8, 1.2 Hz, 1H), 7.26 (dd, J=8.8, 5.1 Hz, 1H), 7.17 (dd, J=9.2, 3.1 Hz, 1H), 7.01 (td, J=8.8, 3.2 Hz, 1H), 6.19 (br. s, 1H), 5.26 (d, J=18.1 Hz, 1H), 5.12 (dd, J=18.7, 1.5 Hz, 1H), 4.59-4.39 (m, 2H).

Compound I-603, Peak 2: 12.2 mg. LCMS: RT 2.02 min, [M+H]⁺ 566.1, LCMS method L. ¹H NMR (400 MHz, DMSO-d6) δ 8.96 (br. s, 1H), 8.92 (t, J=6.0 Hz, 1H), 8.58 (d, J=8.0 Hz, 1H), 8.34 (br. s, 1H), 8.30 (d, J=8.2 Hz, 1H), 7.71 (br. s, 1H), 7.68 (td, J=8.4, 1.2 Hz, 1H), 7.60 (td, J=8.4, 1.2 Hz, 1H), 7.26 (dd, J=8.8, 5.2 Hz, 1H), 7.17 (dd, J=9.2, 3.1 Hz, 1H), 7.00 (td, J=8.0, 3.2 Hz, 1H), 6.19 (br. s, 1H), 5.26 (d, J=18.8 Hz, 1H), 5.12 (dd, J=18.7, 1.4 Hz, 1H), 4.63-4.40 (m, 2H).

### Example 26

### (S)-(4-((1-(benzo[d]isothiazole-3-carboxamido)-8-(2-chloro-5-fluorophenyl)-6-oxo-5,6,7,8-tetrahydroimidazo[1,5-a]pyrazine-3-carboxamido)methyl)-2H-1,2,3-triazol-2-yl)methyl diphenyl phosphate (I-628)

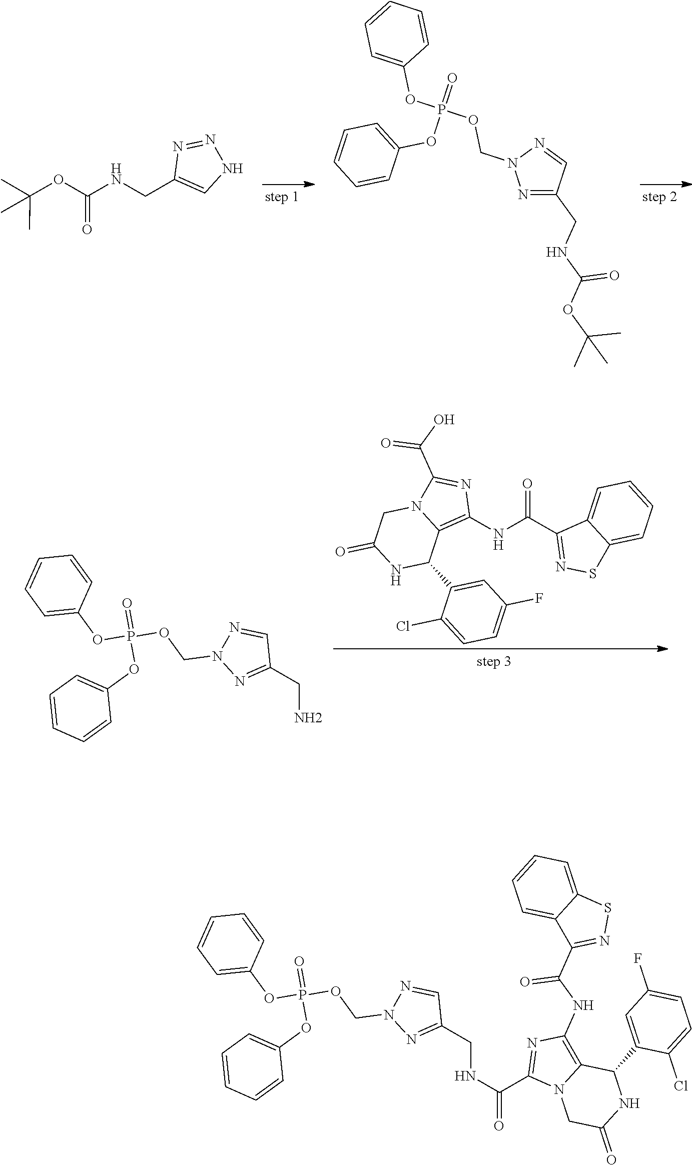 [SMILES: CC(C)(C)OC(=O)NCc1c[nH]nn1 ; CC(C)(C)OC(=O)NCc1cnn(COP(=O)(Oc2ccccc2)Oc2ccccc2)n1 ; NCc1cnn(COP(=O)(Oc2ccccc2)Oc2ccccc2)n1 ; O=C1Cn2c(C(=O)O)nc(NC(=O)c3nsc4ccccc34)c2[C@H](c2cc(F)ccc2Cl)N1 ; O=C1Cn2c(C(=O)NCc3cnn(COP(=O)(Oc4ccccc4)Oc4ccccc4)n3)nc(NC(=O)c3nsc4ccccc34)c2[C@H](c2cc(F)ccc2Cl)N1]

### Step 1. tert-butyl ((2-(((diphenoxyphosphoryl)oxy)methyl)-2H-1,2,3-triazol-4-yl)methyl)carbamate

A round bottom flask was charged with tert-butyl ((1H-1,2,3-triazol-4-yl)methyl)carbamate (500 mg), DMF (5 mL), potassium carbonate (1.05 g), chloromethyl diphenyl phosphate (753 mg) and a stirbar, and the solution was stirred for 1 h at 0° C. The reaction mixture was diluted with water (20 mL), and the aqueous phase was extracted with ethyl acetate (15 mL) three times. The combined organic layers were washed with brine, dried over sodium sulfate, filtered, and concentrated in vacuo. The residue was purified by reverse flash chromatography with the following conditions: Column, C18 silica gel; mobile phase, 10% to 50% acetonitrile in water over 10 min; detector, UV 254 nm to give the desired product (60 mg) as a yellow oil. LCMS: RT 1.19 min, [M+H]⁺ 461.25, LCMS method A.

### Step 2. (4-(aminomethyl)-2H-1,2,3-triazol-2-yl)methyl diphenyl phosphate

A round bottom flask was charged with tert-butyl ((2-(((diphenoxyphosphoryl)oxy)methyl)-2H-1,2,3-triazol-4-yl)methyl)carbamate (55 mg), TFA/DCM (4 mL, 1:1) and a stirbar, and the solution was stirred for 1 h at 25° C. The reaction mixture was concentrated in vacuo to give the desired product (36 mg) as a yellow oil. LCMS: RT 0.95 min, [M+H]⁺ 360.95, LCMS method N.

### Step 3. (S)-(4-((1-(benzo[d]isothiazole-3-carboxamido)-8-(2-chloro-5-fluorophenyl)-6-oxo-5,6,7,8-tetrahydroimidazo[1,5-a]pyrazine-3-carboxamido)methyl)-2H-1,2,3-triazol-2-yl)methyl diphenyl phosphate

A round bottom flask was charged with (4-(aminomethyl)-2H-1,2,3-triazol-2-yl)methyl diphenyl phosphate (30 mg), DMF (4 mL), (S)-1-(benzo[d]isothiazole-3-carboxamido)-8-(2-chloro-5-fluorophenyl)-6-oxo-5,6,7,8-tetrahydroimidazo[1,5-a]pyrazine-3-carboxylic acid (40 mg, prepared, for example, by SFC chiral resolution of the product of Step 3 of Example 25, above), HATU (38 mg), DIEA (32 mg) and a stirbar, and the solution was stirred for 1 h at 25° C. The reaction mixture was diluted with water (10 mL), and the aqueous phase was extracted with ethyl acetate (10 mL) three times. The combined organic layers were washed with brine, dried over sodium sulfate, filtered, and concentrated in vacuo. The resulting crude material was purified by HPLC (Column: XBridge Prep OBD C18 column, 30*150 mm, 5 μm; mobile phase A: water (10 mmol/L NH₄HCO₃), mobile phase B: acetonitrile; flow rate: 60 mL/min; gradient: 35% B to 65% B in 7 min, then 65% B; wavelength: 220 nm; RT: 8.57 min) to give the desired product (10.7 mg) as an off-white amorphous solid. LCMS: RT 1.26 min, [M+H]⁺ 828.20, LCMS method I. ¹H NMR (400 MHz, DMSO-d6) 10.35 (s, 1H), 9.09 (t, J=6.2 Hz, 1H), 8.98 (d, J=2.4 Hz, 1H), 8.60 (d, J=8.2 Hz, 1H), 8.31 (d, J=8.2 Hz, 1H), 7.88 (s, 1H), 7.73-7.65 (m, 1H), 7.61 (t, J=7.6 Hz, 1H), 7.41 (t, J=7.8 Hz, 4H), 7.31-7.21 (m, 3H), 7.18 (td, J=6.1, 2.8 Hz, 5H), 7.02 (td, J=8.4, 3.1 Hz, 1H), 6.42 (d, J=11.9 Hz, 2H), 6.20 (s, 1H), 5.24 (s, 1H), 5.14-5.05 (m, 1H), 4.60-4.45 (m, 2H).

Compound I-705 was also prepared according to the methods of Example 26. Mass spectrometry and ¹H NMR characterization are provided for this compound in Table 1.

### Example 27

### (S)—N-(8-(2-chloro-5-fluorophenyl)-3-(1H-imidazol-2-yl)-6-oxo-5,6,7,8-tetrahydroimidazo[1,5-a]pyrazin-1-yl)-3-fluoro-5-(trifluoromethyl)benzamide (I-160) and (R)—N-(8-(2-chloro-5-fluorophenyl)-3-(1H-imidazol-2-yl)-6-oxo-5,6,7,8-tetrahydroimidazo[1,5-a]pyrazin-1-yl)-3-fluoro-5-(trifluoromethyl)benzamide (I-161)

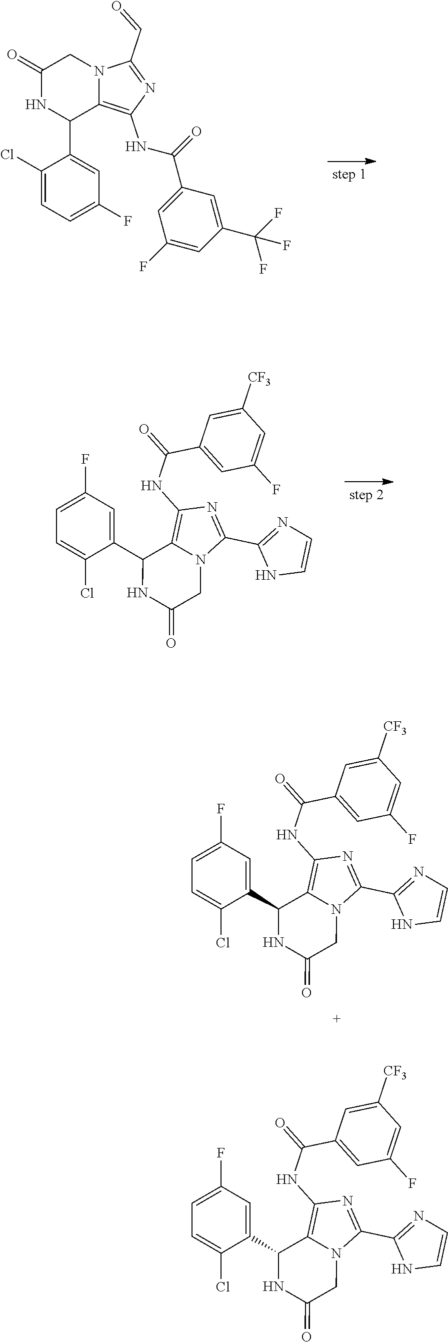 [SMILES: O=Cc1nc(NC(=O)c2cc(F)cc(C(F)(F)F)c2)c2n1CC(=O)NC2c1cc(F)ccc1Cl ; O=C1Cn2c(-c3ncc[nH]3)nc(NC(=O)c3cc(F)cc(C(F)(F)F)c3)c2C(c2cc(F)ccc2Cl)N1 ; O=C1Cn2c(-c3ncc[nH]3)nc(NC(=O)c3cc(F)cc(C(F)(F)F)c3)c2[C@H](c2cc(F)ccc2Cl)N1 ; O=C1Cn2c(-c3ncc[nH]3)nc(NC(=O)c3cc(F)cc(C(F)(F)F)c3)c2[C@@H](c2cc(F)ccc2Cl)N1]

### Step 1. N-(8-(2-chloro-5-fluorophenyl)-3-(1H-imidazol-2-yl)-6-oxo-5,6,7,8-tetrahydroimidazo[1,5-a]pyrazin-1-yl)-3-fluoro-5-(trifluoromethyl)benzamide

To a stirred solution of N-(8-(2-chloro-5-fluorophenyl)-3-formyl-6-oxo-5,6,7,8-tetrahydroimidazo[1,5-a]pyrazin-1-yl)-3-fluoro-5-(trifluoromethyl)benzamide (450 mg) in ethanol (5 mL) at 0° C. were added glyoxal (1.55 mL, 40% weight in water) and ammonia in water (1.81 mL, 29% weight). The resulting solution was stirred at room temperature for 5 hours. The reaction mixture was diluted with ethyl acetate and water (5 mL each). Phases were separated and the aqueous phase was extracted with ethyl acetate (2×30 mL). The combined organic layers were dried over Na₂SO₄ and concentrated in vacuo. The crude residue was first purified by flash column chromatography (SiO₂ 25 g; eluent: 0-6% MeOH in dichloromethane) then re-purified on a reverse C18 column (12 g); eluent A: 10 mM ammonium formate solution; B: acetonitrile; gradient: 0-80% B) to afford the desired product (35 mg) as a light yellow solid. LCMS: RT 2.01 min, [M+H]⁺ 537.2, LCMS method V. ¹H NMR (400 MHz, DMSO-d₆) δ 12.80 (br. s, 1H), 10.36 (s, 1H), 8.93 (d, J=2.0 Hz, 1H), 7.93 (d, J=8.5 Hz, 1H), 7.83 (overlapping s, 1H), 7.80 (submerged d, J=8.5 Hz, 1H), 7.36 (dd, J=8.8, 5.2 Hz, 1H), 7.23 (d, J=1.1 Hz, 1H), 7.15 (dd, J=9.2, 3.1 Hz, 1H), 7.13 (d, J=1.1 Hz, 1H), 7.10 (td, J=8.7, 3.1 Hz, 1H), 6.03 (br. s, 1H), 5.33 (d, J=18.3 Hz, 1H), 5.12 (dd, J=18.6, 1.3 Hz, 1H).

### Step 2. (S)—N-(8-(2-chloro-5-fluorophenyl)-3-(1H-imidazol-2-yl)-6-oxo-5,6,7,8-tetrahydroimidazo[1,5-a]pyrazin-1-yl)-3-fluoro-5-(trifluoromethyl)benzamide and (R)—N-(8-(2-chloro-5-fluorophenyl)-3-(1H-imidazol-2-yl)-6-oxo-5,6,7,8-tetrahydroimidazo[1,5-a]pyrazin-1-yl)-3-fluoro-5-(trifluoromethyl)benzamide

N-(8-(2-chloro-5-fluorophenyl)-3-(1H-imidazol-2-yl)-6-oxo-5,6,7,8-tetrahydroimidazo[1,5-a]pyrazin-1-yl)-3-fluoro-5-(trifluoromethyl)benzamide (25.4 mg) was chirally resolved using the chiral SFC condition below to give the two enantiomers. Column: ChiralPak IC—H 21×250 mm; mobile phase: 20% methanol in CO₂, flow rate: 70 mL/min, sample: 25.4 mg of sample was dissolved in 2 mL methanol+2 mL dichloromethane; injection: 2 mL; detection wavelength: 254 nm.

Compound I-161, Peak 1, 9.1 mg. Chiral SFC RT 1.20 min. LCMS: RT 0.976 min, [M+H]⁺ 537.0, LCMS method G.

Compound I-160, Peak 2, 9.0 mg. Chiral SFC RT 1.50 min. LCMS: RT 0.976 min, [M+H]⁺ 537.0, LCMS method G.

### Example 28

### N-(8-(2-chloro-5-fluorophenyl)-6-oxo-3-(thiazol-5-yl)-5,6,7,8-tetrahydroimidazo[1,5-a]pyrazin-1-yl)-3-fluoro-5-(trifluoromethyl)benzamide (I-183)

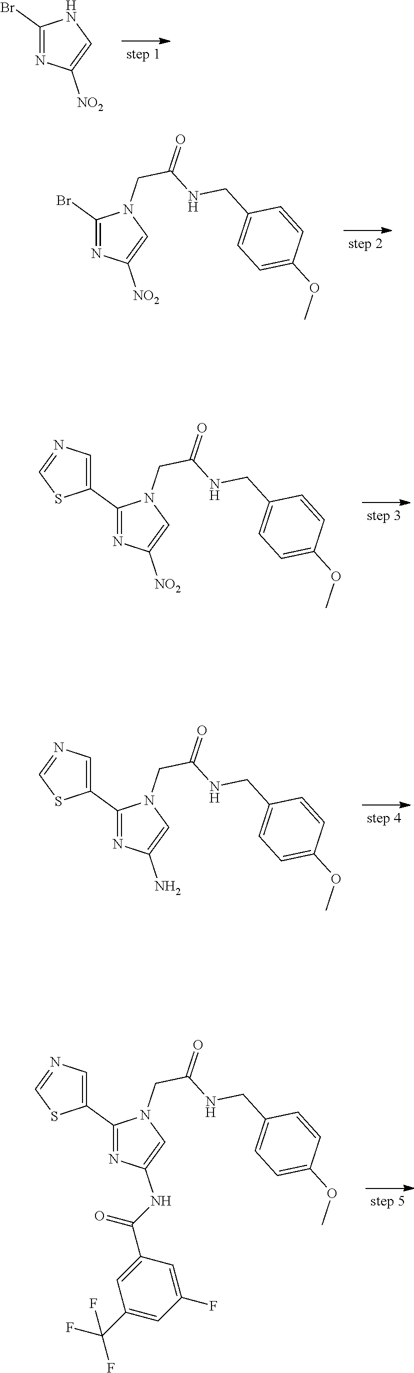 [SMILES: O=[N+]([O-])c1c[nH]c(Br)n1 ; COc1ccc(CNC(=O)Cn2cc([N+](=O)[O-])nc2Br)cc1 ; COc1ccc(CNC(=O)Cn2cc([N+](=O)[O-])nc2-c2cncs2)cc1 ; COc1ccc(CNC(=O)Cn2cc(N)nc2-c2cncs2)cc1 ; COc1ccc(CNC(=O)Cn2cc(NC(=O)c3cc(F)cc(C(F)(F)F)c3)nc2-c2cncs2)cc1]

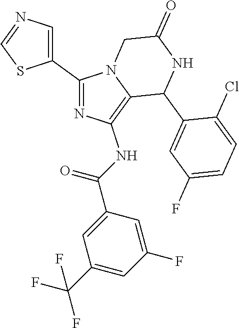 [SMILES: O=C1Cn2c(-c3cncs3)nc(NC(=O)c3cc(F)cc(C(F)(F)F)c3)c2C(c2cc(F)ccc2Cl)N1]

### Step 1. 2-(2-bromo-4-nitro-1H-imidazol-1-yl)-N-(4-methoxybenzyl)acetamide

A 50 mL round bottom flask was charged with 2-bromo-4-nitro-1H-imidazole (2.30 g). Acetonitrile (10 mL) was added, followed by dropwise addition of 2-(tert-butyl)-1,1,3,3-tetramethylguanidine (2.26 g). The resulting yellow solution was stirred for 5 minutes prior to the addition of 2-bromo-N-(4-methoxybenzyl)acetamide (3.09 g) in portions. The reaction was stirred for 16 hours. The solution was concentrated under reduced pressure and diluted with ethyl acetate. HCl (0.5 N, 50 mL) was added, and the aqueous phase was extracted with ethyl acetate. A precipitate formed during the extraction which was filtered and washed with diethyl ether. The organic layers were combined, dried over Na₂SO₄ and concentrated under reduced pressure. The beige solid was triturated with dichloromethane:Et₂O (1:5), collected by filtration, washed with Et₂O and dried to give the desired product (3.59 g). LCMS: RT 1.30 min, [M+H]⁺ 369.0/371.0, LCMS method Q.

### Step 2. N-(4-methoxybenzyl)-2-(4-nitro-2-(thiazol-5-yl)-1H-imidazol-1-yl)acetamide

A 50 mL round bottom flask vial was charged with 2-(2-bromo-4-nitro-1H-imidazol-1-yl)-N-(4-methoxybenzyl)acetamide (600 mg), 5-(4,4,5,5-tetramethyl-1,3,2-dioxaborolan-2-yl)thiazole (100 mg), potassium carbonate (674 mg) and palladiumtetrakis (282 mg). The vial was flushed with N₂ for 2 minutes. Dioxane (5 mL) and water (1 mL) were added. Nitrogen gas was bubbled through the reaction media for 2 minutes. The flask was sealed and heated at 90° C. A solution of 5-(4,4,5,5-tetramethyl-1,3,2-dioxaborolan-2-yl)thiazole (586 mg) in dioxane (5 mL) was added dropwise over 3 hours. The reaction was cooled to room temperature and quenched with saturated aqueous NH₄Cl solution. The aqueous layer was extracted three times with ethyl acetate (5 mL×3). The organic layers were combined, dried over Na₂SO₄ and concentrated under reduced pressure. To the crude material was added cold acetonitrile and Et₂O (2:1) and the white solid was triturated for 5 minutes before filtration. The collected solid was washed with cold acetonitrile and dried to afford the desired product (316 mg) as a grey powder. LCMS: RT 0.79 min, [M+H]⁺ 374.2, LCMS method U.

### Step 3. 2-(4-amino-2-(thiazol-5-yl)-1H-imidazol-1-yl)-N-(4-methoxybenzyl)acetamide

To a suspension of N-(4-methoxybenzyl)-2-(4-nitro-2-(thiazol-5-yl)-1H-imidazol-1-yl)acetamide (290 mg) in MeOH (5 mL) was added palladium on carbon (90.9 mg, 10% weight). The mixture was stirred at room temperature under 1 atm. of hydrogen (balloon) for 2 hours. The reaction mixture was purged with N₂, filtered through a syringe filter and concentrated under reduced pressure to afford the crude product as a brown oil. The crude material was purified using reverse phase column chromatography (30 g column, Eluent A: 10 mM ammonium formate solution; B: acetonitrile; gradient: 5-50% B in 16 minutes) to give the desired product (50 mg). LCMS: RT 1.02 min, [M+H]⁺ 344.1, LCMS method Q.

### Step 4. 3-fluoro-N-(1-(2-((4-methoxybenzyl)amino)-2-oxoethyl)-2-(thiazol-5-yl)-1H-imidazol-4-yl)-5-(trifluoromethyl)benzamide

A flame-dried microwave vial was charged with 2-(4-amino-2-(thiazol-5-yl)-1H-imidazol-1-yl)-N-(4-methoxybenzyl)acetamide (50 mg). Dichloromethane (1 mL) was added, followed by pyridine (46 mg) at room temperature. 3-Fluoro-5-(trifluoromethyl)benzoyl chloride (33 mg) was added dropwise under N₂ atmosphere. The solution was stirred for 20 minutes. The crude mixture was concentrated, diluted in DMF (1 mL) and directly loaded on a C18 column for purification (eluant A: 10 mM ammonium formate solution; B: acetonitrile; gradient: 5 to 50% B) to give the desired product (33 mg) as a white solid. LCMS: RT 1.61 min, [M+H]⁺ 534.1, LCMS method Q.

### Step 5. N-(8-(2-chloro-5-fluorophenyl)-6-oxo-3-(thiazol-5-yl)-5,6,7,8-tetrahydroimidazo[1,5-a]pyrazin-1-yl)-3-fluoro-5-(trifluoromethyl)benzamide

A microwave vial was charged with 3-fluoro-N-(1-(2-((4-methoxybenzyl)amino)-2-oxoethyl)-2-(thiazol-5-yl)-1H-imidazol-4-yl)-5-(trifluoromethyl)benzamide (33 mg) and 2-chloro-5-fluorobenzaldehyde (15 mg). Eaton's reagent (1 mL) was added, and the reaction was heated at 100° C. under microwave for 20 minutes. The material was diluted in ethyl acetate (5 mL) and quenched with cold saturated NaHCO₃ solution until no more bubbling was observed. The aqueous phase was extracted with ethyl acetate twice. The organic layers were combined, dried and concentrated under vacuum to afford the crude product as a brown oil. The crude material was dissolved in DMF (1 mL) and loaded on a 30 g reverse phase column for purification (10 mM ammonium formate solution:acetonitrile 95:5 to 35:65) to give the desired product (11.9 mg). LCMS: RT 2.89 min, [M+H]⁺ 554.2, LCMS method R. ¹H NMR (400 MHz, DMSO-d6) δ 10.52 (br. s, 1H), 9.17 (s, 1H), 9.00 (d, J=2.2 Hz, 1H), 8.41 (s, 1H), 7.93 (br. d, J=8.4 Hz, 1H), 7.86 (overlapping s, 1H), 7.85 (submerged br. d, J=9.1 Hz, 1H), 7.37 (dd, J=9.4, 5.1 Hz, 1H), 7.13-7.07 (m, 2H), 6.06 (s, 1H), 5.18 (d, J=17.1 Hz, 1H), 5.10 (dd, J=17.2, 1.0 Hz, 1H).

### Example 29

### (S)—N-(8-(2-chloro-5-fluorophenyl)-6-oxo-3-(4H-1,2,4-triazol-3-yl)-5,6,7,8-tetrahydroimidazo[1,5-a]pyrazin-1-yl)-3-fluoro-5-(trifluoromethyl)benzamide (I-262)

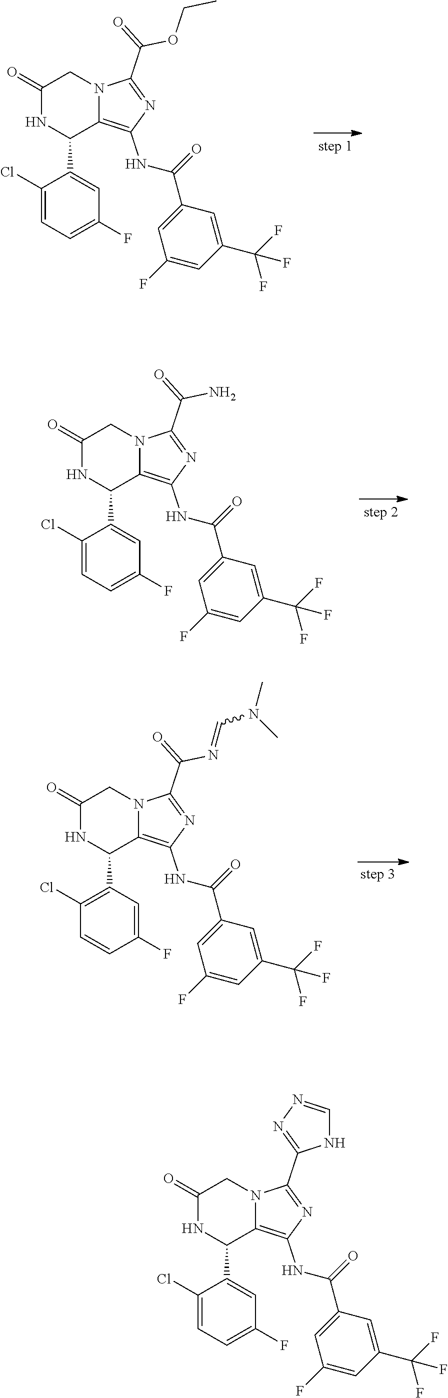 [SMILES: CCOC(=O)c1nc(NC(=O)c2cc(F)cc(C(F)(F)F)c2)c2n1CC(=O)N[C@H]2c1cc(F)ccc1Cl ; NC(=O)c1nc(NC(=O)c2cc(F)cc(C(F)(F)F)c2)c2n1CC(=O)N[C@H]2c1cc(F)ccc1Cl ; CN(C)C=NC(=O)c1nc(NC(=O)c2cc(F)cc(C(F)(F)F)c2)c2n1CC(=O)N[C@H]2c1cc(F)ccc1Cl ; O=C1Cn2c(-c3nnc[nH]3)nc(NC(=O)c3cc(F)cc(C(F)(F)F)c3)c2[C@H](c2cc(F)ccc2Cl)N1]

### Step 1. (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxamide

A round bottom flask was charged with ethyl (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (100 mg), NH₃ in MeOH (2 M, 1 mL) and MeOH (2 mL). The solution was stirred at 70° C. for 5 hours. The solvent was removed under reduced pressure. The resulting crude material was purified by HPLC (mobile phase A: water, mobile phase B: acetonitrile) to give the desired product (80 mg) as a yellow amorphous solid. LCMS: RT 0.98 min, [M+H]⁺ 513.90, LCMS method A

### Step 2. (S)-8-(2-chloro-5-fluorophenyl)-N-((dimethylamino)methylene)-1-(3-fluoro-5-(trifluoromethyl)benzamido)-6-oxo-5,6,7,8-tetrahydroimidazo[1,5-a]pyrazine-3-carboxamide

A round bottom flask was charged with (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxamide (80 mg), 1,1-dimethoxy-N,N-dimethylmethanamine (28 mg) and THF (2 mL). The solution was stirred at 25° C. for 2 hours and concentrated under reduced pressure. The resulting crude material was purified by HPLC (mobile phase A: water, mobile phase B: acetonitrile) to give the desired product (80 mg) as a yellow amorphous solid. LCMS: RT 0.93 min, [M+H]⁺ 569.00, LC method A.

### Step 3. (S)—N-(8-(2-chloro-5-fluorophenyl)-6-oxo-3-(4H-1,2,4-triazol-3-yl)-5,6,7,8-tetrahydroimidazo[1,5-a]pyrazin-1-yl)-3-fluoro-5-(trifluoromethyl)benzamide

A round bottom flask was charged with (S)-8-(2-chloro-5-fluorophenyl)-N-((dimethylamino)methylene)-1-(3-fluoro-5-(trifluoromethyl)benzamido)-6-oxo-5,6,7,8-tetrahydroimidazo[1,5-a]pyrazine-3-carboxamide (150 mg) and N₂H₄-H₂O (0.5 mL). Acetic acid (3 mL) was added, and the solution was stirred at 90° C. for 2 hours. The reaction mixture was diluted with H₂O (10 mL), and the aqueous phase was extracted with ethyl acetate (10 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (100 mg) as a white amorphous solid. LCMS: RT 0.803 min, [M+H]⁺ 538.0, LC Method E. ¹HNMR (400 MHz, DMSO-d6) δ 14.50 (s, 1H), 10.50 (s, 1H), 8.96 (s, 1H), 8.58 (s, 1H), 7.93 (d, J=8.5 Hz, 1H), 7.84 (d, J=11.4 Hz, 2H), 7.36 (dd, J=8.9, 5.3 Hz, 1H), 7.21-6.98 (m, 2H), 6.08 (s, 1H), 5.46-4.92 (m, 2H).

### Example 30

(S)—N-(8-(2-chloro-5-fluorophenyl)-6-oxo-3-phenyl-5,6,7,8-tetrahydroimidazo[1,5-a]pyrazin-1-yl)-3-fluoro-5-(trifluoromethyl)benzamide (I-304)

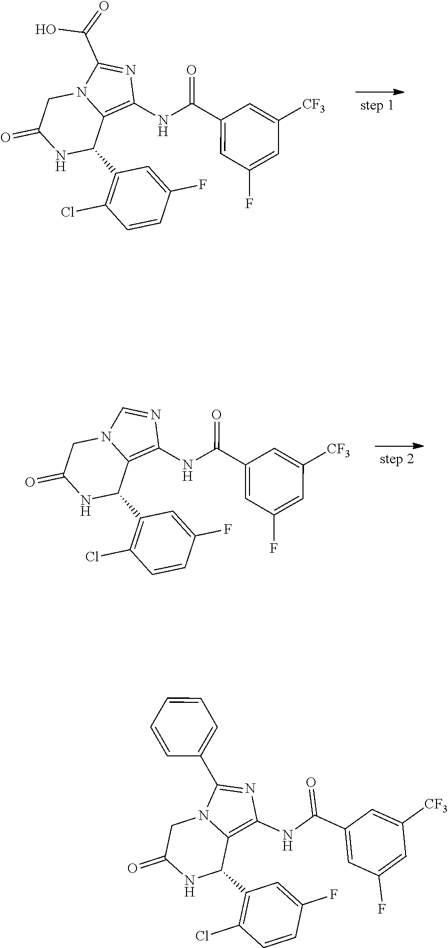 [SMILES: O=C1Cn2c(C(=O)O)nc(NC(=O)c3cc(F)cc(C(F)(F)F)c3)c2[C@H](c2cc(F)ccc2Cl)N1 ; O=C1Cn2cnc(NC(=O)c3cc(F)cc(C(F)(F)F)c3)c2[C@H](c2cc(F)ccc2Cl)N1 ; O=C1Cn2c(-c3ccccc3)nc(NC(=O)c3cc(F)cc(C(F)(F)F)c3)c2[C@H](c2cc(F)ccc2Cl)N1]

### Step 1. (S)—N-(8-(2-chloro-5-fluorophenyl)-6-oxo-5,6,7,8-tetrahydroimidazo[1,5-a]pyrazin-1-yl)-3-fluoro-5-(trifluoromethyl)benzamide

In a screw-cap vial equipped with a stir bar was charged (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylic acid (930 mg) and sodium chloride (106 mg) in DMSO (5 mL). The reaction mixture was sealed and heated in an oil bath (preheated at 140° C.) for 30 minutes. Addition of water at 15° C. to the reaction mixture initiated the precipitation, and the precipitate was collected by filtration and washed with generous amount of water. The precipitate was triturated with minimal amount of acetonitrile to give the desired product (609 mg) as a white powder, which was used in the next step without further purification. LCMS: RT 0.98 min, [M+H]⁺ 471.2, LCMS method U.

### Step 2. (S)—N-(8-(2-chloro-5-fluorophenyl)-6-oxo-3-phenyl-5,6,7,8-tetrahydroimidazo[1,5-a]pyrazin-1-yl)-3-fluoro-5-(trifluoromethyl)benzamide

In a screw-cap vial equipped with a stir bar was charged (S)—N-(8-(2-chloro-5-fluorophenyl)-6-oxo-5,6,7,8-tetrahydroimidazo[1,5-a]pyrazin-1-yl)-3-fluoro-5-(trifluoromethyl)benzamide (50 mg), diacetoxypalladium (4.8 mg), copper(I) iodide (40 mg) and iodobenzene (22 mg) in DMF (1 mL). The reaction mixture was sonicated, and degassed by bubbling nitrogen gas for 3-5 minutes, before being sealed. The reaction mixture was heated in an oil bath (pre-heated at 140° C.) overnight. The mixture was purified by reverse phase column chromatography (C18 30 g, eluent A: 10 mM ammonium formate solution, B: acetonitrile; gradient: 0-100% B) to give the desired product (6.2 mg) as a tan powder. LCMS: RT 1.19 min, [M+H]⁺ 547.3, LCMS method U. ¹H NMR: (400 MHz, DMSO-d6) δ 10.48 (s, 1H), 8.98 (d, J=2.4 Hz, 1H), 7.93 (d, J=8.4 Hz, 1H), 7.89 (s, 1H), 7.86 (overlapping d, J=8.9 Hz, 1H), 7.85-7.73 (m, 2H), 7.56-7.50 (m, 2H), 7.49-7.43 (m, 1H), 7.37 (dd, J=8.5, 5.0 Hz, 1H), 7.15-7.07 (m, 2H), 6.09 (s, 1H), 5.08 (d, J=17.1 Hz, 1H), 4.99 (d, J=17.3 Hz, 1H).

Compound I-305 was also prepared according to the methods of Example 30. Mass spectrometry and ¹H NMR characterization are provided for this compound in Table 1.

### Example 31

### (S)—N-(8-(2-chloro-5-fluorophenyl)-6-oxo-3-(1H-1,2,3-triazol-5-yl)-5,6,7,8-tetrahydroimidazo[1,5-a]pyrazin-1-yl)-3-fluoro-5-(trifluoromethyl)benzamide (I-310)

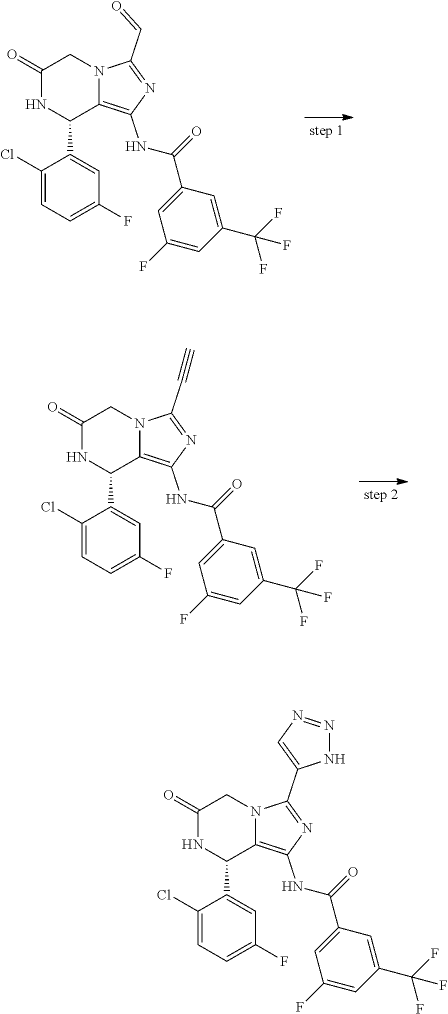 [SMILES: O=Cc1nc(NC(=O)c2cc(F)cc(C(F)(F)F)c2)c2n1CC(=O)N[C@H]2c1cc(F)ccc1Cl ; C#Cc1nc(NC(=O)c2cc(F)cc(C(F)(F)F)c2)c2n1CC(=O)N[C@H]2c1cc(F)ccc1Cl ; O=C1Cn2c(-c3cnn[nH]3)nc(NC(=O)c3cc(F)cc(C(F)(F)F)c3)c2[C@H](c2cc(F)ccc2Cl)N1]

### Step 1. (S)—N-(8-(2-chloro-5-fluorophenyl)-3-ethynyl-6-oxo-5,6,7,8-tetrahydroimidazo[1,5-a]pyrazin-1-yl)-3-fluoro-5-(trifluoromethyl)benzamide

A round bottom flask was charged with (S)—N-(8-(2-chloro-5-fluorophenyl)-3-formyl-6-oxo-5,6,7,8-tetrahydroimidazo[1,5-a]pyrazin-1-yl)-3-fluoro-5-(trifluoromethyl)benzamide (110 mg), dimethyl (1-diazo-2-oxopropyl)phosphonate (127 mg), K₂CO₃ (91.3 mg) and MeOH (2 mL). The solution was stirred at 25° C. for 3 hours. The reaction mixture was diluted with H₂O (20 mL), and the aqueous phase was extracted with ethyl acetate (15 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (80 mg) as a white amorphous solid. LCMS: RT 1.08 min, [M+H]⁺ 494.90, LCMS method A.

### Step 2. (S)—N-(8-(2-chloro-5-fluorophenyl)-6-oxo-3-(1H-1,2,3-triazol-5-yl)-5,6,7,8-tetrahydroimidazo[1,5-a]pyrazin-1-yl)-3-fluoro-5-(trifluoromethyl)benzamide

A round bottom flask was charged with (S)—N-(8-(2-chloro-5-fluorophenyl)-3-ethynyl-6-oxo-5,6,7,8-tetrahydroimidazo[1,5-a]pyrazin-1-yl)-3-fluoro-5-(trifluoromethyl)benzamide (70 mg), TMSN₃ (69 mg), sodium ascorbate (79 mg) and CuSO₄ (46 mg). Dimethylacetamide (2 mL) and H₂O (2 mL) were added, and the solution was stirred at 25° C. for 1 hour. The reaction mixture was diluted with H₂O (20 mL), and the aqueous phase was extracted with ethyl acetate (15 mL) three times. The combined organic layers were washed with brine, dried over sodium sulfate, filtered and concentrated in vacuo. The resulting crude material was purified by prep-HPLC (Column: Xselect CSH C18 OBD Column 30*150 mm, 5 μm; mobile phase A: water (0.1% formic acid), mobile phase B: acetonitrile; flow rate: 60 mL/min; gradient: 40% B to 45% B in 7 min; wavelength: 220 nm; RT 5.02 min) to give the desired product (20 mg) as a white amorphous solid. LCMS: RT 1.089 min, [M+H]⁺ 538.15, LCMS method M. ¹H NMR (400 MHz, DMSO-d6) δ 10.49 (s, 1H), 8.93 (s, 1H), 8.40 (s, 1H), 8.17 (s, 1H), 7.95-7.82 (m, 3H), 7.36 (t, J=6.7 Hz, 1H), 7.10 (d, J=8.9 Hz, 2H), 6.07 (s, 1H), 5.16 (q, J=18.2 Hz, 2H).

### Example 32

### (S)—N-(8-(2-chloro-5-fluorophenyl)-6-oxo-3-(1H-pyrazol-5-yl)-5,6,7,8-tetrahydroimidazo[1,5-a]pyrazin-1-yl)-3-fluoro-5-(trifluoromethyl)benzamide (I-359)

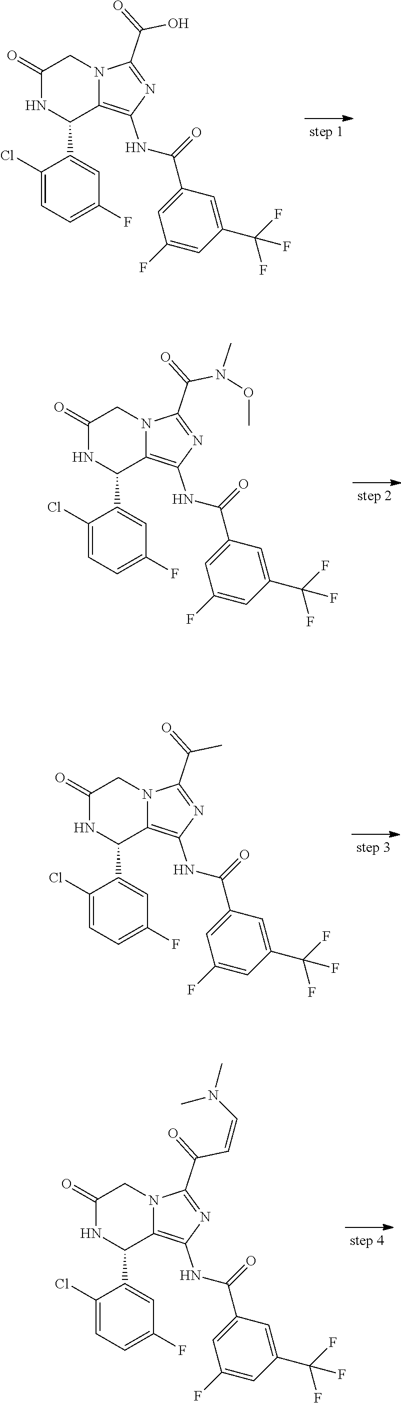 [SMILES: O=C1Cn2c(C(=O)O)nc(NC(=O)c3cc(F)cc(C(F)(F)F)c3)c2[C@H](c2cc(F)ccc2Cl)N1 ; CON(C)C(=O)c1nc(NC(=O)c2cc(F)cc(C(F)(F)F)c2)c2n1CC(=O)N[C@H]2c1cc(F)ccc1Cl ; CC(=O)c1nc(NC(=O)c2cc(F)cc(C(F)(F)F)c2)c2n1CC(=O)N[C@H]2c1cc(F)ccc1Cl ; CN(C)/C=C\C(=O)c1nc(NC(=O)c2cc(F)cc(C(F)(F)F)c2)c2n1CC(=O)N[C@H]2c1cc(F)ccc1Cl]

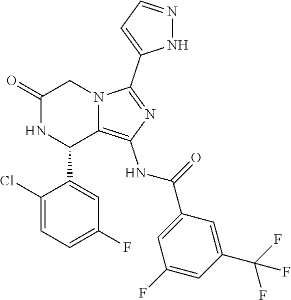 [SMILES: O=C1Cn2c(-c3ccn[nH]3)nc(NC(=O)c3cc(F)cc(C(F)(F)F)c3)c2[C@H](c2cc(F)ccc2Cl)N1]

### Step 1. (S)-8-(2-chloro-5-fluorophenyl)-1-(3-fluoro-5-(trifluoromethyl)benzamido)-N-methoxy-N-methyl-6-oxo-5,6,7,8-tetrahydroimidazo[1,5-a]pyrazine-3-carboxamide

A round bottom flask was charged with (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylic acid (400 mg), N,O-dimethylhydroxylamine (47.5 mg), HATU (354 mg), DIEA (301 mg) and DMF (5 mL). The solution was stirred at 25° C. for 1 hour. The reaction mixture was diluted with H₂O (20 mL), and the aqueous phase was extracted with ethyl acetate (15 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (410 mg) as a white amorphous solid. LCMS: RT 1.03 min, [M+H]⁺ 558.00, LCMS method A.

### Step 2. (S)—N-(3-acetyl-8-(2-chloro-5-fluorophenyl)-6-oxo-5,6,7,8-tetrahydroimidazo[1,5-a]pyrazin-1-yl)-3-fluoro-5-(trifluoromethyl)benzamide

A round bottom flask was charged with (S)-8-(2-chloro-5-fluorophenyl)-1-(3-fluoro-5-(trifluoromethyl)benzamido)-N-methoxy-N-methyl-6-oxo-5,6,7,8-tetrahydroimidazo[1,5-a]pyrazine-3-carboxamide (400 mg), methylmagnesium bromide (256 mg) and THF (5 mL). The solution was stirred at 25° C. for 1 hour. The reaction mixture was diluted with H₂O (20 mL), and the aqueous phase was extracted with ethyl acetate (15 mL) three times. The combined organic layers were washed with brine, dried over sodium sulfate, filtered and concentrated in vacuo. The resulting crude material was purified by HPLC (Eluent A: water with 0.1% formic acid, B: acetonitrile with 0.1% formic acid) to give the desired product (260 mg) as a white amorphous solid. LCMS: RT 1.05 min, [M+H]⁺ 512.95, LCMS method A.

### Step 3. (S)—N-(8-(2-chloro-5-fluorophenyl)-3-(3-(dimethylamino)acryloyl)-6-oxo-5,6,7,8-tetrahydroimidazo[1,5-a]pyrazin-1-yl)-3-fluoro-5-(trifluoromethyl)benzamide

A round bottom flask was charged with (S)—N-(3-acetyl-8-(2-chloro-5-fluorophenyl)-6-oxo-5,6,7,8-tetrahydroimidazo[1,5-a]pyrazin-1-yl)-3-fluoro-5-(trifluoromethyl)benzamide (250 mg), 1,1-dimethoxy-N,N-dimethylmethanamine (290 mg) and THF (5 mL). The solution was stirred at 70° C. for 1 hour. The reaction mixture was diluted with H₂O (20 mL) and the aqueous phase was extracted with ethyl acetate (15 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (220 mg) as a white amorphous solid. LCMS: RT 0.91 min, [M+H]⁺ 568.15, LCMS method E.

### Step 4. (S)—N-(8-(2-chloro-5-fluorophenyl)-6-oxo-3-(1H-pyrazol-5-yl)-5,6,7,8-tetrahydroimidazo[1,5-a]pyrazin-1-yl)-3-fluoro-5-(trifluoromethyl)benzamide

A round bottom flask was charged with (S)—N-(8-(2-chloro-5-fluorophenyl)-3-(3-(dimethylamino)acryloyl)-6-oxo-5,6,7,8-tetrahydroimidazo[1,5-a]pyrazin-1-yl)-3-fluoro-5-(trifluoromethyl)benzamide (100 mg), N₂H₄-H₂O (176 mg). Acetic acid (4 mL) was added, and the solution was stirred at 25° C. for 1 hour. The reaction mixture was diluted with H₂O (20 mL), and the aqueous phase was extracted with ethyl acetate (15 mL) three times. The combined organic layers were washed with brine, dried over sodium sulfate, filtered and concentrated in vacuo. The resulting crude material was purified by prep-HPLC (Column: XBridge Prep OBD C18 Column, 30*150 mm, 5 μm; mobile phase A: 10 mM NH₄HCO₃ solution, mobile phase B: acetonitrile; flow rate: 60 mL/min; Gradient: 30% B to 50% B in 8 min; wavelength: 220 nm; RT: 7.23 min) to give the desired product (30 mg) as a white amorphous solid. LCMS: RT 1.00 min, [M+H]⁺=537.15, LCMS method M. ¹H NMR (400 MHz, DMSO-d6) δ 13.22 (s, 1H), 10.46 (s, 1H), 8.91 (d, J=2.4 Hz, 1H), 7.96-7.81 (m, 4H), 7.36 (dd, J=9.5, 5.2 Hz, 1H), 7.13-7.04 (m, 2H), 6.67 (t, J=2.1 Hz, 1H), 6.06 (d, J=2.1 Hz, 1H), 5.18 (s, 1H), 5.12 (s, 1H).

### Example 33

### N-((5S,8R)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide (I-162, I-163, I-164, or I-165), N-((5R,8S)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide (I-162, I-163, I-164, or I-165), N-((5S,8S)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide (I-162, I-163, I-164, or I-165) and N-((5R,8R)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide (I-162, I-163, I-164, or I-165)

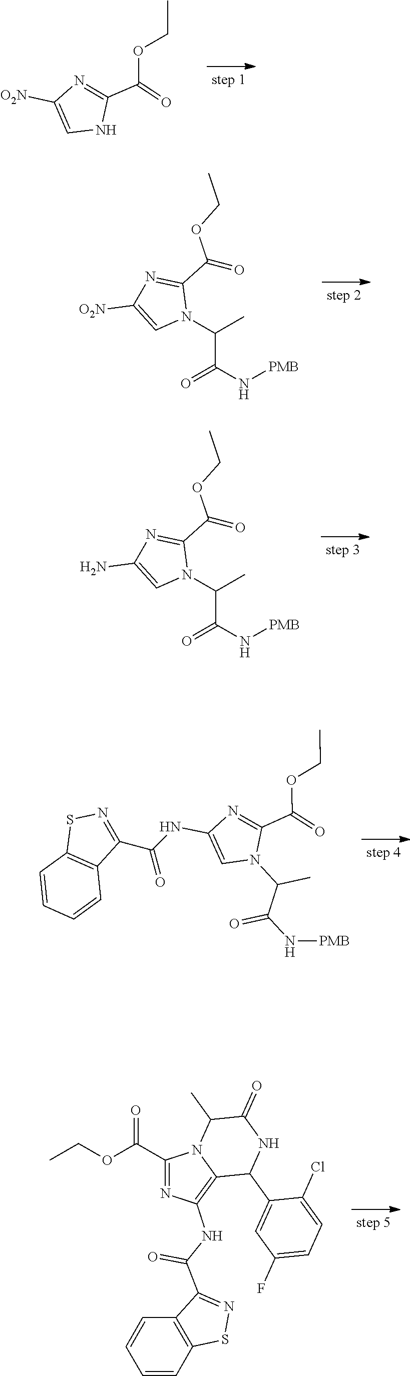

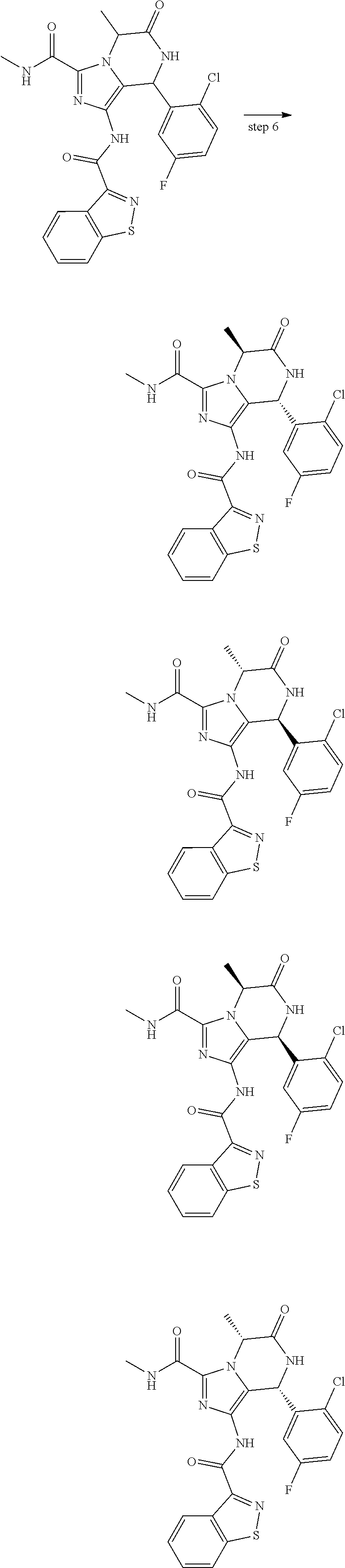 [SMILES: CNC(=O)c1nc(NC(=O)c2nsc3ccccc23)c2n1C(C)C(=O)NC2c1cc(F)ccc1Cl ; CNC(=O)c1nc(NC(=O)c2nsc3ccccc23)c2n1[C@@H](C)C(=O)N[C@@H]2c1cc(F)ccc1Cl ; CNC(=O)c1nc(NC(=O)c2nsc3ccccc23)c2n1[C@H](C)C(=O)N[C@H]2c1cc(F)ccc1Cl ; CNC(=O)c1nc(NC(=O)c2nsc3ccccc23)c2n1[C@@H](C)C(=O)N[C@H]2c1cc(F)ccc1Cl ; CNC(=O)c1nc(NC(=O)c2nsc3ccccc23)c2n1[C@H](C)C(=O)N[C@@H]2c1cc(F)ccc1Cl]

### Step 1. ethyl 1-(1-((4-methoxybenzyl)amino)-1-oxopropan-2-yl)-4-nitro-1H-imidazole-2-carboxylate

A round bottom flask was charged with ethyl 4-nitro-1H-imidazole-2-carboxylate (10 g), 2-bromo-N-(4-methoxybenzyl) propanamide (15 g), 2-(tert-butyl)-1,1,3,3-tetramethylguanidine (9.3 g) and acetonitrile (200 mL). The solution was stirred at 50° C. for 24 hours. The precipitate was collected by filtration and dried to give ethyl 1-(1-((4-methoxybenzyl)amino)-1-oxopropan-2-yl)-4-nitro-1H-imidazole-2-carboxylate (15 g, 40 mmol) as a white amorphous solid. Due to the presence of the nitro group [M+H]⁺ was not observed in LCMS.

### Step 2. ethyl 4-amino-1-(1-((4-methoxybenzyl)amino)-1-oxopropan-2-yl)-1H-imidazole-2-carboxylate

A round bottom flask was charged with ethyl 1-(1-((4-methoxybenzyl)amino)-1-oxopropan-2-yl)-4-nitro-1H-imidazole-2-carboxylate (10 g), Pd/C (10%, 4.8 g) and MeOH (100 mL). The flask was evacuated and flushed three times with nitrogen, followed by flushing with hydrogen. The mixture was stirred for 1 hour at room temperature under an atmosphere of hydrogen (balloon). The mixture was filtered through a Celite pad. The filtrate was concentrated under vacuum. The resulting crude material was purified by HPLC (mobile phase A: water, mobile phase B: acetonitrile) to give the desired product (6 g) as a yellow amorphous solid. LCMS: RT 0.79 min, [M+H]⁺ 347.05, LCMS method A.

### Step 3. ethyl 4-(benzo[d]isothiazole-3-carboxamido)-1-(1-((4-methoxybenzyl)amino)-1-oxopropan-2-yl)-1H-imidazole-2-carboxylate

A round bottom flask was charged with ethyl 4-amino-1-(1-((4-methoxybenzyl) amino)-1-oxopropan-2-yl)-1H-imidazole-2-carboxylate (6 g), benzo[d]isothiazole-3-carboxylic acid (5 g), HATU (10 g), DIEA (7 g) and DMF (100 mL). The solution was stirred at 25° C. for 1 hour. The reaction mixture was diluted with H₂O (200 mL), and the aqueous phase was extracted with ethyl acetate (150 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (5.2 g) as a white amorphous solid. LCMS: RT 1.15 min, [M+H]⁺=508.05, LCMS method A.

### Step 4. ethyl 1-(benzo[d]isothiazole-3-carboxamido)-8-(2-chloro-5-fluorophenyl)-5-methyl-6-oxo-5,6,7,8-tetrahydroimidazo[1,5-a]pyrazine-3-carboxylate

A round bottom flask was charged with ethyl 4-(benzo[d]isothiazole-3-carboxamido)-1-(1-((4-methoxybenzyl)amino)-1-oxopropan-2-yl)-1H-imidazole-2-carboxylate (5.2 g), 2-chloro-5-fluorobenzaldehyde (1.6 g) and Eaton's reagent (50 mL). The solution was stirred at 80° C. for 1 hour. The reaction mixture was poured into 200 mL of saturated NaHCO₃ solution at 0° C. The aqueous phase was extracted with ethyl acetate (100 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (2.4 g) as a white amorphous solid. LCMS: RT 1.12 min, [M+H]⁺ 528.00, LCMS method A.

### Step 5. N-(8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide

A round bottom flask was charged with ethyl 1-(benzo[d]isothiazole-3-carboxamido)-8-(2-chloro-5-fluorophenyl)-5-methyl-6-oxo-5,6,7,8-tetrahydroimidazo[1,5-a]pyrazine-3-carboxylate (2.4 g), methylamine (140 mg) in THF (10 mL), AlMe₃ (324 mg) and dichloromethane (20 mL) under a nitrogen atmosphere. The solution was stirred at 50° C. for 16 hours. The reaction mixture was diluted with H₂O (100 mL), and the aqueous phase was extracted with ethyl acetate (50 mL) three times. The combined organic layers were washed with brine, dried over sodium sulfate, filtered and concentrated in vacuo. The resulting crude material was purified by HPLC (mobile phase A: water, mobile phase B: acetonitrile) to give the desired product (1.3 g) as a white amorphous solid. LCMS: RT 1.03 min, [M+H]⁺ 512.95, LCMS method A.

### Step 6. N-((5S,8R)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide, N-((5R,8S)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide, N-((5S,8S)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide and N-((5R,8R)-8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide

N-(8-(2-chloro-5-fluorophenyl)-5-methyl-3-(methylcarbamoyl)-6-oxo-5,6,7,8-tetrahydroimidazo[1,5-a]pyrazin-1-yl)benzo[d]isothiazole-3-carboxamide (48 mg) was purified by chiral SFC (Column: ChiralPak IC—H 21×250 mm; mobile phase: 40% methanol in CO₂; flow rate: 70 mL/min; sample was dissolved in 2 mL methanol+2 mL dichloromethane; Injection: 0.75 mL; Detection: 254 nm) to give 4 peaks, all as a yellow amorphous solid after drying.

Compound I-162 or I-163, Peak 1: 11.3 mg. Chiral SFC RT 1.53 min. LCMS: RT 1.355 min, [M+H]⁺ 513.10, LCMS method M. ¹H NMR (400 MHz, DMSO-d6) δ 10.14 (s, 1H), 8.84 (s, 1H), 8.61 (d, J=8.2 Hz, 1H), 8.50 (q, J=4.7 Hz, 1H), 8.28 (dt, J=8.3, 1.0 Hz, 1H), 7.67 (ddd, J=8.2, 6.9, 1.3 Hz, 1H), 7.60 (ddd, J=8.1, 6.9, 1.1 Hz, 1H), 7.18 (dd, J=9.3, 3.1 Hz, 1H), 6.87 (td, J=8.4, 3.1 Hz, 1H), 6.31 (s, 1H), 5.47 (q, J=6.9 Hz, 1H), 2.78 (d, J=4.7 Hz, 3H), 1.67 (d, J=6.9 Hz, 3H).

Compound I-162 or I-163, Peak 2: 11 mg. Chiral SFC RT 1.82 min. LCMS: RT 1.355 min, [M+H]⁺ 513.10 LCMS method M. ¹H NMR (400 MHz, DMSO-d6) δ 10.14 (s, 1H), 8.84 (s, 1H), 8.61 (d, J=8.2 Hz, 1H), 8.50 (q, J=4.7 Hz, 1H), 8.28 (dt, J=8.3, 1.0 Hz, 1H), 7.67 (ddd, J=8.2, 6.9, 1.3 Hz, 1H), 7.60 (ddd, J=8.1, 6.9, 1.1 Hz, 1H), 7.18 (dd, J=9.3, 3.1 Hz, 1H), 6.87 (td, J=8.4, 3.1 Hz, 1H), 6.31 (s, 1H), 5.47 (q, J=6.9 Hz, 1H), 2.78 (d, J=4.7 Hz, 3H), 1.67 (d, J=6.9 Hz, 3H).

Compound I-164 or I-165, Peak 3: 8.4 mg. Chiral SFC RT 3.23 min. LCMS: RT 1.431 min, [M+H]⁺ 513.10, LCMS method M. ¹H NMR (400 MHz, DMSO-d6) δ 10.36 (br. s, 1H), 9.02 (d, J=3.2 Hz, 1H), 8.53 (d, J=3.2 Hz, 2H), 8.31 (d, J=8.4 Hz, 1H), 7.70-7.57 (m, 2H), 7.37 (dd, J=8.8, 5.2 Hz, 1H), 7.16-7.11 (m, 1H), 6.85 (dd, J=11.2, 9.6 Hz, 1H), 6.31 (d, J=9.6 Hz, 1H), 5.48 (dd, J=14.0, 6.8 Hz, 1H), 2.79 (d, J=4.8 Hz, 3H), 1.77 (d, J=6.8 Hz, 3H).

Compound I-164 or I-165, Peak 4: 8.7 mg. Chiral SFC RT 3.65 min. LCMS: RT 1.430 min, [M+H]⁺ 513.10, LCMS method M. ¹H NMR (400 MHz, DMSO-d6) δ 10.36 (br. s, 1H), 9.02 (d, J=3.2 Hz, 1H), 8.53 (d, J=3.2 Hz, 2H), 8.31 (d, J=8.4 Hz, 1H), 7.70-7.57 (m, 2H), 7.37 (dd, J=8.8, 5.2 Hz, 1H), 7.16-7.11 (m, 1H), 6.85 (dd, J=11.2, 9.6 Hz, 1H), 6.31 (d, J=9.6 Hz, 1H), 5.48 (dd, J=14.0, 6.8 Hz, 1H), 2.79 (d, J=4.8 Hz, 3H), 1.77 (d, J=6.8 Hz, 3H).

Additional compounds prepared according to the methods of Example 32 are listed in Table 10 below. Corresponding ¹H NMR and mass spectrometry characterization for these compounds are described in Table 1. Certain compounds in Table 10 below were prepared with other compounds whose preparation is described in the Examples herein.

TABLE 10
Additional Exemplary Compounds
Compound

I-7
I-8
I-113
I-146
I-147
I-148
I-149
I-154
I-155
I-156
I-157
I-649
I-650
I-664
I-665

### Example 34

### (±)—(S)-8-(2-chloro-5-fluorophenyl)-1-((S)-5-fluoro-3-hydroxy-3-(trifluoromethyl)indoline-1-carboxamido)-N-methyl-6-oxo-5,6,7,8-tetrahydroimidazo[1,5-a]pyrazine-3-carboxamide (I-241) and (±)—(R)-8-(2-chloro-5-fluorophenyl)-1-((S)-5-fluoro-3-hydroxy-3-(trifluoromethyl)indoline-1-carboxamido)-N-methyl-6-oxo-5,6,7,8-tetrahydroimidazo[1,5-a]pyrazine-3-carboxamide (I-242)

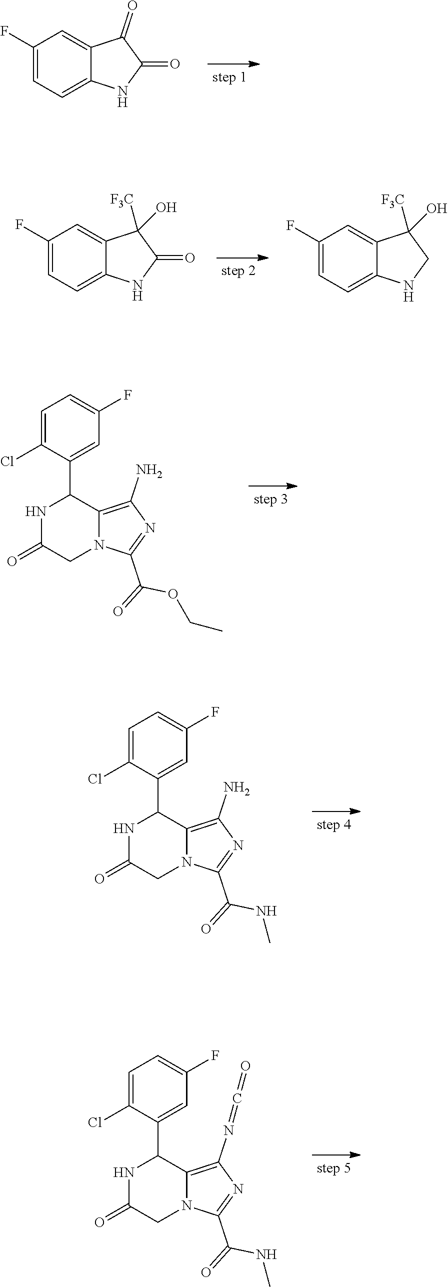 [SMILES: O=C1Nc2ccc(F)cc2C1=O ; O=C1Nc2ccc(F)cc2C1(O)C(F)(F)F ; OC1(C(F)(F)F)CNc2ccc(F)cc21 ; CCOC(=O)c1nc(N)c2n1CC(=O)NC2c1cc(F)ccc1Cl ; CNC(=O)c1nc(N)c2n1CC(=O)NC2c1cc(F)ccc1Cl ; CNC(=O)c1nc(N=C=O)c2n1CC(=O)NC2c1cc(F)ccc1Cl]

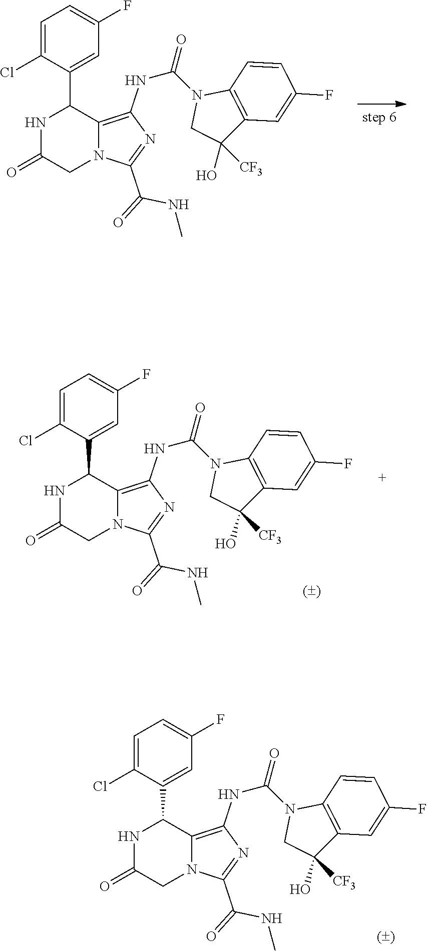 [SMILES: CNC(=O)c1nc(NC(=O)N2CC(O)(C(F)(F)F)c3cc(F)ccc32)c2n1CC(=O)NC2c1cc(F)ccc1Cl ; CNC(=O)c1nc(NC(=O)N2C[C@](O)(C(F)(F)F)c3cc(F)ccc32)c2n1CC(=O)N[C@H]2c1cc(F)ccc1Cl ; CNC(=O)c1nc(NC(=O)N2C[C@](O)(C(F)(F)F)c3cc(F)ccc32)c2n1CC(=O)N[C@@H]2c1cc(F)ccc1Cl]

### Step 1. 5-fluoro-3-hydroxy-3-(trifluoromethyl)indolin-2-one

To a solution of 5-fluoroindoline-2,3-dione (100 g) and TMSCF₃ (258 g) in THF (2.00 L) was added t-BuOK (1.0 M in THF, 1.82 L) at −65° C. under N₂. The mixture was stirred at 20° C. for 2 hours under N₂. The reaction mixture was poured into H₂O (5.0 L). The aqueous phase was extracted with ethyl acetate (3.0 L X 5). The combined organic phase was washed with brine (5.00 L X 3), dried over anhydrous Na₂SO₄, filtered and concentrated in vacuo to give the crude product. The residue was triturated with petroleum ether:ethyl acetate 30:1 (300 mL) at 20° C. for 16 hours. The solid was collected by filtration and dried to give the crude product (255 g) as a yellow solid, which was purified by silica gel chromatography (petroleum ether:ethyl acetate 20: 1 to 1: 1) to give the desired product (130 g) as a yellow solid. LCMS: RT 0.669 min, [M+H]⁺ 236.1, LCMS method X. ¹H NMR (400 MHz, DMSO-d6) δ 10.85 (s, 1H), 7.77 (s, 1H), 7.26-7.21 (m, 2H), 6.93-6.90 (m, 1H).

### Step 2. 5-fluoro-3-(trifluoromethyl)indolin-3-ol

To a solution of lithium aluminum hydride (56.6 g) in THF (800 mL) was added a solution of 5-fluoro-3-hydroxy-3-(trifluoromethyl)indolin-2-one (108 g) in THF (200 mL) at 20-25° C. The mixture was stirred at 25° C. for 2 hours. The reaction was quenched with H₂O (56.6 mL), followed by NaOH (15% by weight, 56.6 mL) and H₂O (170 mL). The mixture was filtered and the filtrate was concentrated to give a residue. The residue was purified by column chromatography (SiO₂, petroleum ether:ethyl acetate 10:1 to 1:1) to give the desired product (83.0 g) as an off-white solid. LCMS Rt=0.369 min, [M+H]⁺ not observed, LCMS method X. ¹H NMR (400 MHz, CDCl₃) δ 7.07-7.05 (m, 1H), 6.95-6.92 (m, 1H), 6.68-6.65 (m, 1H), 5.95 (t, J=55.6 Hz, 1H), 3.84 (d, J=11.6 Hz, 1H), 3.77 (s, 1H), 3.57 (d, J=11.2 Hz, 1H), 2.71 (s, 1H).

### Step 3. 1-amino-8-(2-chloro-5-fluorophenyl)-N-methyl-6-oxo-5,6,7,8-tetrahydroimidazo[1,5-a]pyrazine-3-carboxamide

In a round bottom flask, to a solution of ethyl 1-amino-8-(2-chloro-5-fluorophenyl)-6-oxo-5,6,7,8-tetrahydroimidazo[1,5-a]pyrazine-3-carboxylate (680 mg) in MeOH (5 mL) and THF (10 mL) was added MeNH₂ (40% weight in H₂O, 18.4 mL). The reaction mixture was heated at 50° C. for 2 hours. Volatiles were removed under reduced pressure and the crude residue was diluted with 2-methyl THF and water (30 mL each). Phases were separated and the aqueous phase was extracted with 2-methyl THF (2×30 mL). The combined organic layers were dried over anhydrous Na₂SO₄ and concentrated in vacuo. The crude residue was purified on a reverse phase C18 column (30 g, eluent: 10 mM ammonium formate solution (A) and acetonitrile (B); gradient: 0-100% B) to afford the desired product (145 mg) as an off-white solid. LC-MS RT 0.54 min, [M+H]⁺ 338.2 (LCMS method U).

### Step 4. 8-(2-chloro-5-fluorophenyl)-1-isocyanato-N-methyl-6-oxo-5,6,7,8-tetrahydroimidazo[1,5-a]pyrazine-3-carboxamide

In a flame-dried microwave vial, to a solution of 1-amino-8-(2-chloro-5-fluorophenyl)-N-methyl-6-oxo-5,6,7,8-tetrahydroimidazo[1,5-a]pyrazine-3-carboxamide (50 mg) and DIPEA (26 μL) in THF (1.0 mL) was added a solution of triphosgene (22 mg) in THF (0.5 mL) at 0° C. The resulting solution was stirred at room temperature for 1 hour. The mixture was used directly as 0.1 M solution of the desired product in the subsequent step. Isocyanate formation was confirmed by quenching the solution above with MeOH to show the presence of the corresponding methylcarbamate. LC-MS: RT 0.59 min, [M+H]⁺ 396.2, LCMS method U.

### Step 5. 8-(2-chloro-5-fluorophenyl)-1-(5-fluoro-3-hydroxy-3-(trifluoromethyl)indoline-1-carboxamido)-N-methyl-6-oxo-5,6,7,8-tetrahydroimidazo[1,5-a]pyrazine-3-carboxamide

In a flame-dried microwave vial, to a solution of 5-fluoro-3-(trifluoromethyl)indolin-3-ol (30 mg), DMAP (0.17 mg) and pyridine (44 μL) in THF (0.7 mL) at 0° C. was gradually added a 0.1 M THF solution of 8-(2-chloro-5-fluorophenyl)-1-isocyanato-N-methyl-6-oxo-5,6,7,8-tetrahydroimidazo[1,5-a]pyrazine-3-carboxamide (1.4 mL). The resulting solution was warmed to room temperature and stirred for 1 hour. The reaction mixture was quenched with saturated aqueous solution of NH₄Cl (5 mL). Water was added to dissolve the precipitated inorganic salts, and the solution was extracted with ethyl acetate (3×15 mL). The combined organic layers were dried over anhydrous Na₂SO₄ and concentrated in vacuo. The crude residue was taken up in DMF and purified on a C18 reverse phase column (30 g, eluent A: 10 mM ammonium formate solution, eluent B: acetonitrile; gradient 0-100% B) to afford the desired product (18 mg) as an off-white solid. LC-MS RT 1.84 and 1.86 min, [M+H]⁺ 585.3, LCMS method V. ¹H NMR (400 MHz, DMSO-d₆) δ 8.92 (d, J=1.6 Hz, 0.6H), 8.90 (d, J=0.4 Hz, 0.4H), 8.54 (br. s, 1H), 8.39-8.31 (2 overlapping quartets; J=4.7 Hz, 1H), 7.93 (dd, J=9.0, 4.7 Hz, 0.4H), 7.83 (dd, J=8.9, 4.7 Hz, 0.6H), 7.42 (br. s, 1H), 7.39-7.31 (m, 1H), 7.29-7.23 (m, 1H), 7.23-7.16 (m, 1.6H), 7.17-7.07 (m, 1.4H), 6.08 (br. s, 0.6H), 6.03 (br. s, 0.4H), 5.24 (d, J=18.7 Hz, 0.6H), 5.23 (d, J=18.7 Hz, 0.4H), 5.05 (d, J=18.8 Hz, 1H), 4.24 (d, J=12.2 Hz, 0.6H), 3.88 (d, J=12.3 Hz, 0.4H), 3.79 (d, J=12.2 Hz, 0.6H), 3.59 (d, J=12.3 Hz, 0.4H), 2.75 (d, J=4.7 Hz, 1.3H), 2.74 (d, J=4.7 Hz, 1.7H); ˜1.3:1 mixture of two diastereomers; partial ammonium formate salt (20%) at 8.29 ppm.

### Step 6. (±)—(S)-8-(2-chloro-5-fluorophenyl)-1-((S)-5-fluoro-3-hydroxy-3-(trifluoromethyl)indoline-1-carboxamido)-N-methyl-6-oxo-5,6,7,8-tetrahydroimidazo[1,5-a]pyrazine-3-carboxamide and (±)—(R)-8-(2-chloro-5-fluorophenyl)-1-((S)-5-fluoro-3-hydroxy-3-(trifluoromethyl)indoline-1-carboxamido)-N-methyl-6-oxo-5,6,7,8-tetrahydroimidazo[1,5-a]pyrazine-3-carboxamide

8-(2-chloro-5-fluorophenyl)-1-(5-fluoro-3-hydroxy-3-(trifluoromethyl)indoline-1-carboxamido)-N-methyl-6-oxo-5,6,7,8-tetrahydroimidazo[1,5-a]pyrazine-3-carboxamide (13.2 mg) was partially resolved by chiral SFC (Column: Regis Whelk 0-1 (S,S) 21×250 mm; mobile phase: 30% ethanol in CO₂; flow Rate: 70 mL/min; sample: 13.2 mg of sample dissolved in 2.0 mL methanol+2.0 mL dichloromethane; Injection: 2.0 mL; detection wavelength: 254 nm) to give both products as an off-white solid.

Compound I-242, Peak 1, 4.0 mg. Chiral SFC RT 2.28 min. LCMS: RT 0.992 min, [M+H]⁺ 585.1, LCMS method G.

Compound I-241, Peak 2, 3.8 mg. Chiral SFC RT 2.79 min. LCMS: RT 0.991 min, [M+H]⁺ 585.0, LCMS method G.

### Example 35

### (R)-8-(2-chloro-5-fluorophenyl)-1-(5,6-difluoroindoline-1-carboxamido)-N-methyl-6-oxo-5,6,7,8-tetrahydroimidazo[1,5-a]pyrazine-3-carboxamide (I-353 or I-354) and (S)-8-(2-chloro-5-fluorophenyl)-1-(5,6-difluoroindoline-1-carboxamido)-N-methyl-6-oxo-5,6,7,8-tetrahydroimidazo[1,5-a]pyrazine-3-carboxamide (I-353 or I-354)

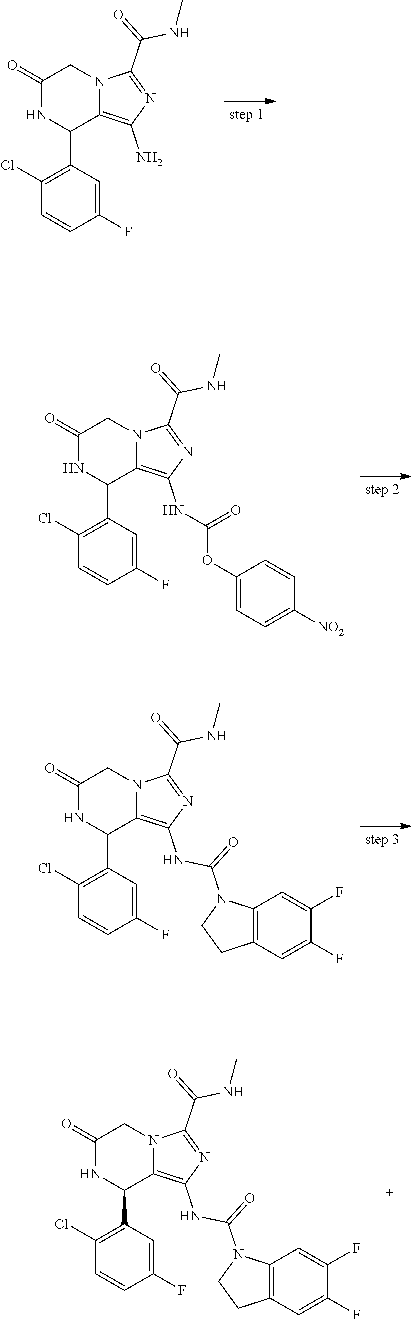 [SMILES: CNC(=O)c1nc(N)c2n1CC(=O)NC2c1cc(F)ccc1Cl ; CNC(=O)c1nc(NC(=O)Oc2ccc([N+](=O)[O-])cc2)c2n1CC(=O)NC2c1cc(F)ccc1Cl ; CNC(=O)c1nc(NC(=O)N2CCc3cc(F)c(F)cc32)c2n1CC(=O)NC2c1cc(F)ccc1Cl ; CNC(=O)c1nc(NC(=O)N2CCc3cc(F)c(F)cc32)c2n1CC(=O)N[C@@H]2c1cc(F)ccc1Cl]

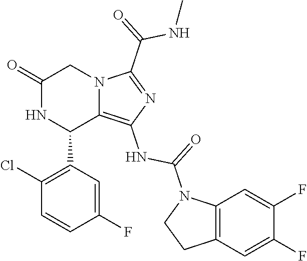 [SMILES: CNC(=O)c1nc(NC(=O)N2CCc3cc(F)c(F)cc32)c2n1CC(=O)N[C@H]2c1cc(F)ccc1Cl]

### Step 1. 4-nitrophenyl (8-(2-chloro-5-fluorophenyl)-3-(methylcarbamoyl)-6-oxo-5,6,7,8-tetrahydroimidazo[1,5-a]pyrazin-1-yl)carbamate

A reaction vial was charged with 1-amino-8-(2-chloro-5-fluorophenyl)-N-methyl-6-oxo-5,6,7,8-tetrahydroimidazo[1,5-a]pyrazine-3-carboxamide (70 mg), 4-nitrophenyl carbonochloridate (63 mg) and a stir bar before being evacuated and purged with nitrogen three times. THF (10 mL) was added, and the reaction mixture was stirred at 70° C. for 1 hour. The resulting crude material was used in the next step directly without purification. LCMS: RT 0.96 min, [M+H]⁺ 503.2, LCMS method A.

### Step 2. 8-(2-chloro-5-fluorophenyl)-1-(5,6-difluoroindoline-1-carboxamido)-N-methyl-6-oxo-5,6,7,8-tetrahydroimidazo[1,5-a]pyrazine-3-carboxamide

A mixture of 5,6-difluoroindoline (48.6 mg) and triethylamine (106 mg) in THF (10 mL) was stirred at 70° C. for 1 hour. It was then stirred at 25° C. for 2 minutes before the addition of a THF solution of 4-nitrophenyl (8-(2-chloro-5-fluorophenyl)-3-(methylcarbamoyl)-6-oxo-5,6,7,8-tetrahydroimidazo[1,5-a]pyrazin-1-yl)carbamate from the previous step. The mixture was stirred at 70° C. for 1 hour under nitrogen. The reaction mixture was diluted with water (15 mL), and the aqueous phase was extracted with ethyl acetate (15 mL×3). The combined organic layers were washed with brine, dried over sodium sulfate, filtered and concentrated in vacuo. The resulting crude material was purified by HPLC (mobile phase A: water, mobile phase B: acetonitrile) to give the desired product (55 mg) as an off-white amorphous solid. LCMS: RT 1.00 min, [M+H]⁺ 519.2, LCMS method A.

### Step 3. (R)-8-(2-chloro-5-fluorophenyl)-1-(5,6-difluoroindoline-1-carboxamido)-N-methyl-6-oxo-5,6,7,8-tetrahydroimidazo[1,5-a]pyrazine-3-carboxamide and (S)-8-(2-chloro-5-fluorophenyl)-1-(5,6-difluoroindoline-1-carboxamido)-N-methyl-6-oxo-5,6,7,8-tetrahydroimidazo[1,5-a]pyrazine-3-carboxamide

8-(2-chloro-5-fluorophenyl)-1-(5,6-difluoroindoline-1-carboxamido)-N-methyl-6-oxo-5,6,7,8-tetrahydroimidazo[1,5-a]pyrazine-3-carboxamide (55 mg) was chirally resolved by chiral-HPLC (Column: CHIRALPAK IH-3, 4.6*50 mm, 5 μm; mobile phase A: hexane (0.2% diethylamine), mobile phase B: EtOH:dichloromethane 1:1; flow rate: 1 mL/min; gradient: 60% B isocratic; wavelength: 220/254 nm; sample dissolved in EtOH: dichloromethane 1:1; injection volume: 2.25 mL) to give both enantiomers as an off-white amorphous solid.

Compound I-354, Peak 1: 22 mg. Chiral SFC RT 3.20 min. LCMS: RT 1.29 min, [M+H]⁺ 519.11, LCMS method M. ¹H NMR (400 MHz, DMSO-d₆) δ 8.89 (d, J=2.2 Hz, 1H), 8.42 (s, 1H), 8.33 (q, J=4.7 Hz, 1H), 7.69 (dd, J=12.6, 7.5 Hz, 1H), 7.43-7.35 (m, 1H), 7.27 (dd, J=10.2, 8.3 Hz, 1H), 7.15 (t, J=8.3 Hz, 2H), 6.03 (s, 1H), 5.21 (s, 1H), 5.05 (dd, J=18.8, 1.6 Hz, 1H), 3.92-3.81 (m, 1H), 3.44 (td, J=10.2, 7.1 Hz, 1H), 3.14-3.03 (m, 1H), 3.03 (s, 1H), 2.76 (d, J=4.7 Hz, 3H).

Compound I-353, Peak 2: 19.8 mg. Chiral SFC RT 10.99 min. LCMS: RT 1.29 min, [M+H]⁺ 519.11, LCMS method M. ¹H NMR (400 MHz, DMSO-d₆) δ 8.89 (d, J=2.2 Hz, 1H), 8.42 (s, 1H), 8.33 (q, J=4.7 Hz, 1H), 7.69 (dd, J=12.6, 7.5 Hz, 1H), 7.43-7.35 (m, 1H), 7.31-7.22 (m, 1H), 7.20-7.09 (m, 2H), 6.06-6.01 (m, 1H), 5.23 (dd, J=18.8, 1.1 Hz, 1H), 5.05 (dd, J=18.8, 1.6 Hz, 1H), 3.87 (td, J=10.1, 7.0 Hz, 1H), 3.44 (td, J=10.3, 7.1 Hz, 1H), 3.14-3.03 (m, 1H), 3.02 (d, J=8.7 Hz, 1H), 2.76 (d, J=4.7 Hz, 3H).

Additional compounds prepared according to the methods of Example 35 are listed in Table 11 below. Corresponding ¹H NMR and mass spectrometry characterization for these compounds are described in Table 1. Certain compounds in Table 11 below were prepared with other compounds whose preparation is described in the Examples herein.

TABLE 11
Additional Exemplary Compounds
Compound

I-591
I-592
I-593
I-594
I-595
I-596
I-597
I-672
I-673

### Example 36

### (S)-8-(2-chloro-5-fluorophenyl)-1-(5-fluoro-1H-indole-1-carboxamido)-N-methyl-6-oxo-5,6,7,8-tetrahydroimidazo[1,5-a]pyrazine-3-carboxamide (I-531) and (R)-8-(2-chloro-5-fluorophenyl)-1-(5-fluoro-1H-indole-1-carboxamido)-N-methyl-6-oxo-5,6,7,8-tetrahydroimidazo[1,5-a]pyrazine-3-carboxamide (I-575)

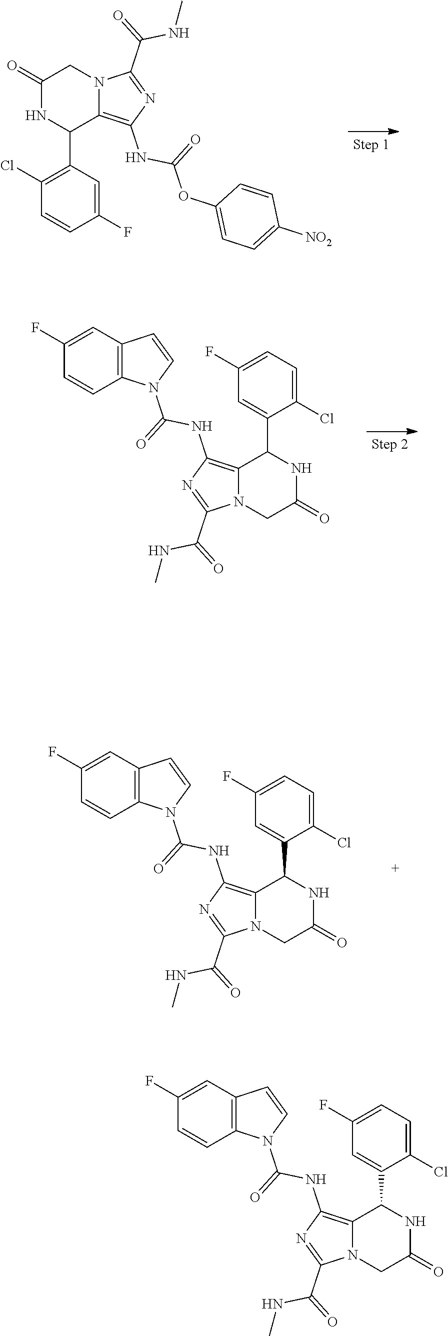 [SMILES: CNC(=O)c1nc(NC(=O)Oc2ccc([N+](=O)[O-])cc2)c2n1CC(=O)NC2c1cc(F)ccc1Cl ; CNC(=O)c1nc(NC(=O)n2ccc3cc(F)ccc32)c2n1CC(=O)NC2c1cc(F)ccc1Cl ; CNC(=O)c1nc(NC(=O)n2ccc3cc(F)ccc32)c2n1CC(=O)N[C@@H]2c1cc(F)ccc1Cl ; CNC(=O)c1nc(NC(=O)n2ccc3cc(F)ccc32)c2n1CC(=O)N[C@H]2c1cc(F)ccc1Cl]

### Step 1. 8-(2-chloro-5-fluorophenyl)-1-(5-fluoro-1H-indole-1-carboxamido)-N-methyl-6-oxo-5,6,7,8-tetrahydroimidazo[1,5-a]pyrazine-3-carboxamide

A mixture of 5-fluoro-1H-indole (60.1 mg) and LiHMDS (14.6 mg) in THF (6 mL) was stirred at 70° C. for 1 hour. The mixture was stirred for 2 min at 25° C. prior to the addition of a 10 mL THF solution of 4-nitrophenyl (8-(2-chloro-5-fluorophenyl)-3-(methylcarbamoyl)-6-oxo-5,6,7,8-tetrahydroimidazo[1,5-a]pyrazin-1-yl)carbamate (298 mg). The mixture was stirred at 70° C. for 1 hour under nitrogen, cooled to room temperature and quenched with water. The reaction mixture was diluted with water (15 mL), and the aqueous phase was extracted with ethyl acetate (15 mL×3). The combined organic layers were washed with brine, dried over sodium sulfate, filtered and concentrated in vacuo. The resulting crude material was purified by HPLC (mobile phase A: water, mobile phase B: acetonitrile) to give the desired product (26 mg) as an off-white amorphous solid. LCMS: RT 1.01 min, [M+H]⁺ 499.3, LCMS method A.

### Step 2. (R)-8-(2-chloro-5-fluorophenyl)-1-(5-fluoro-1H-indole-1-carboxamido)-N-methyl-6-oxo-5,6,7,8-tetrahydroimidazo[1,5-a]pyrazine-3-carboxamide and (S)-8-(2-chloro-5-fluorophenyl)-1-(5-fluoro-1H-indole-1-carboxamido)-N-methyl-6-oxo-5,6,7,8-tetrahydroimidazo[1,5-a]pyrazine-3-carboxamide

8-(2-chloro-5-fluorophenyl)-1-(5-fluoro-1H-indole-1-carboxamido)-N-methyl-6-oxo-5,6,7,8-tetrahydroimidazo[1,5-a]pyrazine-3-carboxamide (23 mg) was chirally resolved by CHIRAL-HPLC (Column: CHIRALPAK IH, 2*25 cm, 5 μm; mobile phase A: hexane (0.2% triethylamine), mobile phase B: EtOH:dichloromethane 1:1; flow rate: 20 mL/min; Gradient: 65% B isocratic; wavelength: 220/254 nm; peak 1 RT 3.35 min, peak 2 RT 7.65 min; sample dissolved in EtOH:dichloromethane 1:1; injection volume: 1.4 mL) to give both enantiomers as an off-white amorphous solid.

Compound I-575, Peak 1: 6 mg. LCMS: RT 2.16 min, [M+H]⁺ 499.20, LCMS method F. ¹H NMR (400 MHz, DMSO-d₆) δ 9.76 (s, 1H), 8.95 (s, 1H), 8.45 (d, J=5.0 Hz, 1H), 8.14 (dd, J=9.1, 4.9 Hz, 1H), 7.68 (d, J=3.7 Hz, 1H), 7.39 (dd, J=9.3, 2.6 Hz, 1H), 7.29 (dd, J=8.8, 5.0 Hz, 1H), 7.23-7.01 (m, 3H), 6.64 (d, J=3.7 Hz, 1H), 6.11 (s, 1H), 5.25 (s, 1H), 5.11 (s, 1H), 2.80-2.71 (m, 3H).

Compound I-531, Peak 2: 4 mg. LCMS: RT 1.33 min, [M+H]⁺=499.15, LCMS method M. ¹H NMR (400 MHz, DMSO-d₆) δ 9.76 (s, 1H), 8.95 (d, J=2.2 Hz, 1H), 8.45 (d, J=5.1 Hz, 1H), 8.14 (dd, J=9.0, 4.8 Hz, 1H), 7.68 (d, J=3.7 Hz, 1H), 7.39 (dd, J=9.2, 2.7 Hz, 1H), 7.29 (dd, J=8.8, 5.0 Hz, 1H), 7.19 (dd, J=9.2, 3.0 Hz, 1H), 7.17-7.08 (m, 1H), 7.06 (td, J=8.4, 3.2 Hz, 1H), 6.64 (d, J=3.7 Hz, 1H), 6.11 (s, 1H), 5.30 (s, 1H), 5.06 (s, 1H), 2.77 (d, J=4.7 Hz, 3H).

### Example 37

### (S)-8-(2-chloro-5-fluorophenyl)-N-methyl-6-oxo-1-(3,3,5,6-tetrafluoroindoline-1-carboxamido)-5,6,7,8-tetrahydroimidazo[1,5-a]pyrazine-3-carboxamide (I-619)

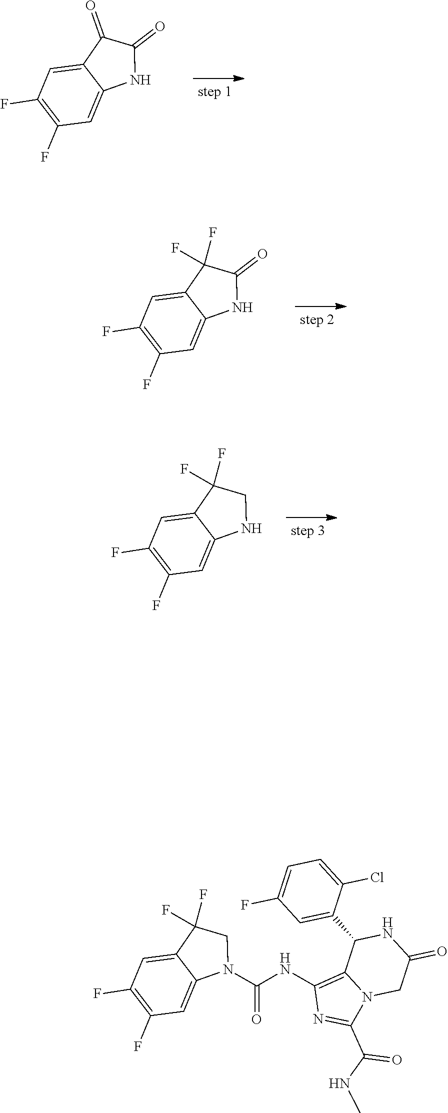 [SMILES: O=C1Nc2cc(F)c(F)cc2C1=O ; O=C1Nc2cc(F)c(F)cc2C1(F)F ; Fc1cc2c(cc1F)C(F)(F)CN2 ; CNC(=O)c1nc(NC(=O)N2CC(F)(F)c3cc(F)c(F)cc32)c2n1CC(=O)N[C@H]2c1cc(F)ccc1Cl]

### Step 1. 3,3,5,6-tetrafluoroindolin-2-one

A round bottom flask was charged with 5,6-difluoroindoline-2,3-dione (500 mg) and dichloromethane (6 mL). DAST (1.74 g) was added and the solution was stirred at 25° C. for 1 hour. The reaction mixture was diluted with H₂O (20 mL) and the aqueous phase was extracted with ethyl acetate (15 mL) three times. The combined organic layers were washed with brine, dried over sodium sulfate, filtered and concentrated in vacuo. The resulting crude material was purified by HPLC (mobile phase A: water with 0.1% formic acid, mobile phase B: acetonitrile with 0.1% formic acid) to give the desired product (400 mg) as a white amorphous solid. LCMS: RT 0.64 min, [M+H]⁺ 206.15, LCMS method P.

### Step 2. 3,3,5,6-tetrafluoroindoline

A solution of 3,3,5,6-tetrafluoroindolin-2-one (200 mg) in THF (100 mL) was stirred at 0° C. BH₃ in THF (1 mL) was added dropwise. The ice-water bath was removed after the addition was completed and the mixture was stirred at room temperature for 1 hour. The reaction was quenched with 10% citric acid solution (50 mL) at 0° C. Water (20 mL) was added and extracted with ethyl acetate (20 mL×3). The organic phase was washed with brine (20 mL×3), dried over anhydrous Na₂SO₄, filtered and concentrated in vacuo until about 30 mL remained. The solution containing the desired product was used immediately in the next step without further purification.

### Step 3. (S)-8-(2-chloro-5-fluorophenyl)-N-methyl-6-oxo-1-(3,3,5,6-tetrafluoroindoline-1-carboxamido)-5,6,7,8-tetrahydroimidazo[1,5-a]pyrazine-3-carboxamide

A round bottom flask was charged with 4-nitrophenyl (S)-(8-(2-chloro-5-fluorophenyl)-3-(methylcarbamoyl)-6-oxo-5,6,7,8-tetrahydroimidazo[1,5-a]pyrazin-1-yl)carbamate (50 mg), an ethyl acetate solution of 3,3,5,6-tetrafluoroindoline (19 mg), triethylamine (30 mg) and THF (1 mL). The solution was stirred at 70° C. for 1 hour. The reaction mixture was diluted with H₂O (20 mL), and the aqueous phase was extracted with ethyl acetate (15 mL) three times. The combined organic layers were washed with brine, dried over sodium sulfate, filtered and concentrated in vacuo. The resulting crude material was purified by prep-HPLC (Column: XBridge Prep OBD C18 Column, 30*150 mm, 5 μm; mobile phase A: 10 mM NH₄HCO₃ solution, mobile phase B: acetonitrile; flow rate: 60 mL/min; gradient: 35% B to 47% B in 8 min, then 47% B; wavelength: 220 nm; RT: 7.32 min) to give the desired product (10 mg) as a white amorphous solid. LCMS: RT 1.403 min, [M+H]⁺ 555.20, LC Method M. ¹H NMR (400 MHz, DMSO-d6) δ 8.90 (d, J=2.0 Hz, 1H), 8.76 (s, 1H), 8.33 (q, J=4.7 Hz, 1H), 7.98-7.84 (m, 2H), 7.36 (dd, J=8.8, 5.1 Hz, 1H), 7.20 (dd, J=9.3, 3.1 Hz, 1H), 7.12 (td, J=8.4, 3.1 Hz, 1H), 5.99 (s, 1H), 5.23 (d, J=18.7 Hz, 1H), 5.04 (dd, J=18.7, 1.7 Hz, 1H), 4.30 (dt, J=20.1, 13.9 Hz, 1H), 3.78 (dt, J=20.4, 13.3 Hz, 1H), 2.76 (d, J=4.7 Hz, 3H).

Compounds I-681, I-683, and I-685 were also prepared according to the methods of Example 37. Mass spectrometry and ¹H NMR characterization are provided for these compounds in Table 1.

### Example 38

### (S)-8-(2-chloro-5-fluorophenyl)-1-((S)-3-hydroxy-2,3-dihydro-1H-pyrrolo[3,2-b]pyridine-1-carboxamido)-N-methyl-6-oxo-5,6,7,8-tetrahydroimidazo[1,5-a]pyrazine-3-carboxamide (I-623 or I-624) and (S)-8-(2-chloro-5-fluorophenyl)-1-((R)-3-hydroxy-2,3-dihydro-1H-pyrrolo[3,2-b]pyridine-1-carboxamido)-N-methyl-6-oxo-5,6,7,8-tetrahydroimidazo[1,5-a]pyrazine-3-carboxamide (I-623 or I-624)

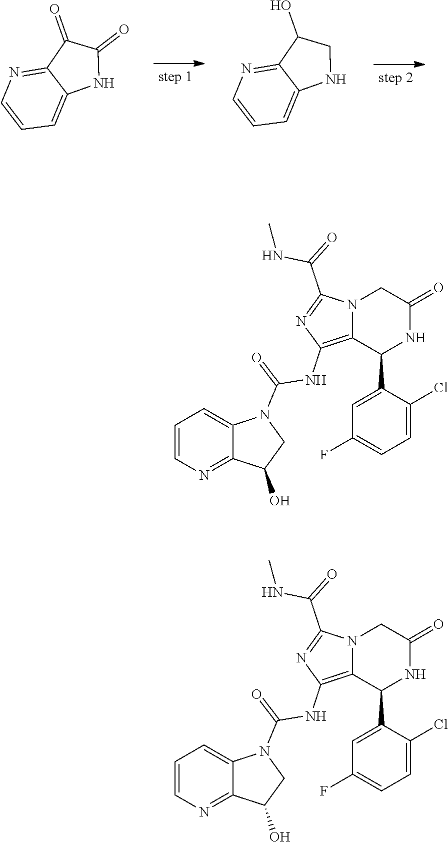 [SMILES: O=C1Nc2cccnc2C1=O ; OC1CNc2cccnc21 ; CNC(=O)c1nc(NC(=O)N2C[C@@H](O)c3ncccc32)c2n1CC(=O)N[C@H]2c1cc(F)ccc1Cl ; CNC(=O)c1nc(NC(=O)N2C[C@H](O)c3ncccc32)c2n1CC(=O)N[C@H]2c1cc(F)ccc1Cl]

### Step 1. 2,3-dihydro-1H-pyrrolo[3,2-b]pyridin-3-ol

A round bottomed flask was charged 1H-pyrrolo[3,2-b]pyridine-2,3-dione (200 mg) and THF (4 mL) at 0° C. LiAlH₄ (0.26 g) was added slowly. The solution was stirred at 25° C. for 1 hour. The reaction was then quenched with water. The reaction mixture was filtered and concentrated in vacuo. The resulting crude material was purified by HPLC (water/ACN). Lyophilization yielded the desired product as a yellow amorphous solid (80 mg). LCMS: RT 0.35 min, [M+H]⁺ 137.20, LCMS method O.

### Step 2. (S)-8-(2-chloro-5-fluorophenyl)-1-((S)-3-hydroxy-2,3-dihydro-1H-pyrrolo[3,2-b]pyridine-1-carboxamido)-N-methyl-6-oxo-5,6,7,8-tetrahydroimidazo[1,5-a]pyrazine-3-carboxamide and (S)-8-(2-chloro-5-fluorophenyl)-1-((R)-3-hydroxy-2,3-dihydro-1H-pyrrolo[3,2-b]pyridine-1-carboxamido)-N-methyl-6-oxo-5,6,7,8-tetrahydroimidazo[1,5-a]pyrazine-3-carboxamide

By analogy to the method of Step 3 of Example 37, 2,3-dihydro-1H-pyrrolo[3,2-b]pyridin-3-ol and 4-nitrophenyl (S)-(8-(2-chloro-5-fluorophenyl)-3-(methylcarbamoyl)-6-oxo-5,6,7,8-tetrahydroimidazo[1,5-a]pyrazin-1-yl)carbamate were condensed to afford S)-8-(2-chloro-5-fluorophenyl)-1-((S)-3-hydroxy-2,3-dihydro-1H-pyrrolo[3,2-b]pyridine-1-carboxamido)-N-methyl-6-oxo-5,6,7,8-tetrahydroimidazo[1,5-a]pyrazine-3-carboxamide and (S)-8-(2-chloro-5-fluorophenyl)-1-((R)-3-hydroxy-2,3-dihydro-1H-pyrrolo[3,2-b]pyridine-1-carboxamido)-N-methyl-6-oxo-5,6,7,8-tetrahydroimidazo[1,5-a]pyrazine-3-carboxamide.

Compound I-624: [M+H]⁺ 500.05. ¹H NMR (400 MHz, DMSO-d6) δ 8.92 (d, J=2.4 Hz, 1H), 8.49 (s, 1H), 8.36 (q, J=4.7 Hz, 1H), 8.12 (dd, J=4.8, 1.4 Hz, 1H), 8.00 (dd, J=8.2, 1.4 Hz, 1H), 7.36 (dd, J=8.8, 5.1 Hz, 1H), 7.19 (ddd, J=15.3, 8.7, 3.9 Hz, 2H), 7.09 (td, J=8.4, 3.1 Hz, 1H), 6.14-6.09 (m, 1H), 5.88 (d, J=5.4 Hz, 1H), 5.23 (s, 1H), 5.15-5.02 (m, 2H), 3.97 (dd, J=11.3, 8.1 Hz, 1H), 3.41 (dd, J=11.3, 3.6 Hz, 1H), 2.76 (d, J=4.7 Hz, 3H).

Compound I-623: [M+H]⁺ 500.05. ¹H NMR (400 MHz, DMSO-d6) δ 8.92 (d, J=2.4 Hz, 1H), 8.49 (s, 1H), 8.36 (q, J=4.7 Hz, 1H), 8.12 (dd, J=4.8, 1.4 Hz, 1H), 8.00 (dd, J=8.2, 1.4 Hz, 1H), 7.36 (dd, J=8.8, 5.1 Hz, 1H), 7.19 (ddd, J=15.3, 8.7, 3.9 Hz, 2H), 7.09 (td, J=8.4, 3.1 Hz, 1H), 6.14-6.09 (m, 1H), 5.88 (d, J=5.4 Hz, 1H), 5.23 (s, 1H), 5.15-5.02 (m, 2H), 3.97 (dd, J=11.3, 8.1 Hz, 1H), 3.41 (dd, J=11.3, 3.6 Hz, 1H), 2.76 (d, J=4.7 Hz, 3H).

### Example 39

### (S)-8-(2-chloro-5-fluorophenyl)-1-((R)-5,6-difluoro-3-(trifluoromethyl)indoline-1-carboxamido)-N-methyl-6-oxo-5,6,7,8-tetrahydroimidazo[1,5-a]pyrazine-3-carboxamide (I-625 or I-626) and (S)-8-(2-chloro-5-fluorophenyl)-1-((S)-5,6-difluoro-3-(trifluoromethyl)indoline-1-carboxamido)-N-methyl-6-oxo-5,6,7,8-tetrahydroimidazo[1,5-a]pyrazine-3-carboxamide (I-625 or I-626)

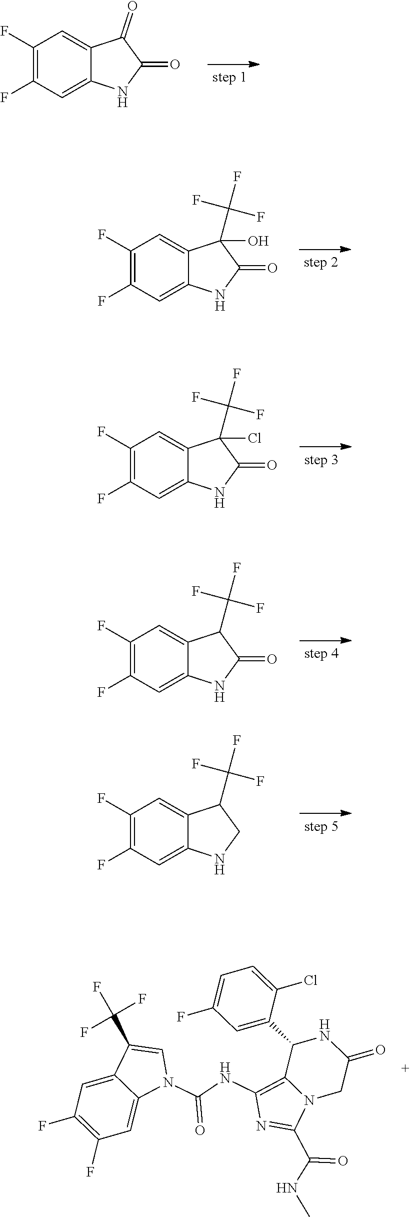

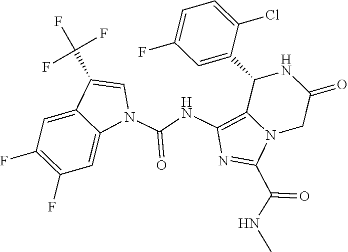

### Step 1. 5,6-difluoro-3-hydroxy-3-(trifluoromethyl)indolin-2-one

A round bottomed flask was charged with 5,6-difluoroindoline-2,3-dione (2.0 g), trimethyl(trifluoromethyl)silane (5.0 g), cesium fluoride (5.0 g) and THF (30 mL). The solution was stirred at 25° C. for 1 hour. The reaction mixture was diluted with H₂O (50 mL) and the aqueous phase was extracted with ethyl acetate (50 mL) three times. The combined organic layers were washed with brine, dried over sodium sulfate, filtered, and concentrated in vacuo. The resulting crude material was purified by HPLC (water/ACN). Lyophilization yielded the desired product as a brown amorphous solid (1.6 g). LCMS: RT 0.67 min, [M+H]⁺ 254.00, LCMS method O.

### Step 2. 3-chloro-5,6-difluoro-3-(trifluoromethyl)indolin-2-one

A round bottomed flask was charged with 5,6-difluoro-3-hydroxy-3-(trifluoromethyl)indolin-2-one (1.0 g), pyridine (0.9 g) and SOCl₂ (15 mL). The solution was stirred at 80° C. for 1 hour. The reaction mixture was quenched with water (50 mL), and the aqueous phase was extracted with ethyl acetate (50 mL) three times. The combined organic layers were washed with brine, dried over sodium sulfate, filtered, and concentrated in vacuo. The resulting crude material was purified by HPLC (water/ACN). Lyophilization yielded the desired product as a yellow amorphous solid (0.56 g). LCMS: RT 0.789 min, [M+H]⁺ 271.95, LCMS method O.

### Step 3. 5,6-difluoro-3-(trifluoromethyl)indolin-2-one

A round bottomed flask was charged with 3-chloro-5,6-difluoro-3-(trifluoromethyl)indolin-2-one (900 mg), Pd/C (0.97 g) and MeOH (10 mL). The flask was evacuated and flushed three times with hydrogen. The mixture was stirred for 1 hour at room temperature under an atmosphere of hydrogen (balloon). The mixture was filtered through a Celite pad. The filtrate was concentrated under vacuum to give the desired product as a yellow amorphous solid (0.56 g). LCMS: RT 0.76 min, [M+H]⁺ 238.05, LCMS method I.

### Step 4. 5,6-difluoro-3-(trifluoromethyl)indoline

A round bottomed flask was charged with 5,6-difluoro-3-(trifluoromethyl)indolin-2-one (260 mg), BH₃ in THF (5.4 mL) and THF (5 mL). The solution was stirred at 70° C. for 1 hour. The reaction mixture was quenched with water (20 mL), and the aqueous phase was extracted with ethyl acetate (20 mL) three times. The combined organic layers were washed with brine, dried over sodium sulfate, filtered, and concentrated in vacuo. The resulting crude material was purified by HPLC (water/ACN). Lyophilization yielded the desired product as a yellow oil (50 mg). LCMS: RT 0.88 min, [M+H]⁺ 223.95, LCMS method I.

### Step 5. (S)-8-(2-chloro-5-fluorophenyl)-1-((R)-5,6-difluoro-3-(trifluoromethyl)indoline-1-carboxamido)-N-methyl-6-oxo-5,6,7,8-tetrahydroimidazo[1,5-a]pyrazine-3-carboxamide and (S)-8-(2-chloro-5-fluorophenyl)-1-((S)-5,6-difluoro-3-(trifluoromethyl)indoline-1-carboxamido)-N-methyl-6-oxo-5,6,7,8-tetrahydroimidazo[1,5-a]pyrazine-3-carboxamide

The desired products were synthesized by analogy to the procedure of Example 37, Step 3 using 5,6-difluoro-3-(trifluoromethyl)indoline (66.6 mg), 4-nitrophenyl (S)-(8-(2-chloro-5-fluorophenyl)-3-(methylcarbamoyl)-6-oxo-5,6,7,8-tetrahydroimidazo[1,5-a]pyrazin-1-yl)carbamate (100 mg) and TEA (60.3 mg), giving a mixture of the two diastereomers (15 mg) as a white amorphous solid. LCMS: RT 0.768 min, [M+H]⁺ 587.15, LCMS method O.

The two isomers (7 mg) were purified by CHIRAL-HPLC (Column: CHIRALPAK IG, 2*25 cm, 5 μm; Mobile Phase A: hexane (0.2% TEA), Mobile Phase B: EtOH:DCM=1:1; Flow rate: 20 mL/min; Gradient: 0% to 50% B in 18 min; Wavelength: 220/254 nm; Sample was dissolved in EtOH: DCM 1:1; Injection volume: 0.95 mL.

Compound I-626, Peak 1, chiral HPLC retention time: 10.69 min; 2.3 mg as a white amorphous solid. LCMS: RT 1.42 min, [M+H]⁺ 587.15, LCMS method M. 1H NMR (400 MHz, DMSO-d6) 8.89 (d, J=2.2 Hz, 1H), 8.62 (s, 1H), 8.35 (d, J=4.9 Hz, 1H), 7.79 (dd, J=12.4, 7.4 Hz, 1H), 7.43 (t, J=9.0 Hz, 1H), 7.36 (dd, J=8.8, 5.1 Hz, 1H), 7.17 (dd, J=9.2, 3.1 Hz, 1H), 7.11 (td, J=8.4, 3.1 Hz, 1H), 6.05 (s, 1H), 5.21 (s, 1H), 5.05 (dd, J=18.8, 1.6 Hz, 1H), 4.51 (s, 1H), 4.12 (t, J=11.0 Hz, 1H), 3.75 (dd, J=11.5, 4.3 Hz, 1H), 2.76 (d, J=4.7 Hz, 3H).

Compound I-625, Peak 2: chiral HPLC retention time: 16.09 min; 3.1 mg as a white amorphous solid. LCMS: RT 0.89 min, [M+H]⁺ 587.25, LCMS method K. 1H NMR (400 MHz, DMSO-d6) 8.91 (d, J=2.2 Hz, 1H), 8.65 (s, 1H), 8.34 (d, J=5.0 Hz, 1H), 7.77 (dd, J=12.3, 7.3 Hz, 1H), 7.45 (t, J=9.0 Hz, 1H), 7.37 (dd, J=8.8, 5.1 Hz, 1H), 7.20 (dd, J=9.3, 3.1 Hz, 1H), 7.14 (td, J=8.4, 3.1 Hz, 1H), 6.05 (s, 1H), 5.22 (s, 1H), 5.05 (dd, J=18.4, 1.5 Hz, 1H), 4.51 (s, 1H), 4.11 (dd, J=11.8, 3.8 Hz, 1H), 3.71 (t, J=11.0 Hz, 1H), 2.76 (d, J=4.7 Hz, 3H).

Additional compounds prepared according to the methods of Example 39 are listed in Table 11 below. Corresponding ¹H NMR and mass spectrometry characterization for these compounds are described in Table 1. Certain compounds in Table 11 below were prepared with other compounds whose preparation is described in the Examples herein.

TABLE 11
Additional Exemplary Compounds
Compound

I-250
I-274
I-275
I-276
I-277
I-352
I-355
I-356
I-411
I-446
I-697
I-698
I-699
I-700

### Example 40

### (R)—N-(8-(2-chloro-5-fluorophenyl)-3-(methylcarbamoyl)-6-oxo-5,6,7,8-tetrahydroimidazo[1,5-a]pyrazin-1-yl)-5-cyanobenzo[d]isothiazole-3-carboxamide (I-412 or I-413) and (S)—N-(8-(2-chloro-5-fluorophenyl)-3-(methylcarbamoyl)-6-oxo-5,6,7,8-tetrahydroimidazo[1,5-a]pyrazin-1-yl)-5-cyanobenzo[d]isothiazole-3-carboxamide (I-412 or I-413)

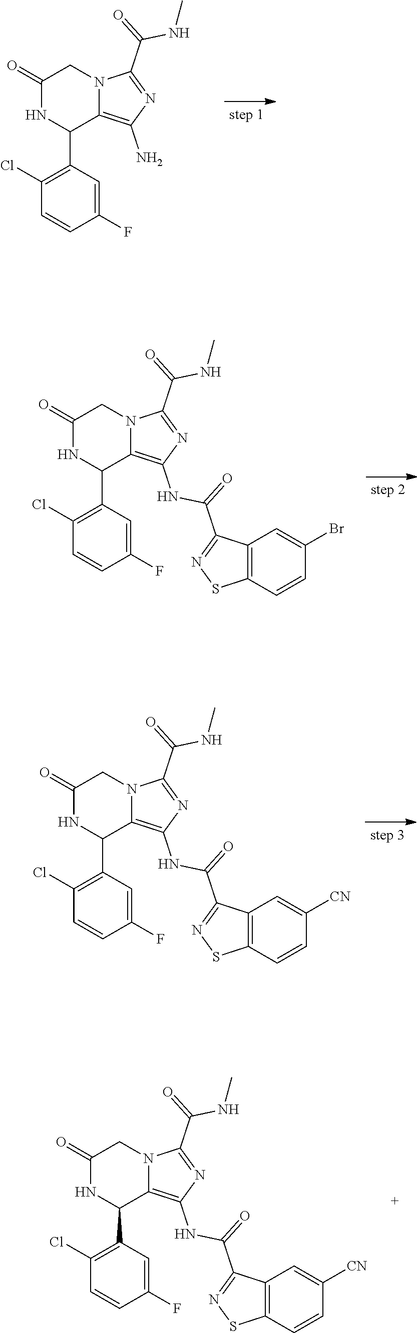 [SMILES: CNC(=O)c1nc(N)c2n1CC(=O)NC2c1cc(F)ccc1Cl ; CNC(=O)c1nc(NC(=O)c2nsc3ccc(Br)cc23)c2n1CC(=O)NC2c1cc(F)ccc1Cl ; CNC(=O)c1nc(NC(=O)c2nsc3ccc(C#N)cc23)c2n1CC(=O)NC2c1cc(F)ccc1Cl ; CNC(=O)c1nc(NC(=O)c2nsc3ccc(C#N)cc23)c2n1CC(=O)N[C@@H]2c1cc(F)ccc1Cl]

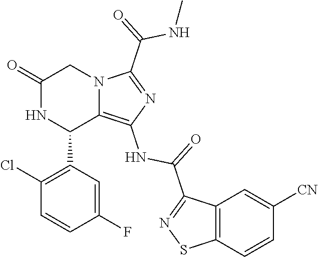 [SMILES: CNC(=O)c1nc(NC(=O)c2nsc3ccc(C#N)cc23)c2n1CC(=O)N[C@H]2c1cc(F)ccc1Cl]

### Step 1. 5-bromo-N-(8-(2-chloro-5-fluorophenyl)-3-(methylcarbamoyl)-6-oxo-5,6,7,8-tetrahydroimidazo[1,5-a]pyrazin-1-yl)benzo[d]isothiazole-3-carboxamide

A reaction vial was charged with 1-amino-8-(2-chloro-5-fluorophenyl)-N-methyl-6-oxo-5,6,7,8-tetrahydroimidazo[1,5-a]pyrazine-3-carboxamide (150 mg), 5-bromobenzo[d]isothiazole-3-carboxylic acid (115 mg), HATU (253 mg) and DIEA (172 mg) before being evacuated and purged with nitrogen three times. DMF (4 mL) was added, and the mixture was stirred at RT overnight. The reaction mixture was purified by reverse phase chromatography (mobile phase A: water, mobile phase B: acetonitrile) to give the desired product (200 mg) as an off-white amorphous solid. LCMS: RT 1.036 min, [M+H]⁺ 579.2, LCMS method P.

### Step 2. N-(8-(2-chloro-5-fluorophenyl)-3-(methylcarbamoyl)-6-oxo-5,6,7,8-tetrahydroimidazo[1,5-a]pyrazin-1-yl)-5-cyanobenzo[d]isothiazole-3-carboxamide

A reaction vial was charged with 5-bromo-N-(8-(2-chloro-5-fluorophenyl)-3-(methylcarbamoyl)-6-oxo-5,6,7,8-tetrahydroimidazo[1,5-a]pyrazin-1-yl)benzo[d]isothiazole-3-carboxamide (200 mg), zinc cyanide (61.0 mg) and Pd(PPh₃)₄ (2.0 mg). The vial was evacuated and purged with nitrogen three times. DMF (4 mL) was added, and the mixture was stirred at 80° C. for 16 hours. The resulting crude material was purified by prep-HPLC (mobile phase A: water, mobile phase B: acetonitrile) to give the desired product (150 mg) as an off-white amorphous solid. LCMS: RT 1.237 min, [M+H]⁺=524.1, LCMS method M. ¹H NMR (400 MHz, DMSO-d6) δ 10.44 (s, 1H), 8.97-8.91 (m, 2H), 8.57 (d, J=8.6 Hz, 1H), 8.44 (d, J=4.9 Hz, 1H), 8.06 (dd, J=8.6, 1.6 Hz, 1H), 7.24 (ddd, J=14.4, 9.0, 4.1 Hz, 2H), 6.98 (td, J=8.3, 3.1 Hz, 1H), 6.16 (s, 1H), 5.23 (s, 1H), 5.11 (dd, J=18.7, 1.6 Hz, 1H), 2.77 (d, J=4.7 Hz, 3H).

### Step 3. (R)—N-(8-(2-chloro-5-fluorophenyl)-3-(methylcarbamoyl)-6-oxo-5,6,7,8-tetrahydroimidazo[1,5-a]pyrazin-1-yl)-5-cyanobenzo[d]isothiazole-3-carboxamide and (S)—N-(8-(2-chloro-5-fluorophenyl)-3-(methylcarbamoyl)-6-oxo-5,6,7,8-tetrahydroimidazo[1,5-a]pyrazin-1-yl)-5-cyanobenzo[d]isothiazole-3-carboxamide

Racemic N-(8-(2-chloro-5-fluorophenyl)-3-(methylcarbamoyl)-6-oxo-5,6,7,8-tetrahydroimidazo[1,5-a]pyrazin-1-yl)-5-cyanobenzo[d]isothiazole-3-carboxamide (40 mg) was chirally resolved by preparative scale chiral HPLC (Column: CHIRALPAK IH, 2*25 cm, 5 μm; mobile phase A: MeOH with 0.1% TFA, mobile phase B: dichloromethane; flow rate: 20 mL/min; Gradient: 30% B isocratic; wavelength: 220/254 nm; sample dissolved in dichloromethane; injection volume: 0.85 mL; number of runs: 3) to give the two enantiomers, both as an off-white amorphous solid.

Compound I-413, Peak 1: 16.5 mg. Chiral HPLC RT: 2.94 min. LCMS: RT 1.241 min, [M+H]⁺ 524.0, LCMS method M. ¹H NMR (400 MHz, DMSO-d6) δ 10.44 (s, 1H), 8.94 (t, J=2.1 Hz, 2H), 8.57 (d, J=8.6 Hz, 1H), 8.43 (d, J=4.9 Hz, 1H), 8.06 (dd, J=8.6, 1.5 Hz, 1H), 7.24 (ddd, J=15.1, 9.1, 4.1 Hz, 2H), 6.98 (td, J=8.4, 3.1 Hz, 1H), 6.16 (s, 1H), 5.23 (s, 1H), 5.16-5.06 (m, 1H), 2.77 (d, J=4.7 Hz, 3H).

Compound I-412, Peak 2: 18.9 mg. Chiral HPLC RT: 4.70 min. LCMS: RT 1.245 min, [M+H]⁺ 524.0, LCMS method M. ¹H NMR (400 MHz, DMSO-d6) δ 10.44 (s, 1H), 8.97-8.91 (m, 2H), 8.57 (dd, J=8.6, 0.8 Hz, 1H), 8.43 (d, J=4.8 Hz, 1H), 8.06 (dd, J=8.6, 1.5 Hz, 1H), 7.30-7.18 (m, 2H), 6.98 (td, J=8.3, 3.1 Hz, 1H), 6.16 (s, 1H), 5.23 (s, 1H), 5.11 (dd, J=18.7, 1.6 Hz, 1H), 2.77 (d, J=4.7 Hz, 3H).

### Example 41

### (R)—N-(8-(2-chloro-5-fluorophenyl)-3-(methylcarbamoyl)-6-oxo-5,6,7,8-tetrahydroimidazo[1,5-a]pyrazin-1-yl)-6-fluorobenzo[d]isothiazole-3-carboxamide (I-414 or I-415) and (S)—N-(8-(2-chloro-5-fluorophenyl)-3-(methylcarbamoyl)-6-oxo-5,6,7,8-tetrahydroimidazo[1,5-a]pyrazin-1-yl)-6-fluorobenzo[d]isothiazole-3-carboxamide (I-414 or I-415)

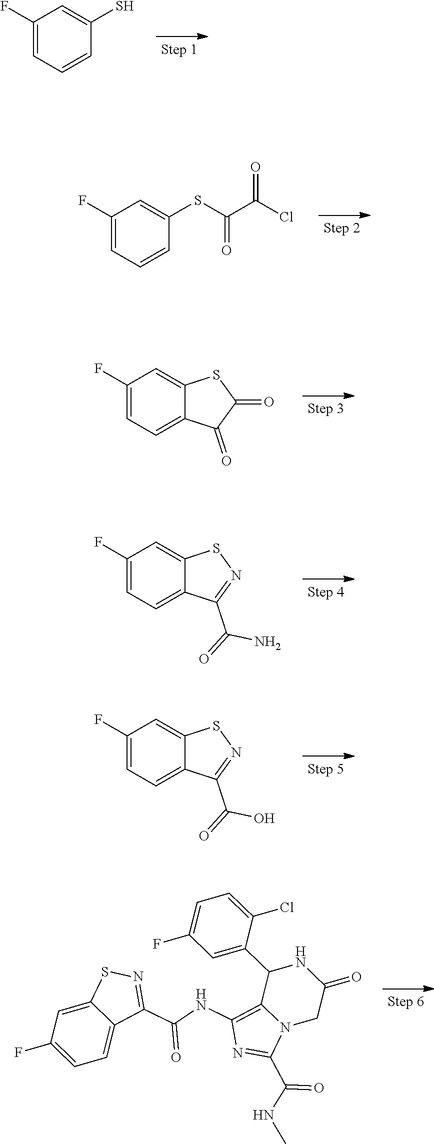 [SMILES: Fc1cccc(S)c1 ; O=C(Cl)C(=O)Sc1cccc(F)c1 ; O=C1Sc2cc(F)ccc2C1=O ; NC(=O)c1nsc2cc(F)ccc12 ; O=C(O)c1nsc2cc(F)ccc12 ; CNC(=O)c1nc(NC(=O)c2nsc3cc(F)ccc23)c2n1CC(=O)NC2c1cc(F)ccc1Cl]

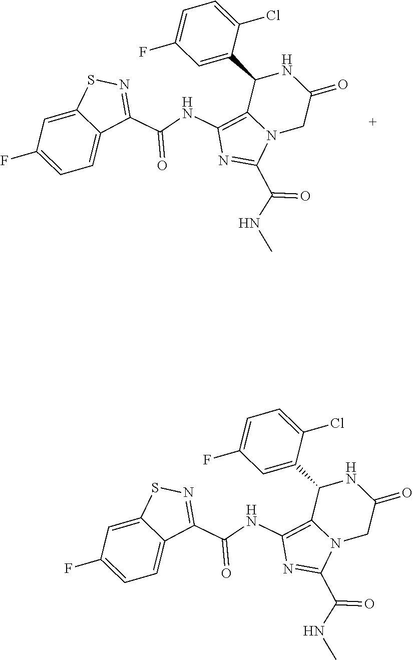 [SMILES: CNC(=O)c1nc(NC(=O)c2nsc3cc(F)ccc23)c2n1CC(=O)N[C@@H]2c1cc(F)ccc1Cl ; CNC(=O)c1nc(NC(=O)c2nsc3cc(F)ccc23)c2n1CC(=O)N[C@H]2c1cc(F)ccc1Cl]

### Step 1. S-(3-fluorophenyl) 2-chloro-2-oxoethanethioate

A reaction vial was charged with 3-fluorobenzenethiol (3.00 g), oxalyl chloride (3.57 g) and a stir bar before being evacuated and purged with nitrogen three times. Et₂O (30 mL) was added, and the mixture was stirred at 40° C. for 2 hours. After concentration in vacuo the crude material was used in the next step without purification.

### Step 2. 6-fluorobenzo[b]thiophene-2,3-dione

A reaction vial was charged with S-(3-fluorophenyl) 2-chloro-2-oxoethanethioate (2 g), AlCl₃ (2 g) and a stir bar before being evacuated and purged with nitrogen three times. Dichloromethane (20 mL) was added, and the mixture was stirred at 70° C. for 30 minutes under nitrogen. After cooling to room temperature the reaction was quenched with water. The aqueous phase was extracted with ethyl acetate (200 mL×3). The combined organic layers were washed with brine, dried over sodium sulfate, filtered and concentrated in vacuo. The crude product was purified by silica gel chromatography (10 g column; eluting with petroleum ether:ethyl acetate 10:1) to give the desired product (300 mg) as an off-white amorphous solid. LCMS: RT 1.051 min, [M+H]⁺ not observed, LCMS method A.

### Step 3. 6-fluorobenzo[d]isothiazole-3-carboxamide

A reaction vial was charged with 6-fluorobenzo[b]thiophene-2,3-dione (300 mg) and a stir bar before being evacuated and purged with nitrogen three times. Ammonium hydroxide (10 mL) and hydrogen peroxide (1.0 mL) were added, and the mixture was stirred at 25° C. for 12 hours under nitrogen. The precipitated solid was collected by filtration and washed with H₂O (5 mL). The resulting solid was purified by HPLC (mobile phase A: water with 0.1% formic acid, mobile phase B: acetonitrile with 0.1% formic acid) to give the desired product (200 mg) as an off-white amorphous solid. LCMS: RT 0.878 min, [M+H]⁺ 196.95, LCMS method A.

### Step 4. 6-fluorobenzo[d]isothiazole-3-carboxylic acid

A reaction vial was charged with 6-fluorobenzo[d]isothiazole-3-carboxamide (200 mg) and a stir bar before being evacuated and purged with nitrogen three times. MeOH (10 mL) and NaOH solution (10 N, 3.3 mL) were added, and the mixture was stirred at 70° C. for 12 hours under nitrogen. The pH was adjusted to weakly acidic with hydrogen chloride. The reaction mixture was diluted with water (15 mL), and the aqueous phase was extracted with ethyl acetate (15 mL×3). The combined organic layers were washed with brine, dried over sodium sulfate, filtered and concentrated in vacuo. The resulting crude material was purified by prep-scale HPLC (mobile phase A: water, mobile phase B: acetonitrile) to give the desired product (120 mg) as an off-white amorphous solid. LCMS: RT 0.889 min, [M+H]⁺ 198.05, LCMS method A.

### Step 5. N-(8-(2-chloro-5-fluorophenyl)-3-(methylcarbamoyl)-6-oxo-5,6,7,8-tetrahydroimidazo[1,5-a]pyrazin-1-yl)-6-fluorobenzo[d]isothiazole-3-carboxamide

A reaction vial was charged with 1-amino-8-(2-chloro-5-fluorophenyl)-N-methyl-6-oxo-5,6,7,8-tetrahydroimidazo[1,5-a]pyrazine-3-carboxamide (25 mg), 6-fluorobenzo[d]isothiazole-3-carboxylic acid (22 mg), HATU (42 mg), DIEA (29 mg) and a stir bar before being evacuated and purged with nitrogen three times. DMF (3 mL) was added, and the mixture was stirred at 25° C. for 1 hour under nitrogen. The resulting crude material was purified by HPLC (mobile phase A: water, mobile phase B: acetonitrile) to give the desired product (25 mg) as an off-white amorphous solid. LCMS: RT 1.032 min, [M+H]⁺ 517.3, LCMS method A.

### Step 6. (R)—N-(8-(2-chloro-5-fluorophenyl)-3-(methylcarbamoyl)-6-oxo-5,6,7,8-tetrahydroimidazo[1,5-a]pyrazin-1-yl)-6-fluorobenzo[d]isothiazole-3-carboxamide and (S)—N-(8-(2-chloro-5-fluorophenyl)-3-(methylcarbamoyl)-6-oxo-5,6,7,8-tetrahydroimidazo[1,5-a]pyrazin-1-yl)-6-fluorobenzo[d]isothiazole-3-carboxamide

Racemic N-(8-(2-chloro-5-fluorophenyl)-3-(methylcarbamoyl)-6-oxo-5,6,7,8-tetrahydroimidazo[1,5-a]pyrazin-1-yl)-6-fluorobenzo[d]isothiazole-3-carboxamide (25 mg) was chirally resolved by CHIRAL-HPLC (Column: DZ-CHIRALPAK IH-3, 4.6*50 mm, 3.0 μm; mobile phase A: hexane with 0.2% diethylamine; phase B: EtOH:DCM 1:1); gradient: 30:70 isocratic; flow rate: 1 mL/min) to give the two enantiomers, both as an off-white amorphous solid.

Compound I-415, Peak 1: 11.7 mg. LCMS: RT 1.363 min, [M+H]⁺=517.15, LCMS method M. ¹H NMR (400 MHz, DMSO-d6) δ 10.30 (s, 1H), 8.95 (s, 1H), 8.59 (dd, J=9.1, 5.2 Hz, 1H), 8.44 (d, J=5.1 Hz, 1H), 8.19 (dd, J=8.9, 2.5 Hz, 1H), 7.56-7.47 (m, 1H), 7.27 (dd, J=8.8, 5.1 Hz, 1H), 7.17 (dd, J=9.2, 3.1 Hz, 1H), 7.06-6.96 (m, 1H), 6.16 (s, 1H), 5.23 (s, 1H), 5.13 (s, 1H), 2.77 (d, J=4.7 Hz, 3H).

Compound I-414, Peak 2: 7.5 mg. LCMS: RT 1.365 min, [M+H]⁺ 517.15, LCMS method M. ¹H NMR (400 MHz, DMSO-d6) 10.31 (s, 1H), 8.96 (d, J=2.4 Hz, 1H), 8.60 (dd, J=9.1, 5.2 Hz, 1H), 8.45 (d, J=4.8 Hz, 1H), 8.19 (dd, J=9.0, 2.4 Hz, 1H), 7.53 (td, J=9.0, 2.4 Hz, 1H), 7.27 (dd, J=8.9, 5.1 Hz, 1H), 7.17 (dd, J=9.2, 3.1 Hz, 1H), 7.02 (td, J=8.4, 3.1 Hz, 1H), 6.16 (s, 1H), 5.23 (s, 1H), 5.11 (dd, J=18.7, 1.6 Hz, 1H), 2.77 (d, J=4.8 Hz, 3H).

### Example 42

### (R)—N-(8-(2-chloro-5-fluorophenyl)-3-(methylcarbamoyl)-6-oxo-5,6,7,8-tetrahydroimidazo[1,5-a]pyrazin-1-yl)-5,7-difluorobenzo[d]isothiazole-3-carboxamide (I-485 or I-486) and (S)—N-(8-(2-chloro-5-fluorophenyl)-3-(methylcarbamoyl)-6-oxo-5,6,7,8-tetrahydroimidazo[1,5-a]pyrazin-1-yl)-5,7-difluorobenzo[d]isothiazole-3-carboxamide (I-485 or I-486)

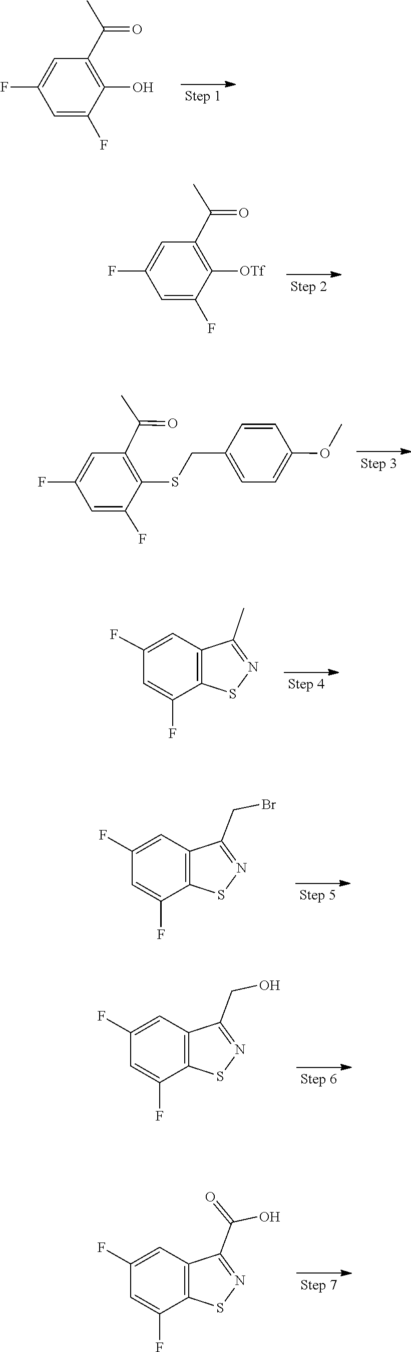

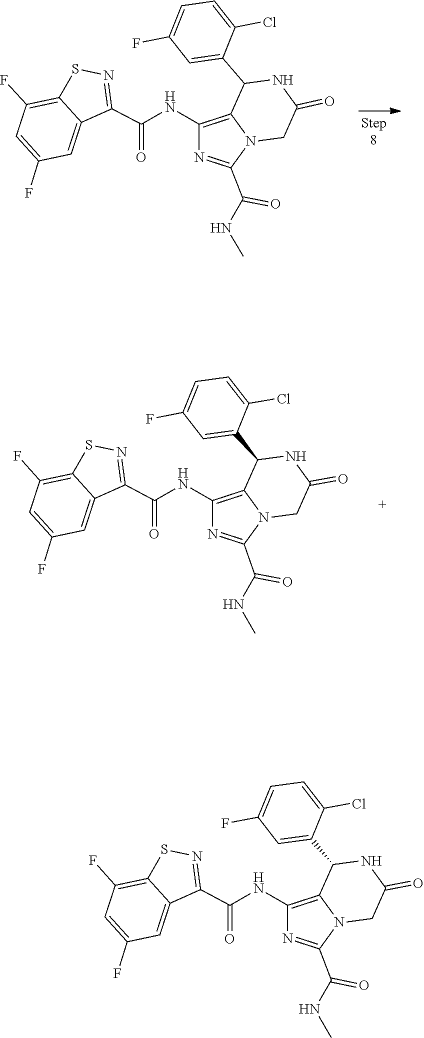 [SMILES: CNC(=O)c1nc(NC(=O)c2nsc3c(F)cc(F)cc23)c2n1CC(=O)NC2c1cc(F)ccc1Cl ; CNC(=O)c1nc(NC(=O)c2nsc3c(F)cc(F)cc23)c2n1CC(=O)N[C@@H]2c1cc(F)ccc1Cl ; CNC(=O)c1nc(NC(=O)c2nsc3c(F)cc(F)cc23)c2n1CC(=O)N[C@H]2c1cc(F)ccc1Cl]

### Step 1. 2-acetyl-4,6-difluorophenyl trifluoromethanesulfonate

A reaction vial was charged with 1-(3,5-difluoro-2-hydroxyphenyl)ethan-1-one (9 g), trifluoromethanesulfonic anhydride (19.7 g), dichloromethane (60 mL), pyridine (8 g) and a stir bar before being evacuated and purged with nitrogen three times. The mixture was stirred at room temperature for 40 minutes. The reaction was quenched with hydrochloric acid (2 N) until the pH is 6-7 and extracted with dichloromethane (3×40 mL). The organic phase was combined, dried over Na₂SO₄ and concentrated in vacuo. The resulting crude material was purified by C18 reverse phase chromatography with 0-100% acetonitrile in water as the eluent to give the desired product (10 g) as an off-white amorphous solid. LCMS: RT 1.217 min, [M+H]⁺ not observed, LCMS method P. ¹H NMR (400 MHz, DMSO-d6) δ 8.04-7.93 (m, 2H), 2.65 (s, 3H).

### Step 2. 1-(3,5-difluoro-2-((4-methoxybenzyl)thio)phenyl)ethan-1-one

A reaction vial was charged with 2-acetyl-4,6-difluorophenyl trifluoromethanesulfonate (9 g), (4-methoxyphenyl) methanethiol (5 g), Pd₂(dba)₃·CHCl₃ (1 g), xantphos (0.7 g), DIEA (8 g) and a stir bar before being evacuated and purged with nitrogen three times. Dioxane (70 mL) was added, and the mixture was stirred at 90° C. for 2 hours. The reaction mixture was diluted with H₂O (60 mL), and the aqueous phase was extracted with dichloromethane (3×50 mL). The combined organic layers were washed with brine, dried over sodium sulfate, filtered and concentrated in vacuo. The resulting crude material was purified by C-18 reverse phase chromatography with water:acetonitrile (0-100% acetonitrile) as the eluent to give the desired product (6 g) as an off-white amorphous solid. LCMS: RT 1.29 min, [M+H]⁺ not observed, LCMS method P. ¹H NMR (400 MHz, DMSO-d6) δ 7.48 (ddd, J=9.6, 8.8, 0.4 Hz, 1H), 7.31-7.27 (m, 1H), 7.03 (dd, J=0.8, 0.4 Hz, 2H), 6.81 (dd, J=0.8, 0.4 Hz, 2H), 3.98 (s, 2H), 3.70 (s, 3H), 2.28 (s, 3H).

### Step 3. 5,7-difluoro-3-methylbenzo[d]isothiazole

To a solution of 1-(3,5-difluoro-2-((4-methoxybenzyl)thio)phenyl)ethan-1-one (5 g) in dichloromethane (63 mL) at room temperature was added dropwise SO₂Cl₂ (2 g). The mixture was stirred at room temperature for 0.5 hour and concentrated in vacuo. The residue was dissolved in tetrahydrofuran (63 mL) and treated with a saturated solution of ammonia in ethanol (63 mL). The resulting mixture was stirred at room temperature for 1 hour, diluted with water (20 mL) and extracted with ethyl acetate (3×20 mL). The combined organic layers were washed with brine (3×20 mL), dried over anhydrous solium sulfate, filtered and concentrated in vacuo. The residue was purified by C18 reverse phase chromatography with water/acetonitrile as the eluent to give the desired product (1.2 g) as a yellow amorphous solid. LCMS: RT 0.943 min, [M+H]⁺ 186.1, LCMS method C.

### Step 4. 3-(bromomethyl)-5,7-difluorobenzo[d]isothiazole

N-bromosuccinimide (1.3 g) was added to a solution of 5,7-difluoro-3-methylbenzo[d]isothiazole (1.2 g) in CCl₄ (10 mL). Ph(CO₂)₂ (0.16 g) was added and the reaction mixture was heated at reflux for 20 hours. The reaction mixture was diluted with H₂O (30 mL), and the aqueous phase was extracted with dichloromethane (3×30 mL). The combined organic layers were washed with brine, dried over sodium sulfate, filtered and concentrated in vacuo. The resulting crude material was purified by C18 reverse phase chromatography with water/acetonitrile as the eluent to give the desired product (900 mg) as a yellow amorphous solid. LCMS: RT 1.05 min, [M+H]⁺ 263.9. LCMS method E.

### Step 5. (5,7-difluorobenzo[d]isothiazol-3-yl)methanol

A solution of 3-(bromomethyl)-5,7-difluorobenzo[d]isothiazole (950 mg) in dimethylsulfoxide (5 mL) was treated with H₂O (1 mL) and the mixture was heated at 80° C. for 1.5 hours. The reaction mixture was diluted with H₂O (30 mL), and the aqueous phase was extracted with dichloromethane (3×30 mL). The combined organic layers were washed with brine, dried over sodium sulfate, filtered and concentrated in vacuo. The resulting crude material was purified by C18 reverse phase chromatography with water/acetonitrile as the eluent to give the desired product (520 mg) as an off-white amorphous solid. LCMS: RT 0.85 min, [M+H]⁺ 202.0, LCMS method A.

### Step 6. 5,7-difluorobenzo[d]isothiazole-3-carboxylic acid

Potassium permanganate (314 mg) and KOH (95 mg) were added to a solution of (5,7-difluorobenzo[d]isothiazol-3-yl)methanol (200 mg) in H₂O (5 mL) and the resulting solution was stirred for 1 hour at room temperature. The reaction mixture was filtered through Celite and extracted with ethyl acetate (3×10 mL). The pH of the combined aqueous layers was adjusted to 2 by the addition of 0.6 N hydrochloric acid. The resulting slurry was stirred for 5 minutes and the solid was collected by filtration to give the desired product (70 mg) as an off-white amorphous solid. LCMS: RT 0.75 min, [M+H]⁺ 216.0, LCMS method E.

### Step 7. N-(8-(2-chloro-5-fluorophenyl)-3-(methylcarbamoyl)-6-oxo-5,6,7,8-tetrahydroimidazo[1,5-a]pyrazin-1-yl)-5,7-difluorobenzo[d]isothiazole-3-carboxamide

A reaction vial was charged with 5,7-difluorobenzo[d]isothiazole-3-carboxylic acid (60 mg), 1-amino-8-(2-chloro-5-fluorophenyl)-N-methyl-6-oxo-5,6,7,8-tetrahydroimidazo[1,5-a]pyrazine-3-carboxamide (0.11 g), HATU (0.13 g), DMF (2 mL), DIEA (0.11 g) and a stir bar before being evacuated and purged with nitrogen three times. The mixture was stirred at room temperature for 1 hour. The resulting crude material was purified by C18 reverse phase chromatography with water/acetonitrile as the eluent to give the desired product (70 mg) as an off-white amorphous solid. LCMS: RT 1.02 min, [M+H]⁺ 535.0, LCMS method P.

### Step 8. (R)—N-(8-(2-chloro-5-fluorophenyl)-3-(methylcarbamoyl)-6-oxo-5,6,7,8-tetrahydroimidazo[1,5-a]pyrazin-1-yl)-5,7-difluorobenzo[d]isothiazole-3-carboxamide and (S)—N-(8-(2-chloro-5-fluorophenyl)-3-(methylcarbamoyl)-6-oxo-5,6,7,8-tetrahydroimidazo[1,5-a]pyrazin-1-yl)-5,7-difluorobenzo[d]isothiazole-3-carboxamide

N-(8-(2-chloro-5-fluorophenyl)-3-(methylcarbamoyl)-6-oxo-5,6,7,8-tetrahydroimidazo[1,5-a]pyrazin-1-yl)-5,7-difluorobenzo[d]isothiazole-3-carboxamide (70 mg) was chirally resolved by chiral-HPLC (Column: DZ-CHIRALPAK IH-3, 4.6*50 mm, 3.0 μm; eluent A: hexane with 0.2% diethylamine; eluent B: EtOH:dichloromethane 1:1; gradient: 50:50 isocratic; flow rate: 1 mL/min) to give the two enantiomers, both as an off-white amorphous solid.

Peak 1: 28.1 mg. LCMS: RT 1.021 min, [M+H]⁺ 535.0, LCMS method P. ¹H NMR (400 MHz, DMSO-d6) δ 10.48 (s, 1H), 8.95 (d, J=2.3 Hz, 1H), 8.44 (d, J=4.9 Hz, 1H), 8.15 (dd, J=8.9, 2.1 Hz, 1H), 7.84 (td, J=9.4, 2.2 Hz, 1H), 7.26 (dd, J=8.9, 5.1 Hz, 1H), 7.19 (dd, J=9.2, 3.1 Hz, 1H), 7.01 (td, J=8.4, 3.1 Hz, 1H), 6.15 (d, J=2.3 Hz, 1H), 5.26 (d, J=18.7 Hz, 1H), 5.10 (dd, J=18.8, 1.6 Hz, 1H), 2.77 (d, J=4.7 Hz, 3H).

Peak 2: 20.3 mg. LCMS: RT 1.028 min, [M+H]⁺ 535.0, LCMS method P. ¹H NMR (400 MHz, DMSO-d6) δ 10.48 (s, 1H), 8.95 (d, J=2.3 Hz, 1H), 8.44 (q, J=4.8 Hz, 1H), 8.15 (dd, J=8.9, 2.1 Hz, 1H), 7.84 (td, J=9.4, 2.1 Hz, 1H), 7.26 (dd, J=8.8, 5.1 Hz, 1H), 7.19 (dd, J=9.2, 3.1 Hz, 1H), 7.01 (td, J=8.4, 3.1 Hz, 1H), 6.15 (d, J=2.3 Hz, 1H), 5.26 (d, J=18.7 Hz, 1H), 5.10 (dd, J=18.7, 1.6 Hz, 1H), 2.77 (d, J=4.8 Hz, 3H).

### Example 43

### N-(1-(2-chloro-5-fluorophenyl)-6-(1-(difluoromethyl)-1H-pyrazol-4-yl)-3-oxo-1,2,3,4-tetrahydropyrrolo[1,2-a]pyrazin-8-yl)benzo[d]isothiazole-3-carboxamide (I-114)

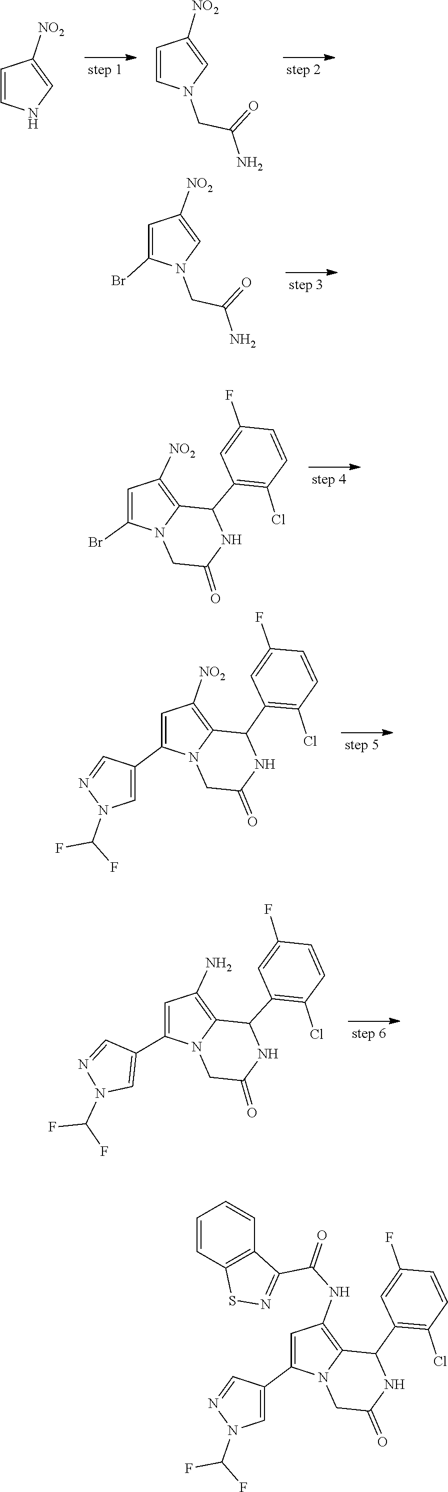

### Step 1. 2-(3-nitro-1H-pyrrol-1-yl)acetamide

To a solution of 3-nitro-1H-pyrrole (80.0 g) in acetonitrile (1.20 L) was added 2-tert-butyl-1,1,3,3-tetramethylguanidine (141 g). The mixture was stirred at 25° C. for 10 minutes, and 2-bromoacetamide (158 g) was added. The reaction mixture was stirred at 25° C. for 1 hour. The precipitate was collected by filtration. The filtrate was concentrated. The precipitate was triturated with water (500 mL) and collected by filtration. The solid was combined and dried under reduced pressure to give the crude product. The filtrate was extracted with ethyl acetate (800 mL*6). The combined organic layers were dried over Na₂SO₄, filtered, concentrated, then purified by column chromatography (SiO₂, petroleum ether:Ethyl acetate 10:1 to methanol:ethyl acetate=1:9) to give another batch of the product. The two batches of product were combined and dried under reduced pressure to give the desired product (130 g) as a light-yellow solid. ¹H NMR (400 MHz, DMSO-d6) δ 7.89 (t, J=2.0 Hz, 1H), 7.58 (s, 1H), 7.30 (s, 1H), 6.85 (dd, J=3.0, 2.4 Hz, 1H), 6.64 (dd, J=3.2, 1.8 Hz, 1H), 4.66 (s, 2H).

### Step 2. 2-(2-bromo-4-nitro-1H-pyrrol-1-yl)acetamide

Two batches were run in parallel. To a solution of 2-(3-nitro-1H-pyrrol-1-yl)acetamide (61.4 g) in DMF (307 mL) was added dropwise a solution of NBS (64.6 g) in DMF (307 mL) at 0° C. The mixture was stirred at 0° C. for 1.5 hours. The two batches were combined and the reaction mixture was poured into water (3.00 L) and filtered. The collected solid was triturated with petroleum ether:ethyl acetate 1:5 (600 mL) for 8 hours and filtered. The solid was dried to give the desired product (154 g) as a light yellow solid. LCMS: RT 0.599 min, [M+H]⁺ 248.0, LCMS method Y. ¹H NMR (400 MHz, DMSO-d6) δ 8.12 (d, J=2.2 Hz, 1H), 7.69 (s, 1H), 7.37 (s, 1H), 6.90 (d, J=2.2 Hz, 1H), 4.69 (s, 2H).

### Step 3. 6-bromo-1-(2-chloro-5-fluorophenyl)-8-nitro-1,2-dihydropyrrolo[1,2-a]pyrazin-3(4H)-one

Two batches were run in parallel. To a solution of 2-(2-bromo-4-nitro-1H-pyrrol-1-yl)acetamide (73.5 g) and 2-chloro-5-fluorobenzaldehyde (45.6 g) in dichloroethane (735 mL) was added Eaton's reagent (311 g) at 25° C. The solution was heated at 80° C. and stirred for 2 hours. The two batches were combined and poured into water (1.00 L), and ethyl acetate (2.00 L) was added. Saturated Na₂CO₃ solution was added, and the pH was adjusted to 6-7. The aqueous layer was extracted with ethyl acetate (3.00 L*4). The combined organic layers were washed with brine (1.00 L*2), dried over Na₂SO₄, filtered and concentrated under reduced pressure. The residue was purified by column chromatography (SiO₂, petroleum ether:Ethyl acetate 10:1 to dichloromethane:methanol 10:1). After most solvent was removed the precipitated solid was collected by filtration. The solid was triturated with ethyl acetate (60.0 mL) for 12 hours then filtered and dried to give the desired product (50.5 g) as a white solid. LCMS: RT 0.792 min, [M+H]⁺ not observed, LCMS method Y. ¹H NMR: (400 MHz, DMSO-d6) δ 9.26 (d, J=2.6 Hz, 1H), 7.48-7.55 (m, 1H), 7.19-7.27 (m, 2H), 7.12 (s, 1H), 6.43 (d, J=1.6 Hz, 1H), 4.90 (d, J=17.6 Hz, 1H), 4.60 (dd, J=17.6, 1.4 Hz, 1H).

### Step 4. 1-(2-chloro-5-fluorophenyl)-6-(1-(difluoromethyl)-1H-pyrazol-4-yl)-8-nitro-1,2-dihydropyrrolo[1,2-a]pyrazin-3(4H)-one

To a mixture of 6-bromo-1-(2-chloro-5-fluorophenyl)-8-nitro-1,2-dihydropyrrolo[1,2-a]pyrazin-3(4H)-one (214.2 mg), 1-(difluoromethyl)-4-(4,4,5,5-tetramethyl-1,3,2-dioxaborolan-2-yl)-1H-pyrazole (215.2 mg) and trans-dichlorobis(triphenylphosphine)palladium(II) (58.04 mg) in dioxane (3.6 mL) in a sealable tube was added a solution of sodium carbonate (175.3 mg) in water (1.2 mL). The mixture was degassed with N₂ before being sealed and stirred at 95° C. After 1 hour, the reaction was cooled to room temperature, diluted with water (35 mL) and brine (25 mL), and extracted with ethyl acetate (2×60 mL). The combined organic layers were dried over sodium sulfate, filtered and concentrated. The crude dark oil was eluted on a 12 gram silica gel column with a dichloromethane to 10:90:1 MeOH/dichloromethane/NH₄OH gradient over 24 minutes to give the desired product (138.9 mg) as a yellow solid. LCMS: RT 1.33 min, [M+H]⁺ 426.0; LCMS method U.

### Step 5. 8-amino-1-(2-chloro-5-fluorophenyl)-6-(1-(difluoromethyl)-1H-pyrazol-4-yl)-1,2-dihydropyrrolo[1,2-a]pyrazin-3(4H)-one

To a mixture of 1-(2-chloro-5-fluorophenyl)-6-(1-(difluoromethyl)-1H-pyrazol-4-yl)-8-nitro-1,2-dihydropyrrolo[1,2-a]pyrazin-3(4H)-one (1.10 g) in THF (19.2 mL), MeOH (6 mL), and water (6 mL) were added sodium dithionite (2.15 g), sodium carbonate (1.31 g) and 1,1′-dibenzyl-4,4′-bipyridiniumdichloride (50.5 mg). The mixture was stirred at room temperature for 16 hours then at 55° C. for 6 hours. The inorganic salts were removed by filtration and washed with ethyl acetate (3×20 mL). The filtrate was diluted with 2-MeTHF (150 mL) and water (80 mL). The layers were separated. The organic layer was washed with brine (2×40 mL), dried over sodium sulfate, filtered, concentrated and dried to yield the desired product (700 mg) as a rust-colored foamy solid, which was used in the next step without further purification. LCMS: RT 0.85 min, [M+H]⁺ 395.9, LCMS method U.

### Step 6. N-(1-(2-chloro-5-fluorophenyl)-6-(1-(difluoromethyl)-1H-pyrazol-4-yl)-3-oxo-1,2,3,4-tetrahydropyrrolo[1,2-a]pyrazin-8-yl)benzo[d]isothiazole-3-carboxamide

To a stirred solution of 8-amino-1-(2-chloro-5-fluorophenyl)-6-(1-(difluoromethyl)-1H-pyrazol-4-yl)-1,2-dihydropyrrolo[1,2-a]pyrazin-3(4H)-one (183.7 mg) and pyridine (936.2 mg) was added a solution formed by stirring for 25 minutes benzo[d]isothiazole-3-carboxylic acid (84.8 mg) and 1-chloro-N,N,2-trimethylprop-1-en-1-amine (63 mg) in toluene (0.5 mL). The reaction was stirred at room temperature and after 35 minutes methanol (1 mL) was added. After stirring for 5 minutes, the quenched reaction was concentrated and dried under high vacuum. The crude product was loaded as a silica gel slurry onto a 24 g silica gel column, which was eluted with a dichloromethane to 10:90:1 MeOH/dichloromethane/NH₄OH gradient over 22 minutes to give the desired product (97.0 mg). LCMS: RT 1.50 min [M+H]⁺ 556.9, LCMS method U. ¹H NMR (400 MHz, DMSO-d6) δ 9.86 (s, 1H), 8.92 (d, J=3.0 Hz, 1H), 8.66 (dt, J=8.1, 1.1 Hz, 1H), 8.59 (d, J=0.7 Hz, 1H), 8.29 (dt, J=8.3, 1.0 Hz, 1H), 8.12 (d, J=0.7 Hz, 1H), 7.85 (t, J=59.2 Hz, 1H), 7.67 (ddd, J=8.2, 6.9, 1.2 Hz, 1H), 7.58 (ddd, J=8.0, 6.9, 1.1 Hz, 1H), 7.32 (dd, J=8.8, 5.2 Hz, 1H), 7.13 (dd, J=9.3, 3.1 Hz, 1H), 7.05 (ddd, J=8.8, 8.0, 3.1 Hz, 1H), 6.52 (s, 1H), 6.28 (d, J=2.8 Hz, 1H), 5.02 (d, J=17.4 Hz, 1H), 4.73 (dd, J=17.5, 1.3 Hz, 1H).

Additional compounds prepared according to the methods of Example 40 are listed in Table 12 below. Corresponding ¹H NMR and mass spectrometry characterization for these compounds are described in Table 1. Certain compounds in Table 12 below were prepared with other compounds whose preparation is described in the Examples herein.

TABLE 12
Additional Exemplary Compounds
Compound

I-91
I-115
I-116
I-120
I-121
I-122
I-128
I-200

### Example 44

### (S)—N-(8-(2-chloro-5-fluorophenyl)-3-((hydroxyimino)methyl)-6-oxo-5,6,7,8-tetrahydroimidazo[1,5-a]pyrazin-1-yl)-3-fluoro-5-(trifluoromethyl)benzamide (I-251)

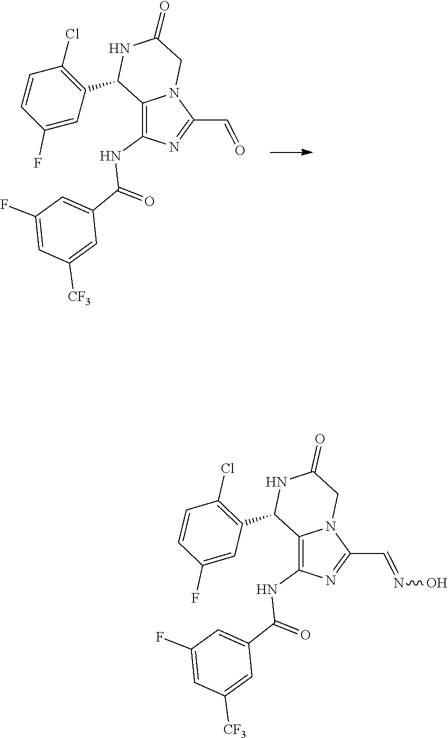 [SMILES: O=Cc1nc(NC(=O)c2cc(F)cc(C(F)(F)F)c2)c2n1CC(=O)N[C@H]2c1cc(F)ccc1Cl ; O=C1Cn2c(C=NO)nc(NC(=O)c3cc(F)cc(C(F)(F)F)c3)c2[C@H](c2cc(F)ccc2Cl)N1]

A flame-dried round bottom flask was charged with (S)—N-(8-(2-chloro-5-fluorophenyl)-3-formyl-6-oxo-5,6,7,8-tetrahydroimidazo[1,5-a]pyrazin-1-yl)-3-fluoro-5-(trifluoromethyl)benzamide (187 mg) and hydroxylamine hydrochloride (78.2 mg) in EtOH (10 mL). The reaction mixture was stirred at 30° C. in an oil bath for 1 hour. The reaction mixture was concentrated under reduced pressure, and the residue was purified by reverse phase column chromatography (C18, 30 g column, mobile phase A: 10 mM ammonium bicarbonate solution, B: acetonitrile; gradient: 0-100% B) to give the desired product (14.3 mg) as a white powder. LCMS: RT 1.99 min, [M+H]⁺ 514.2, LCMS method W. ¹H NMR: (400 MHz, DMSO-d6) δ 11.73 (s, 1H), 10.44 (br s, 1H), 8.92 (d, J=2.1 Hz, 1H), 8.06 (s, 1H), 7.92 (d, J=8.5 Hz, 1H), 7.82 (s, 1H, overlap), 7.81 (dd, J=8.6, 1.5 Hz, 1H, overlap), 7.35 (dd, J=9.5, 5.0 Hz, 1H), 7.12-7.05 (m, 2H), 6.01 (s, 1H), 5.01 (d, J=18.5 Hz, 1H), 4.92 (dd, J=18.2, 1.4 Hz, 1H).

Compound I-679 was also prepared according to the methods of Example 44. Mass spectrometry and ¹H NMR characterization are provided for this compound in Table 1.

### Example 45

### N-(4-(2-chloro-5-fluorophenyl)-1-(2-(methylamino)-2-oxoethyl)-6-oxo-4,5,6,7-tetrahydro-1H-pyrazolo[4,3-c]pyridin-3-yl)-3-fluoro-5-(trifluoromethyl)benzamide (I-263)

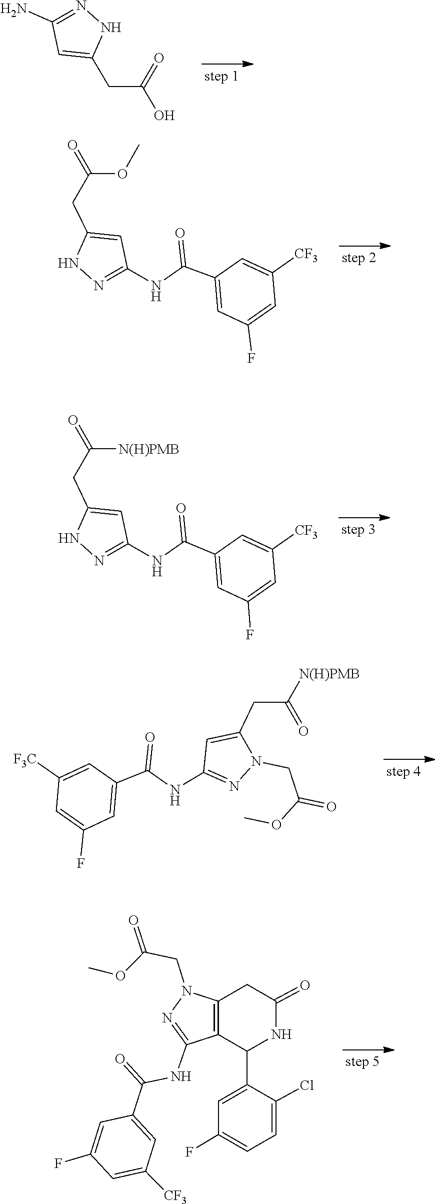

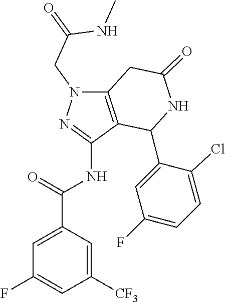 [SMILES: CNC(=O)Cn1nc(NC(=O)c2cc(F)cc(C(F)(F)F)c2)c2c1CC(=O)NC2c1cc(F)ccc1Cl]

### Step 1. methyl 2-(3-(3-fluoro-5-(trifluoromethyl)benzamido)-1H-pyrazol-5-yl)acetate

A 30-mL vial equipped with a stir bar was charged with 2-(3-amino-1H-pyrazol-5-yl) acetic acid (500 mg) in MeOH (6 mL). Thionyl chloride (5.06 g) was added in one portion. The reaction mixture was stirred at 60° C. for 1 hour and concentrated under reduced pressure. The crude methyl ester was diluted with dichloromethane (25 mL). Pyridine (4.20 g) was added, followed by 3-fluoro-5-(trifluoromethyl)benzoyl chloride (1.605 g) in portions. The solution was stirred at room temperature for 1 hour. The reaction mixture was concentrated under reduced pressure. The residue was purified by reverse phase column chromatography (C18, 30 g; mobile phase A: 10 mM ammonium formate solution; mobile phase B: acetonitrile). The fractions containing the product were combined, concentrated, and extracted with ethyl acetate. The organic layers were combined, dried over Na₂SO₄ and concentrated to furnish the desired product (750 mg). LCMS: RT 1.04 min, [M+H]⁺ 346.3, LCMS method U.

### Step 2. 3-fluoro-N-(5-(2-((4-methoxybenzyl)amino)-2-oxoethyl)-1H-pyrazol-3-yl)-5-(trifluoromethyl)benzamide

A 30 mL vial equipped with a stir bar was charged with methyl 2-(3-(3-fluoro-5-(trifluoromethyl)benzamido)-1H-pyrazol-5-yl)acetate (700 mg) in THF (15 mL). (4-Methoxyphenyl)methanamine (417.2 mg) was added in one portion. The reaction mixture was stirred at 45° C. for 8 hours. Additional (4-methoxyphenyl)methanamine (834.4 mg) was added to the reaction mixture, the temperature was raised to 80° C. and the reaction mixture was stirred overnight. After cooling to room temperature the reaction mixture was concentrated under reduced pressure. The residue was purified by reverse phase column chromatography (C18, 12 g; mobile phase A: 10 mM ammonium formate solution; mobile phase B: acetonitrile). The fractions containing the desired product were collected, concentrated and extracted with dichloromethane. The organic layers were combined, dried over Na₂SO₄ and concentrated to give the desired product (500 mg). LCMS: RT 1.12 min; [M+H]⁺ 451.3, LCMS method U.

### Step 3. methyl 2-(3-(3-fluoro-5-(trifluoromethyl)benzamido)-5-(2-((4-methoxybenzyl)amino)-2-oxoethyl)-1H-pyrazol-1-yl)acetate

A 30 mL vial equipped with a stir bar was charged with 3-fluoro-N-(5-(2-((4-methoxybenzyl)amino)-2-oxoethyl)-1H-pyrazol-3-yl)-5-(trifluoromethyl)benzamide (165.0 mg) in acetonitrile (15 mL). To the reaction mixture was added potassium carbonate (151.9 mg) and methyl 2-bromoacetate (112.1 mg), and the reaction mixture was stirred at 50° C. for 1 hour. The reaction mixture was diluted with dichloromethane and washed with brine. The organic layer was dried over Na₂SO₄, concentrated under reduced pressure, and re-dissolved in dichloromethane to reach a final concentration of the desired product at 20 mg/mL. This solution was used in the next step without further purification. LCMS: RT 1.21 min, [M+H]⁺ 523.3, LCMS method U.

### Step 4. methyl 2-(4-(2-chloro-5-fluorophenyl)-3-(3-fluoro-5-(trifluoromethyl)benzamido)-6-oxo-4,5,6,7-tetrahydro-1H-pyrazolo[4,3-c]pyridin-1-yl)acetate

In a screw-cap vial equipped with a stir bar was charged with a dichloromethane solution of methyl 2-(3-(3-fluoro-5-(trifluoromethyl)benzamido)-5-(2-((4-methoxybenzyl)amino)-2-oxoethyl)-1H-pyrazol-1-yl)acetate (20 mg/mL, 1.5 mL). The solution was concentrated under reduced pressure to remove dichloromethane. 2-Chloro-5-fluorobenzaldehyde (14 mg) was added, followed by Eaton's reagent (1.0 mL). The reaction mixture was sealed and heated in an oil bath (preheated to 90° C.) for 30 minutes. After cooling to room temperature the reaction mixture was directly purified by reverse phase column chromatography (C18, 30 g; mobile phase A: 10 mM ammonium formate solution; mobile phase B: acetonitrile; gradient: 0-100% B) to furnish the desired product as a white powder. The white powder was further purified using HILIC column chromatography (12 g), and the desired product was eluted with 100% acetonitrile gradually decreasing to 70% acetonitrile (and 30% 10 mM ammonium formate solution in water) to furnish the desired product (7 mg) as a white powder. LCMS: RT 1.11 min, [M+H]⁺ 543.1, LCMS method U.

### Step 5. N-(4-(2-chloro-5-fluorophenyl)-1-(2-(methylamino)-2-oxoethyl)-6-oxo-4,5,6,7-tetrahydro-1H-pyrazolo[4,3-c]pyridin-3-yl)-3-fluoro-5-(trifluoromethyl)benzamide

In a round bottom flask equipped with a stir bar was charged methyl 2-(4-(2-chloro-5-fluorophenyl)-3-(3-fluoro-5-(trifluoromethyl)benzamido)-6-oxo-4,5,6,7-tetrahydro-1H-pyrazolo[4,3-c]pyridin-1-yl)acetate (7 mg) in toluene (10 mL). To the reaction mixture was added methylamine hydrochloride (0.1 mL, 33 wt % in EtOH) and trimethylaluminum in PhMe (0.1 mL, 2 M). The reaction mixture was heated at 80° C. for 30 minutes. The reaction was quenched by the addition of saturated Rochelle salt solution and ethyl acetate. The solution was stirred overnight until phase separation was observed. The phases were separated, and the organic layer was washed with brine. The organic layer was concentrated under reduced pressure, and the crude residue was purified by reverse phase column chromatography (C18, 30 g; mobile phase A: 10 mM ammonium formate solution; mobile phase B: acetonitrile; gradient: 0-100% B) to furnish the desired product (2.4 mg) as a white powder. LCMS: RT 2.38 min, [M+H]⁺ 542.2, LCMS method W. ¹H NMR: (400 MHz, DMSO-d6) δ 10.56 (s, 1H), 8.38 (d, J=2.1 Hz, 1H), 8.15 (app q, J=8.4 Hz, 1H), 7.92 (d, J=8.4 Hz, 1H), 7.83 (overlapping s, 1H), 7.82 (overlapping d, J=10.6 Hz, 1H), 7.29 (dd, J=8.8, 5.2 Hz, 1H), 7.05 (ddd, J=8.7, 8.0, 3.1 Hz, 1H), 6.88 (dd, J=9.4, 3.1 Hz, 1H), 5.80 (d, J=2.2 Hz, 1H), 4.70 (s, 2H), 3.70 (dd, J=20.8, 2.4 Hz, 1H), 3.63 (dd, J=21.0, 2.8 Hz, 1H), 2.64 (d, J=4.6 Hz, 3H).

### Example 46

### N-(3-(2-chloro-5-fluorophenyl)-7-methyl-5,8-dioxo-4,5,5a,6,7,8-hexahydro-3H-1,2a1,4,7-tetraazaacenaphthylen-2-yl)-3-fluoro-5-(trifluoromethyl)benzamide (I-266)

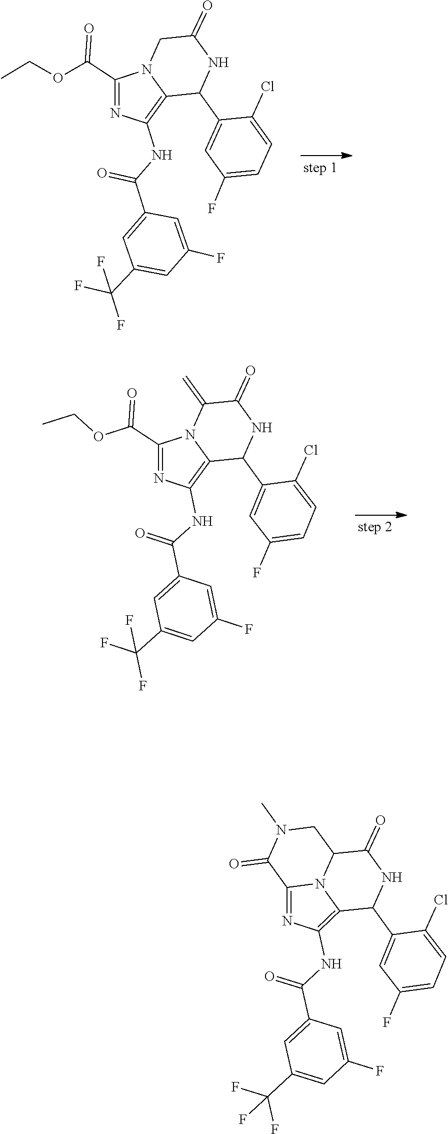 [SMILES: CCOC(=O)c1nc(NC(=O)c2cc(F)cc(C(F)(F)F)c2)c2n1CC(=O)NC2c1cc(F)ccc1Cl ; C=C1C(=O)NC(c2cc(F)ccc2Cl)c2c(NC(=O)c3cc(F)cc(C(F)(F)F)c3)nc(C(=O)OCC)n21 ; CN1CC2C(=O)NC(c3cc(F)ccc3Cl)c3c(NC(=O)c4cc(F)cc(C(F)(F)F)c4)nc(n32)C1=O]

### Step 1. ethyl 8-(2-chloro-5-fluorophenyl)-1-(3-fluoro-5-(trifluoromethyl)benzamido)-5-methylene-6-oxo-5,6,7,8-tetrahydroimidazo[1,5-a]pyrazine-3-carboxylate

A 100 mL round bottom flask vial was charged with ethyl 8-(2-chloro-5-fluorophenyl)-1-(3-fluoro-5-(trifluoromethyl)benzamido)-6-oxo-5,6,7,8-tetrahydroimidazo [1,5-a]pyrazine-3-carboxylate (1.0 g). DMF (6 mL) was added, followed by N,N,N,N-tetramethyldiaminomethane (6.3 mL) and Ac₂O (6 mL). The vial was sealed and heated at 100° C. for 1 hour. The reaction was cooled to room temperature and diluted with ethyl acetate (30 mL) and water (100 mL). The organic layer was washed three times with water, dried over Na₂SO₄ and concentrated under reduced pressure. The crude material was dissolved in dichloromethane and loaded on a 30 g SiO₂ column for purification (eluent hexanes/ethyl acetate 100:0 to 60:40) to give the desired product (727 mg) as a yellow solid. LCMS: RT 1.64 min, [M+H]⁺ 555.1, LCMS method Q.

### Step 2. N-(3-(2-chloro-5-fluorophenyl)-7-methyl-5,8-dioxo-4,5,5a,6,7,8-hexahydro-3H-1,2a1,4,7-tetraazaacenaphthylen-2-yl)-3-fluoro-5-(trifluoromethyl)benzamide

A microwave vial was charged with ethyl 8-(2-chloro-5-fluorophenyl)-1-(3-fluoro-5-(trifluoromethyl)benzamido)-5-methylene-6-oxo-5,6,7,8-tetrahydroimidazo[1,5-a]pyrazine-3-carboxylate (20 mg) and DABCO (0.81 mg). Methylamine (0.18 mL, 2 M in THF) was added and the reaction mixture was stirred for 1 hour at room temperature. The solution was concentrated under reduced pressure and the crude material was dissolved in DMF (1 mL) and loaded one a 30 g reverse phase column for purification (10 mM ammonium formate solution:acetonitrile 95:5 to 35:65) to give the desired product (5.9 mg). LCMS: RT 1.43 min, [M+H]⁺ 540.2, LCMS method Q. ¹H NMR (400 MHz, DMSO-d6) δ 10.65 (overlapping br. s, 0.5H), 10.55 (overlapping br. s, 0.5H), 8.95 (br. s, 0.5H), 8.89 (br. s, 0.5H), 7.90 (d, J=7.7 Hz, 1H), 7.78-7.62 (m, 2H), 7.36 (br. s, 0.5H), 7.21 (submerged br. s, 0.5H), 7.17 (overlapping dd, J=9.3, 1.8 Hz, 1H), 7.05-6.96 (m, 1H), 6.24 (br. s, 0.5H), 5.85 (br. s, 0.5H), 5.43-5.35 (m, 1H), 4.14-4.03 (m, 0.5H), 4.00-3.88 (m, 1.5H), 3.08 (s, 3H). 2 diastereomers in 1:1 ratio.

### Example 47

### (S)—N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide (I-271 or I-272) and (R)—N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide (I-271 or I-272)

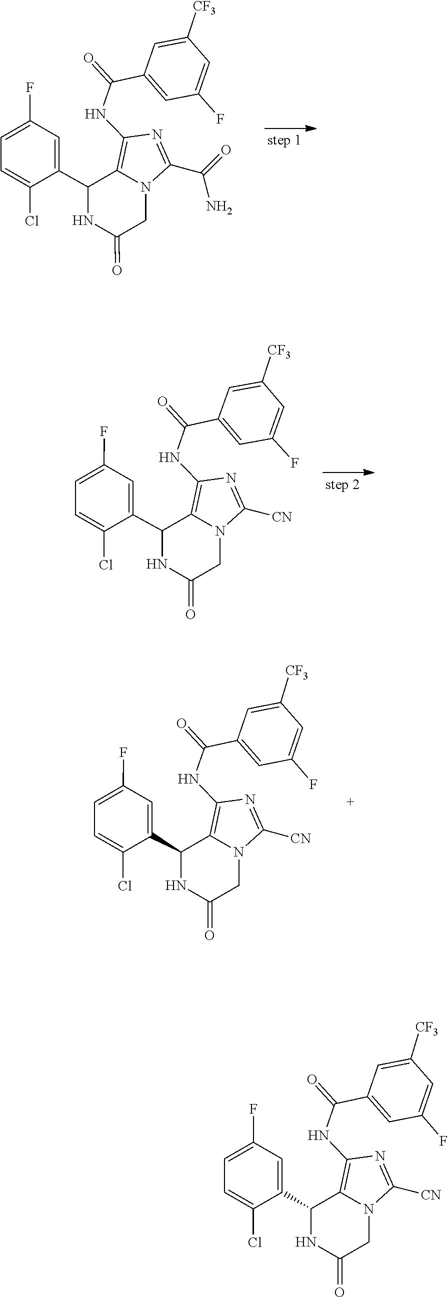 [SMILES: NC(=O)c1nc(NC(=O)c2cc(F)cc(C(F)(F)F)c2)c2n1CC(=O)NC2c1cc(F)ccc1Cl ; N#Cc1nc(NC(=O)c2cc(F)cc(C(F)(F)F)c2)c2n1CC(=O)NC2c1cc(F)ccc1Cl ; N#Cc1nc(NC(=O)c2cc(F)cc(C(F)(F)F)c2)c2n1CC(=O)N[C@H]2c1cc(F)ccc1Cl ; N#Cc1nc(NC(=O)c2cc(F)cc(C(F)(F)F)c2)c2n1CC(=O)N[C@@H]2c1cc(F)ccc1Cl]

### Step 1. N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide

In a flame-dried microwave vial, to the solution of 8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxamide (30 mg) in dichloromethane (0.5 mL) at 0° C. was added Et₃N (16 μL), followed by TFAA (49 μL). The resulting solution was stirred at room temperature for 1 hour. The reaction was quenched with saturated aqueous solution of NaHCO₃ and directly loaded on a reverse phase C18 column (12 g), eluent: 10 mM ammonium formate solution:acetonitrile; gradient: 0-100% acetonitrile) to afford the desired product (19 mg) as a white solid. LC-MS RT 2.25 min, [M+H]⁺ 496.2, LCMS method V. ¹H NMR (400 MHz, DMSO-d6) δ 10.64 (br. s, 1H), 9.06 (s, 1H), 7.95 (d, J=8.5 Hz, 1H), 7.80 (overlapping d, J=8.4 Hz, 1H), 7.79 (overlapping s, 1H), 7.36 (dd, J=8.5, 5.2 Hz, 1H), 7.20-7.03 (m, 2H), 6.02 (s, 1H), 5.11 (d, J=12.1 Hz, 1H), 5.03 (d, J=12.1 Hz, 1H).

### Step 2. (S)—N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide and (R)—N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide

N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide (12.4 mg) was chirally resolved using the chiral SFC condition below to give both enantiomers. Column: Regis Whelk 0-1 (S,S) 21×250 mm, mobile phase: 30% methanol in CO₂, flow rate: 70 mL/min, sample: 12.4 mg of sample was dissolved in 2 mL methanol+2 mL dichloromethane, injection: 2.0 mL, detection: 220 nm. Peak 1: 4.9 mg. Chiral SFC RT 0.9 min. LCMS: RT 1.15 min, [M+H]⁺ 496.0, LCMS method G. Peak 2, 5.2 mg. Chiral SFC RT 1.23 min. LCMS: RT 1.15 min, [M+H]⁺ 496.0, LCMS method G.

### Example 48

### (5R,8S)-8-(2-chloro-5-fluorophenyl)-1-(3-fluoro-5-(trifluoromethyl)benzamido)-5-(methoxymethyl)-N-methyl-6-oxo-5,6,7,8-tetrahydroimidazo[1,5-a]pyrazine-3-carboxamide (I-289)

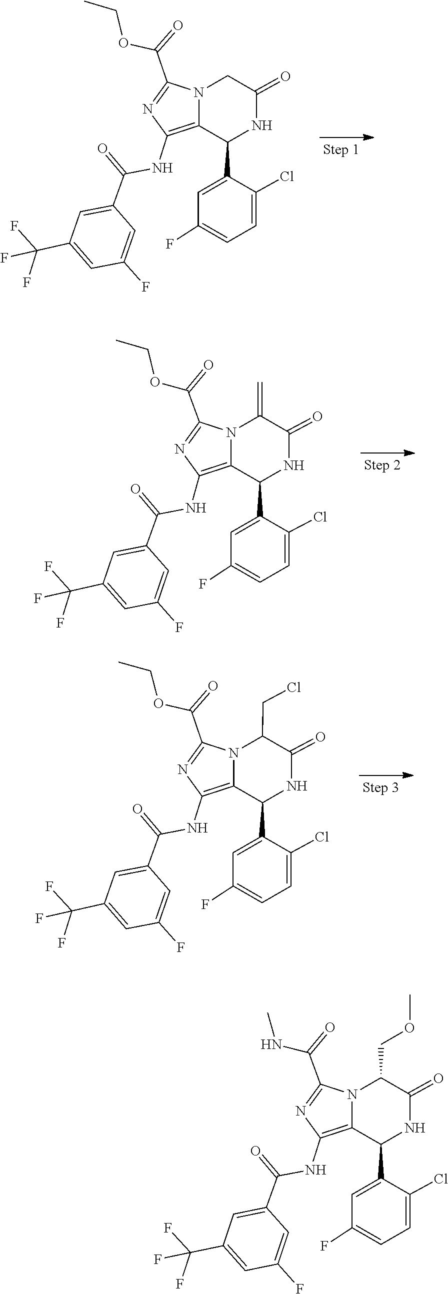 [SMILES: CCOC(=O)c1nc(NC(=O)c2cc(F)cc(C(F)(F)F)c2)c2n1CC(=O)N[C@H]2c1cc(F)ccc1Cl ; C=C1C(=O)N[C@@H](c2cc(F)ccc2Cl)c2c(NC(=O)c3cc(F)cc(C(F)(F)F)c3)nc(C(=O)OCC)n21 ; CCOC(=O)c1nc(NC(=O)c2cc(F)cc(C(F)(F)F)c2)c2n1C(CCl)C(=O)N[C@H]2c1cc(F)ccc1Cl ; CNC(=O)c1nc(NC(=O)c2cc(F)cc(C(F)(F)F)c2)c2n1[C@H](COC)C(=O)N[C@H]2c1cc(F)ccc1Cl]

### Step 1. ethyl (S)-8-(2-chloro-5-fluorophenyl)-1-(3-fluoro-5-(trifluoromethyl)benzamido)-5-methylene-6-oxo-5,6,7,8-tetrahydroimidazo[1,5-a]pyrazine-3-carboxylate

A round bottom flask was charged with ethyl (S)-8-(2-chloro-5-fluorophenyl)-1-(3-fluoro-5-(trifluoromethyl)benzamido)-6-oxo-5,6,7,8-tetrahydroimidazo[1,5-a]pyrazine-3-carboxylate (1500 mg,). DMF (9 mL) was added, followed by N,N,N,N-tetramethyldiaminomethane (5.647 g) and Ac₂O (9 mL) at 0° C. The flask was heated at 100° C. for 2 hours. The reaction was cooled to room temperature and diluted with ethyl acetate and NH₄Cl solution. The aqueous layer was extracted twice with ethyl acetate. The organic layers were combined, dried over Na₂SO₄ and concentrated under reduced pressure. The crude residue was purified on a normal phase silica gel column (60 g); eluent: hexane:ethyl acetate 1:0 to 0:1 to give the desired product (790 mg). LCMS: RT 1.24 min, [M+H]⁺ 555.2, LCMS method U.

### Step 2. ethyl (8S)-8-(2-chloro-5-fluorophenyl)-5-(chloromethyl)-1-(3-fluoro-5-(trifluoromethyl)benzamido)-6-oxo-5,6,7,8-tetrahydroimidazo[1,5-a]pyrazine-3-carboxylate

A 20 mL microwave vial was charged with ethyl (S)-8-(2-chloro-5-fluorophenyl)-1-(3-fluoro-5-(trifluoromethyl)benzamido)-5-methylene-6-oxo-5,6,7,8-tetrahydroimidazo[1,5-a]pyrazine-3-carboxylate (100 mg), THF (5 mL) and 12 N hydrogen chloride (1 mL). The reaction was stirred at room temperature for 2 hours. The reaction solution was concentrated. The residue was suspended in acetonitrile/water, and freeze-dried to afford the desired product (110 mg). LCMS: RT 1.51 and 1.53 min (2 diastereomers), [M+H]⁺ 591.1, LCMS method S.

### Step 3. (5R,8S)-8-(2-chloro-5-fluorophenyl)-1-(3-fluoro-5-(trifluoromethyl)benzamido)-5-(methoxymethyl)-N-methyl-6-oxo-5,6,7,8-tetrahydroimidazo[1,5-a]pyrazine-3-carboxamide

A flame-dried 5 mL microwave vial was charged with ethyl (8S)-8-(2-chloro-5-fluorophenyl)-5-(chloromethyl)-1-(3-fluoro-5-(trifluoromethyl)benzamido)-6-oxo-5,6,7,8-tetrahydroimidazo[1,5-a]pyrazine-3-carboxylate (104 mg), MeOH (1 mL), and potassium carbonate (29.2 mg). The vial was irradiated under microwave at 100° C. for 20 minutes. After cooling to room temperature, methylamine in water (244 mg, 40% by weight) was added. The vial was then irradiated under microwave at 100° C. for 15 minutes. The solution was concentrated, and the resulting residue was purified on a C18 column (60 g, 10-100% acetonitrile in 10 mM ammonium formate solution) to afford the desired product. The compound was re-purified over C18 column (60 g, 10-100% acetonitrile in 10 mM ammonium formate solution) to afford the desired product (8.6 mg) as a white powder. LCMS: RT 1.37 min, [M+H]⁺ 572.1, LCMS method S. ¹H NMR (400 MHz, DMSO-d6) δ 10.25 (br s, 1H), 8.89 (s, 1H), 8.50 (app q, J=4.2 Hz, 1H), 7.90 (d, J=8.4 Hz, 1H), 7.68 (overlapped s, 1H), 7.67 (overlapped d, J=9.2 Hz, 1H), 7.25 (br s, 1H), 7.18 (dd, J=9.3, 3.0 Hz, 1H), 6.98 (td, J=8.5, 3.1 Hz, 1H), 5.89 (br s, 1H), 5.65 (app t, J=2.1 Hz, 1H), 4.06 (dd, J=10.1, 2.2 Hz, 1H), 3.89 (dd, J=10.1, 2.0 Hz, 1H), 3.20 (s, 3H), 2.77 (d, J=4.8 Hz, 3H). Single diastereomer.

### Example 49

### N-((5S,8S)-8-(2-chloro-5-fluorophenyl)-3-(methylcarbamoyl)-6-oxo-5-(piperazin-1-ylmethyl)-5,6,7,8-tetrahydroimidazo[1,5-a]pyrazin-1-yl)benzo[d]isothiazole-3-carboxamide (I-617) and N-((5R,8S)-8-(2-chloro-5-fluorophenyl)-3-(methylcarbamoyl)-6-oxo-5-(piperazin-1-ylmethyl)-5,6,7,8-tetrahydroimidazo[1,5-a]pyrazin-1-yl)benzo[d]isothiazole-3-carboxamide (I-618)

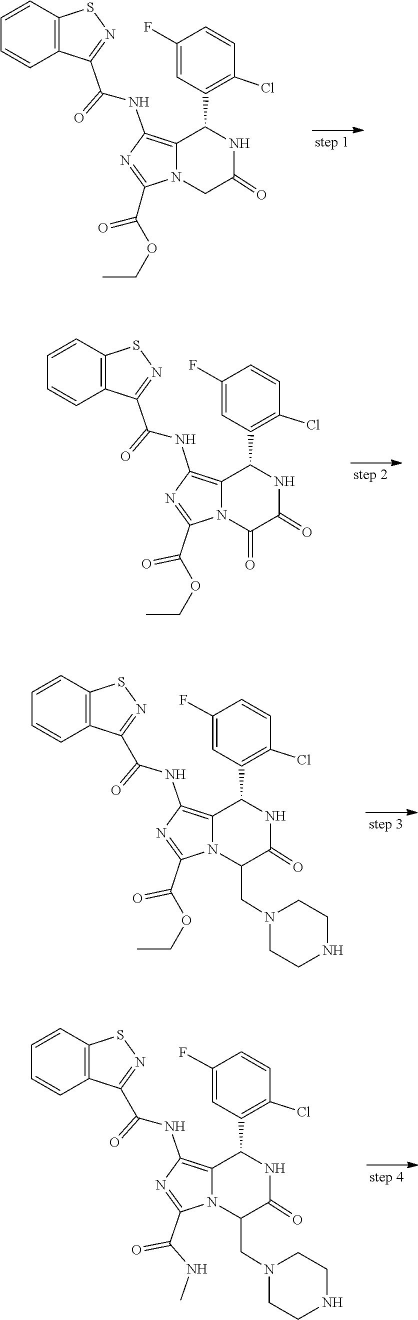 [SMILES: CCOC(=O)c1nc(NC(=O)c2nsc3ccccc23)c2n1CC(=O)N[C@H]2c1cc(F)ccc1Cl ; CCOC(=O)c1nc(NC(=O)c2nsc3ccccc23)c2n1C(=O)C(=O)N[C@H]2c1cc(F)ccc1Cl ; CCOC(=O)c1nc(NC(=O)c2nsc3ccccc23)c2n1C(CN1CCNCC1)C(=O)N[C@H]2c1cc(F)ccc1Cl ; CNC(=O)c1nc(NC(=O)c2nsc3ccccc23)c2n1C(CN1CCNCC1)C(=O)N[C@H]2c1cc(F)ccc1Cl]

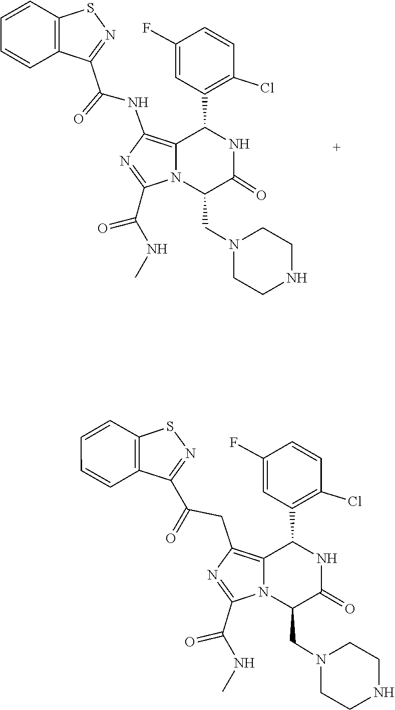 [SMILES: CNC(=O)c1nc(NC(=O)c2nsc3ccccc23)c2n1[C@@H](CN1CCNCC1)C(=O)N[C@H]2c1cc(F)ccc1Cl ; CNC(=O)c1nc(CC(=O)c2nsc3ccccc23)c2n1[C@H](CN1CCNCC1)C(=O)N[C@H]2c1cc(F)ccc1Cl]

### Step 1. ethyl (S)-1-(benzo[d]isothiazole-3-carboxamido)-8-(2-chloro-5-fluorophenyl)-5-methylene-6-oxo-5,6,7,8-tetrahydroimidazo[1,5-a]pyrazine-3-carboxylate

A 100 mL flask was charged with ethyl (S)-1-(benzo[d]isothiazole-3-carboxamido)-8-(2-chloro-5-fluorophenyl)-6-oxo-5,6,7,8-tetrahydroimidazo[1,5-a]pyrazine-3-carboxylate (500 mg). DMF (3 mL) was added, followed by N,N,N,N-tetramethyldiaminomethane (2.49 g) and slow addition of Ac₂O (3 mL). The flask was sealed and heated to 100° C. for 90 minutes. The reaction media was cooled down to room temperature and diluted with ethyl acetate (50 mL) and water (150 mL). The organic layer was washed twice with water. The organic layer was dried over Na₂SO₄ and concentrated under reduced pressure. The crude was dissolved in DCM and loaded on a 30 g SiO₂ column for purification (Eluant: hexanes:ethyl acetate 100:0 to 60:40) to give the desired product (341 mg) as a dark yellow solid. LCMS RT 1.60 min, [M+H]⁺ 526.0, LCMS method Q.

### Step 2. ethyl (8S)-1-(benzo[d]isothiazole-3-carboxamido)-8-(2-chloro-5-fluorophenyl)-6-oxo-5-(piperazin-1-ylmethyl)-5,6,7,8-tetrahydroimidazo[1,5-a]pyrazine-3-carboxylate

A reaction vial was charged with ethyl (S)-1-(benzo[d]isothiazole-3-carboxamido)-8-(2-chloro-5-fluorophenyl)-5-methylene-6-oxo-5,6,7,8-tetrahydroimidazo[1,5-a]pyrazine-3-carboxylate (200 mg) and piperazine (131 mg) before being evacuated and purged with nitrogen three times. THF (5 mL) was added, and the mixture was stirred at 25° C. for 1 hour. The resulting crude material was purified by reverse phase chromatography (water/acetonitrile) to give the desired product (300 mg) as an off-white amorphous solid. It was further purified by prep-HPLC (Column: XBridge Shield RP18 OBD column, 30*150 mm, 5 μm; mobile phase A: 10 mM NH₄HCO₃ solution+0.1% NH₃·H₂O, mobile phase B: acetonitrile; flow rate: 60 mL/min; gradient: 25% to 40% B in 11 minutes, wavelength: 220 nm; RT: 10.45 min) to give the desired product (120 mg) as an off-white amorphous solid. LCMS: RT 0.953 min, [M+H]⁺ 612.0, LCMS method P.

### Step 3. N-((8S)-8-(2-chloro-5-fluorophenyl)-3-(methylcarbamoyl)-6-oxo-5-(piperazin-1-ylmethyl)-5,6,7,8-tetrahydroimidazo[1,5-a]pyrazin-1-yl)benzo[d]isothiazole-3-carboxamide

A reaction vial was charged with ethyl (8S)-1-(benzo[d]isothiazole-3-carboxamido)-8-(2-chloro-5-fluorophenyl)-6-oxo-5-(piperazin-1-ylmethyl)-5,6,7,8-tetrahydroimidazo[1,5-a]pyrazine-3-carboxylate (110 mg) and THF (4 mL) before being evacuated and purged with nitrogen three times. AlMe₃ (38.9 mg) was added at 0° C., and the mixture was stirred for 0.5 hour. Methylamine (2 M in THF, 0.45 mL) was added, and the mixture was stirred at 50° C. for 16 hours. After cooling to room temperature, the reaction mixture was partitioned between CH₂Cl₂ and water. The aqueous layer was extracted with CH₂Cl₂. The organic layers were combined and filtered to remove the solid. The filtrate was dried over Na₂SO₄ and concentrated under vacuum. The resulting crude material was purified by prep-HPLC (Column: XBridge Prep OBD C18 Column, 30*150 mm, 5 μm; mobile phase A: 10 mM NH₄HCO₃ solution, mobile phase B: acetonitrile; flow rate: 60 mL/min; gradient: 25% B to 35% B in 8 minutes, then 35% B; wavelength: 220 nm; RT: 6.17 min) to give the desired product (40.0 mg) as an off-white amorphous solid. LCMS: RT 0.953 min, [M+H]⁺ 597.15, LCMS method P.

### Step 4. N-((5S,8S)-8-(2-chloro-5-fluorophenyl)-3-(methylcarbamoyl)-6-oxo-5-(piperazin-1-ylmethyl)-5,6,7,8-tetrahydroimidazo[1,5-a]pyrazin-1-yl)benzo[d]isothiazole-3-carboxamide and N-((5R,8S)-8-(2-chloro-5-fluorophenyl)-3-(methylcarbamoyl)-6-oxo-5-(piperazin-1-ylmethyl)-5,6,7,8-tetrahydroimidazo[1,5-a]pyrazin-1-yl)benzo[d]isothiazole-3-carboxamide

N-((8S)-8-(2-chloro-5-fluorophenyl)-3-(methylcarbamoyl)-6-oxo-5-(piperazin-1-ylmethyl)-5,6,7,8-tetrahydroimidazo[1,5-a]pyrazin-1-yl)benzo[d]isothiazole-3-carboxamide (40 mg) was chirally resolved by prep-CHIRAL-HPLC (Column: CHIRALPAK IH, 2*25 cm, 5 μm; mobile phase A: hexane (with 0.2% triethylamine), mobile phase B: EtOH:dichloromethane 1:1; flow rate: 20 mL/min; gradient: 50% B isocratic, wavelength: 220/254 nm; sample solvent: EtOH:dichloromethane 1:1; injection volume: 1.6 mL) to give the two stereoisomers, both as an off-white amorphous solid.

Compound I-618, Peak 1: 9.8 mg. Chiral HPLC RT: 5.18 min. LCMS: RT 1.325 min, [M+H]⁺ 597.1, LCMS method M. ¹H NMR (400 MHz, DMSO-d6) δ 10.22 (s, 1H), 9.03 (d, J=3.0 Hz, 1H), 8.52 (dd, J=15.0, 6.6 Hz, 2H), 8.30 (d, J=8.2 Hz, 1H), 7.74 (dd, J=9.1, 3.1 Hz, 1H), 7.67 (ddd, J=8.2, 6.9, 1.2 Hz, 1H), 7.58 (dd, J=8.3, 6.8 Hz, 1H), 7.19 (dd, J=8.9, 5.1 Hz, 1H), 7.09 (td, J=8.4, 3.1 Hz, 1H), 6.32 (d, J=2.3 Hz, 1H), 5.45 (s, 1H), 3.21 (td, J=13.6, 12.9, 4.3 Hz, 1H), 3.11 (dd, J=14.3, 4.1 Hz, 1H), 2.79 (d, J=4.7 Hz, 3H), 2.63 (s, 4H), 2.32 (s, 2H), 2.14 (s, 2H), 1.24 (s, 1H).

Compound I-617, Peak 2: 10.4 mg. Chiral HPLC RT: 14.07 min. LCMS: RT 1.295 min, [M+H]⁺ 597.1, LCMS method M. ¹H NMR (400 MHz, DMSO-d6) δ 10.12 (s, 1H), 8.85 (s, 1H), 8.60 (d, J=8.2 Hz, 1H), 8.47 (d, J=5.2 Hz, 1H), 8.27 (d, J=8.2 Hz, 1H), 7.67 (ddd, J=8.3, 7.0, 1.3 Hz, 1H), 7.60 (t, J=7.6 Hz, 1H), 7.12 (s, 2H), 6.85 (dd, J=9.1, 6.3 Hz, 1H), 6.44 (s, 1H), 5.56 (d, J=3.0 Hz, 1H), 3.08 (d, J=13.7 Hz, 1H), 2.97 (d, J=14.0 Hz, 1H), 2.78 (d, J=4.8 Hz, 5H), 2.70-2.53 (m, 2H), 2.32 (s, 2H), 2.00 (s, 2H).

Additional compounds prepared according to the methods of Example 49 are listed in Table 13 below. Corresponding ¹H NMR and mass spectrometry characterization for these compounds are described in Table 1. Certain compounds in Table 13 below were prepared with other compounds whose preparation is described in the Examples herein.

TABLE 13
Additional Exemplary Compounds
Compound

I-227
I-228
I-229
I-264
I-265
I-288
I-289
I-292
I-293
I-294
I-295
I-296
I-297
I-302
I-303
I-315
I-316
I-317
I-396
I-397
I-425
I-469
I-470
I-471
I-557
I-567
I-568
I-569
I-570
I-571
I-572
I-573
I-574
I-617
I-618
I-689
I-694
I-695
I-696
I-701
I-702

### Example 50

### (S)—N-(8-(2-chloro-5-fluorophenyl)-3-(N-methylcarbamimidoyl)-6-oxo-5,6,7,8-tetrahydroimidazo[1,5-a]pyrazin-1-yl)-3-fluoro-5-(trifluoromethyl)benzamide (I-290 or I-291) and (R)—N-(8-(2-chloro-5-fluorophenyl)-3-(N-methylcarbamimidoyl)-6-oxo-5,6,7,8-tetrahydroimidazo[1,5-a]pyrazin-1-yl)-3-fluoro-5-(trifluoromethyl)benzamide (I-290 or I-291)

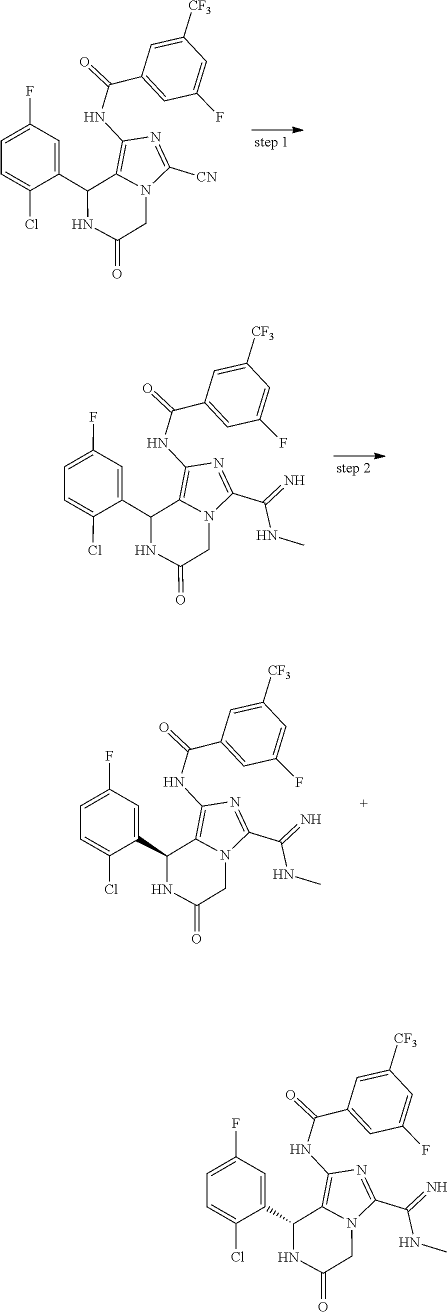 [SMILES: N#Cc1nc(NC(=O)c2cc(F)cc(C(F)(F)F)c2)c2n1CC(=O)NC2c1cc(F)ccc1Cl ; CNC(=N)c1nc(NC(=O)c2cc(F)cc(C(F)(F)F)c2)c2n1CC(=O)NC2c1cc(F)ccc1Cl ; CNC(=N)c1nc(NC(=O)c2cc(F)cc(C(F)(F)F)c2)c2n1CC(=O)N[C@H]2c1cc(F)ccc1Cl ; CNC(=N)c1nc(NC(=O)c2cc(F)cc(C(F)(F)F)c2)c2n1CC(=O)N[C@@H]2c1cc(F)ccc1Cl]

### Step 1. N-(8-(2-chloro-5-fluorophenyl)-3-(N-methylcarbamimidoyl)-6-oxo-5,6,7,8-tetrahydroimidazo[1,5-a]pyrazin-1-yl)-3-fluoro-5-(trifluoromethyl)benzamide

In a microwave vial, to a solution of MeNH₂·HCl (0.10 g) in toluene (0.4 mL) at 0° C. was added a 2 M solution of Me₃Al in heptane (0.78 mL). The resulting solution was stirred at room temperature for 1 hour. In a separate flame-dried microwave vial, N-(8-(2-chloro-5-fluorophenyl)-3-cyano-6-oxo-5,6,7,8-tetrahydroimidazo[1,5-a]pyrazin-1-yl)-3-fluoro-5-(trifluoromethyl)benzamide (70 mg) was dissolved in toluene (0.4 mL) and to it the solution from the previous vial (methylamine: trimethylaluminum complex) was added slowly. The vial was sealed and heated at 85° C. for 20 hours. The reaction mixture was passed through a short Celite plug and rinsed with plenty of methanol. The filtrate was concentrated and loaded on a reverse phase C18 column (30 g), eluent: 10 mM ammonium formate solution (A) and acetonitrile (B), gradient: 0-100% B to afford the desired product (19 mg) as an off-white solid. LC-MS RT 1.56 min, [M+H]⁺ 527.3, LCMS method V. ¹H NMR (400 MHz, DMSO-d₆) δ 10.43 (br. s, 1H), 8.96 (d, J=7.9 Hz, 1H), 8.26 (d, J=8.9 Hz, 1H), 7.93 (d, J=8.6 Hz, 1H), 7.84 (overlapping s, 1H), 7.82 (overlapping br. d, J=8.6 Hz, 1H), 7.37 (dd, J=8.7, 5.7 Hz, 1H), 7.16-7.04 (m, 2H), 6.03 (d, J=4.5 Hz, 1H), 5.27 (dd, J=18.5, 6.6 Hz, 1H), 5.07 (d, J=18.3 Hz, 1H), 2.93 (d, J=4.0 Hz, 3H); contains 4% of DMF (2.73 & 2.89 ppm).

### Step 2. (S)—N-(8-(2-chloro-5-fluorophenyl)-3-(N-methylcarbamimidoyl)-6-oxo-5,6,7,8-tetrahydroimidazo[1,5-a]pyrazin-1-yl)-3-fluoro-5-(trifluoromethyl)benzamide and (R)—N-(8-(2-chloro-5-fluorophenyl)-3-(N-methylcarbamimidoyl)-6-oxo-5,6,7,8-tetrahydroimidazo[1,5-a]pyrazin-1-yl)-3-fluoro-5-(trifluoromethyl)benzamide

N-(8-(2-chloro-5-fluorophenyl)-3-(N-methylcarbamimidoyl)-6-oxo-5,6,7,8-tetrahydroimidazo[1,5-a]pyrazin-1-yl)-3-fluoro-5-(trifluoromethyl)benzamide (22 mg) was chirally resolved using the chiral SFC conditions below to give the two enantiomers. Column: Nanomicro AS-5H 21×250 mm, mobile phase: 35% methanol+0.25% diethylamine in CO₂, flow rate: 70 mL/min, sample was dissolved in 2 mL methanol+2 mL dichloromethane, injection: 2 mL, detection: 254 nm.

Peak 1, 9.1 mg. Chiral SFC RT 0.81 min. LCMS: RT 0.725 min, [M−H]⁻ 525.2, LCMS method G.

Peak 2, 8.2 mg. Chiral SFC RT 2.17 min. LCMS: RT 0.725 min, [M−H]⁻ 525.2, LCMS method G.

Additional compounds prepared according to the methods of Example 50 are listed in Table 14 below. Corresponding ¹H NMR and mass spectrometry characterization for these compounds are described in Table 1. Certain compounds in Table 14 below were prepared with other compounds whose preparation is described in the Examples herein.

TABLE 14
Additional Exemplary Compounds
Compound

I-267
I-268

### Example 51

### (R)—N-(8-(2-chloro-5-fluorophenyl)-3-((methyl-d3)carbamoyl)-6-oxo-5,6,7,8-tetrahydroimidazo[1,5-a]pyrazin-1-yl-8-d)-5-fluorobenzo[d]isothiazole-3-carboxamide (I-589) and (S)—N-(8-(2-chloro-5-fluorophenyl)-3-((methyl-d3)carbamoyl)-6-oxo-5,6,7,8-tetrahydroimidazo[1,5-a]pyrazin-1-yl-8-d)-5-fluorobenzo[d]isothiazole-3-carboxamide (I-590)

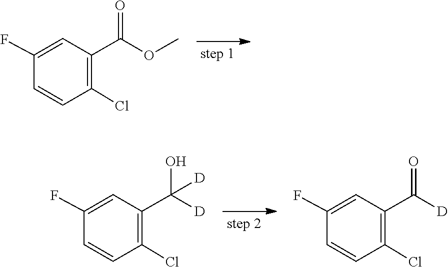

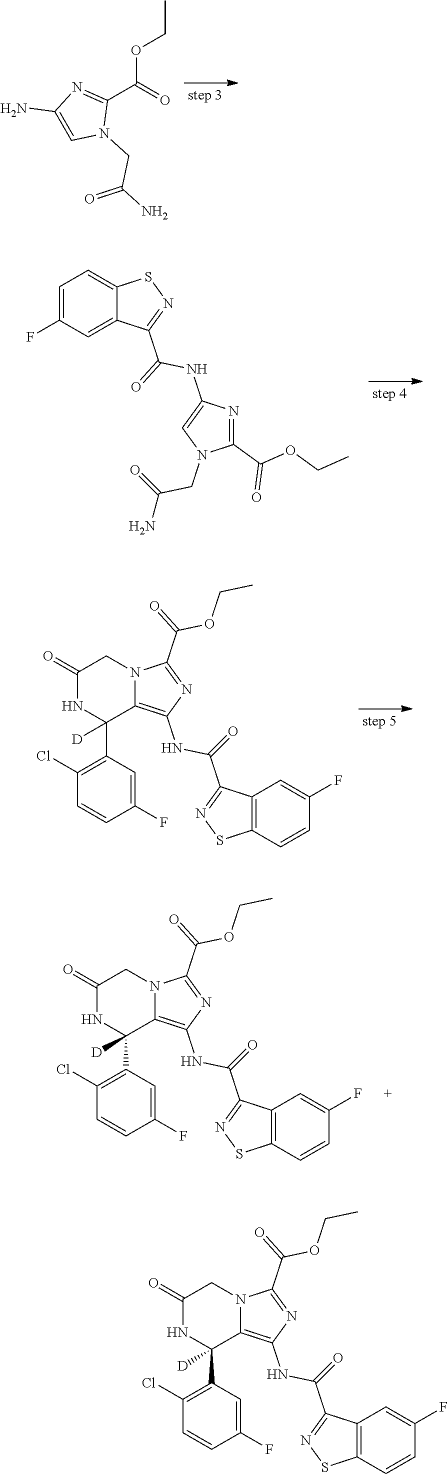 [SMILES: CCOC(=O)c1nc(N)cn1CC(N)=O ; CCOC(=O)c1nc(NC(=O)c2nsc3ccc(F)cc23)cn1CC(N)=O ; [2H]C1(c2cc(F)ccc2Cl)NC(=O)Cn2c(C(=O)OCC)nc(NC(=O)c3nsc4ccc(F)cc34)c21 ; [2H][C@@]1(c2cc(F)ccc2Cl)NC(=O)Cn2c(C(=O)OCC)nc(NC(=O)c3nsc4ccc(F)cc34)c21 ; [2H][C@]1(c2cc(F)ccc2Cl)NC(=O)Cn2c(C(=O)OCC)nc(NC(=O)c3nsc4ccc(F)cc34)c21]

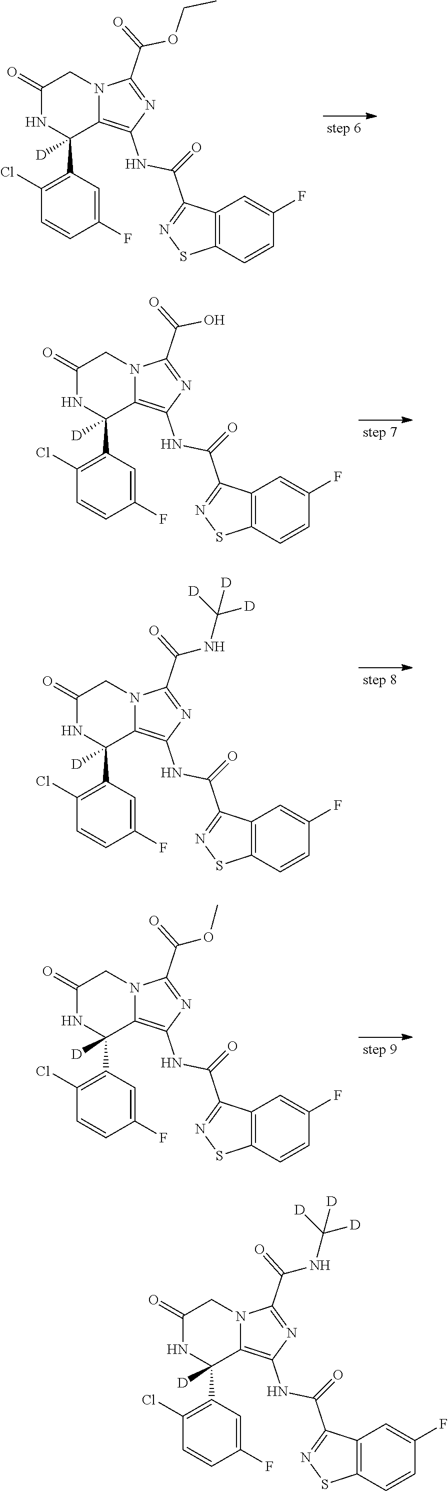

### Step 1. (2-chloro-5-fluorophenyl)methan-d2-ol

A round bottom flask was charged with methyl 2-chloro-5-fluorobenzoate (1.0 g). THF (50 mL) was added, and the mixture was stirred at 0° C. LiAlD₄ (1.0 g) was added, and the mixture was stirred at 25° C. for 2 hours. The reaction was then quenched by the addition of water (1 mL), 15% sodium hydroxide solution (1 mL) and then more water (3 mL) at 0° C. The mixture was warmed to 25° C. and stirred for 15 minutes. The resulting mixture was filtered through a Celite pad. The solid was washed with ethyl acetate. The filtrate was concentrated under vacuum. The resulting crude material was purified by reverse phase column chromatography (water/acetonitrile) to give the desired product (700 mg) as an off-white solid. ¹H NMR (400 MHz, DMSO-d6) δ 7.43 (dd, J=8.4, 5.2 Hz, 1H), 7.34-7.32 (m, 1H), 7.14-7.10 (m, 1H).

### Step 2. 2-chloro-5-fluorobenzaldehyde-1-d

A reaction vial was charged with (2-chloro-5-fluorophenyl)methan-d2-ol (700 mg) and pyridinium chlorochromate (1.84 g) before being evacuated and purged with nitrogen three times. Dichloromethane (20 mL) was added, and the mixture was stirred at 25° C. for 4 hours under nitrogen. The product was purified by silica gel chromatography (10 g column; eluting with petroleum ether:ethyl acetate 10:1) to give the desired product (600 mg) as an off-white solid. ¹H NMR (400 MHz, DMSO-d6) δ 7.70-7.66 (m, 1H), 7.61-7.56 (m, 2H)

### Step 3. ethyl 1-(2-amino-2-oxoethyl)-4-(5-fluorobenzo[d]isothiazole-3-carboxamido)-1H-imidazole-2-carboxylate

A reaction vial was charged with ethyl 4-amino-1-(2-amino-2-oxoethyl)-1H-imidazole-2-carboxylate (600 mg), 5-fluorobenzo[d]isothiazole-3-carboxylic acid (669 mg), HATU (1.29 g), DIEA (1.09 g) and a stir bar before being evacuated and purged with nitrogen three times. DMF (6 mL) was added, and the mixture was stirred at 25° C. for 1 hour under nitrogen. The resulting crude material was purified by HPLC (mobile phase A: water with 0.1% formic acid, mobile phase B: acetonitrile with 0.1% formic acid). Lyophilization yielded the desired product (700 mg) as an off-white amorphous solid. LCMS: RT 0.971 min, [M+H]⁺ 392.0, LCMS method A.

### Step 4. Ethyl 8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylate-8-d

A reaction vial was charged with ethyl 1-(2-amino-2-oxoethyl)-4-(5-fluorobenzo[d]isothiazole-3-carboxamido)-1H-imidazole-2-carboxylate (700 mg) and 2-chloro-5-fluorobenzaldehyde-1-d (285 mg) before being evacuated and purged with nitrogen three times. Eaton's reagent (15 mL) was added, and the mixture was stirred at 100° C. for 1 hour under nitrogen. The reaction was quenched with saturated NaHCO₃ solution. The reaction mixture was diluted with water (10 mL), and the aqueous phase was extracted with ethyl acetate (50 mL) three times. The combined organic layers were washed with brine, dried over sodium sulfate, filtered and concentrated in vacuo. The resulting crude material was purified by pre-HPLC (mobile phase A: water, mobile phase B: acetonitrile) to give the desired product (500 mg) as an off-white amorphous solid. LCMS: RT 2.403 min, [M+H]⁺ 533.1, LCMS method F.

### Step 5. Ethyl (S)-8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylate-8-d and ethyl (R)-8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylate-8-d

Ethyl 8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylate-8-d (500 mg) was chirally resolved by CHIRAL-HPLC (Column: DZ-CHIRALPAK IG-3, 4.6*50 mm, 3.0 μm; mobile phase A: hexane (with 0.2% isopropylamine), mobile phase B: EtOH:DCM 1:1; gradient: 50:50 isocratic; flow rate: 1 mL/min) to give both enantiomers as an off-white amorphous solid.

Peak 1: 200 mg. LCMS: RT 2.398 min, [M+H]⁺ 533.10, LCMS method M. ¹H NMR (400 MHz, DMSO-d6) δ 10.53 (s, 1H), 9.00 (s, 1H), 8.39 (dd, J=9.1, 4.8 Hz, 1H), 8.29 (dd, J=9.6, 2.6 Hz, 1H), 7.69-7.57 (m, 1H), 7.29 (dd, J=8.8, 5.1 Hz, 1H), 7.16 (dd, J=9.2, 3.0 Hz, 1H), 7.02 (td, J=8.4, 3.1 Hz, 1H), 5.17 (s, 1H), 5.12 (s, 1H), 4.35 (qd, J=7.1, 2.5 Hz, 2H), 1.34 (t, J=7.1 Hz, 3H).

Peak 2: 150 mg. LCMS: RT 2.401 min, [M+H]⁺ 533.10, LCMS method M. ¹H NMR (400 MHz, DMSO-d6) δ 10.53 (s, 1H), 9.00 (s, 1H), 8.39 (dd, J=9.0, 4.8 Hz, 1H), 8.29 (dd, J=9.6, 2.5 Hz, 1H), 7.64 (td, J=8.8, 2.5 Hz, 1H), 7.29 (dd, J=8.8, 5.1 Hz, 1H), 7.16 (dd, J=9.2, 3.1 Hz, 1H), 7.02 (ddd, J=8.8, 8.0, 3.1 Hz, 1H), 5.17 (s, 1H), 5.12 (s, 1H), 4.35 (qd, J=7.1, 2.5 Hz, 2H), 1.34 (t, J=7.1 Hz, 3H).

### Step 6. (R)-8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylic-8-d acid

To a solution of ethyl (R)-8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylate-8-d (40 mg) in MeOH (1 mL) was added a solution of NaOH (9.0 mg) in H₂O (0.5 mL), and the mixture was stirred for 1.5 hours at room temperature. The reaction mixture's pH was adjusted to 3 with 1 N HCl. The precipitate was collected by filtration and the collected solid was dried under vacuum to give the desired product (25 mg) as an off-white amorphous solid. LCMS: RT 0.753 min, [M+H]⁺ 505.0, LCMS method E.

### Step 7. (R)—N-(8-(2-chloro-5-fluorophenyl)-3-((methyl-d3)carbamoyl)-6-oxo-5,6,7,8-tetrahydroimidazo[1,5-a]pyrazin-1-yl-8-d)-5-fluorobenzo[d]isothiazole-3-carboxamide

A reaction vial was charged with (R)-8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylic-8-d acid (20 mg), methan-d3-amine hydrochloride (3.4 mg), HATU (18 mg) and DIEA (15 mg) before being evacuated and purged with nitrogen three times. DMF (1 mL) was added, and the mixture was stirred for 1 hour at room temperature. The resulting crude material was purified by prep-HPLC (Column: YMC-Actus Triart C18, 30*150 mm, 5 μm; mobile phase A: 10 mM NH₄HCO₃, mobile phase B: acetonitrile; flow rate: 60 mL/min; gradient: 33% B to 45% B in 8 min, then 45% B; wavelength: 220/254 nm; RT 7.85 min) to give the desired product (5.8 mg) as an off-white amorphous solid. LCMS: RT 0.97 min, [M+H]⁺ 521.05, LCMS method P. ¹H NMR (400 MHz, DMSO-d6) δ 10.32 (s, 1H), 8.94 (s, 1H), 8.44-8.28 (m, 2H), 8.25 (dd, J=9.6, 2.5 Hz, 1H), 7.64 (td, J=8.9, 2.6 Hz, 1H), 7.27 (dd, J=8.9, 5.1 Hz, 1H), 7.18 (dd, J=9.2, 3.1 Hz, 1H), 7.00 (td, J=8.4, 3.1 Hz, 1H), 5.25 (d, J=18.7 Hz, 1H), 5.11 (d, J=18.8 Hz, 1H).

### Steps 8 and 9. (S)—N-(8-(2-chloro-5-fluorophenyl)-3-((methyl-d3)carbamoyl)-6-oxo-5,6,7,8-tetrahydroimidazo[1,5-a]pyrazin-1-yl-8-d)-5-fluorobenzo[d]isothiazole-3-carboxamide

(S)—N-(8-(2-chloro-5-fluorophenyl)-3-((methyl-d3)carbamoyl)-6-oxo-5,6,7,8-tetrahydroimidazo[1,5-a]pyrazin-1-yl-8-d)-5-fluorobenzo[d]isothiazole-3-carboxamide was prepared from ethyl (S)-8-(2-chloro-5-fluorophenyl)-1-(5-fluorobenzo[d]isothiazole-3-carboxamido)-6-oxo-5,6,7,8-tetrahydroimidazo[1,5-a]pyrazine-3-carboxylate-8-d according to the procedures described above for steps 6 and 7. LCMS: RT 0.97 min, [M+H)+521.05. LCMS method P. ¹HNMR (400 MHz, DMSO-d6): 10.32 (s, 1H), 8.94 (s, 1H), 8.48-8.34 (m, 2H), 8.25 (dd, J=9.6, 2.6 Hz, 1H), 7.64 (td, J=8.9, 2.6 Hz, 1H), 7.26 (dd, J=8.8, 5.1 Hz, 1H), 7.18 (dd, J=9.2, 3.1 Hz, 1H), 7.00 (td, J=8.3, 3.0 Hz, 1H), 5.25 (d, J=18.8 Hz, 1H), 5.11 (d, J=18.7 Hz, 1H).

Additional compounds prepared according to the methods of Example 51 are listed in Table 15 below. Corresponding ¹H NMR and mass spectrometry characterization for these compounds are described in Table 1. Certain compounds in Table 15 below were prepared with other compounds whose preparation is described in the Examples herein.

TABLE 15
Additional Exemplary Compounds
Compound

I-211
I-212
I-652
I-653
I-670
I-671
I-674
I-675

### Example 52

### (R)—N-(5′-fluoro-3-(methylcarbamoyl)-2′,6-dioxo-6,7-dihydro-5H-spiro[imidazo[1,5-a]pyrazine-8,3′-indolin]-1-yl)benzo[d]isothiazole-3-carboxamide (I-599 or I-600) and (S)—N-(5′-fluoro-3-(methylcarbamoyl)-2′,6-dioxo-6,7-dihydro-5H-spiro[imidazo[1,5-a]pyrazine-8,3′-indolin]-1-yl)benzo[d]isothiazole-3-carboxamide (I-599 or I-600)

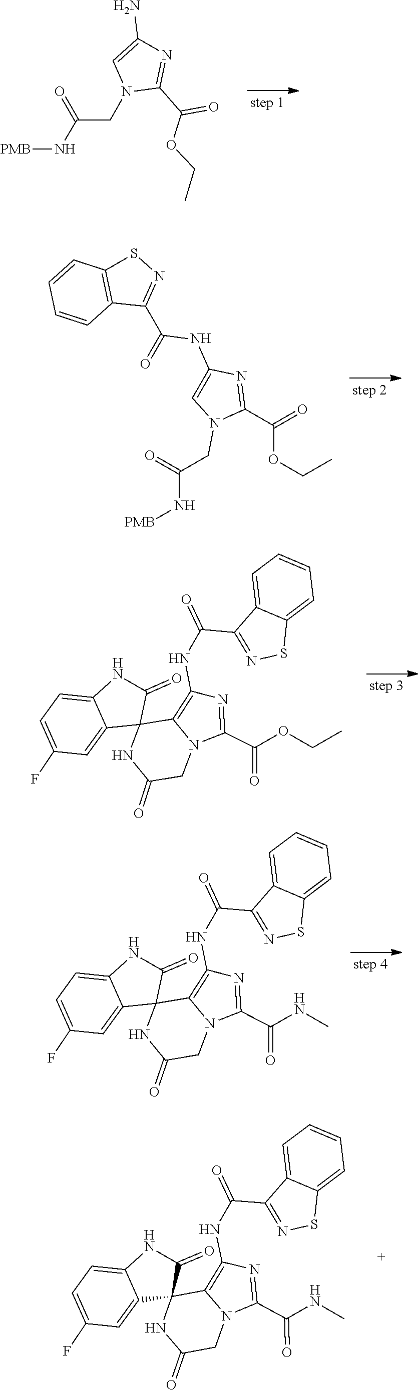

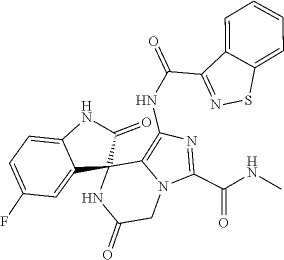

### Step 1. ethyl 4-(benzo[d]isothiazole-3-carboxamido)-1-(2-((4-methoxybenzyl)amino)-2-oxoethyl)-1H-imidazole-2-carboxylate

A 250 mL round bottom flask was charged with ethyl 4-amino-1-(2-((4-methoxybenzyl)amino)-2-oxoethyl)-1H-imidazole-2-carboxylate (2.5 g), followed by benzo[d]isothiazole-3-carboxylic acid (1.55 g), chloro-N,N,N′,N′-tetramethylformamidinium hexafluorophosphate (2.74 g), acetonitrile (50 mL) and dichloromethane (50 mL). To this reaction mixture was added 1-methyl-1H-imidazole (3.09 g). After 1 hour, the solution was concentrated to −50 mL, and saturated NaHCO₃ solution (100 mL) was added. After stirring for 5 min, the solid was collected by filtration, washed with 1 N HCl (100 mL), water (100 mL), acetonitrile (2×10 mL) and hexanes (3×50 mL). Drying under vacuum gave the desired product (2.35 g) as an off-white powder, which was used in the next step without further purification. LCMS: RT 1.46 min, [M+H]⁺ 494.2, LCMS method S.

### Step 2. ethyl 1-(benzo[d]isothiazole-3-carboxamido)-5′-fluoro-2′,6-dioxo-6,7-dihydro-5H-spiro[imidazo[1,5-a]pyrazine-8,3′-indoline]-3-carboxylate

A 20 mL microwave vial was charged with ethyl 4-(benzo[d]isothiazole-3-carboxamido)-1-(2-((4-methoxybenzyl)amino)-2-oxoethyl)-1H-imidazole-2-carboxylate (600 mg), followed by 5-fluoro-2,3-dihydro-1H-indole-2,3-dione (402 mg) and Eaton's reagent (15.05 g). The vial was placed in a pre-heated oil bath (105° C.), stirred for 90 minutes, cooled to room temperature and poured into a stirring biphasic solution of ethyl acetate (150 mL) and water (100 mL). To this solution was carefully added NaHCO₃, until bubbling ceased. Water (200 mL) was added and the layers were separated. The aqueous layer was extracted twice more with ethyl acetate (2×500 mL). The organic layers were combined, dried over Na₂SO₄, filtered and concentrated to afford a dark brown powder. The powder was dissolved in dichloromethane (25 mL) and added to stirred hexanes (200 mL). After stirring for 10 minutes the suspension was filtered. The collected solid was washed with hexanes (50 mL) and dried under vacuum to afford the desired product (520 mg) as a pale brown powder. LCMS: RT 1.17 min, [M+H]⁺ 521.2, LCMS method S.

### Step 3. N-(5′-fluoro-3-(methylcarbamoyl)-2′,6-dioxo-6,7-dihydro-5H-spiro[imidazo[1,5-a]pyrazine-8,3′-indolin]-1-yl)benzo[d]isothiazole-3-carboxamide

A 100 mL round bottom flask was charged with ethyl 1-(benzo[d]isothiazole-3-carboxamido)-5′-fluoro-2′,6-dioxo-6,7-dihydro-SH-spiro[imidazo[1,5-a]pyrazine-8,3′-indoline]-3-carboxylate (520 mg), followed by methanol (20 mL) and methylamine in water (8.1 g, 40% weight). The flask was placed in a pre-heated oil bath (55° C.), stirred for 30 minutes and cooled to room temperature. The reaction mixture was concentrated, and the resulting residue was purified using reverse phase chromatography (80 g column, 5-100% acetonitrile in 10 mM ammonium formate solution) to afford the desired product (90 mg) as a pale pink powder. LCMS: RT 1.99 min, [M+H]⁺ 506.2, LCMS method T. ¹H NMR (400 MHz, DMSO-d6) δ 10.72 (br s, 1H), 9.93 (s, 1H), 9.14 (s, 1H), 8.58 (q, J=4.6 Hz, 1H), 8.53 (d, J=8.2 Hz, 1H), 8.26 (d, J=8.2 Hz, 1H), 7.69-7.62 (m, 1H), 7.60-7.53 (m, 1H), 7.08 (dd, J=8.0, 2.6 Hz, 1H), 6.49 (td, J=9.4, 2.7 Hz, 1H), 6.33 (dd, J=8.5, 4.3 Hz, 1H), 5.48 (d, J=18.4 Hz, 1H), 4.99 (d, J=18.4 Hz, 1H), 2.76 (d, J=4.8 Hz, 3H).

### Step 4. (R)—N-(5′-fluoro-3-(methylcarbamoyl)-2′,6-dioxo-6,7-dihydro-5H-spiro[imidazo[1,5-a]pyrazine-8,3′-indolin]-1-yl)benzo[d]isothiazole-3-carboxamide and (S)—N-(5′-fluoro-3-(methylcarbamoyl)-2′,6-dioxo-6,7-dihydro-5H-spiro[imidazo[1,5-a]pyrazine-8,3′-indolin]-1-yl)benzo[d]isothiazole-3-carboxamide

N-(5′-fluoro-3-(methylcarbamoyl)-2′,6-dioxo-6,7-dihydro-5H-spiro[imidazo[1,5-a]pyrazine-8,3′-indolin]-1-yl)benzo[d]isothiazole-3-carboxamide (86 mg) was chirally resolved using the chiral HPLC condition below to give the two enantiomers. Column: ChiralPak IB, 250 mm×4.6 mm ID, 5 μm; mobile phase: 8:24:68 MeOH:dichloromethane:hexane (0.1% diethylamine); isocratic; flow: 0.8 mL/min,; run time: 25 min; wavelength: 254 nm.

Compound I-600, Peak 1: 23.5 mg. Chiral HPLC RT: 17.2 min. LCMS: RT 2.01 min, [M+H]⁺ 506.2, LCMS method T. ¹H NMR (400 MHz, DMSO-d6) δ 10.74 (br s, 1H), 9.95 (s, 1H), 9.14 (s, 1H), 8.59 (q, J=4.5 Hz, 1H), 8.53 (d, J=8.2 Hz, 1H), 8.26 (d, J=8.2 Hz, 1H), 7.68-7.63 (m, 1H), 7.59-7.53 (m, 1H), 7.08 (dd, J=8.0, 2.6 Hz, 1H), 6.49 (td, J=9.4, 2.7 Hz, 1H), 6.33 (dd, J=8.5, 4.3 Hz, 1H), 5.48 (d, J=18.4 Hz, 1H), 4.98 (d, J=18.4 Hz, 1H), 2.76 (d, J=4.8 Hz, 3H). Contains ˜1.1 equiv. of diethylammonium formate salt.

Compound I-599, Peak 2: 21 mg. Chiral HPLC RT: 20.8 min. LCMS: RT 2.00 min, [M+H]⁺ 506.2, LCMS method T. ¹H NMR (400 MHz, DMSO-d6) δ 10.70 (br s, 1H), 9.95 (s, 1H), 9.14 (br s, 1H), 8.59 (q, J=4.6 Hz, 1H), 8.53 (d, J=8.2 Hz, 1H), 8.27 (d, J=8.2 Hz, 1H), 7.69-7.62 (m, 1H), 7.59-7.53 (m, 1H), 7.08 (dd, J=8.0, 2.6 Hz, 1H), 6.49 (td, J=9.1, 2.7 Hz, 1H), 6.33 (dd, J=8.5, 4.3 Hz, 1H), 5.48 (d, J=18.4 Hz, 1H), 4.98 (d, J=18.4 Hz, 1H), 2.76 (d, J=4.7 Hz, 3H).

Additional compounds prepared according to the methods of Example 52 are listed in Table 16 below. Corresponding ¹H NMR and mass spectrometry characterization for these compounds are described in Table 1. Certain compounds in Table 16 below were prepared with other compounds whose preparation is described in the Examples herein.

TABLE 16
Additional Exemplary Compounds
Compound

I-490
I-668
I-725
I-726
I-727
I-728
I-729
I-730
I-731
I-732

### Example 53

### 1-(benzo[d]isothiazole-3-carboxamido)-8-(2-chloro-5-fluorophenyl)-6-oxo-5,6,7,8-tetrahydroimidazo[1,5-a]pyrazin-3-yl acetate (I-620) and N-(8-(2-chloro-5-fluorophenyl)-3-(6-hydroxypyridin-2-yl)-6-oxo-5,6,7,8-tetrahydroimidazo[1,5-a]pyrazin-1-yl)benzo[d]isothiazole-3-carboxamide (I-621)

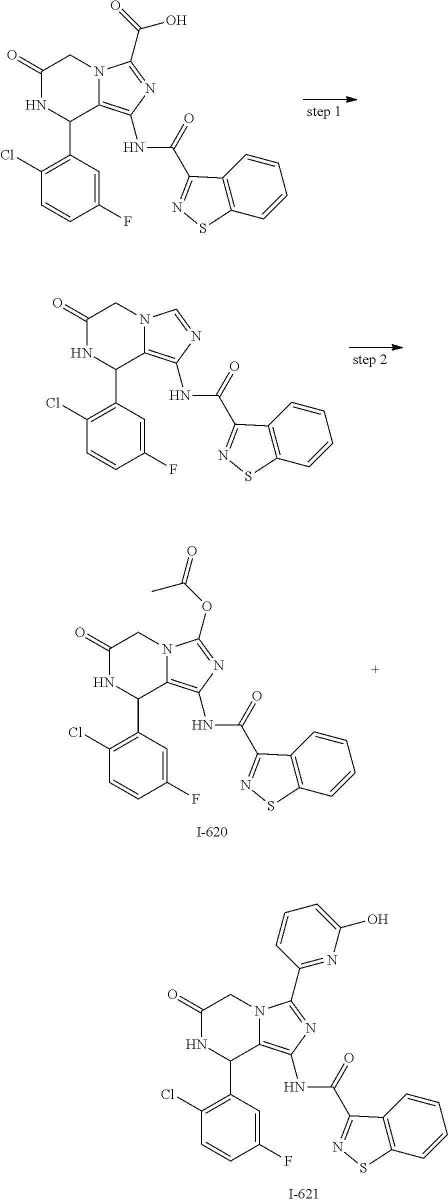 [SMILES: O=C1Cn2c(C(=O)O)nc(NC(=O)c3nsc4ccccc34)c2C(c2cc(F)ccc2Cl)N1 ; O=C1Cn2cnc(NC(=O)c3nsc4ccccc34)c2C(c2cc(F)ccc2Cl)N1 ; CC(=O)Oc1nc(NC(=O)c2nsc3ccccc23)c2n1CC(=O)NC2c1cc(F)ccc1Cl ; O=C1Cn2c(-c3cccc(O)n3)nc(NC(=O)c3nsc4ccccc34)c2C(c2cc(F)ccc2Cl)N1]

### Step 1. N-(8-(2-chloro-5-fluorophenyl)-6-oxo-5,6,7,8-tetrahydroimidazo[1,5-a]pyrazin-1-yl)benzo[d]isothiazole-3-carboxamide

A screw-cap vial equipped with a stir bar was charged with 1-(benzo[d]isothiazole-3-carboxamido)-8-(2-chloro-5-fluorophenyl)-6-oxo-5,6,7,8-tetrahydroimidazo[1,5-a]pyrazine-3-carboxylic acid (530 mg), sodium chloride (191 mg) in DMSO (5 mL). The vial was sealed and heated in an oil bath at 140° C. for 30 min. The desired product was precipitated by addition of water (20 mL), then filtered and washed generously with water to furnish a brown powder (440 mg), which was used in the next step without further purification. LCMS: RT 0.92 min, [M+H]⁺ 442.2, LCMS method Q.

### Step 2. N-(8-(2-chloro-5-fluorophenyl)-3-(6-hydroxypyridin-2-yl)-6-oxo-5,6,7,8-tetrahydroimidazo[1,5-a]pyrazin-1-yl)benzo[d]isothiazole-3-carboxamide and 1-(benzo[d]isothiazole-3-carboxamido)-8-(2-chloro-5-fluorophenyl)-6-oxo-5,6,7,8-tetrahydroimidazo[1,5-a]pyrazin-3-yl acetate

A screw-cap vial equipped with a stir bar was charged with N-(8-(2-chloro-5-fluorophenyl)-6-oxo-5,6,7,8-tetrahydroimidazo[1,5-a]pyrazin-1-yl)benzo[d]isothiazole-3-carboxamide (100 mg), 6-bromopyridin-2-ol (118 mg), and copper(I) iodide (129 mg) in DMF (1.0 mL). The reaction mixture was sonicated for 5 minutes, then nitrogen gas was bubbled through the reaction mixture for 5-10 minutes, before addition of palladium diacetate (50.8 mg). The vial was sealed and heated at 130° C. (oil bath) for about 5 hours. The reaction media was passed through a Celite plug, and the Celite plug was washed with generous amount of DCM. The organic fraction was washed with brine. The layers were separated, and the DCM layer was set aside. The Celite plug was washed one more time with ethyl acetate. The ethyl acetate was washed with brine. The layers were separated, and the ethyl acetate layer was combined with the DCM layer from the first extraction. The volatiles from the combined organic layers were evaporated, and the crude residue was purified by reverse-phase column chromatography (C18, 30 g) eluting with 10 mM AmF solution in water and ACN to furnish a white powder as a mixture of three compounds. It was purified further by Prep HPLC (two runs) using the following conditions: CSH Prep C18 OBD, 5 μm, 30×75 mm (column), XBridge Prep C18, 5 μm 19×10 mm (pre-column), mobile phase A: 10 mM NH₄HCO₃ solution, mobile phase B: acetonitrile. Gradient: 30% B isocratic for 1 min, 30% B to 50% B in 8 minutes, 50% B to 100% B for 0.1 minute, hold 100% B for 2.9 minutes. The pure fractions containing the two desired products were lyophilized to furnish the two products, both as white powders.

1-(benzo[d]isothiazole-3-carboxamido)-8-(2-chloro-5-fluorophenyl)-6-oxo-5,6,7,8-tetrahydroimidazo[1,5-a]pyrazin-3-yl acetate (1.3 mg), LCMS: RT 1.94 min, [M+H]⁺ 500.2, LCMS method V; ¹H-NMR (400 MHz, CD₃CN): δ 9.03 (br s, 1H), 8.81 (d, J=8.0 Hz, 1H), 8.13 (d, J=8.2 Hz, 1H), 7.66 (ddd, J=8.2, 7.0, 1.4 Hz, 1H), 7.60 (ddd, J=8.2, 7.0, 1.4 Hz, 1H), 7.20 (dd, J=8.8, 5.1 Hz, 1H), 7.08 (br s, 1H), 7.05 (dd, J=9.2, 3.0 Hz, 1H), 6.86 (ddd, J=8.8, 8.0, 3.0 Hz, 1H), 6.33 (s, J=11.8 Hz, 1H), 5.14 (dd, J=18.8, 1.1 Hz, 1H), 5.04 (dd, J=18.7, 1.7 Hz, 1H), 3.91 (s, 3H).

N-(8-(2-chloro-5-fluorophenyl)-3-(6-hydroxypyridin-2-yl)-6-oxo-5,6,7,8-tetrahydroimidazo[1,5-a]pyrazin-1-yl)benzo[d]isothiazole-3-carboxamide (2.1 mg), LCMS: RT 1.87 min, [M+H]⁺ 535.3, LCMS method V. ¹H-NMR (400 MHz, CD₃CN): δ 8.99 (brs, 1H), 8.82 (d, J=8.1 Hz, 1H), 8.14 (d, J=8.0 Hz, 1H), 7.69-7.64 (m, 2H), 7.63-7.58 (m, 1H), 7.38 (d, J=6.3 Hz, 1H), 7.20 (dd, J=8.8, 5.1 Hz, 1H), 7.08 (dd, J=9.2, 3.1 Hz, 1H, overlapping), 7.09 (brs, 1H), 6.86 (ddd, J=8.7, 8.2, 3.0 Hz, 1H), 6.60 (d, J=8.3 Hz, 1H), 6.39 (s, 1H), 5.36 (d, J=18.7 Hz, 1H), 5.27 (d, J=18.4 Hz, 1H).

### Example 54

### N-(8-cyclohexyl-6-oxo-5,6,7,8-tetrahydroimidazo[1,5-a]pyrazin-1-yl)-3-fluoro-5-(trifluoromethyl)benzamide (I-622)

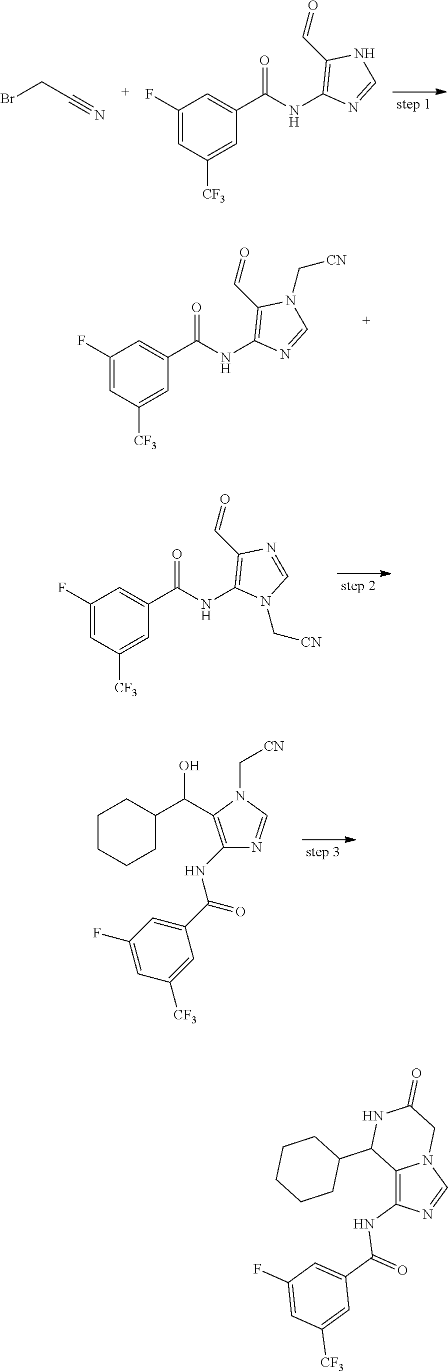

### Step 1. N-(1-(cyanomethyl)-5-formyl-1H-imidazol-4-yl)-3-fluoro-5-(trifluoromethyl)benzamide and N-(1-(cyanomethyl)-4-formyl-1H-imidazol-5-yl)-3-fluoro-5-(trifluoromethyl)benzamide

A round-bottom flask equipped with a stir bar was charged with 3-fluoro-N-(5-formyl-1H-imidazol-4-yl)-5-(trifluoromethyl)benzamide (543 mg) in DMF (10 mL). The reaction solution was cooled to 0° C., then sodium hydride (159 mg, 60% weight) was added in one portion. The reaction mixture was stirred at 0° C. for about 30 min until bubbling ceased, and then 2-bromoacetonitrile (1 g) was added dropwise. The temperature was allowed to reach room temperature, and the mixture was stirred for 2 more hours. The reaction was quenched by the addition of saturated ammonium chloride solution, and the mixture was extracted with ethyl acetate. The crude residue after evaporation under reduced pressure was purified by reversed phase column chromatography (C18, 30 g), eluting with 10 mM AmF in water and ACN to furnish a mixture of the two products as a light yellow powder (484 mg), which was used in the next step without further purification. LCMS: RT 0.99 min, [M+H]⁺ 341.4 and RT 1.03 min, [M+H]⁺ 341.3, LCMS method U.

### Step 2. N-(1-(cyanomethyl)-5-(cyclohexyl(hydroxy)methyl)-1H-imidazol-4-yl)-3-fluoro-5-(trifluoromethyl)benzamide

A flame-dried round-bottom flask equipped with a stir bar was charged with a mixture of N-(1-(cyanomethyl)-5-formyl-1H-imidazol-4-yl)-3-fluoro-5-(trifluoromethyl)benzamide and N-(1-(cyanomethyl)-4-formyl-1H-imidazol-5-yl)-3-fluoro-5-(trifluoromethyl)benzamide (213 mg) in anhydrous THF (5 mL). Cyclohexylmagnesium chloride in THF (1.3 molar, 963 μL) was then added dropwise at room temperature. The reaction mixture was left stirring under nitrogen gas for about 30 min. The reaction media was quenched by the addition of saturated ammonium chloride solution, and the desired product was extracted by ethyl acetate. After evaporation of the volatiles under reduced pressure, the crude residue was purified by reverse-phase column chromatography (C18, 30 g) eluting with 10 mM of AmF in water and ACN to furnish a semi-pure (60-70% purity) mixture containing the desired product as a brown powder (43 mg). The material was used in the next step without further purification. LCMS: RT 1.22 min, [M−H]⁺ 423.4, LCMS method U.

### Step 3. N-(8-cyclohexyl-6-oxo-5,6,7,8-tetrahydroimidazo[1,5-a]pyrazin-1-yl)-3-fluoro-5-(trifluoromethyl)benzamide

A round-bottom flask equipped with a stir bar was charged with semi-pure N-(1-(cyanomethyl)-5-(cyclohexyl(hydroxy)methyl)-1H-imidazol-4-yl)-3-fluoro-5-(trifluoromethyl)benzamide (43 mg) in concentrated sulfuric acid (0.5 mL). The reaction mixture was stirred at room temperature, then slowly warmed to 70° C. and stirred for 5 min. The reaction mixture was allowed to stir further for about 30 min and cooled to RT before dilution with water and extraction with ethyl acetate. The organic layer was washed with brine and concentrated under reduced pressure. The crude material was purified by reverse-phase column chromatography (3 repeats, C18, 30 g) each eluting with 10 mM AmF in water and ACN to furnish the desired product (0.47 mg) as a white powder. LCMS: RT 2.45 min, [M−H]⁺423.4, LCMS method V. HNMR (400 MHz, CD₃CN): δ 8.68 (br s, 1H), 8.05 (s, 1H), 7.90 (d, J=9.2 Hz, 1H), 7.70 (d, J=8.3 Hz, 1H), 7.54 (s, 1H), 5.16 (d, J=18.1 Hz, 1H), 5.08 (d, J=18.1 Hz, 1H), 3.87 (br s, 1H), 4.46-4.40 (m, 1H), 1.78-1.69 (m, 2H), 1.68-1.58 (m, 2H), 1.33-1.06 (m, 5H), 1.05-0.78 (m, 2H).

### Example 55

### (S)-8-(2-chloro-5-fluorophenyl)-1-((7-fluoro-5-(trifluoromethyl)isoquinolin-1-yl)amino)-N-methyl-6-oxo-5,6,7,8-tetrahydroimidazo[1,5-a]pyrazine-3-carboxamide (I-629)

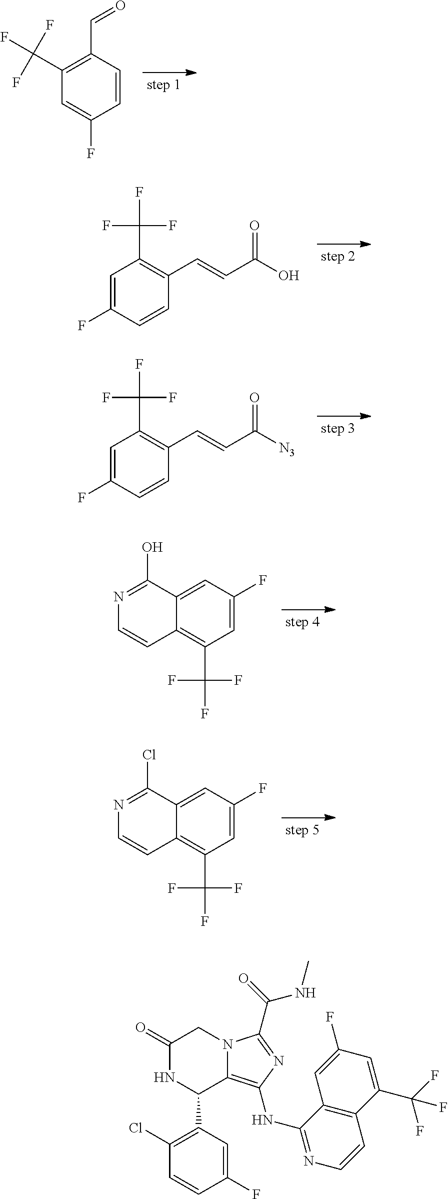

### Step 1. (E)-3-(4-fluoro-2-(trifluoromethyl)phenyl)acrylic acid

A round bottom flask was charged with 4-fluoro-2-(trifluoromethyl)benzaldehyde (5.0 g), malonic acid (5.0 g), and a stirbar. Pyridine (15 mL) was added, and the solution was stirred at 100° C. for 16 h. The reaction mixture was diluted with H₂O (100 mL), then adjusted to pH 6-7 with hydrochloric acid. The aqueous phase was extracted with DCM (150 mL) three times. The combined organic layers were washed with brine, dried over sodium sulfate, filtered, and concentrated in vacuo. The resulting solution was purified using C18 flash chromatography with the following conditions (mobile phase A: water, mobile phase B: acetonitrile; flow rate: 60 mL/min; gradient: 0% B to 100% B in 40 min; detection wavelength: 254/220 nm) to give the desired product (1.2 g) as an off-white amorphous solid. LCMS RT 0.13 min, no MS peak observed, LCMS method I. ¹H NMR (400 MHz, DMSO-d₆) 12.73 (s, 4H), 8.12 (dd, J=8.8, 5.4 Hz, 3H), 7.77 (s, 2H), 7.75-7.67 (m, 5H), 7.65-7.57 (m, 4H), 7.24-7.15 (m, OH), 6.63 (d, J=15.7 Hz, 3H), 3.61 (s, 1H).

### Step 2. (E)-3-(4-fluoro-2-(trifluoromethyl)phenyl)acryloyl azide

A round bottom flask was charged with (E)-3-(4-fluoro-2-(trifluoromethyl)phenyl)acrylic acid (1.3 g), Et₃N (1.7 g), benzene (8 mL) and a stirbar. DPPA (1.8 g) was added, and the solution was stirred at room temperature for 16 h. The crude product was purified by column chromatography on silica gel (petroleum ether/ethyl acetate) to give the desired product as a yellow solid (1.2 g). LCMS RT 1.30 min, no MS peak observed, LCMS method I.

### Step 3. 7-fluoro-5-(trifluoromethyl)isoquinolin-1-ol

A round bottom flask was charged with (E)-3-(4-fluoro-2-(trifluoromethyl)phenyl)acryloyl azide (1.2 g) and a stirbar. PhCH₂Ph (10 mL) was added, and the solution was stirred at 80° C. for 1 h. Then the solution was stirred at 280° C. for 4 h in a sand bath. After cooling to room temperature the mixture was purified by column chromatography on silica gel (petroleum ether/ethyl acetate) to afford the desired product as a yellow solid (350 mg). LCMS: RT 0.77 min, [M+H]⁺ 232.15, LCMS method I.

### Step 4. 1-chloro-7-fluoro-5-(trifluoromethyl)isoquinoline

A round bottom flask was charged with 7-fluoro-5-(trifluoromethyl)isoquinolin-1-ol (350 mg) and a stirbar. POCl₃ (6 mL) was added, and the solution was stirred at 90° C. for 1 h. The reaction was quenched with ice water, diluted with H₂O (10 mL), and the aqueous phase was extracted with ethyl acetate (15 mL) three times. The combined organic layers were washed with brine, dried over sodium sulfate, filtered, and concentrated in vacuo. The crude product was purified using C18 flash chromatography with the following conditions: mobile phase A: water, mobile phase B: acetonitrile; flow rate: 60 mL/min; gradient: 0% B to 100% B in 40 min; detection wavelength: 254/220 nm. Lyophilization yielded 1-chloro-7-fluoro-5-(trifluoromethyl)isoquinoline as an off-white amorphous solid (50 mg). LCMS: RT 1.26 min, [M+H]⁺ 250.0, LCMS method A.

### Step 5. (S)-8-(2-chloro-5-fluorophenyl)-1-((7-fluoro-5-(trifluoromethyl)isoquinolin-1-yl)amino)-N-methyl-6-oxo-5,6,7,8-tetrahydroimidazo[1,5-a]pyrazine-3-carboxamide

A round bottom flask was charged with 1-chloro-7-fluoro-5-(trifluoromethyl)isoquinoline (50 mg), (S)-1-amino-8-(2-chloro-5-fluorophenyl)-N-methyl-6-oxo-5,6,7,8-tetrahydroimidazo[1,5-a]pyrazine-3-carboxamide (0.11 g), RuPhos Pd G3 (17 mg), RuPhos (9.5 mg), Cs₂CO₃ (0.20 g), dioxane (1 mL) and a stirbar before being evacuated and purged with nitrogen three times. The solution was stirred at 100° C. for 1 h. The reaction mixture was diluted with H₂O (10 mL), and the aqueous phase was extracted with ethyl acetate (15 mL) three times. The combined organic layers were washed with brine, dried over sodium sulfate, filtered, and concentrated in vacuo. The resulting solution was purified using C18 flash chromatography with the following conditions (mobile phase A: water, mobile phase B: acetonitrile; flow rate: 60 mL/min; gradient: 0% B to 100% B in 40 min; detection wavelength 254/220 nm) to give the desired product (16.1 mg). LCMS: RT 0.85 min, [M+H]⁺ 551.15, LCMS method E. ¹H NMR (400 MHz, DMSO-d₆) δ 12.68 (s, 1H), 9.45 (s, 1H), 7.34-7.24 (m, 4H), 5.51 (s, 1H), 5.23 (s, 1H), 3.49 (s, 1H), 3.27 (s, 1H), 2.55 (s, 3H), 2.44 (s, 3H).

### Example 56

### (S)—N-(8-(2-chloro-5-fluorophenyl)-3-ethyl-6-oxo-5,6,7,8-tetrahydroimidazo[1,5-a]pyrazin-1-yl)-3-fluoro-5-(trifluoromethyl)benzamide (I-630) and (R)—N-(8-(2-chloro-5-fluorophenyl)-3-ethyl-6-oxo-5,6,7,8-tetrahydroimidazo[1,5-a]pyrazin-1-yl)-3-fluoro-5-(trifluoromethyl)benzamide (I-631)

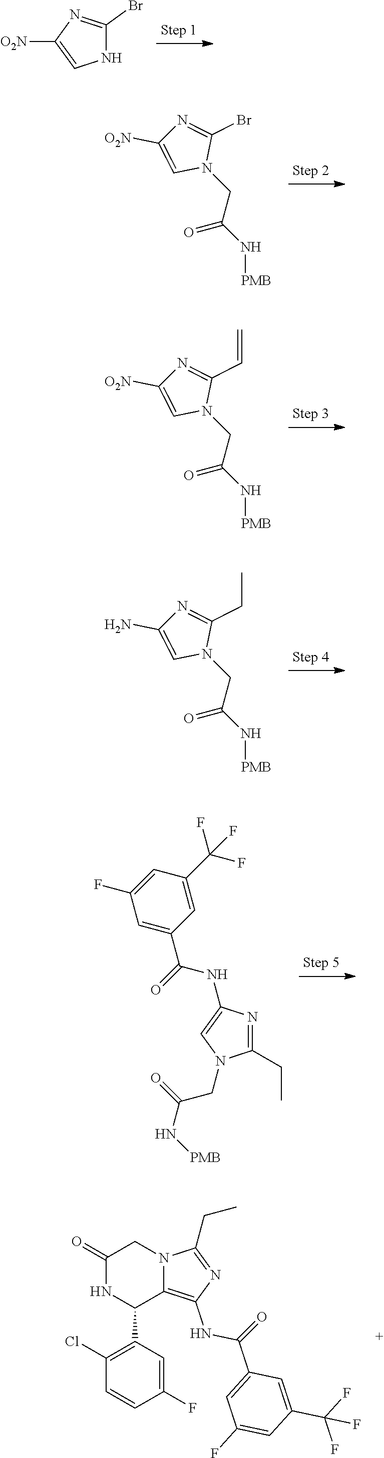

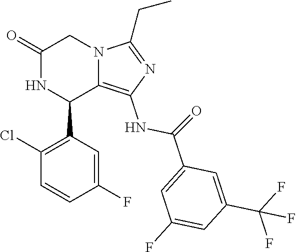 [SMILES: CCc1nc(NC(=O)c2cc(F)cc(C(F)(F)F)c2)c2n1CC(=O)N[C@@H]2c1cc(F)ccc1Cl]

### Step 1. 2-(2-bromo-4-nitro-1H-imidazol-1-yl)-N-(4-methoxybenzyl)acetamide

A reaction vial was charged with 2-bromo-4-nitro-1H-imidazole (2.0 g), 2-(tert-butyl)-1,1,3,3-tetramethylguanidine (1.78 g), 2-bromo-N-(4-methoxybenzyl)acetamide (4.0 g), MeCN (20 mL) and a stirbar before being evacuated and purged with nitrogen three times. The mixture was stirred at room temperature for 20 h. The reaction mixture was filtered and concentrated in vacuo. The resulting crude material was purified by HPLC (acetonitrile/water) to give the desired product as an off-white solid (3.5 g). LCMS RT 0.92 min, [M+H]⁺ 371.10, LCMS method O.

### Step 2. N-(4-methoxybenzyl)-2-(4-nitro-2-vinyl-1H-imidazol-1-yl)acetamide

A reaction vial was charged with 2-(2-bromo-4-nitro-1H-imidazol-1-yl)-N-(4-methoxybenzyl)acetamide (2.0 g), 4,4,5,5-tetramethyl-2-vinyl-1,3,2-dioxaborolane (3.0 g), Dppf Pd G3 (0.5 g), Dppf (0.6 g), K₃PO₄ (3.0 g), dioxane:H₂O 8:1 (20 mL) and a stirbar before being evacuated and purged with nitrogen three times. The mixture was stirred at 90° C. for 20 h. The reaction mixture was diluted with water and extracted with ethyl acetate three times. The combined organic layers were washed with brine, dried over sodium sulfate, filtered, and concentrated in vacuo. The resulting crude material was purified by HPLC (acetonitrile/water) to give the desired product (450 mg) as an off-white solid. LCMS RT 0.90 min, [M+H]⁺ 317.15, LCMS method A.

### Step 3. 2-(4-amino-2-ethyl-1H-imidazol-1-yl)-N-(4-methoxybenzyl)acetamide

To a suspension of N-(4-methoxybenzyl)-2-(4-nitro-2-vinyl-1H-imidazol-1-yl)acetamide (220 mg) in THF (5 mL) was added Pd/C (90.9 mg, 10% wt). The mixture was degassed and purged with hydrogen and stirred at room temperature for 1 h under an atmosphere of hydrogen (balloon). The reaction mixture was purged with N₂, filtered through a syringe filter, and concentrated under reduced pressure to afford the crude desired product as a brown oil. The resulting crude material was used directly in the next step. LCMS RT 0.73 min, [M+H]⁺ 289.15, LCMS method A.

### Step 4. N-(2-ethyl-1-(2-((4-methoxybenzyl)amino)-2-oxoethyl)-1H-imidazol-4-yl)-3-fluoro-5-(trifluoromethyl)benzamide

A reaction vial was charged with 2-(4-amino-2-ethyl-1H-imidazol-1-yl)-N-(4-methoxybenzyl)acetamide (200 mg). THF (2 mL) was added, followed by TEA (211 mg) at room temperature. 3-Fluoro-5-(trifluoromethyl)benzoyl chloride (156.63 mg) was added dropwise under a nitrogen atmosphere. After 1 hour of stirring the resulting crude material was purified by HPLC (acetonitrile/water) to give the desired product (80 mg) as an off-white amorphous solid. LCMS RT 0.58 min, [M+H]⁺ 479.20, LCMS method K.

### Step 5. (S)—N-(8-(2-chloro-5-fluorophenyl)-3-ethyl-6-oxo-5,6,7,8-tetrahydroimidazo[1,5-a]pyrazin-1-yl)-3-fluoro-5-(trifluoromethyl)benzamide and (R)—N-(8-(2-chloro-5-fluorophenyl)-3-ethyl-6-oxo-5,6,7,8-tetrahydroimidazo[1,5-a]pyrazin-1-yl)-3-fluoro-5-(trifluoromethyl)benzamide

A reaction vial was charged with N-(2-ethyl-1-(2-((4-methoxybenzyl)amino)-2-oxoethyl)-1H-imidazol-4-yl)-3-fluoro-5-(trifluoromethyl)benzamide (80 mg) and 2-chloro-5-fluorobenzaldehyde (40 mg). Eaton's reagent (1 mL) was added, and the reaction media was heated at 100° C. for one hour. The reaction mixture was diluted with H₂O (20 mL), and the aqueous phase was extracted with ethyl acetate (30 mL) three times. The combined organic layers were washed with brine, dried over sodium sulfate, filtered, and concentrated in vacuo. The resulting crude material was purified by HPLC (acetonitrile/water) to give the desired product (32 mg) as a white amorphous solid. LCMS RT 1.06 min, [M+H]⁺ 499.15, LCMS method M.

The two enantiomers were separated by CHIRAL-HPLC (Column: CHIRALPAK IH, 2*25 cm, 5 μm; mobile phase A: hexane:DCM 3:1 (0.1% Et₃N), mobile phase B: EtOH; flow rate: 20 mL/min; gradient: 50% B; wavelength: 220/254 nm; sample solvent: EtOH) to give the two desired products, RT 2.86 and 10.80 min, respectively.

Compound I-631, Peak 1, 14.5 mg as an off-white amorphous solid. LCMS RT 1.38 min, [M+H]⁺=499.15, LCMS method M. ¹H NMR (400 MHz, Chloroform-d) δ 9.13 (s, 1H), 7.76 (s, 1H), 7.67 (d, J=8.7 Hz, 1H), 7.46 (d, J=8.0 Hz, 1H), 7.34 (dd, J=8.8, 5.0 Hz, 1H), 6.95 (ddd, J=8.8, 7.5, 3.0 Hz, 1H), 6.90 (d, J=2.9 Hz, 1H), 6.76 (dd, J=8.7, 3.0 Hz, 1H), 6.63 (d, J=2.9 Hz, 1H), 4.65 (dd, J=17.5, 1.0 Hz, 1H), 4.58 (d, J=17.4 Hz, 1H), 2.66 (q, J=7.5 Hz, 2H), 1.24 (t, J=7.5 Hz, 3H).

Compound I-630, Peak 2, 13.2 mg as an off-white amorphous solid. LCMS RT 1.38 min, [M+H]⁺=499.15, LCMS method M. ¹H NMR (400 MHz, Chloroform-d) δ 9.13 (s, 1H), 7.76 (s, 1H), 7.67 (d, J=8.7 Hz, 1H), 7.46 (d, J=8.0 Hz, 1H), 7.34 (dd, J=8.8, 5.0 Hz, 1H), 7.02-6.87 (m, 2H), 6.76 (dd, J=8.7, 3.0 Hz, 1H), 4.74-4.45 (m, 2H), 2.66 (q, J=7.5 Hz, 2H), 1.24 (t, J=7.5 Hz, 3H).

Additional compounds prepared according to the methods of Example 56 are listed in Table 17 below. Corresponding ¹H NMR and mass spectrometry characterization for these compounds are described in Table 1. Certain compounds in Table 17 below were prepared with other compounds whose preparation is described in the Examples herein.

TABLE 17
Additional Exemplary Compounds
Compound

I-632
I-633

### Example 57

### 8-(2-chloro-5-fluorophenyl)-1-((5-chloro-7-fluoroisoquinolin-1-yl)amino)-N-methyl-6-oxo-5,6,7,8-tetrahydroimidazo[1,5-a]pyrazine-3-carboxamide (I-634)

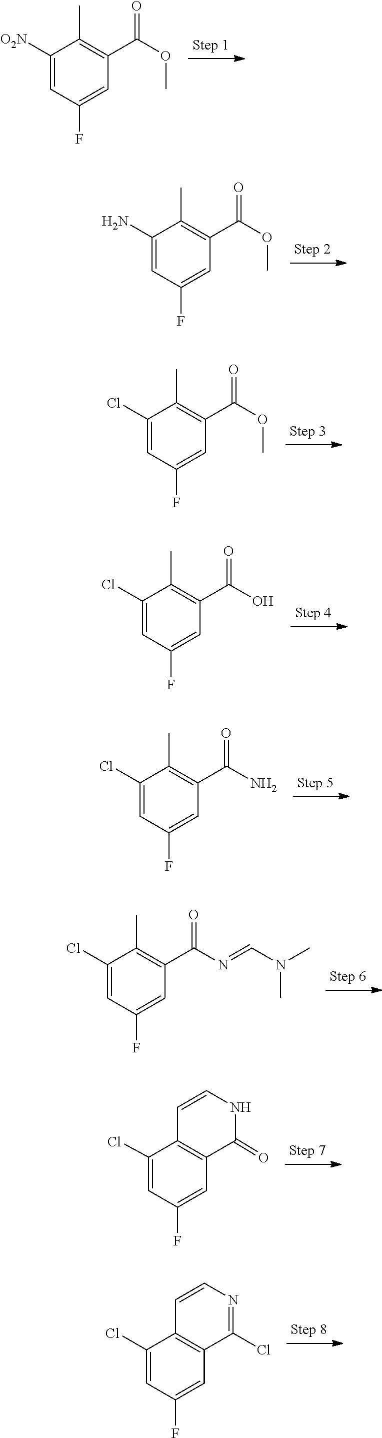

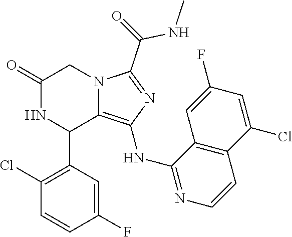 [SMILES: CNC(=O)c1nc(Nc2nccc3c(Cl)cc(F)cc23)c2n1CC(=O)NC2c1cc(F)ccc1Cl]

### Step 1. methyl 3-amino-5-fluoro-2-methylbenzoate

A reaction vial was charged with methyl 5-fluoro-2-methyl-3-nitrobenzoate (2.0 g), iron powder (4.0 g), NH₄Cl (2.0 g), and a stirbar before being evacuated and purged with nitrogen three times. Methanol (24 mL) and water (8 mL) were added, and the mixture was stirred at 25° C. for 1 hour under nitrogen. The reaction mixture was filtered through a pad of Celite. The pad was washed with MeOH (24 mL), and the filtrate was concentrated in vacuo. The resulting crude material was purified by HPLC (acetonitrile/water) to give the desired product (1.5 g) as an off-white amorphous solid. LCMS RT 0.74 min, [M+H]⁺ 184.07, LCMS method A.

### Step 2. methyl 3-chloro-5-fluoro-2-methylbenzoate

To a mixture of methyl 3-amino-5-fluoro-2-methylbenzoate (1.5 g) and sodium nitrite (735 mg) in water (10 mL), concentrated HCl (10 mL) was added dropwise at 0° C. under a nitrogen atmosphere. The mixture was stirred at 0° C. for 30 min, then copper (I) chloride (1.63 g) was added. The mixture was stirred at 0° C. for 1.5 hours. The reaction mixture was diluted with water (50 mL), and the aqueous phase was extracted with ethyl acetate (50 mL) three times. The combined organic layers were washed with brine, dried over sodium sulfate, filtered, and concentrated in vacuo. The resulting crude material was purified by HPLC (acetonitrile/water) to give the desired product (1.1 g) as an off-white amorphous solid. LCMS RT 0.96 min, [M+H]⁺ 203.16, LCMS method C.

### Step 3. 3-chloro-5-fluoro-2-methylbenzoic acid

A reaction vial was charged with methyl 3-chloro-5-fluoro-2-methylbenzoate (1.1 g), sodium hydroxide (0.65 g), and a stirbar before being evacuated and purged with nitrogen three times. Methanol (12 mL) and water (4 mL) were added, and the mixture was stirred at 25° C. for 1 hour under nitrogen. The pH of the solution was adjusted to 2˜3 with HCl. The precipitated solid was collected by filtration, washed with water (4 mL) and dried in vacuo to give the desired product (950 mg) as an off-white solid. LCMS RT 0.78 min, [M+H]⁺ 189.00, LCMS method C.

### Step 4. 3-chloro-5-fluoro-2-methylbenzamide

A reaction vial was charged with 3-chloro-5-fluoro-2-methylbenzoic acid (900 mg), NH₄Cl (306 mg), HATU (2.72 g), DIEA (1.85 g), DMF (10 mL), and a stirbar before being evacuated and purged with nitrogen three times. The mixture was stirred at 25° C. for 1 hour under a nitrogen atmosphere. The resulting crude material was purified by HPLC (acetonitrile/water) to give the desired product (750 mg) as an off-white amorphous solid. LCMS RT 0.85 min, [M+H]⁺ 188.02, LCMS method A.

### Step 5. (E)-3-chloro-N-((dimethylamino)methylene)-5-fluoro-2-methylbenzamide

A reaction vial was charged with 3-chloro-5-fluoro-2-methylbenzamide (700 mg), DMF-DMA (888 mg), and a stirbar before being evacuated and purged with nitrogen three times. THF (10 mL) was added, and the mixture was stirred at 25° C. for 2 hours under nitrogen. The reaction mixture was diluted with water (50 mL), and the aqueous phase was extracted with ethyl acetate (50 mL) three times. The combined organic layers were washed with brine, dried over sodium sulfate, filtered, and concentrated in vacuo. The resulting crude material was purified by HPLC (acetonitrile/water) to give the desired product (850 mg) as an off-white solid. LCMS RT 1.11 min, [M+H]⁺ 243.06, LCMS method N.

### Step 6. 5-chloro-7-fluoroisoquinolin-1(2H)-one

A reaction vial was charged with (E)-3-chloro-N-((dimethylamino)methylene)-5-fluoro-2-methylbenzamide (800 mg), potassium tert-butoxide in THF (0.5 M, 8 mL), and a stirbar before being evacuated and purged with nitrogen three times. The mixture was stirred at 25° C. for 2 hours under nitrogen. The reaction mixture was diluted with water (10 mL), and the aqueous phase was extracted with ethyl acetate (20 mL) three times. The combined organic layers were washed with brine, dried over sodium sulfate, filtered, and concentrated in vacuo. The resulting crude material was purified by HPLC (acetonitrile/water) to give the desired product (300 mg) an off-white amorphous solid. LCMS RT 0.86 min, [M+H]⁺ 198.00, LCMS method P.

### Step 7. 1,5-dichloro-7-fluoroisoquinoline

A reaction vial was charged with 5-chloro-7-fluoroisoquinolin-1(2H)-one (250 mg) and a stirbar before being evacuated and purged with nitrogen three times. POCl₃ (4 mL) was added, and the mixture was stirred at 90° C. for 2 hours under nitrogen. The mixture was quenched with water at 0° C. The reaction mixture was diluted with water (10 mL), and the aqueous phase was extracted with ethyl acetate (20 mL) three times. The combined organic layers were washed with brine, dried over sodium sulfate, filtered, and concentrated in vacuo. The resulting crude material was purified by HPLC (acetonitrile/water) to give the desired product (200 mg) as a yellow solid. LCMS RT 0.93 min, [M+H]⁺ 216.04, LCMS method C.

### Step 8. 8-(2-chloro-5-fluorophenyl)-1-((5-chloro-7-fluoroisoquinolin-1-yl)amino)-N-methyl-6-oxo-5,6,7,8-tetrahydroimidazo[1,5-a]pyrazine-3-carboxamide

A reaction vial was charged with 1,5-dichloro-7-fluoroisoquinoline (50 mg), 1-amino-8-(2-chloro-5-fluorophenyl)-N-methyl-6-oxo-5,6,7,8-tetrahydroimidazo [1,5-a]pyrazine-3-carboxamide (94 mg), Cs₂CO₃ (0.23 g), RuPhos Pd (19 mg), RuPhos (11 mg), 1,4-dioxane (4 mL), and a stirbar before being evacuated and purged with nitrogen three times. The mixture was stirred at 100° C. for 1 hour under a nitrogen atmosphere. The mixture was diluted with water (10 mL), and the aqueous phase was extracted with ethyl acetate (10 mL) three times. The combined organic layers were washed with brine, dried over sodium sulfate, filtered, and concentrated in vacuo. The resulting crude material was purified by HPLC (Column: XBridge Shield RP18 OBD Column, 30*150 mm, 5 μm; mobile phase A: water (0.1% formic acid), mobile phase B: acetonitrile; flow rate: 60 mL/min; gradient: 44% B to 55% B in 7 min, then 55% B; wavelength: 254/220 nm; RT: 5.87 min) to give the desired product (8 mg) as an off-white amorphous solid. LCMS RT 0.74 min, [M+H]⁺ 517.07, LCMS method E. ¹H NMR (400 MHz, DMSO-d₆) δ 12.29 (s, 1H), 8.98 (s, 1H), 8.82 (s, 1H), 7.89 (s, 1H), 7.71 (s, 1H), 7.55 (s, 1H), 7.47 (s, 1H), 7.31 (s, 1H), 7.19 (s, 1H), 6.48 (s, 1H), 6.23 (s, 1H), 5.24 (s, 1H), 5.05 (s, 1H), 2.85 (s, 3H).

### Example 58

### (S)—N-(8-(2-chloro-5-fluorophenyl)-3-((methylsulfonyl)methyl)-6-oxo-5,6,7,8-tetrahydroimidazo[1,5-a]pyrazin-1-yl)-3-fluoro-5-(trifluoromethyl)benzamide (I-635)

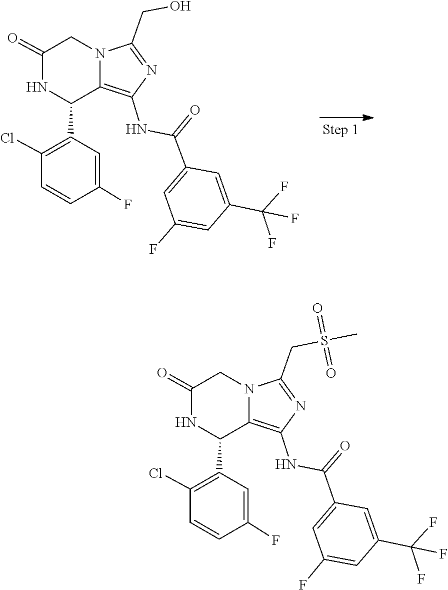 [SMILES: O=C1Cn2c(CO)nc(NC(=O)c3cc(F)cc(C(F)(F)F)c3)c2[C@H](c2cc(F)ccc2Cl)N1 ; CS(=O)(=O)Cc1nc(NC(=O)c2cc(F)cc(C(F)(F)F)c2)c2n1CC(=O)N[C@H]2c1cc(F)ccc1Cl]

### Step 1. (S)—N-(8-(2-chloro-5-fluorophenyl)-3-((methylsulfonyl)methyl)-6-oxo-5,6,7,8-tetrahydroimidazo[1,5-a]pyrazin-1-yl)-3-fluoro-5-(trifluoromethyl)benzamide

A reaction vial was charged with (S)—N-(8-(2-chloro-5-fluorophenyl)-3-(hydroxymethyl)-6-oxo-5,6,7,8-tetrahydroimidazo[1,5-a]pyrazin-1-yl)-3-fluoro-5-(trifluoromethyl)benzamide (120 mg), MsCl (82.7 mg), TEA (121 mg), THF (7 mL), and a stirbar before being evacuated and purged with nitrogen three times. The mixture was stirred at room temperature for 1 hour. Then sodium methanesulfinate (97.8 mg) and tetrabutylammonium iodide (8.85 mg) were added to the solution. The vial was purged with nitrogen three times. The reaction was stirred at room temperature for 1 hour. The reaction liquid was dried under vacuum. The resulting crude material was purified by prep-HPLC (Column: YMC-Actus Triart C18 ExRS, 30*150 mm, 5 μm; mobile phase A: 10 mmol/L NH₄HCO₃ in water, mobile phase B: acetonitrile; flow rate: 60 mL/min; gradient: 27% B to 510% B in 8 min, then 510% B; wavelength: 220 nm; RT: 7.7 min) to give the desired product (10.4 mg) as an off-white amorphous solid. LCMS RT 1.42 min, [M+H]⁺ 563.0, LCMS method P. ¹H NMR (400 MHz, DMSO-d6) δ 10.44 (s, 1H), 8.94 (d, J=2.2 Hz, 1H), 7.92 (dt, J=8.6, 2.1 Hz, 1H), 7.82 (dq, J=3.9, 2.3, 1.8 Hz, 2H), 7.37 (dd, J=8.8, 5.1 Hz, 1H), 7.09 (td, J=8.4, 3.1 Hz, 1H), 7.00 (dd, J=9.2, 3.1 Hz, 1H), 5.98 (d, J=2.1 Hz, 1H), 5.04-4.75 (m, 4H), 3.09 (s, 3H).

### Example 59

### N-(8-(2-chloro-5-fluorophenyl)-6-oxo-3-(pyridin-3-yloxy)-5,6,7,8-tetrahydroimidazo[1,5-a]pyrazin-1-yl)-3-fluoro-5-(trifluoromethyl)benzamide (I-636)

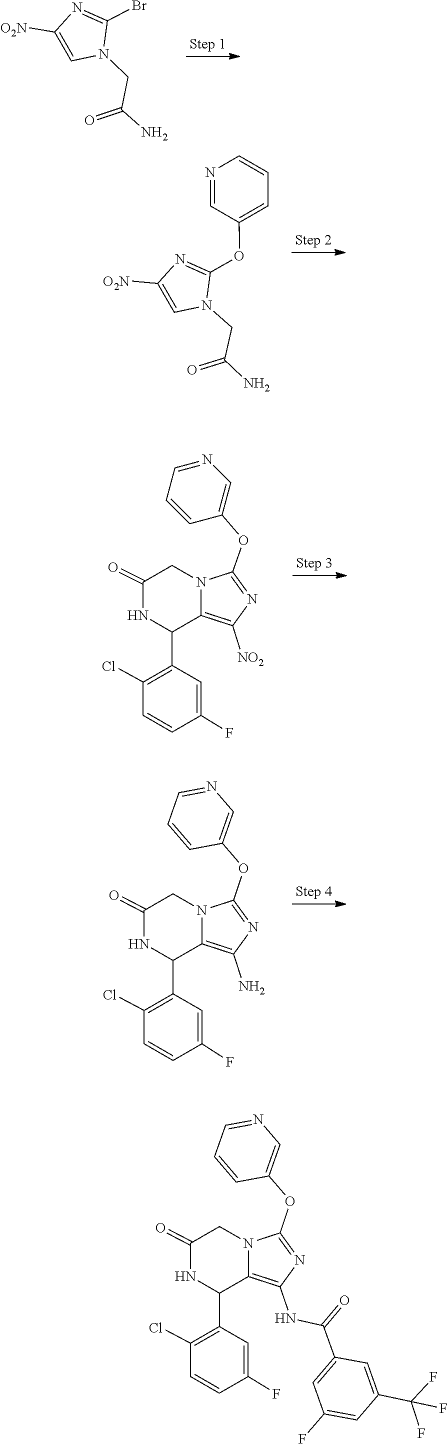 [SMILES: NC(=O)Cn1cc([N+](=O)[O-])nc1Br ; NC(=O)Cn1cc([N+](=O)[O-])nc1Oc1cccnc1 ; O=C1Cn2c(Oc3cccnc3)nc([N+](=O)[O-])c2C(c2cc(F)ccc2Cl)N1 ; Nc1nc(Oc2cccnc2)n2c1C(c1cc(F)ccc1Cl)NC(=O)C2 ; O=C1Cn2c(Oc3cccnc3)nc(NC(=O)c3cc(F)cc(C(F)(F)F)c3)c2C(c2cc(F)ccc2Cl)N1]

### Step 1. 2-(4-nitro-2-(pyridin-3-yloxy)-1H-imidazol-1-yl)acetamide

A resealable reaction vial was charged with 2-(2-bromo-4-nitro-1H-imidazol-1-yl)acetamide (3.0 g), pyridin-3-ol (3.0 g), MeCN (20 mL), K₂CO₃ (7.0 g), and a stirbar before being evacuated and purged with nitrogen three times. The resulting mixture was stirred for 16 hours at 80 degrees C. under a nitrogen atmosphere. The solid was removed by filtration. The filtrate was concentrated under vacuum. The resulting crude material was purified by HPLC (acetonitrile/water) to give the desired product (0.3 g) as a red solid. LCMS RT 0.48 min, [M+H]⁺ 264.1, LCMS method O.

### Step 2. 8-(2-chloro-5-fluorophenyl)-1-nitro-3-(pyridin-3-yloxy)-7,8-dihydroimidazo[1,5-a]pyrazin-6(5H)-one

A reaction vial was charged with 2-(4-nitro-2-(pyridin-3-yloxy)-1H-imidazol-1-yl)acetamide (50 mg), 2-chloro-5-fluorobenzaldehyde (27 mg), Eaton's reagent (1 mL), and a stirbar before being evacuated and purged with nitrogen three times. The resulting mixture was stirred for 1 h at 120 degrees C. under a nitrogen atmosphere. The reaction mixture was diluted with H₂O (20 mL), and the aqueous phase was extracted with ethyl acetate (30 mL) three times. The combined organic layers were washed with brine, dried over sodium sulfate, filtered, and concentrated in vacuo to give the desired product. The crude product was used in the next step without further purification. LCMS RT 0.64 min, [M+H]⁺ 404.1, LCMS method O.

### Step 3. 1-amino-8-(2-chloro-5-fluorophenyl)-3-(pyridin-3-yloxy)-7,8-dihydroimidazo[1,5-a]pyrazin-6(5H)-one

A reaction vial was charged with 8-(2-chloro-5-fluorophenyl)-1-nitro-3-(pyridin-3-yloxy)-7,8-dihydroimidazo[1,5-a]pyrazin-6(5H)-one (20 mg), Pd/C (5.9 mg), ethyl acetate (1 mL), and a stirbar before being evacuated and purged with hydrogen three times. The reaction mixture was stirred for 15 minutes at room temperature under a hydrogen atmosphere. The resulting mixture was used in the next step directly, after filtration through a pad of Celite. LCMS RT 0.55 min, [M+H]⁺=374.15, LCMS method O.

### Step 4. N-(8-(2-chloro-5-fluorophenyl)-6-oxo-3-(pyridin-3-yloxy)-5,6,7,8-tetrahydroimidazo[1,5-a]pyrazin-1-yl)-3-fluoro-5-(trifluoromethyl)benzamide

A reaction vial was charged with 1-amino-8-(2-chloro-5-fluorophenyl)-3-(pyridin-3-yloxy)-7,8-dihydroimidazo[1,5-a]pyrazin-6(5H)-one (6 mg) in ethyl acetate (1 mL), 3-fluoro-5-(trifluoromethyl)benzoyl chloride (4 mg) in DCM (1 mL), and a stirbar, before being evacuated and purged with nitrogen three times. The resulting mixture was stirred for 15 minutes at room temperature. The crude product was purified by prep-HPLC (column: XBridge Prep OBD C18 Column, 30*150 mm, 5 μm; mobile phase A: 10 mM NH₄HCO₃ in water, mobile phase B: acetonitrile; flow rate: 60 mL/min; gradient: 25% B to 55% B in 8 min, then 55% B; wavelength: 220 nm; RT: 7.68 min) to give the desired product (0.7 mg) as an off-white solid. LCMS RT 0.78 min, [M+H]⁺ 564.0, LCMS method F. ¹H NMR (400 MHz, Chloroform-d) δ 8.72 (s, 1H), 8.54 (s, 1H), 7.87 (s, 2H), 7.79 (d, J=8.9 Hz, 1H), 7.66 (s, 1H), 7.60 (d, J=8.4 Hz, 1H), 7.52-7.34 (m, 2H), 7.04-6.94 (m, 1H), 6.85 (dd, J=8.8, 3.0 Hz, 1H), 6.64 (s, 2H), 4.77 (d, J=17.7 Hz, 1H), 4.65 (d, J=17.8 Hz, 1H).

### Example 60

### (S)—N-(8-(2-chloro-5-fluorophenyl)-6-oxo-3-((pyridin-2-yloxy)methyl)-5,6,7,8-tetrahydroimidazo[1,5-a]pyrazin-1-yl)-3-fluoro-5-(trifluoromethyl)benzamide (I-637)

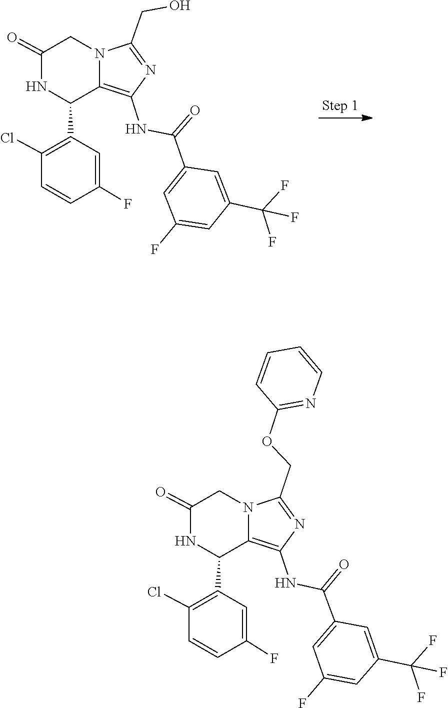 [SMILES: O=C1Cn2c(CO)nc(NC(=O)c3cc(F)cc(C(F)(F)F)c3)c2[C@H](c2cc(F)ccc2Cl)N1 ; O=C1Cn2c(COc3ccccn3)nc(NC(=O)c3cc(F)cc(C(F)(F)F)c3)c2[C@H](c2cc(F)ccc2Cl)N1]

### Step 1. (S)—N-(8-(2-chloro-5-fluorophenyl)-6-oxo-3-((pyridin-2-yloxy)methyl)-5,6,7,8-tetrahydroimidazo[1,5-a]pyrazin-1-yl)-3-fluoro-5-(trifluoromethyl)benzamide

A reaction vial was charged with (S)—N-(8-(2-chloro-5-fluorophenyl)-3-(hydroxymethyl)-6-oxo-5,6,7,8-tetrahydroimidazo[1,5-a]pyrazin-1-yl)-3-fluoro-5-(trifluoromethyl)benzamide (200 mg), DMF (4 mL), and a stirbar. NaH (29 mg) was added at 0° C., and the vial was evacuated and purged with nitrogen three times. The mixture was stirred at room temperature for 0.5 h. Then 2-fluoropyridine (46.5 mg) was added, and the vial was purged with nitrogen three times. The mixture was stirred at room temperature for 1 hour and poured into ice water. The aqueous phase was extracted with DCM (3×50 mL). The combined organic layers were washed with brine, dried over sodium sulfate, filtered, and concentrated in vacuo. The resulting crude material was purified by prep-HPLC (Column: XSelect CSH Prep C18 OBD Column, 19*250 mm, 5 μm; mobile phase A: water (0.1% formic acid), mobile phase B: acetonitrile; flow rate: 30 mL/min; gradient: 37% B to 47% B in 7 minutes, then 47% B; wavelength: 254/220 nm; RT: 6.65 min) to give the desired product (10 mg) as an off-white amorphous solid. LCMS RT 0.78 min, [M+H]⁺ 578.2, LCMS method K. ¹H NMR (400 MHz, DMSO-d6) δ 10.35 (s, 1H), 8.89 (d, J=2.4 Hz, 1H), 7.89 (d, J=8.4 Hz, 1H), 7.86-7.76 (m, 3H), 7.44 (ddd, J=8.9, 6.6, 2.1 Hz, 1H), 7.34 (dd, J=8.8, 5.1 Hz, 1H), 7.07 (td, J=8.4, 3.1 Hz, 1H), 6.97 (dd, J=9.2, 3.1 Hz, 1H), 6.40 (d, J=9.1 Hz, 1H), 6.28 (td, J=6.7, 1.4 Hz, 1H), 5.96 (t, J=1.5 Hz, 1H), 5.20-5.08 (m, 3H), 5.03 (d, J=17.5 Hz, 1H).

### Example 61

### N-(8-(2-chloro-5-fluorophenyl)-6-oxo-3-(pyridin-4-ylmethyl)-5,6,7,8-tetrahydroimidazo[1,5-a]pyrazin-1-yl)-3-fluoro-5-(trifluoromethyl)benzamide (I-638)

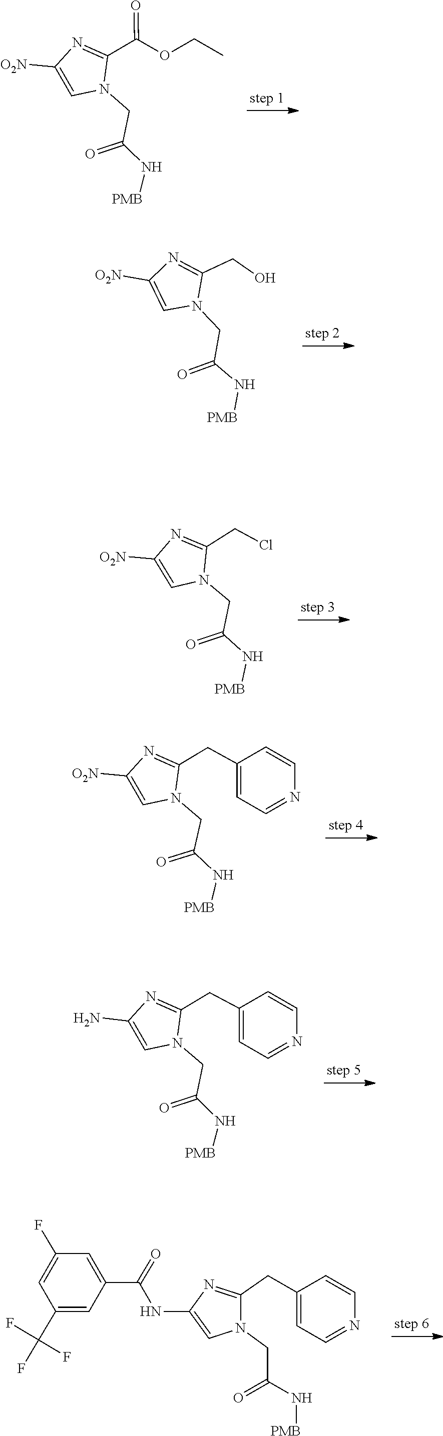

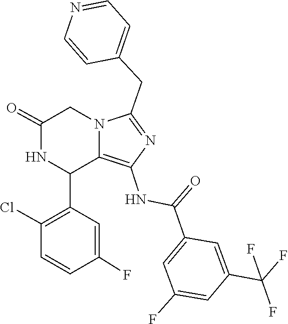 [SMILES: O=C1Cn2c(Cc3ccncc3)nc(NC(=O)c3cc(F)cc(C(F)(F)F)c3)c2C(c2cc(F)ccc2Cl)N1]

### Step 1. 2-(2-(hydroxymethyl)-4-nitro-1H-imidazol-1-yl)-N-(4-methoxybenzyl)acetamide

A reaction vial was charged with ethyl 1-(2-(4-methoxybenzylamino)-2-oxoethyl)-4-nitro-1H-imidazole-2-carboxylate (3.6 g) in MeOH (40 mL). After cooling to 0° C., NaBH₄ (756 mg) was added, and the mixture was slowly warmed to room temperature over 1 hour. The reaction mixture was diluted with H₂O (100 mL), then extracted with ethyl acetate (150 mL) three times. The combined organic layers were washed with brine, dried over sodium sulfate, filtered, and concentrated in vacuo. The resulting crude material was purified by HPLC (acetonitrile/water) to give the desired product (600 mg) as an off-white solid. LCMS RT 0.83 min, [M+H]⁺ 321.10, LCMS method A.

### Step 2. 2-(2-(chloromethyl)-4-nitro-1H-imidazol-1-yl)-N-(4-methoxybenzyl)acetamide

To a suspension of 2-(2-(hydroxymethyl)-4-nitro-1H-imidazol-1-yl)-N-(4-methoxybenzyl)acetamide (600 mg) in DCM (5 mL) was added SOCl₂ (600 mg). The mixture was stirred at room temperature for one hour. The mixture was diluted with H₂O (20 mL). The resulting solution was extracted with 3×10 mL of ethyl acetate. The organic layers were combined, washed with sodium carbonate solution in water and brine, dried, and concentrated under vacuum. The resulting crude material was purified by C18 column chromatography (acetonitrile/water) to give the desired product (400 mg) as an off-white amorphous solid. LCMS RT 0.93 min, [M+H]⁺ 339.30, LCMS method A.

### Step 3. N-(4-methoxybenzyl)-2-(4-nitro-2-(pyridin-4-ylmethyl)-1H-imidazol-1-yl)acetamide

A reaction vial was charged with 2-(2-(chloromethyl)-4-nitro-1H-imidazol-1-yl)-N-(4-methoxybenzyl)acetamide (420 mg), pyridin-4-ylboronic acid (305 mg), Pd(PPh₃)₄ (143.1 mg), K₃PO₄ (788 mg), DMF/H₂O=8:1 (10 mL), and a stirbar before being evacuated and purged with nitrogen three times. The mixture was stirred at 90° C. for 1 h. The mixture was diluted with H₂O (20 mL) and extracted with ethyl acetate (20 mL) three times. The combined organic layers were washed with brine, dried over sodium sulfate, filtered, and concentrated in vacuo. The resulting crude material was purified by HPLC (acetonitrile/water) to give the desired product (130 mg) as a yellow solid. LCMS RT 0.53 min, [M+H]⁺ 382.10, LCMS method A.

### Step 4. 2-(4-amino-2-(pyridin-4-ylmethyl)-1H-imidazol-1-yl)-N-(4-methoxybenzyl)acetamide

To a suspension of N-(4-methoxybenzyl)-2-(4-nitro-2-(pyridin-4-ylmethyl)-1H-imidazol-1-yl)acetamide (50 mg) in MeOH (2 mL) was added palladium on carbon (22.7 mg, 10% wt). The mixture was degassed with hydrogen and stirred at room temperature for 1 h under an atmosphere of hydrogen (balloon). The reaction mixture was purged with N₂, filtered through a syringe filter, and concentrated under reduced pressure to afford the crude desired product as a brown oil. The resulting crude material was used directly in the next step. LCMS RT 0.367 min, [M+H]⁺ 352.20, LCMS method I.

### Step 5. 3-fluoro-N-(1-(2-((4-methoxybenzyl)amino)-2-oxoethyl)-2-(pyridin-4-ylmethyl)-1H-imidazol-4-yl)-5-(trifluoromethyl)benzamide

A reaction vial was charged with 3-fluoro-5-(trifluoromethyl)benzoic acid (35.6 mg) in DCM (2 mL) and a stirbar before being evacuated and purged with nitrogen three times. 1-Chloro-N,N,2-trimethylprop-1-en-1-amine (27.5 mg) was added to the mixture under N₂ at 0° C. After 0.5 h 2-(4-amino-2-(pyridin-4-ylmethyl)-1H-imidazol-1-yl)-N-(4-methoxybenzyl)acetamide in DCM (2 mL) was added, and the mixture was stirred at 25° C. for 1 hour under nitrogen. The mixture was diluted with H₂O (10 mL). The solution was extracted with ethyl acetate (10 mL) three times. The organic layer was dried over Na₂SO₄ and evaporated to dryness. The resulting crude material was purified by HPLC (acetonitrile/water) to give the desired product (12 mg) as an off-white solid. LCMS RT 0.978 min, [M+H]⁺ 542.15, LCMS method A.

### Step 6. N-(8-(2-chloro-5-fluorophenyl)-6-oxo-3-(pyridin-4-ylmethyl)-5,6,7,8-tetrahydroimidazo[1,5-a]pyrazin-1-yl)-3-fluoro-5-(trifluoromethyl)benzamide

A reaction vial was charged with 3-fluoro-N-(1-(2-(4-methoxybenzylamino)-2-oxoethyl)-2-(pyridin-4-ylmethyl)-1H-imidazol-4-yl)-5-(trifluoromethyl)benzamide (12 mg) and 2-chloro-5-fluorobenzaldehyde (5.3 mg). Eaton's reagent (1 mL) was added, and the reaction media was heated at 100° C. for one hour. The reaction mixture was diluted with H₂O (10 mL), and the aqueous phase was extracted with ethyl acetate (10 mL) three times. The combined organic layers were washed with brine, dried over sodium sulfate, filtered, and concentrated in vacuo. The resulting crude material was purified by HPLC (Column: XBridge Prep OBD C18 Column, 30*150 mm, 5 μm; mobile phase A: 10 mM NH₄HCO₃ in water, mobile phase B: acetonitrile; flow rate: 60 mL/min; gradient: 25% B to 50% B in 7 min, then 50% B; wavelength: 220 nm; RT: 7.47 min) to give the desired product (4.3 mg) as a white amorphous solid. LCMS RT 0.713 min, [M+H]⁺ 562.05, LCMS method N. ¹H NMR (400 MHz, DMSO-d₆) δ 10.33 (s, 1H), 8.88 (d, J=2.3 Hz, 1H), 8.56-8.41 (m, 2H), 7.88 (d, J=8.4 Hz, 1H), 7.80 (d, J=12.1 Hz, 2H), 7.39-7.26 (m, 3H), 7.12-6.98 (m, 2H), 5.96 (s, 1H), 4.75 (s, 2H), 4.15 (s, 2H).

### Example 62

### (S)—N-(8-(2-chloro-5-fluorophenyl)-3-((2-(4,4-difluoropiperidin-1-yl)ethyl)carbamoyl)-6-oxo-5,6,7,8-tetrahydroimidazo[1,5-a]pyrazin-1-yl)benzo[d]isothiazole-3-carboxamide (I-655)

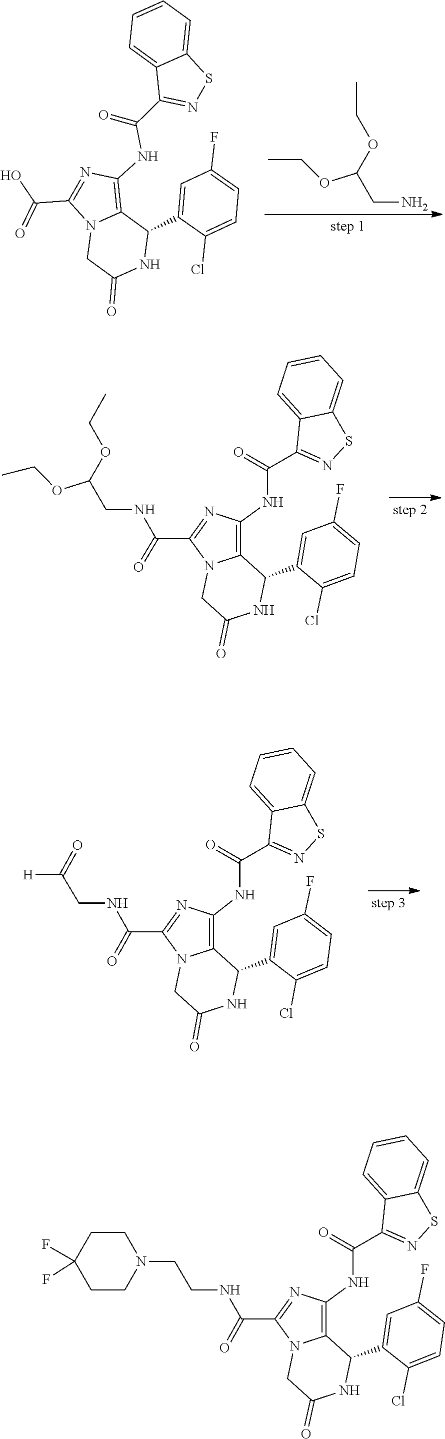

### Step 1. (S)—N-(8-(2-chloro-5-fluorophenyl)-3-((2,2-diethoxyethyl)carbamoyl)-6-oxo-5,6,7,8-tetrahydroimidazo[1,5-a]pyrazin-1-yl)benzo[d]isothiazole-3-carboxamide

(S)-1-(benzo[d]isothiazole-3-carboxamido)-8-(2-chloro-5-fluorophenyl)-6-oxo-5,6,7,8-tetrahydroimidazo[1,5-a]pyrazine-3-carboxylic acid (200 mg) and 2,2-diethoxyethan-1-amine (54.8 mg) were dissolved in DMF (3 mL). HATU (235 mg) and DIEA (160 mg) were added. The resulting mixture was stirred for 1 hour at room temperature. EtOAc and water were added, and the two layers were separated. The organic layer was washed three times with water, dried over Na₂SO₄, and concentrated. The crude product was purified by flash chromatography to afford the desired product (200 mg) as a white solid. LCMS: RT 0.83 min, [M+H]⁺ 601.0, LCMS method C.

### Step 2. (S)—N-(8-(2-chloro-5-fluorophenyl)-6-oxo-3-((2-oxoethyl)carbamoyl)-5,6,7,8-tetrahydroimidazo[1,5-a]pyrazin-1-yl)benzo[d]isothiazole-3-carboxamide

A resealable reaction vial was charged with (S)—N-(8-(2-chloro-5-fluorophenyl)-3-((2,2-diethoxyethyl)carbamoyl)-6-oxo-5,6,7,8-tetrahydroimidazo[1,5-a]pyrazin-1-yl)benzo[d]isothiazole-3-carboxamide (50 mg) and a stirbar before being evacuated and purged with nitrogen three times. THF (0.5 mL) and HCl (1 M, 0.5 mL) were added, and the mixture was stirred at 25° C. for 1 hour. EtOAc and saturated NaHCO₃ solution were added, the two layers were separated, and the aqueous layer was extracted twice with EtOAc. The organic layers were combined, dried over Na₂SO₄, and concentrated to give the desired product, which was used in the next step without further purification. LCMS: RT 0.70 min, [M+H]⁺ 526.95, LCMS method C.

### Step 3. (S)—N-(8-(2-chloro-5-fluorophenyl)-3-((2-(4,4-difluoropiperidin-1-yl)ethyl)carbamoyl)-6-oxo-5,6,7,8-tetrahydroimidazo[1,5-a]pyrazin-1-yl)benzo[d]isothiazole-3-carboxamide

A solution of 4,4-difluoropiperidine hydrochloride (30 mg) and DIEA (74 mg) in 1,2-dichloroethane (0.5 mL) was added to a solution of (S)—N-(8-(2-chloro-5-fluorophenyl)-6-oxo-3-((2-oxoethyl)carbamoyl)-5,6,7,8-tetrahydroimidazo[1,5-a]pyrazin-1-yl)benzo[d]isothiazole-3-carboxamide (0.10 g) in 1,2-dichloroethane (0.5 mL), followed by NaBH₃CN (35 mg) at 0° C. The reaction mixture was allowed to warm up to room temperature and stirred for 1 h. Saturated NaHCO₃ solution was added, and the two layers were separated. The aqueous layer was extracted twice with EtOAc. The organic layers were combined, dried over Na₂SO₄, and concentrated. The residue was purified by prep-HPLC (Column: YMC-Actus Triart C18 ExRS, 30*150 mm, 5 μm; mobile phase A: water (10 mmol/L NH₄HCO₃+0.1% NH₃·H₂O), mobile phase B: acetonitrile; flow rate: 60 mL/min; gradient: 30% B to 60% B in 8 min, then 60% B; wavelength: 220 nm; RT: 7.23 min) to give the desired product (17.4 mg). LCMS: RT 0.93 min, [M+H]⁺ 632.30, LCMS method B.

Additional compounds prepared according to the methods of Examples 62 are listed in Table 17 below. Corresponding ¹H NMR and mass spectrometry characterization for these compounds are described in Table 1. Certain compounds in Table 17 below were prepared with other compounds whose preparation is described in the Examples herein.

TABLE 17
Additional Exemplary Compounds
Compound

I-390
I-497
I-654
I-655
I-656
I-657
I-658
I-659
I-676
I-677

### Example 63

Selected compounds of the present disclosure were tested in an ADP-Glo Biochemical PIK3CA Kinase Assay. Compounds to be assayed were plated in 16 doses of 1:2 serial dilutions (20 nL volume each well) on a 1536-well plate, and the plate w armed to room temperature. PIK3CA enzyme (e.g. H1047R, E542K, E545K, or wild-type) (1 μL of 2 nM solution in Enzyme Assay Buffer (comprising 50 mM HEPES pH 7.4, 50 mM NaCl, 6 mM MgCl₂, 5 mM DTT and 0.03% CHAPS)) was added and shaken for 10 seconds and preincubated for 30 minutes. To the well was added 1 μL of 200 μM ATP and 20 μM of diC8-PIP2 in Substrate Assay Buffer (50 mM HEPES pH-7.4, 50 mM NaCl, 5 mM DTT and 0.03% CHAPS) to start the reaction, and the plate was shaken for 10 seconds, then spun briefly at 1500 rpm, and then incubated for 60 minutes at room temperature. The reaction was stopped by adding 2 μL of ADP-Glo reagent (Promega), and spinning briefly at 1500 rpm, and then incubating for 40 minutes. ADP-Glo Detection reagent (Promega) was added and the plate spun briefly at 1500 rpm, then incubated for 30 minutes. The plate was read on an Envision 2105 (Perkin Elmer), and the IC₅₀ values were calculated using Genedata software.

Results of the ADP-Glo Biochemical PIK3CA Kinase Assay using H1047R PIK3CA enzyme are presented in Table 1. Compounds having an IC₅₀ less than or equal to 100 nM are represented as “A”; compounds having an IC₅₀ greater than 100 nM but less than or equal to 500 nM are represented as “B”; compounds having an IC₅₀ greater than 500 nM but less than or equal to 1 μM are represented as “C”; compounds having an IC₅₀ greater than 1 μM but less than or equal to 10 μM are represented as “D”; and compounds having an IC₅₀ greater than 10 PM but less than or equal to 100 μM are represented as “E”.

### Example 64

Selected compounds of the present disclosure were tested in a MCF10A Cell-Based PIK3CA Kinase Assay, namely the CisBio Phospho-AKT (Ser473) HTRF assay, to measure the degree of PIK3CA-mediated AKT phosphorylation. MCF10A cells (immortalized non-transformed breast cell line) overexpressing hotspot PIK3CA mutations (including H1047R, E542K, and E545K mutations) were used. Cells were seeded at 5,000 cells per well in DMEM/F12 (Thermo Fisher Scientific) supplemented with 0.5 mg/mL hydrocortisone, 100 ng/mL Cholera Toxin, 10 μg/mL insulin, and 0.5% horse serum. Once plated, cells were placed in a 5% CO₂, 37° C. incubator to adhere overnight.

The following day, compounds were added to the cell plates in 12 doses of 1:3 serial dilutions. The dose response curves were run in duplicate. Compound addition was carried out utilizing an Echo 55 Liquid Handler acoustic dispenser (Labcyte). The cell plates were incubated for 2 hours in a 5% CO₂, 37° C. incubator. Following compound incubation, the cells were lysed for 60 min at room temperature. Finally, a 4-hour incubation with the HTRF antibodies was performed at room temperature. All reagents, both lysis buffer and antibodies, were used from the CisBio pAKT S473 HTRF assay kit, as per the manufacturers protocol. Plates were read on an Envision 2105 (Perkin Elmer), and the IC₅₀ values were calculated using Genedata software.

Results of the MCF10A Cell-Based PIK3CA Kinase Assay are presented in Table 1. Compounds having an IC₅₀ less than or equal to 1 μM are represented as “A”; compounds having an IC₅₀ greater than 1 IM but less than or equal to 5 μM are represented as “B”; compounds having an IC₅₀ greater than 5 μM but less than or equal to 10 μM are represented as “C”; compounds having an IC₅₀ greater than 10 μM but less than or equal to 36 μM are represented as “D”; and compounds having an IC₅₀ greater than 36 μM but less than or equal to 100 μM are represented as “E”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

LENGTHY TABLES
The patent application contains a lengthy table section. A copy of the table is available in electronic form from the USPTO web site (https://seqdata.uspto.gov/?pageRequest=docDetail&DocID=US20250019380A1). An electronic copy of the table will also be available from the USPTO upon request and payment of the fee set forth in 37 CFR 1.19(b)(3).

1. A compound of formula I: 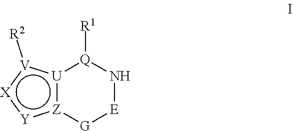
or a pharmaceutically acceptable salt thereof, wherein:
E is —C(O)—, —C(R^{E})₂—, —C(R^{E})₂C(R^{E})₂—, —C(S)—, —S(O)₂—, —OC(O)—, —N(R^{E})C(O)—, —C(O)N(R^{E})—, or —C(R^{E})₂C(O)—;
G is CH₂, CH(R^{G}), C(R^{G})₂, or a covalent bond;
Q is CH, C(R^{Q}), or N;
X is CH, C(R^{X}), or N;
Y is CH, C(R^{Y}), N, or N(R^{Y});
Z is C or N;
U is C or N;
V is C or N; provided that at least one of X, Y, Z, U, and V is N;
R¹ is -L¹-R^{1A};
R² is -L²-R^{2A};
each instance of R^{E} is independently H or -L^{E}-R^{EA};
each instance of R^{G} is independently -LG-R^{GA};
R^{Q} is -L^{Q}-R^{QA};
R^{X} is -LX-R^{XA};
R^{Y} is -LY-R^{YA}; or
two instances of R^{E} are taken together with their intervening atoms to form a 3-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with n instances of R^{EEC};
R^{Q} and R¹ are taken together with their intervening atoms to form a 4-8 membered saturated or partially unsaturated monocyclic ring having 0-4 heteroatoms independently selected from nitrogen, oxygen, and sulfur; or an 8-12 membered saturated or partially unsaturated bicyclic ring having 0-4 heteroatoms independently selected from nitrogen, oxygen, and sulfur; wherein each ring is substituted with p instances of R^{Q1C};
each of L¹, L², L^{E}, L^{G}, L^{Q}, L^{X}, and L^{Y} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{1A} is R^{A} or R^{B} substituted by r¹ instances of R^{1C};
R^{2A} is R^{A} or R^{B} substituted by r² instances of R^{2C};
each instance of R^{EA} is independently R^{A} or R^{B} substituted by r instances of R^{E}C;
each instance of R^{GA} is independently R^{A} or R^{B} substituted by r⁴ instances of R^{GC};
R^{QA} is R^{A} or R^{B} substituted by r⁵ instances of R^{QC};
R^{XA} is R^{A} or R^{B} substituted by r⁶ instances of R^{XC};
R^{YA} is R^{A} or R^{B} substituted by r⁷ instances of R^{Y};
R^{L} is R^{A} or R^{B} substituted by r⁸ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{EC}, R^{GC}, R^{QC}, R^{XC}, R^{YC}, R^{LC}, R^{EEC}, and R^{Q1C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, p, r¹, r², r³, r⁴, r⁵, r⁶, r⁷, and r⁸ is independently 0, 1, 2, 3, 4, or 5.

2. The compound of claim 1, wherein the compound is a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, or XLVIII: 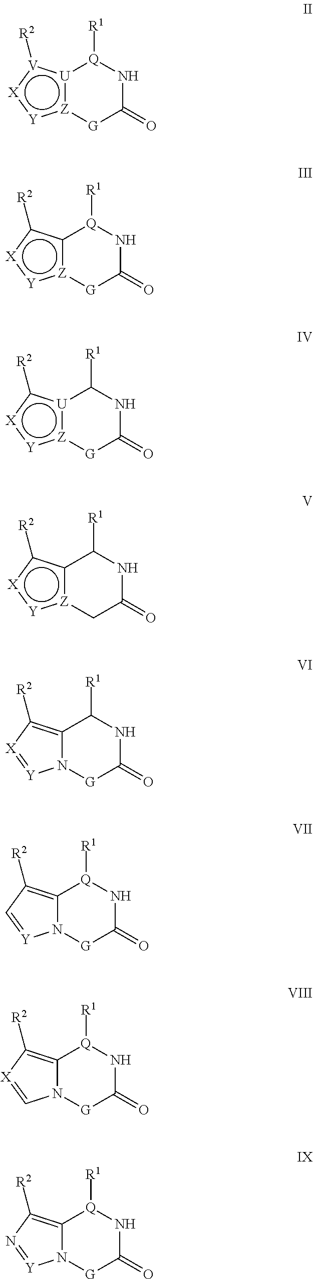 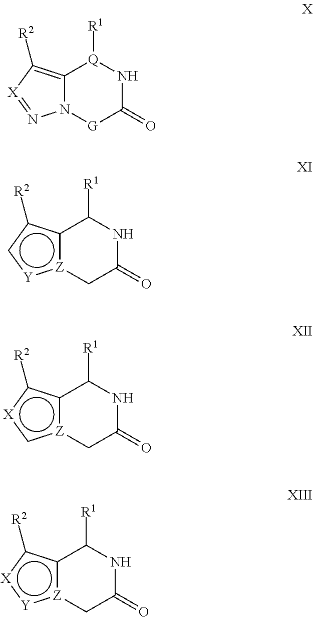 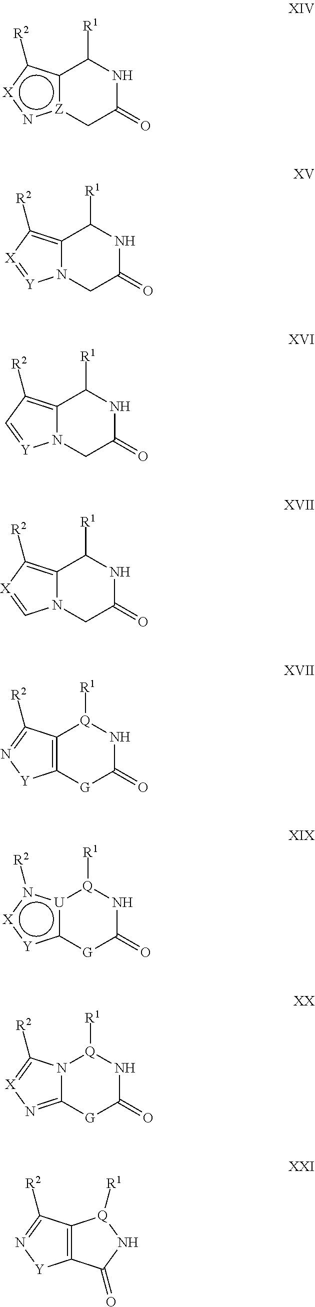 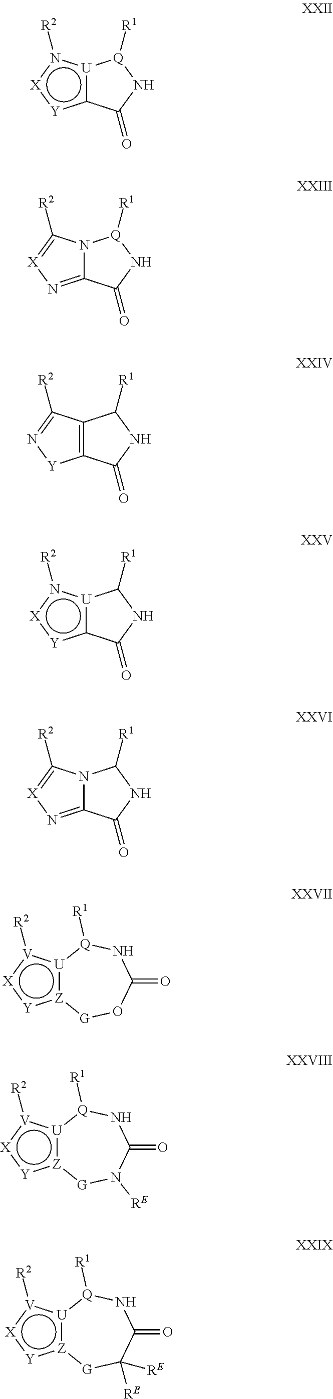 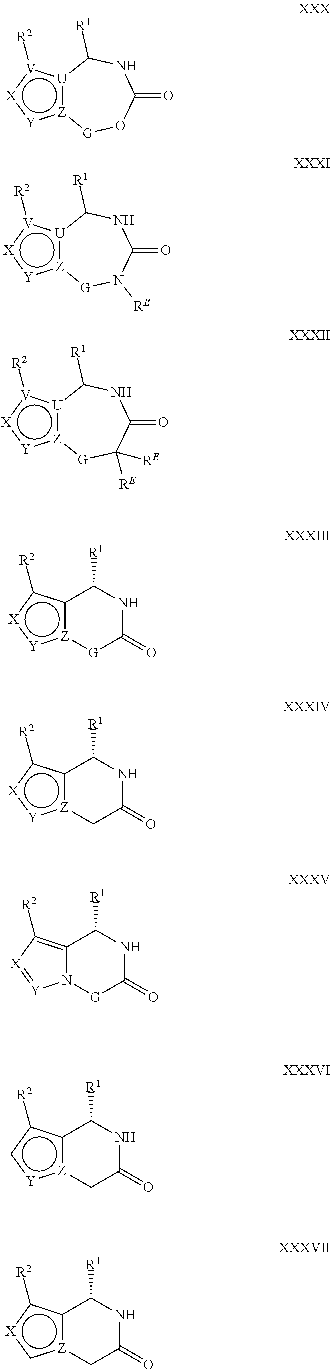 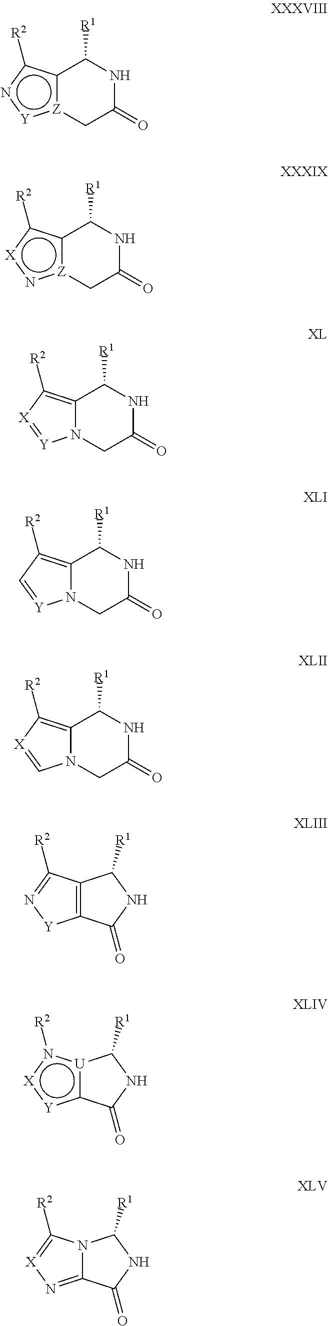 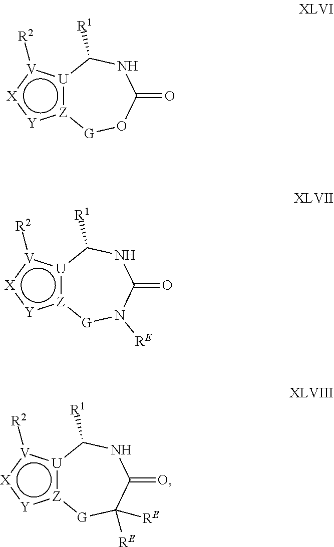
or a pharmaceutically acceptable salt thereof.

3-8. (canceled)

9. The compound of claim 1, wherein X is CH or N.

10. (canceled)

11. The compound of claim 1, wherein Z is N.

12. The compound of claim 1, wherein L¹ is a covalent bond.

13. The compound of claim 1, wherein R^{1A} is R^{B}, or R^{1A} is phenyl or an 8-10 membered bicyclic heteroaryl ring having 1-4 heteroatoms independently selected from nitrogen, oxygen, and sulfur; wherein R^{1A} is substituted by r¹ instances of R^{1C}.

14.-15. (canceled)

16. The compound of claim 1, wherein R¹ is

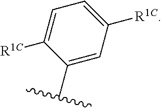

17. The compound of claim 1, wherein each instance of R^{1C} is independently halogen, —CN, —O—(C₁₋₆ aliphatic), or C₁₋₆ aliphatic; wherein each C₁₋₆ aliphatic is optionally substituted with one or more halogen atoms.

18. (canceled)

19. The compound of claim 1, wherein G is CH₂.

20. (canceled)

21. The compound of claim 1, wherein R^{Q} and R¹ are taken together with their intervening atoms to form an 8-12 membered saturated or partially unsaturated bicyclic ring having 0-4 heteroatoms independently selected from nitrogen, oxygen, and sulfur; wherein said ring is substituted with p instances of R^{Q1C}.

22. (canceled)

23. The compound of claim 1, wherein each instance of R^{Q1C} is independently oxo, halogen, —CN, —O—(C₁₋₆ aliphatic), or C₁₋₆ aliphatic; wherein each C₁₋₆ aliphatic is optionally substituted with one or more halogen atoms.

24. The compound of claim 1, wherein R² is —N(H)C(O)—R^{2A}, —N(H)C(O)N(H)—R^{2A}, —C(O)N(H)—R^{2A}, —N(H)—R^{2A}, —S(O)₂CH₂—R^{2A}, —CH₂S(O)₂—R^{2A}, or —C(H)(CH₃)OH.

25. (canceled)

26. The compound of claim 1, wherein R^{2A} is R^{B}, or R^{2A} is phenyl, naphthyl, an 8-10 membered bicyclic heteroaryl ring having 1-4 heteroatoms independently selected from nitrogen, oxygen, and sulfur, or a 7-12 membered saturated or partially unsaturated bicyclic heterocyclic ring having 1-4 heteroatoms independently selected from nitrogen, oxygen, and sulfur: wherein R^{2A} is substituted by r² instances of R^{2C}.

27.-29. (canceled)

30. The compound of claim 1, wherein each instance of R^{2C} is independently halogen, —CN, —O—(C₁₋₆ aliphatic), or C₁₋₆ aliphatic; wherein each C₁₋₆ aliphatic is optionally substituted with one or more halogen atoms.

31. (canceled)

32. The compound of claim 1, wherein Y is C(R^{Y}).

33. The compound of claim 1, wherein R^{Y} is —C(O)N(H)—R^{YA}, —C(O)N(H)CH₂—R^{YA}, or —R^{YA}.

34. The compound of claim 1, wherein R^{YA} is a 5-6 membered monocyclic heteroaryl ring having 1-4 heteroatoms independently selected from nitrogen, oxygen, and sulfur; or R^{YA} is a 3-7 membered saturated or partially unsaturated monocyclic heterocyclic ring having 1-2 heteroatoms independently selected from nitrogen, oxygen, and sulfur; wherein said ring is substituted by r⁷ instances of R^{YC}; or R^{YA} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium.

35.-37. (canceled)

38. The compound of claim 1, wherein each instance of R^{YC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms.

39. A compound selected from those set forth in Table 1, or a pharmaceutically acceptable salt thereof.

40. A pharmaceutical composition, comprising the compound of claim 1, or a pharmaceutically acceptable salt thereof, and a pharmaceutically acceptable carrier.

41.-42. (canceled)

43. A method of inhibiting PI3Kα signaling activity, treating a PI3Kα-mediated disorder, or treating a cellular proliferative disease in a subject, comprising administering a therapeutically effective amount of the compound of claim 1, or a pharmaceutically acceptable salt thereof, to a subject in need thereof.

44.-48. (canceled)
